(19) United States
(12) Patent Application Publication
(10) Pub. No.: US 2020/0231633 A1
(43) Pub. Date: Jul. 23, 2020

(54) MGAT1-Deficient Cells for Production of Vaccines and Biopharmaceutical Products

(71) Applicant: The Regents of the University of California, Oakland, CA (US)

(72) Inventors: Phillip W. Berman, Santa Cruz, CA (US); Gabriel Byrne, Santa Cruz, CA (US)

(21) Appl. No.: 16/631,354

(22) PCT Filed: Jul. 16, 2018
(86) PCT No.: PCT/US2018/042335

§ 371 Date: Jan. 15, 2020

Related U.S. Application Data
(60) Provisional application No. 62/534,594, filed on Jul. 19, 2017.

(51) Int. Cl. C07K 14/16; C12N 9/10; C12N 5/071; C12N 15/85

(52) U.S. Cl. CPC C07K 14/162; C12N 9/1051; C12N 5/0682; C12N 15/85; A61K 38/00; C07K 2319/02; C07K 2319/40; C12N 2740/16134; C12Y 204/01101

(57) ABSTRACT

Mannosyl (alpha-1,3)-glycoprotein beta-1,2-N-Acetylglucosaminyltransferase (Mgat1)-deficient cell lines and methods for use of same for producing human immunodeficiency virus (HIV) envelope glycoprotein polypeptides or fragment thereof with terminal mannose-5 glycans are provided.

### CROSS-REFERENCE TO RELATED APPLICATIONS

This application claims the benefit of U.S. Provisional Patent Application No. 62/534,594 filed on Jul. 19, 2017, which application is incorporated herein by reference in its entirety.

### STATEMENT REGARDING FEDERALLY SPONSORED RESEARCH OR DEVELOPMENT

This invention was made with government support under grant no. R01 AI113893, awarded by the National Institutes of Health. The government has certain rights in the invention.

### INTRODUCTION

Human immunodeficiency virus type 1 (HIV-1) entry into a host cell is dependent on envelope glycoprotein (Env), which consists of two noncovalently bound subunits, the external gp120 and the transmembrane gp41. Env is present on virion surfaces as trimers of gp120-gp41 complexes and is involved in the binding of the virus to the host receptor and co-receptor(s). Env is also the target for the binding of neutralizing antibodies.

The development of a vaccine able to provide protection from HIV-1 infection has long been a global public health priority. To achieve this goal, vaccine development efforts have focused on the discovery of immunogens able to elicit cellular immune responses (e.g., cytotoxic lymphocytes) or broadly neutralizing antibody (bNAb) responses. Cellular immune responses are detected soon after infection in most HIV-1 infected individuals, whereas bNAb responses are found in only 10-20% of infected individuals. Unfortunately, after more than 30 years of research, none of the candidate vaccines described to date have been effective in eliciting bNAbs.

The recent isolation and characterization of multiple human bNAbs from HIV-1 infected subjects has now identified the epitopes responsible for much of the neutralizing activity in sera from HIV-1-infected humans. Over the past several years, the structures of several bNAbs in complexes with gp120 fragments have been elucidated. Several of these bNAbs, including PG9, PG16, CH01, CH03, and PGT145 appear to target glycan-dependent epitopes (GDEs) in the V1/V2 domain of gp120. PG9 and PG9-like antibodies are particularly interesting, since the epitope they recognize appears to overlap with an epitope associated with protection from HIV-1 infection in the RV144 HIV-1 vaccine trial. Structural studies showed that the binding of PG9 was highly dependent on mannose-5 glycans at positions 156 and 160, as well as basic amino acid side chains at positions 167-169 and 171 and that this region is required for the binding of multiple neutralizing and non-neutralizing antibodies to the V1/V2 domain.

Mannosyl (alpha-1,3)-glycoprotein beta-1,2-N-Acetylglucosaminyltransferase (Mgat1, also known as Gnt1) adds N-Acetylglucosamine to the Man₅GlcNAc₂ (Man5)N-glycan structure as part of complex N-glycan synthesis and expressed by eukaryotic cell lines such as CHO cell lines.

Thus, there remains a need for the development of cell lines that do not have Mgat1 activity and can express exogenous polypeptides stably and in sufficient quantities.

### SUMMARY

The present disclosure provides mannosyl (alpha-1,3)-glycoprotein beta-1,2-N-Acetylglucosaminyltransferase (Mgat1)-deficient cell lines and methods for use of same for producing human immunodeficiency virus (HIV) envelope glycoprotein polypeptides or fragment thereof with terminal mannose-5 glycans (Man₅GlcNAc₂).

In certain aspects, a genetically modified Chinese hamster ovary (CHO) cell line is provided. The cell line includes a heterologous nucleic acid comprising a nucleotide sequence encoding a human immunodeficiency virus (HIV) envelope glycoprotein polypeptide comprising an N-linked glycosylation site; and a mutation of an endogenous gene encoding mannosyl (alpha-1,3)-glycoprotein beta-1,2-N-Acetylglucosaminyltransferase (Mgat1), where the mutation prevents Mgat1-mediated addition of a N-acetylglucosamine moiety to a terminal mannose residue present at the N-linked glycosylation site of the HIV envelope glycoprotein polypeptide such that at least 75% of the HIV envelope glycoprotein polypeptides produced by the genetically modified cell line comprise terminal mannose-5, manse-8, or mannose-9 glycans at the N-linked glycosylation site. The mutation may be a targeted mutation.

In certain aspects, the polypeptide is gp120 or an N-linked glycosylation site containing fragment thereof. The fragment may comprise variable regions 1 and 2 (V1/V2). The gp120 or an N-linked glycosylation site containing fragment thereof or the V1/V2 fragment may be a monomer. In certain aspects, the fragment comprising variable regions 1 and 2 may be at least 50 amino acids long (e.g., 50-100 amino acids) and may include a contiguous sequence having at least 60% sequence identity (e.g. at least 65%, 70%, 75%, 80%, 85%, 90%, 95% identity, or 100% identity) to the V1/V2 domain sequence set forth in SEQ ID NO: 70:

(SEQ ID NO: 70)
CVTLHCTNANLTKANLTNVNNRTNVSNIIGNITDEVRNCSFNMTTELRDKK
QKVHALFYKLDIVPIEDNNDSSEYRLINCNTSVIKQAC.

In certain aspects, the fragment of gp120 may comprise a 50-100 amino acids long sequence at least 60% identical (e.g. having at least 65%, 70%, 75%, 80%, 85%, 90%, 95% identity, or 100% identity) to SEQ ID NO:70.

In other embodiments, the fragment may comprise variable region 3 (V3). In other embodiments, the fragment comprising V3 region or domain may be at least 35 amino acids in length (e.g. 35-50 amino acids) and may include a contiguous sequence having at least 60% sequence identity (e.g. at least 65%, 70%, 75%, 80%, 85%, 90%, 95% identity, or 100% identity) to the V3 domain sequence set forth in SEQ ID NO: 71:

(SEQ ID NO: 71)
QINCTRPNNNTRKRIHIGPGRAFYTTKNIKGTIRQAHCNISRAKWN

In certain aspects, the fragment of gp120 may comprise a 35-50 amino acids long sequence at least 60% identical (e.g. having at least 65%, 70%, 75%, 80%, 85%, 90%, 95% identity, or 100% identity) to SEQ ID NO:71.

In certain cases, the V3 region may comprise glycan residue N301 and N332. In certain cases, the V3 region may comprise glycan residue N301 and N332 and may extend from residue 291-342 or 296-337 of A244 gp120. The gp120 or an N-linked glycosylation site containing fragment thereof or the V1/V2 fragment may be a monomer. The numbering of the amino acid residues N301, N332, and N334 is with reference to the amino acid sequence of HIV-1 envelope polyprotein of HIV HXB having GenBank Accession No. AAB50262. AAB50262 provides a 856 amino acids long HIV-1 Env protein sequence; amino acids 34-511 define gp120 and amino acids 530 to 726 define gp41. Within gp120, the following domains are present: V1 (amino acid position 126-156); V2 (amino acid position 157-205); V3 (amino acid position 292-339); V4 (amino acid position 385-418) and V5 (amino acid position 461-471). Amino acid sequence of envelope polyprotein of HIV HXB having GenBank Accession No. AAB50262 is as follows:

(SEQ ID NO: 72)
MRVKEKYQHLWRWGWRWGTMLLGMLMICSATEKLWVTVYYGVPVWKEATTT
LFCASDAKAYDTEVHNVWATHACVPTDPNPQEVVLVNVTENFNMWKNDMVE
QMHEDIISLWDQSLKPCVKLTPLCVSLKCTDLKNDTNTNSSSGRMIMEKGE
IKNCSFNISTSIRGKVQKEYAFFYKLDIIPIDNDTTSYKLTSCNTSVITQA
CPKVSFEPIPIHYCAPAGFAILKCNNKTFNGTGPCTNVSTVQCTHGIRPVV
STQLLLNGSLAEEEVVIRSVNFTDNAKTIIVQLNTSVEINCTRPNNNTRKR
IRIQRGPGRAFVTIGKIGNMRQAHCNISRAKWNNTLKQIASKLREQFGNNK
TIIFKQSSGGDPEIVTHSFNCGGEFFYCNSTQLFNSTWFNSTWSTEGSNNT
EGSDTITLPCRIKQIINMWQKVGKAMYAPPISGQIRCSSNITGLLLTRDGG
NSNNESEIFRPGGGDMRDNWRSELYKYKVVKIEPLGVAPTKAKRRVVQREK
RAVGIGALFLGFLGAAGSTMGAASMTLTVQARQLLSGIVQQQNNLLRAIEA
QQHLLQLTVWGIKQLQARILAVERYLKDQQLLGIWGCSGKLICTTAVPWNA
SWSNKSLEQIWNHTTWMEWDREINNYTSLIHSLIEESQNQQEKNEQELLEL
DKWASLWNWFNITNWLWYIKLFIMIVGGLVGLRIVFAVLSIVNRVRQGYSP
LSFQTHLPTPRGPDRPEGIEEEGGERDRDRSIRLVNGSLALIWDDLRSLCL
FSYHRLRDLLLIVTRIVELLGRRGWEALKYWWNLLQYWSQELKNSAVSLLN
ATAIAVAEGTDRVIEVVQGACRAIRHIPRRIRQGLERILL.

In certain aspects, the polypeptide is gp140 or an N-linked glycosylation site containing fragment thereof. In certain aspects, the gp140 polypeptide may be a trimer.

In certain aspects, the polypeptide may be fused to a signal sequence. The signal sequence may be a native signal sequence or a heterologous signal sequence. In certain aspects, the heterologous signal sequence may be cleaved off from the secreted polypeptide. In certain cases, the signal sequence may be linked to the polypeptide via a linker which may be a cleavable linker. In other embodiments, the signal sequence may not be cleaved off the secreted polypeptide.

In certain aspects, the heterologous signal sequence comprises the amino acid sequence set forth in one of SEQ ID NOs: 44-47.

In certain aspects, the polypeptide may be a fusion protein comprising a purification tag. The purification tag may be present at the N-terminus and/or the C-terminus of the polypeptide. In certain aspects, the purification tag may be present at the N-terminus, where the polypeptide comprises from the N-terminus to the C-terminus: native or heterologous signal sequence, purification tag, an optional linker sequence, and the envelope glycoprotein.

In certain aspects, the polypeptide comprises the amino acid sequence set forth in SEQ ID NO: 1, 2, 3, 5, 7, 9, 10, 12, 13, 15, 17, 18, 20, 22, 23, 25, 26, 28, 30, 32, 34, 36, 38, 40, or 42.

In certain aspects, the cell line produces the polypeptide at a concentration of at least 50 mg/L after 5 days of culturing.

In certain aspects, the cell line is of CHO K1 lineage or of CHO-S lineage.

In certain aspects, the cell line comprises an endogenous gene encoding glutamine synthetase (GS). In certain aspects, the cell line comprises an endogenous gene encoding dihydrofolate reductase (DHFR).

In other aspects, the cell line does not express a GS and/or a DHFR. For example, the cell line may include an inactivation, e.g., deletion, of an endogenous gene encoding glutamine synthetase (GS) and/or an endogenous gene encoding dihydrofolate reductase (DHFR).

Also provided herein is a genetically modified Chinese hamster ovary (CHO) cell line comprising a mutation of gene encoding mannosyl (alpha-1,3)-glycoprotein beta-1,2-N-Acetylglucosaminyltransferase (Mgat1), and expressing gp120 polypeptide, wherein the genetically modified cell line is deposited with American Type Culture Collection (ATCC) as PTA-124141; or PTA-124142. The mutation may be a targeted mutation.

In addition, a method of producing a human immunodeficiency virus (HIV) envelope glycoprotein polypeptide comprising terminal mannose-5 glycans is disclosed. The method may include: a) introducing a nucleic acid comprising a nucleotide sequence encoding the HIV envelope glycoprotein polypeptide into a genetically modified Chinese hamster ovary (CHO) cell line comprising a mutation of a gene encoding mannosyl (alpha-1,3)-glycoprotein beta-1,2-N-Acetylglucosaminyltransferase (Mgat1), wherein the mutation prevents Mgat1 mediated addition of a N-acetylglucosamine moiety to a terminal mannose residue such that at least 75% of the HIV envelope glycoprotein polypeptide produced by the genetically modified cell line comprises terminal mannose-5 glycans; and b) culturing the cell line in a liquid culture medium under conditions sufficient for production of the HIV envelope glycoprotein polypeptide comprising terminal mannose-5, mannose-8, or mannose-9 glycans. The mutation may be a targeted mutation. In certain cases, introducing the nucleic acid into the cell line may include electroporation.

The method may include screening individual clones of the cell line to identify clones expressing high levels of the polypeptide. The polypeptide may be the envelope glycoprotein gp120 or an N-linked glycosylation site containing fragment thereof such as a N-linked glycosylation site containing fragment comprising variable regions 1 and 2 (V1/V2). The gp120 or an N-linked glycosylation site containing fragment thereof such as a N-linked glycosylation site containing fragment comprising V1/V2 may be a monomer. The polypeptide may be the envelope glycoprotein gp140. In certain aspects, the cell line may produce the gp140 polypeptide as a trimer.

The method may include screening by plating the clones in a semisolid matrix and contacting the clones with a detectably labeled antibody that binds to the polypeptide. In certain cases, the contacting comprises contacting the clones with a plurality of fluorescently labeled antibodies that bind to the polypeptide and form a precipitate around the clones, wherein the precipitate is visible under fluorescent light. In certain cases, the method further includes identifying clones surrounded by precipitate “halo” meeting a selection threshold and isolating the identified clones. The contacting may be carried out by including the detectably labeled antibody (e.g., affinity purified polyclonal antibodies) in the semisolid matrix on which the cells are plated. The polypeptide comprises the amino acid sequence set forth in SEQ ID NO: 1, 2, 3, 5, 7, 9, 10, 12, 13, 15, 17, 18, 20, 22, 23, 25, 26, 28, 30, 32, 34, 36, 38, 40, or 42.

The method may include recovering the HIV envelope glycoprotein polypeptide comprising terminal mannose-5 glycans from the culture medium.

As disclosed herein is the use of HIV envelope gp comprising terminal mannose-5 glycans produced using the cell lines and methods disclosed herein for inducing an immune response to HIV. In certain cases, the method may include administering the purified HIV gp, produced using the cell lines and methods disclosed herein, in a method for treating or preventing HIV infection.

Also provided herein is a recombinant HIV envelope glycoprotein polypeptide or a fragment thereof comprising at least one N-linked glycosylation site, wherein the polypeptide or the fragment comprises terminal mannose-5, mannose-8, or mannose-9 glycans at the N-linked glycosylation site.

The recombinant HIV envelope glycoprotein polypeptide or a fragment thereof may comprise a plurality of N-linked glycosylation sites, wherein the polypeptide or the fragment comprises terminal mannose-5, mannose-8, or mannose-9 glycans at the plurality of N-linked glycosylation sites. For example, the polypeptide or the fragment may include 2-20, e.g., 2-15, 2-12, 2-10, 2-8, 2-6, or 2-4, N-linked glycosylation sites and at least 50%-75% of these N-linked glycosylation sites of the polypeptide or the fragment comprise terminal mannose-5, mannose-8, or mannose-9 glycans.

The recombinant HIV envelope glycoprotein polypeptide or a fragment thereof may be as provided herein. For example, the polypeptide is gp120 or a fragment thereof, wherein the fragment comprises variable regions 1 and 2 (V1/V2) or V3 domain comprising N-linked glycosylation sites N301 and N332. For example, the fragment comprising variable regions 1 and 2 is a monomer. The polypeptide or fragment thereof may be gp140. The gp140 fragment may be a trimer. The polypeptide or the fragment may be fused to a heterologous signal sequence. The heterologous signal sequence comprises the amino acid sequence set forth in one of SEQ ID NOs: 44-47. The polypeptide or the fragment comprises a purification tag. The purification tag comprises the amino acid sequence set forth in one of SEQ ID NOs: 48-56.

In certain aspects, the polypeptide or the fragment comprises the amino acid sequence set forth in SEQ ID NO: 1, 2, 3, 5, 7, 9, 10, 12, 13, 15, 17, 18, 20, 22, 23, 25, 26, 28, 30, 32, 34, 36, 38, 40, or 42 or comprises an amino acid sequence at least 85% (e.g., 90%, 95%, 96%, 97%, 98%, or 99%) identical to the amino acid sequence set forth in SEQ ID NO: 1, 2, 3, 5, 7, 9, 10, 12, 13, 15, 17, 18, 20, 22, 23, 25, 26, 28, 30, 32, 34, 36, 38, 40, or 42.

Also provided herein is a composition comprising the polypeptide or the fragment of any one of claims 38-50 and a pharmaceutically acceptable excipient.

In addition, a method for inducing an immune response to HIV in a mammal by administering the polypeptide and compositions disclosed herein is provided.

### BRIEF DESCRIPTION OF THE FIGURES

FIG. 1 depicts a simplified view of the N-linked glycosylation pathway.

FIG. 2 shows the GeneArt® CRISPR Nuclease vector.

FIG. 3A provides the sequence of the CHO Mgat1 gene (SEQ ID NO:64). A target of a guideRNA (gRNA) is underlined with the requisite protospacer adjacent motif in bold. FIG. 3B depicts the GeneArt CRISPR nuclease vector used to edit the CHO Mgat1 gene. GGGCATTCCAGCCCACAAAGGTTTT (SEQ ID NO: 65) and the complementary sequence (CTTTGTGGGCTGGAATGCCCCGGTG: SEQ ID NO:66) for facilitating cloning into the vector are depicted.

FIG. 4 provides a flow chart of Mgat1 gene editing and the cell line selection strategy.

FIG. 5 shows results from a GNA lectin binding assay used to find cells with high mannose surface glycoproteins following CRISPR/Cas9 targeted cleavage of Mgat1.

FIG. 6A illustrates the native sequence at the region of Mgat1 gene targeted by gRNA. FIG. 6B-6D illustrate NHEJR induced changes to the Mgat1 gene. Nucleotides different from the native sequence are underlined.

FIG. 7A shows the cell doubling time of Mgat1⁻ CHO cell lines. FIG. 7B shows the transient expression of gp120 in Mgat1⁻ CHO cell lines (3.5D9, 3.5D8, 3.4F10, 3.5A2′) and in CHO-S and Gnt1-cell lines.

FIGS. 8A-8C illustrate the expression of gp120 in a GB Mgat1⁻ CHO cell line. FIG. 8A shows purified A244 produced by WT CHO-S, GB Mgat1 CHO, and 293 HEK Gnt1⁻ cells. FIG. 8B shows samples of the same proteins digested with Endo H. FIG. 8C shows samples of the same proteins digested with PNGase F.

FIGS. 9A and 9B illustrates isoelectric focusing of CHO-S and Mgat1⁻gp120. FIG. 9A illustrates the isoelectric focusing of gp120 expressed in CHO-S. FIG. 9B illustrates the isoelectric focusing of gp120 in expressed in Mgat1⁻.

FIGS. 10A and 10B show PG9 binding to monomeric gp120 and V1V2 scaffold was improved by Mgat1 knockout (Mgat1⁻) in CHO cells. FIG. 10A shows PG9 binding to monomeric gp120. FIG. 10B shows PG9 binding to V1/V2 fragment protein.

FIG. 11 provides a diagram of the UCSC1331 plasmid used to express A244_N332-rgp120.

FIG. 12 provides a diagram of the chimeric gene used for the expression of A244_N332-rgp120.

FIG. 13 provides the Emboss Needle pairwise sequence alignment of the amino acid sequence of the A244_N332-rgp120 transcription product with the A244-rgp120 transcription product used to produce rgp120 for the RV144 clinical trial. A is A244_{UCSC} rgp120 (SEQ ID NO:71) and B is A244_{GNE} rgp120 (SEQ ID NO:72).

FIG. 14 depicts the comparison of the wild-type A244-rgp120 transcription product with the A244-N332-rgp120 transcription product and the mature processed form of the 244_N332-rgp120 protein.

FIGS. 15A and 15B provide the Emboss Needle pairwise sequence alignment of the nucleotide sequence of the codon optimized A244_N332-rgp120 gene (SEQ ID NO:73) and the A244-rgp120 gene (SEQ ID NO:74) used to produce A244-rgp120 for the RV144 clinical trial.

FIG. 16 depicts an SDS-PAGE gel of gp120 proteins used for goat immunization.

FIGS. 17A-17D illustrate the measurement of antibodies to A244-, MN-, and CN97001 gp120s and to the HSV1 glycoprotein purification tag during the course of immunization of Goat 577.

FIGS. 18A-18C illustrate the comparison of ClonePix2 images obtained with protein G purified, Alexa 488 labeled goat IgG and with gp120-affinity-purified, Alexa 488 labeled IgG. FIG. 18A shows images of cells after a 14 day incubation of Mgat1-cells expressing A244-N332-rgp120 with polyclonal immuno-affinity purified Alexa 488 labeled goat IgG. FIG. 18B shows images of cells after a 14 day incubation of Mgat1-cells expressing A244_N332-rgp120 with 10 μg/ml of Alexa 488 labeled, protein G purified, goat IgG. FIG. 18C shows images of cells from a control experiment where of Mgat1-cells expressing A244_N332-rgp120 were incubated for 14 days without added antibody.

FIG. 19 provides a diagram of a method for rapid production of cell lines expressing recombinant gp120.

FIG. 20 shows GFP expression after MaxCyte STX electroporation of CHO-S cells.

FIG. 21 shows white and fluorescent images from a single well of UCSC_CHO.A244N332 transfected cells on the ClonePix 2.

FIGS. 22A-22E provide ClonePix 2 Clone images at Day 16. FIG. 22A illustrates a single 35 mm well of UCSC_CHO.A244N332 transfected colonies illuminated by white light alone. FIG. 22B shows the same well as in A but FITC imaged. FIG. 22C illustrates the superimposition of white and FITC images. FIG. 22D shows six colonies picked on Day 16, expanded, and visualized with white light and FITC. FIG. 22E shows Clone 5F recloned at 25 cells/ml and visualized with white light and FITC.

FIGS. 23A and 23B illustrate the expression of proteins in 2 ml wells. FIG. 23A provides a Western blot of tissue culture supernatant from 2 ml wells. FIG. 23B provides indirect ELISA quantification of rgp120 A244N332.

FIGS. 24A and 24B show batch fed culture expression of Clone 5F: accumulation of rgp120 during 600 ml protein expression trial culture. FIG. 24A shows a SDS/PAGE gel with 10 μl DTT reduced tissue culture supernatant (days 0-5) loaded per lane. FIG. 24B shows a SDS/PAGE gel with 1 μl DTT reduced tissue culture supernatant (days 0-5) loaded per lane and western blotted with an antigen specific polyclonal rabbit serum.

FIGS. 25A-25F illustrate indirect ELISA results showing raw dilution data of tissue culture supernatant collected during a batch fed protein expression assay.

FIG. 26A depicts protein yield from 600 ml batch fed cultures pre and post purification by immunoaffinity capture.

FIG. 26B shows a western blot of protein purified by affinity chromatography from 600 ml batch fed cultures.

FIGS. 27A-27H illustrates direct binding of purified MGAT gp120 HIV-1 proteins to bNAbs.

FIGS. 28A-28J provide the comparison of bNAb binding to CHO A244_{GNE}-rgp120 produced in normal CHO cells and used in the RV144 trial, and improved A244-N332-rgp120 produced in Mgat1⁻ cells.

FIGS. 29A-29F show data from 2-dimensional isoelectric focusing gel analysis of MN-rgp120 produced in CHO and 293 HEK cells.

FIG. 30 illustrates the steps for purification of A244_N332-rgp120 by column chromatography.

FIG. 31 shows the comparison of A244_N332-rgp120 recovered by an immunoaffinity recovery process dependent of the 5B6 monoclonal antibody and column chromatography (Desalting-IEXHP-SEC) recovery process.

FIG. 32 shows the steps for purification of A244_N332-rgp120 by immunoaffinity chromatography and size exclusion chromatography.

FIG. 33 provides the comparison of the recovered yields of A244_N332-rgp120 obtained from the recovery process containing an immunoaffinity step and the recovery process depending only on column chromatography.

### DEFINITIONS

The practice of the present invention will employ, unless otherwise indicated, conventional methods of medicine, chemistry, biochemistry, immunology, cell biology, molecular biology and recombinant DNA techniques, within the skill of the art. Such techniques are explained fully in the literature. All publications, patents and patent applications cited herein are hereby incorporated by reference in their entireties.

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a cell” includes a mixture of two or more such cells, and the like. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The term “heterologous” refers to two biological components that are not found together in nature. The components may be host cells, genes, or regulatory regions, such as promoters. Although the heterologous components are not found together in nature, they can function together, as when a promoter heterologous to a gene is operably linked to the gene. “Heterologous” in the context of recombinant cells can refer to the presence of a nucleic acid (or gene product, such as a polypeptide) that is of a different genetic origin than the host cell in which it is present. For example, a recombinant cell expressing a heterologous polypeptide refers to a cell that is genetically modified to introduce a nucleic acid encoding the polypeptide which nucleic acid is not naturally present in the cell.

“Endogenous” as used herein to describe a gene or a nucleic acid in a cell means that the gene or nucleic acid is native to the cell (e.g., a non-recombinant host cell) and is in its normal genomic and chromatin context, and which is not heterologous to the cell. Mgat1, glutamine synthetase, dihydrofolate reductase are examples of genes that are endogenous to mammalian cells, such as, CHO cells. When added to a cell, a recombinant nucleic acids would also be heterologous to the endogenous genes of the cell. Thus, in a chromosome, a heterologous nucleic acid would include an non-native (non-naturally occurring) nucleic acid that has integrated into the chromosome, or a non-native (non-naturally occurring) extrachromosomal nucleic acid. In contrast, a naturally translocated piece of chromosome would not be considered heterologous in the context of this patent application, as it comprises an endogenous nucleic acid sequence that is native to the mutated cell.

“Recombinant” as used herein to describe a nucleic acid molecule means a polynucleotide of genomic, cDNA, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions. Thus, for example, recombinant cells, such as a recombinant host cell, express genes that are not found within the native (naturally occurring) form of the cell or express a second copy of a native gene that is otherwise normally or abnormally expressed, under expressed or not expressed at all.

The term “transformation” or “genetic modification” refers to a permanent or transient genetic change induced in a cell following introduction of an new nucleic acid. Thus, a “genetically modified host cell” is a host cell into which a new (e.g., exogenous; heterologous) nucleic acid has been introduced. Genetic change (“modification”) can be accomplished either by incorporation of the new DNA into the genome of the host cell, or by transient or stable maintenance of the new DNA as an episomal element. In eukaryotic cells, a permanent genetic change is generally achieved by introduction of the DNA into the genome of the cell.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

“Encode,” as used in reference to a nucleotide sequence of nucleic acid encoding a gene product, e.g., a polypeptide, of interest, is meant to include instances in which a nucleic acid contains a nucleotide sequence that is the same as in a cell or genome that, when transcribed and/or translated into a polypeptide, produces the gene product. In some instances, a nucleotide sequence or nucleic acid encoding a gene product does not include intronic sequences.

“Substantially purified” generally refers to isolation of a substance (compound, polynucleotide, protein, or polypeptide) such that the substance comprises the majority percent of the sample in which it resides. Typically in a sample, a substantially purified component comprises 50%, 80%-85%, or 90-95% of the sample. Techniques for purifying polynucleotides, oliognucleotides, and polypeptides of interest are well-known in the art and include, for example, ion-exchange chromatography, affinity chromatography and sedimentation according to density.

The term “operably linked” refers to a juxtaposition wherein the components so described are in a relationship permitting them to function in their intended manner. For instance, a promoter is operably linked to a nucleotide sequence if the promoter affects the transcription or expression of the nucleotide sequence.

A “host cell,” as used herein, denotes an in vitro eukaryotic cell (e.g., a mammalian cell, such as, a CHO cell line), which eukaryotic cell can be, or has been, used as a recipient for a nucleic acid, and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a subject eukaryotic host cell is a genetically modified eukaryotic host cell, by virtue of introduction into of a heterologous nucleic acid, e.g., an exogenous nucleic acid that is foreign to the eukaryotic host cell, or a recombinant nucleic acid that is not normally found in the eukaryotic host cell.

As used herein, the term “cell line” refers to a population of cells produced from a single cell and therefore consisting of cells with a uniform genetic makeup.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a polynucleotide refers to a nucleic acid molecule devoid, in whole or part, of sequences normally associated with it in nature; or a sequence, as it exists in nature, but having heterologous sequences in association therewith; or a molecule disassociated from the chromosome.

The terms “polynucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term also includes modifications, such as by methylation and/or by capping, and unmodified forms of the polynucleotide. More particularly, the terms “polynucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “nucleic acid” and “nucleic acid molecule,” and these terms will be used interchangeably.

The terms “label” and “detectable label” refer to a molecule capable of being detected, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the invention include, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

### DETAILED DESCRIPTION

The present disclosure provides cell lines and methods for producing HIV envelope glycoprotein polypeptides that possess terminal mannose-5 glycans. The HIV envelope glycoproteins produced by the cell lines and methods provided herein are suitable for eliciting antibodies effective in prevention and/or treatment of HIV infection. In certain cases, the antibodies elicited by the HIV envelope glycoproteins produced by the cell lines disclosed herein are broadly neutralizing antibodies. Further details of the cell lines and methods are provided below.

### Cell Lines

Provided herein are recombinant cell lines for producing biopharmaceuticals, such as, HIV envelope glycoprotein polypeptides comprising terminal mannose-5 glycans. In certain embodiments, the cell line is derived from a CHO cell line that lacks or has limited expression of or function of the endogenous gene encoding mannosyl (alpha-1,3)-glycoprotein beta-1,2-N-Acetylglucosaminyltransferase (Mgat1). Mgat1 is also referred to as N-Glycosyl-Oligosaccharide-Glycoprotein N-Acetylglucosaminyltransferase I, Alpha-1,3-Mannosyl-Glycoprotein 2-Beta-N-Acetylglucosaminyltransferas, GlcNAc-T I, GLYT1, GLCT1, GNT-1, GLCNAC-TI, and Gnt1. Deletion of Mgat1 prevents glycosylation from advancing beyond the Man₅GlcNAc₂ state in the modified cell lines disclosed herein.

In certain embodiments, the CHO cell line has been genetically modified to delete the endogenous mgat1 gene. In such embodiments, the deletion of the endogenous mgat1 gene may be carried out by using CRISPER/Cas9 mediated gene editing. In certain embodiments, the CRISPER/Cas9 mediated deletion of mgat1 gene prevents Mgat1-mediated addition of a N-acetylglucosamine moiety to a terminal mannose residue present at the N-linked glycosylation site of the HIV envelope glycoprotein polypeptide produced in the cell line, resulting in expression of the HIV envelope glycoprotein polypeptide with one or more terminal mannose, e.g, mannose-5, mannose-8, or mannose-9.

In certain embodiments, the Mgat1 deficient cell lines may include a Mgat1 encoding gene sequence that has been completely or partially inactivated. In certain embodiments, two copies of the mgat1 gene has been inactivated. In some embodiments, three or more copies of mgat1 gene has been inactivated. Inactivation of mgat1 gene may be due to deletion of a part or entire sequence of the of mgat1 gene and/or due to insertion of at least one nucleotide. The inactivation may result in reduced expression or reduced activity of Mgat1. In some embodiments, the inactivation may result in lack of expression of Mgat1. In some examples, the inactivation of mgat1 gene results in expression of a truncated or otherwise mutated Mgat1 that lacks detectable activity.

In certain aspects, the Mgat1 deficient cell lines may include an insertion in the mgat1 gene resulting in a frame shift mutation and a premature stop codon. In certain aspects, the premature stop codon may result in production of a truncated Mgat polypeptide that has no detectable activity. In certain aspects, the truncated Mgat may be an N-terminal fragment of full length Mgat1 and may be 10-50 amino acids or 20-50 amino acids, such as, 20, 30 or 40 amino acids long. In certain embodiments, the Mgat1 deficient cell line may include mgat gene in which nucleotides have been deleted. The deletion may be in the sequence encoding the transmembrane region of Mgat1. The deletion may result in a Mgat1 polypeptide having a deletion of 8-30 amino acids in the transmembrane region, such as, deletion of 6 to 10 amino acids, 25-35 amino acids, such as, 8 or 30 amino acids, resulting in a Mgat1 polypeptide with reduced activity.

In certain cases, the mgat1 gene targeted for inactivation may have the sequence set forth in SEQ ID NO:64. The Mgat1 polypeptide may have the amino acid sequence set forth in SEQ ID NO: 75. In certain embodiments, the cell lines disclosed herein may comprise an inactivated mgat1 gene having the sequence set forth in SEQ ID NO:76, where the inactivated mgat1 gene encodes a truncated Mgat1 polypeptide having the sequence set forth in SEQ ID NO:77.

In certain aspects, the glycosylation heterogeneity of the polypeptides produced by cell lines provided herein is markedly reduced such that a majority of the polypeptides have one or more terminal mannose, mannose-5, mannose-8, or mannose-9 glycans. In certain embodiments, the genetic modification to delete the endogenous mgat1 gene results in at least 75% of the HIV envelope glycoprotein polypeptides produced by the genetically modified cell line having terminal mannose glycans at the N-linked glycosylation site. In certain cases, at least 75% or more, such as, 75%-95%, 75%-96%, 75%-97%, 75%-98%, 80%-98%, 85%-99%, e.g., 80%, 85%, 90%, 95%, 98%, 99%, or more of the HIV envelope glycoprotein polypeptides produced by the genetically modified cell line have terminal mannose glycans at the N-linked glycosylation site. As used herein, the term “terminal mannose” or “terminal mannose glycans” refers to N-glycans having one or more mannose residues at the terminus of the N-glycan. This term encompasses, N-glycans having 5, 8, or 9 terminal mannose residues.

The CHO cell line from which the cell lines disclosed herein are derived may be a CHO cell line adapted for growth in suspension culture, adherent culture, or both. In certain aspects, the genetically modified CHO cell line may be derived from a parent CHO cell line, such as, CHO S, CHO K1, CHO-DXB11 (also known as CHO-DUKX), CHO-PRO3, CHO-PRO5, or CHO-DG44 cell line, and the like.

In certain aspects, the genetically modified CHO cell line is not deficient in markers commonly used for selection of transfected CHO cells, such as, glutamine synthetase (GS), dihyropfolate reductase (DHFR), and the like. In certain aspects, the genetically modified CHO cell line is derived from a parental CHO cell line that includes a gene encoding GS, DHFR, or both. As such, in certain examples, the generation of the genetically modified CHO cell line does not require transfection of a nucleic acid encoding GS and/or DHFR. In certain aspects, the genetically modified CHO cell line is derived from a parental CHO S or CHO K1 cell line that includes a gene encoding GS, DHFR, or both. In certain aspects, the parental cell line is CHO S that expresses GS. In other embodiments, the parental cell line is CHO K1 that expresses GS. In certain embodiments, the genetically modified CHO cell line of the present disclosure is not derived from CHO Lec1 cells. In certain embodiments, the genetically modified CHO cell line of the present disclosure does not produce Mgat1 or fragments thereof. In certain embodiments, the Mgat1 encoding gene has been deleted from the cell lines disclosed herein such that the cell line has no detectable Mgat1 activity. In certain embodiments, the Mgat1 encoding gene has been disrupted from the cell lines disclosed herein such that the cell line has no detectable Mgat1 activity. In other aspects, the cell line may also be deficient in GS and/or DHFR.

In certain aspects, the cell lines provided herein produce the exogenous polypeptide at a concentration of at least 50 milligrams/Liter (mg/L), such as, at least 75 mg/L, 100 mg/L, 150 mg/L, 175 mg/L, 200 mg/L, 250 mg/L, 300 mg/L, e.g., 50-300 mg/L, 50-250 mg/L, or 50-200 mg/L. The cell line may express the exogenous polypeptide at a concentration of at least 50 mg/L after 1-30 days of culturing, e.g., 1 day, 2, days, 3 days, 5 days, 7 days, 10 days, 15 days, 20 days, or more.

A subject genetically modified host cell is generated using standard methods well known to those skilled in the art. In some cases, the nucleic acid encoding Mgat1 is disrupted (e.g., deleted) using a CRISPR/Cas9 system comprising: i) an RNA-guided endonuclease; and ii) a guide RNA (e.g., a single molecule guide RNA; or a double-molecule guide RNA) that provides for deletion of endogenous Mgat1 gene; and iii) a donor DNA template. Suitable RNA-guided endonucleases include an RNA-guided endonuclease comprising an amino acid sequence having at least 85%, at least 90%, at least 95%, at least 98%, at least 99%, or 100%, amino acid sequence identity to the amino acid sequence of Streptococcus pyogenes Cas9 (GenBank Accession No.: AKP81606.1) or Staphylococcus aureus Cas9 (NCBI Reference Sequence: WP_001573634.1). The guide RNA comprises a targeting sequence. A suitable targeting sequence can be determined by those skilled in the art. The donor template comprises a nucleotide sequence complementary to Mgat1-encoding nucleotide sequence.

In certain aspects, a genetically modified Chinese hamster ovary (CHO) cell line comprising a targeted mutation of gene encoding mannosyl (alpha-1,3)-glycoprotein beta-1,2-N-Acetylglucosaminyltransferase (Mgat1) and expressing gp120 glycoprotein, wherein the genetically modified cell line is deposited with American Type Culture Collection (ATCC) as PTA-124141; or PTA-124142 is also disclosed.

### Compositions and Methods for Producing Exogenous Polypeptide

The present disclosure provides a composition comprising: a) a genetically modified host cell line as described above or elsewhere herein; and b) a culture medium.

The present disclosure provides a method of producing a polypeptide of interest. The method may include culturing the composition for a time period and under conditions suitable for production of the exogenous polypeptide, where the composition comprises: a) a genetically modified host cell line of the present disclosure; and b) a culture medium; and separating the genetically modified host cell line from the culture medium, to generate a cell culture comprising secreted polypeptide of interest. Separating the genetically modified host cells from the culture medium can be accomplished by methods known in the art, such as centrifugation, filtration, and the like.

The exogenous polypeptide secreted into the culture medium may be purified using any standard process. For example, the exogenous polypeptide, such as, an envelope glycoprotein, e.g., gp140 trimer, secreted into the culture medium may be purified using the process disclosed in Sanders R W, Moore J P. Immunological reviews. 2017 Jan. 1; 275(1):161-82; Sanders R W, et al., PLoS pathogens. 2013 Sep. 19; 9(9):e1003618; Sharma S K, et al., Cell reports. 2015 Apr. 28; 11(4):539-50; or Karlsson Hedestam G B, et al., Immunological reviews. 2017 Jan. 1; 275(1):183-202.

In certain embodiments, production of exogenous polypeptides using the cell lines provided herein does not require culturing in the presence of inhibitors that prevent glycosylation from proceeding beyond Man₅GlcNAc₂ state. As such, the culture medium for culturing the cell lines for expressing an exogenous polypeptide does not include inhibitors such as kifunensine.

In certain embodiments, a method of producing a human immunodeficiency virus (HIV) envelope glycoprotein polypeptide comprising terminal mannose-5 glycans is disclosed. The method may include: a) introducing a nucleic acid comprising a nucleotide sequence encoding the HIV envelope glycoprotein polypeptide into a genetically modified Chinese hamster ovary (CHO) cell line comprising a mutation of the gene encoding mannosyl (alpha-1,3)-glycoprotein beta-1,2-N-Acetylglucosaminyltransferase (Mgat1), wherein the mutation prevents Mgat1 mediated addition of a N-acetylglucosamine moiety to a terminal mannose residue such that at least 75% of the HIV envelope glycoprotein polypeptide produced by the genetically modified cell line comprises terminal mannose-5, mannose-8, or mannose-9 glycans; and b) culturing the cell line in a liquid culture medium under conditions sufficient for production of the HIV envelope glycoprotein polypeptide comprising terminal mannose-5, mannose-8, or mannose-9 glycans.

The method may include screening by plating the clones in a semisolid matrix and contacting the clones with a detectably labeled antibody that binds to the polypeptide. In certain cases, the contacting comprises contacting the clones with a plurality of fluorescently labeled antibodies that bind to the polypeptide and form a precipitate around the clones, wherein the precipitate is visible under fluorescent light. In certain cases, the method further includes identifying clones surrounded by precipitate meeting a selection threshold and isolating the identified clones. The polypeptide comprises the amino acid sequence set forth in SEQ ID NO: 1, 2, 3, 5, 7, 9, 10, 12, 13, 15, 17, 18, 20, 22, 23, 25, 26, 28, 30, 32, 34, 36, 38, 40, or 42.

In certain aspects, identification of a Mgat1 deficient (Mgat1⁻) cell line may be carried out using a positive selection method. In certain embodiments, the method may include contacting cells suspected of being Mgat1 deficient (Mgat1⁻) with a GNA lectin, where the GNA lectin is a mannose binding lectin with a preference for α1,3 linked mannose residues. In certain aspects, the method for identifying Mgat1⁻ cells does not involve using ricin lectins, such as Ricinus communis agglutinin-I and II.

In certain cases, Mgat1⁻ cells expressing an exogenous polypeptide may be identified using polyclonal antibodies that have been purified based on their ability to bind to the exogenous polypeptide. For example, the exogenous polypeptide may be used to immunize an animal and elicit antibodies to the exogenous polypeptide. The antibodies may be affinity purified using a solid substrate (e.g., a bead, a column, etc.) to which the exogenous polypeptide is conjugated. The affinity purified antibodies may be conjugated to a detectable label and used for identifying cells expressing the exogenous polypeptide. In certain embodiments, the affinity purified polyclonal antibodies bind to exogenous polypeptide secreted by the cells expressing the polypeptide. In certain embodiments, the binding of the affinity purified antibodies to the exogenous polypeptide secreted by the cells expressing the polypeptide may be detected by visualizing the detectable label. In certain embodiments, the detectable label may be a fluorescent label, such as, alexa dye. In certain embodiments, the affinity purified polyclonal antibodies form a fluorescent halo around the cells expressing the polypeptide thereby facilitating rapid identification of cells expressing high levels of the polypeptide.

### Exogenous Polypeptide

Any exogenous polypeptide of interest can be produced using the cell lines described herein. In some embodiments, the exogenous polypeptide may be a polypeptide that can be used to elicit an immune response in a mammal. In certain embodiments, the immune response may result in prevention or treatment of HIV infection.

In certain embodiments, the exogenous polypeptide is a polypeptide that undergoes glycosylation when expressed in a eukaryotic host cell. In certain embodiments, the exogenous polypeptide includes a N-linked glycosylation site comprising the consensus sequence Asn-X-Ser/Thr, where X is any amino acid except proline (Pro). In certain embodiments, expressing the exogenous polypeptide in the cell lines provided herein prevents prevents glycosylation from advancing beyond the Man₅GlcNAc₂ state.

In certain embodiments, the exogenous polypeptide is a HIV-1 envelope glycoprotein (gp) or a fragment thereof, provided that the fragment contains an N-linked glycosylation site containing fragment thereof. In certain cases, the envelope gp is gp160, gp120 (e.g., gp120 monomer), gp140 (e.g., gp140 trimer) or an envelope gp fragment containing variable regions 1 and 2 (V1/V2).

In certain embodiments, the exogenous polypeptide is an envelope glycoprotein or a fragment thereof, provided that the fragment contains an N-linked glycosylation site containing fragment thereof and may comprise an amino acid sequence set forth below.

Clade CRF01_AE: A244_ N332_{c} rgp120
(SEQ ID NO: 1)
VPVWKEADTTLFCASDAKAHETEVHNVWATHACVPTDPNPQEIDLENVTEN
FNMWKNNMVEQMQEDVISLWDQSLKPCVKLTPPCVTLHCTNANLTKANLTN
VNNRTNVSNIIGNITDEVRNCSFNMTTELRDKKQKVHALFYKLDIVPIEDN
NDSSEYRLINCNTSVIKQACPKISFDPIPIHYCTPAGYAILKCNDKNFNGT
GPCKNVSSVQCTHGIKPVVSTQLLLNGSLAEEEIIIRSENLTNNAKTIIVH
LNKSVVINCTRPSNNTRTSITIGPGQVFYRTGDIIGDIRKAYCNISGTEWN
KALKQVTEKLKEHFNNKPIIFQPPSGGDLEITMHHFNCRGEFFYCNTTRLF
NNTCIANGTIEGCNGNITLPCKIKQIINMWQGAGQAMYAPPISGTINCVSN
ITGILLTRDGGATNNTNNETFRPGGGNIKDNWRNELYKYKVVQIEPLGVAP
TRAKRRVVEREKR

The V1/V2 domain is double underlined and starts at amino acid position 83 and ends at position 171 and V3 domain is underlined and starts at amino acid position 259 and ends at amino acid position 304 in SEQ ID NO:1.

Clade CRF01_AE: A244_N332_{c} rgp120
(SEQ ID NO: 2)
VPVWKEADTTLFCASDAKAHETEVHNVWATHACVPTDPNPQEIDLENVTEN
FNMWKNNMVEQMQEDVISLWDQSLKPCVKLTPPCVTLHCTNANLTKANLTN
VNNRTNVSNIIGNITDEVRNCSFNMTTELRDKKQKVHALFYKLDIVPIEDN
NDSSEYRLINCNTSVIKQACPKISFDPIPIHYCTPAGYAILKCNDKNFNGT
GPCKNVSSVQCTHGIKPVVSTQLLLNGSLAEEEIIIRSENLTNNAKTIIVH
LNKSVVINCTRPSNNTRTSITIGPGQVFYRTGDIIGDIRKAYCNISGTEWN
KALKQVTEKLKEHFNNKPIIFQPPSGGDLEITMHHFNCRGEFFYCNTTRLF
NNTCIANGTIEGCNGNITLPCKIKQIINMWQGAGQAMYAPPISGTINCVSN
ITGILLTRDGGATNNTNNETFRPGGGNIKDNWRNELYKYKVVQIEPLGVAP
TRA

V1/V2 domain is double underlined and V3 domain is underlined.

Clade CRF01_AE: gD_A244_N332e rgp120 (UCSC1250)
(SEQ ID NO: 3)
MGGAAARLGAVILFVVIVGLHGVRGKYALADASLKMADPNRFRGKDLPVLD
QLLEVPVWKEADTTLFCASDAKAHETEVHNVWATHACVPTDPNPQEIDLEN
VTENFNMWKNNMVEQMQEDVISLWDQSLKPCVKLTPPCVTLHCTNANLTKA
NLTNVNNRTNVSNIIGNITDEVRNCSFNMTTELRDKKQKVHALFYKLDIVP
IEDNNDSSEYRLINCNTSVIKQACPKISPDPIPIHYCTPAGYAILKCNDKN
FNGTGPCKNVSSVQCTHGIKPVVSTQLLLNGSLAEEEIIIRSENLTNNAKT
IIVHLNKSVVINCTRPSNNTRTSITIGPGQVFYRTGDIIGDIRKAYCNISG
TEWNKALKQVTEKLKEHFNNKPIIFQPPSGGDLEITMHHFNCRGEFFYCNT
TRLFNNTCIANGTIEGCNGNITLPCKIKQIINMWQGAGQAMYAPPISGTIN
CVSNITGILLTRDGGATNNTNNETFRPGGGNIKDNWRNELYKYKVVQIEPL
GVAPTRA

gD signal sequence is underlined; mature N-terminal gD purification tag is italicized; linker sequence is in bold. V1/V2 domain is double underlined and V3 domain is underlined.

The exogenous polypeptide comprising an amino acid sequence set forth in SEQ ID NO:3 may be encoded by the nucleic acid sequence set forth in SEQ ID NO:4:

(SEQ ID NO: 4)
ATGGGGGGGGCTGCCGCCAGGTTGGGGGCCGTGATTTTGTTTGTCGTCATA
GTGGGCCTCCATGGGGTCCGCGGCAAATATGCCTTGGCGGATGCCTCTCTC
AAGATGGCCGACCCCAATCGATTTCGCGGCAAAGACCTTCCGGTCCTGGAC
CAGCTGCTCGAGGTACCAGTGTGGAAGGAAGCCGACACAACCCTCTTCTGC
GCCAGCGATGCCAAGGCCCACGAGACGGAGGTCCACAATGTGTGGGCCACC
CATGCCTGTGTGCCCACGGACCCCAACCCCCAGGAGATTGACCTGGAGAAT
GTCACGGAGAACTTCAACATGTGGAAGAACAACATGGTGGAGCAGATGCAG
GAGGACGTCATCTCCCTGTGGGACCAGAGCCTGAAACCCTGCGTCAAACTG
ACACCCCCCTGTGTGACCCTGCACTGCACGAACGCCAACCTGACCAAGGCC
AACCTCACCAACGTGAACAATCGGACCAACGTGTCCAACATCATCGGGAAC
ATCACAGATGAGGTGAGGAACTGCAGCTTCAATATGACAACCGAGCTCCGG
GACAAAAAGCAGAAGGTGCACGCGTTGTTCTACAAACTGGATATCGTCCCC
ATCGAGGACAATAATGACAGcTCCGAGTATCGCCTGATCAACTGCAACACC
AGcGTCATCAAACAGGCCTGCCCCAAAATTTCCTTCGACCCCATCCCCATC
CACTACTGCACCCCAGCTGGGTACGCCATCCTGAAGTGCAATGACAAGAAC
TTCAACGGCACAGGGCCCTGCAAGAATGTGAGCTCCGTCCAGTGCACCCAC
GGCATCAAGCCAGTGGTCTCCACCCAGCTCCTCCTGAATGGGAGCCTGGCA
GAGGAAGAGATCATCATCCGCTCCGAGAACCTGACCAACAATGCCAAGACC
ATCATCGTCCACCTGAATAAGTCCGTGGTCATCAACTGCACCAGACCCAGC
AACAACACGCGGACCAGCATCACCATCGGCCCAGGGCAGGTCTTCTATAGG
ACGGGGGACATCATTGGGGACATCAGGAAGGCCTACTGCAACATCAGTGGG
ACCGAGTGGAACAAAGCCCTGAAACAGGTGACCGAAAAACTCAAGGAGCAC
TTCAACAACAAGCCAATCATCTTCCAGCCCCCCAGCGGGGGGGACCTGGAG
ATCACCATGCACCATTTCAACTGCCGGGGGGAATTCTTCTACTGCAACACC
ACCCGCCTGTTCAACAACACCTGCATCGCCAACGGCACCATCGAGGGCTGC
AATGGCAACATCACCCTCCCATGCAAAATCAAGCAGATCATCAACATGTGG
CAGGGGGCAGGCCAGGCCATGTACGCCCCCCCCATCTCCGGCACGATCAAC
TGCGTGTCCAACATCACGGGGATCCTGCTGACCCGGGATGGGGGGGCTACC
AACAATACGAACAATGAGACCTTCAGGCCAGGGGGGGGGAACATCAAAGAC
AACTGGCGCAATGAGCTCTACAAGTACAAAGTGGTGCAGATCGAGCCCCTG
GGGGTGGCCCCCACCCGGGCCAAACGCAGGGTGGTGGAGCGGGAGAAGCGG

Nucleotides encoding the gD signal sequence are underlined; nucleotides encoding the mature N-terminal gD purification tag are italicized; nucleotides encoding linker sequence are in bold.

Clade B: gD-MN468-rgp120; UCSC468
(SEQ ID NO: 9)
VPVWKEATTTLFCASDAKAYDTEAHNVWATHACVPTDPNPQEVELVNVTEN
FNMWKNNMVEQMHEDIISLWDQSLKPCVKLTPLCVTLNCTDLRNTTNTNNS
TDNNNSKSEGTIKGGEMKNCSFNITTSIGDKMQKEYALLYKLDIEPIDNDS
TSYRLISCNTSVITQACPKISFEPIPIHYCAPAGFAIXKCNDKKFSGKGSC
KNVSTVQCTHGIRPVVSTQLLLNGSLAEEEVVIRSEDFTDNAKTIIVHLNE
SVQINCTRPNNNTRKRIHIGPGRAFYTTKNIKGTIRQAHCNISRAKWNDTL
RQIVSKLKEQFKNKTIVFNPSSGGDPEIVMHSFNCGGEFFYCNTSPLFNSI
WNGNNTWNNTTGSNNNITLQCKIKQIINMWQKVGKAMYAPPIEGQIRCSSN
ITGLLLTRDGGEDTDTNDTEIFRPGGGDMRDNWRSELYKYKVVTIEPLGVA
PTKA

V1/V2 domain is double underlined and V3 domain is underlined.

gD-MN468-rgp120; UCSC468
(SEQ ID NO: 10)
MGGAAARLGAVILFVVIVGLHGVRGKYALADASLKMADPNRFRGKDLPVLD
QLLEVPVWKEATTTLFCASDAKAYDTEAHNVWATHACVPTDPNPQEVELVN
VTENFNMWKNNMVEQMHEDIISLWDQSLKPCVKLTPLCVTLNCTDLRNTTN
TNNSTDNNNSKSEGTIKGGEMKNCSFNITTSIGDKMQKEYALLYKLDIEPI
DNDSTSYRLISCNTSVITQACPKISFEPIPIHYCAPAGFAIXKCNDKKFSG
KGSCKNVSTVQCTHGIRPVVSTQLLLNGSLAEEEVVIRSEDFTDNAKTIIV
HLNESVQINCTRPNNNTRKRIHIGPGRAFYTTKNIKGTIRQAHCNISRAKW
NDTLRQIVSKLKEQFKNKTIVFNPSSGGDPEIVMHSFNCGGEFFYCNTSPL
FNSIWNGNNTWNNTTGSNNNITLQCKIKQIINMWQKVGKAMYAPPIEGQIR
CSSNITGLLLTRDGGEDTDTNDTEIFRPGGGDMRDNWRSELYKYKVVTIEP
LGVAPTKAKRRVVQRE

gD signal sequence is underlined; mature N-terminal gD purification tag is italicized; linker sequence is in bold. V1/V2 domain is double underlined and V3 domain is underlined.

gD_MN468_rgp120; UCSC468
(SEQ ID NO: 11)
ATGGGGGGGGCTGCCGCCAGGTTGGGGGCCGTGATTTTGTTTGTCGTCATA
GTGGGCCTCCATGGGGTCCGCGGCAAATATGCCTTGGCGGATGCCTCTCTC
AAGATGGCCGACCCCAATCGATTTCGCGGCAAAGACCTTCCGGTCCTGGAC
CAGCTGCTCGAGGTACCTGTGTGGAAAGAAGCAACCACCACTCTATTTTGT
GCATCAGATGCTAAAGCATATGATACAGAGGCACATAATGTTTGGGCCACA
CATGCCTGTGTACCCACAGACCCCAACCCACAAGAAGTAGAATTGGTAAAT
GTGACAGAAAATTTTAACATGTGGAAAAATAACATGGTAGAACAGATGCAT
GAGGATATAATCAGTTTATGGGATCAAAGCCTAAAGCCATGTGTAAAATTA
ACCCCACTCTGTGTTACTTTAAATTGCACTGATTTGAGGAATACTACTAAT
ACCAATAATAGTACTGATAATAACAATAGTAAAAGCGAGGGAACAATAAAG
GGAGGAGAAATGAAAAACTGCTCTTTCAATATCACCACAAGCATAGGAGAT
AAGATGCAGAAAGAATATGCACTTCTTTATAAACTTGATATAGAACCAATA
GATAATGATAGTACCAGCTATAGGTTGATAAGTTGTAATACCTCAGTCATT
ACACAAGCTTGTCCAAAGATATCCTTTGAGCCAATTCCCATACACTATTGT
GCCCCGGCTGGTTTTGCGATTNTAAAGTGTAACGATAAAAAGTTCAGTGGA
AAAGGATCATGTAAAAATGTCAGCACAGTACAATGTACACATGGAATTAGG
CCAGTAGTATCAACTCAACTGCTGTTAAATGGCAGTCTAGCAGAAGAAGAG
GTAGTAATTAGATCTGAGGATTTCACTGATAATGCTAAAACCATCATAGTA
CATCTGAACGAATCTGTACAAATTAATTGTACAAGACCCAACAACAATACC
AGAAAAAGGATACATATAGGACCAGGGAGAGCATTTTATACAACAAAAAAT
ATAAAAGGAACTATAAGACAAGCACATTGTAACATTAGTAGAGCAAAATGG
AATGACACTTTAAGACAGATAGTTAGCAAGTTAAAAGAACAATTTAAGAAT
AAAACAATAGTCTTTAATCCATCCTCAGGAGGGGACCCAGAAATTGTAATG
CACAGTTTTAATTGTGGAGGGGAATTTTTCTACTGTAATACATCACCACTG
TTTAATAGTATTTGGAATGGTAATAATACTTGGAATAATACTACAGGGTCA
AATAACAATATCACACTTCAATGCAAAATAAAACAAATTATAAACATGTGG
CAGAAAGTAGGAAAAGCAATGTATGCCCCTCCCATTGAAGGACAAATTAGA
TGTTCATCAAATATTACAGGGCTACTATTAACAAGAGATGGTGGTGAGGAC
ACGGACACGAACGACACCGAGATCTTCAGACCTGGAGGAGGAGATATGAGG
GACAATTGGAGAAGTGAATTATATAAATATAAAGTAGTAACAATTGAACCA
TTAGGAGTAGCACCCACCAAGGCAAAGAGAAGAGTGGTGCAGAGAGAA

gD signal sequence encoding sequence is underlined; mature N-terminal gD purification tag encoding sequence is italicized; linker sequence encoding sequence is in bold.

gD_MN-rgp120_N301_N332
(SEQ ID NO: 12)
VPVWKEATTTLFCASDAKAYDTEAHNVWATHACVPTDPNPQEVELVNVTEN
FNMWKNNMVEQMHEDIISLWDQSLKPCVKLTPLCVTLNCTDLRNTTNTNNS
TDNNNSKSEGTIKGGEMKNCSFNITTSIGDKMQKEYALLYKLDIEPIDNDS
TSYRLISCNTSVITQACPKISFEPIPIHYCAPAGFAILKCNDKKFSGKGSC
KNVSTVQCTHGIRPVVSTQLLLNGSLAEEEVVIRSEDFTDNAKTIIVHLKE
SVQINCTRPNNNTRKRIHIGPGRAFYTTKNIKGTIRQAHCNISRAKWNDTL
RQIVSKLKEQFKNKTIVFNPSSGGDPEIVMHSFNCGGEFFYCNTSPLFNSI
WNGNNTWNNTTGSNNNITLQCKIKQIINMWQKVGKAMYAPPIEGQIRCSSN
ITGLLLTRDGGEDTDTNDTEIFRPGGGDMRDNWRSELYKYKVVTIEPLGVA
PT

V1/V2 domain is double underlined and V3 domain is underlined.

gD_MN-rgp120_N301_N332; UCSC 1320;
(SEQ ID NO: 13)
MGGAAARLGAVILFVVIVGLHGVRGKYALADASLKMADPNRFRGKDLP
VLDQLLEVPVWKEATTTLFCASDAKAYDTEAHNVWATHACVPTDPNPQ
EVELVNVTENFNMWKNNMVEQMHEDIISLWDQSLKPCVKLTPLCVTLN
CTDLRNTTNTNNSTDNNNSKSEGTIKGGEMKNCSFNITTSIGDKMQKE
YALLYKLDIEPIDNDSTSYRLISCNTSVITQACPKISFEPIPIHYCAP
AGFAILKCNDKKFSGKGSCKNVSTVQCTHGIRPVVSTQLLLNGSLAEE
EVVIRSEDFTDNAKTIIVHLKESVQINCTRPNNNTRKRIHIGPGRAFY
TTKNIKGTIRQAHCNISRAKWNDTLRQIVSKLKEQFKNKTIVFNPSSG
GDPEIVMHSFNCGGEFFYCNTSPLFNSIWNGNNTWNNTTGSNNNITLQ
CKIKQIINMWQKVGKAMYAPPIEGQIRCSSNITGLLLTRDGGEDTDTN
DTEIFRPGGGDMRDNWRSELYKYKVVTIEPLGVAPTKAKRRVVQRE

gD signal sequence is underlined; mature N-terminal gD purification tag is italicized; linker sequence is in bold. V1/V2 domain is double underlined and V3 domain is underlined.

gD-MN-rgp120_N301_N332; UCSC1320;
(SEQ ID NO: 14)
ATGGGGGGGGCTGCCGCCAGGTTGGGGGCCGTGATTTTGTTTGTCGTCA
TAGTGGGCCTCCATGGGGTCCGCGGCAAATATGCCTTGGCGGATGCCTC
TCTCAAGATGGCCGACCCCAATCGATTTCGCGGCAAAGACCTTCCGGTC
CTGGACCAGCTGCTCGAGGTACCTGTGTGGAAAGAAGCAACCACCACTC
TATTTTGTGCATCAGATGCTAAAGCATATGATACAGAGGCACATAATGT
TTGGGCCACACATGCCTGTGTACCCACAGACCCCAACCCACAAGAAGTA
GAATTGGTAAATGTGACAGAAAATTTTAACATGTGGAAAAATAACATGG
TAGAACAGATGCATGAGGATATAATCAGTTTATGGGATCAAAGCCTAAA
GCCATGTGTAAAATTAACCCCACTCTGTGTTACTTTAAATTGCACTGAT
TTGAGGAATACTACTAATACCAATAATAGTACTGATAATAACAATAGTA
AAAGCGAGGGAACAATAAAGGGAGGAGAAATGAAAAACTGCTCTTTCAA
TATCACCACAAGCATAGGAGATAAGATGCAGAAAGAATATGCACTTCTT
TATAAACTTGATATAGAACCAATAGATAATGATAGTACCAGCTATAGGT
TGATAAGTTGTAATACCTCAGTCATTACACAAGCTTGTCCAAAGATATC
CTTTGAGCCAATTCCCATACACTATTGTGCCCCGGCTGGTTTTGCGATT
CTAAAGTGTAACGATAAAAAGTTCAGTGGAAAAGGATCATGTAAAAATG
TCAGCACAGTACAATGTACACATGGAATTAGGCCAGTAGTATCAACTCA
ACTGCTGTTAAATGGCAGTCTAGCAGAAGAAGAGGTAGTAATTAGATCT
GAGGATTTCACTGATAATGCTAAAACCATCATAGTACATCTGAAAGAAT
CTGTACAAATTAATTGTACAAGACCCAACAACAATACCAGAAAAAGGAT
ACATATAGGACCAGGGAGAGCATTTTATACAACAAAAAATATAAAAGGA
ACTATAAGACAAGCACATTGTAACATTAGTAGAGCAAAATGGAATGACA
CTTTAAGACAGATAGTTAGCAAGTTAAAAGAACAATTTAAGAATAAAAC
AATAGTCTTTAATCCATCCTCAGGAGGGGACCCAGAAATTGTAATGCAC
AGTTTTAATTGTGGAGGGGAATTTTTCTACTGTAATACATCACCACTGT
TTAATAGTATTTGGAATGGTAATAATACTTGGAATAATACTACAGGGTC
AAATAACAATATCACACTTCAATGCAAAATAAAACAAATTATAAACATG
TGGCAGAAAGTAGGAAAAGCAATGTATGCCCCTCCCATTGAAGGACAAA
TTAGATGTTCATCAAATATTACAGGGCTACTATTAACAAGAGATGGTGG
TGAGGACACGGACACGAACGACACCGAGATCTTCAGACCTGGAGGAGGA
GATATGAGGGACAATTGGAGAAGTGAATTATATAAATATAAAGTAGTAA
CAATTGAACCATTAGGAGTAGCACCCACCAAGGCAAAGAGAAGAGTGGT
GCAGAGAGAA

Nucleotides encoding the gD signal sequence are underlined; nucleotides encoding the mature N-terminal gD purification tag are italicized; nucleotides encoding linker sequence are in bold.

gD_BAL-rgp120; codon optimized
(SEQ ID NO: 17)
VPVWKEATTTLFCASDAKAYDTEVHNVWATHACVPTDPNPQEVALENV
TENFNMWKNNMVEQMHEDIISLWDQSLKPCVKLTPLCVTLNCTDLRNA
TSRNVTNTTSSSRGMVGGGEMKNCSFNITTGIRGKVQKEYALFYELDI
VPIDNKIDRYRLISCNTSVITQACPKVSFEPIPIHYCAPAGFAILKCK
DKKFNGKGPCSNVSTVQCTHGIRPVVSTQLLLNGSLAEEEVVIRSENF
TNNAKTIIVQLNESVEINCTRPNNNTRKSINIGPGRAFYTTGEIIGDI
RQAHCNLSRAKWNDTLNKIVIKLREQFGNKTIVFKHSSGGDPEIVTHS
FNCGGEFFYCNSTQLFNSTWNVTEESNNTVENNTITLPCRIKQIINMW
QEVGRAMYAPPIRGQIRCSSNITGLLLTRDGGPEDNKTEVFRPGGGDM
RDNWRSELYKYKVVKIEPLGVAPTKAKRRVVQRE

V1/V2 domain is double underlined and V3 domain is underlined.

gD_BAL-rgp120; UCSC 1375; codon optimized
(SEQ ID NO: 18)
MGGAAARLGAVILFVVIVGLHGVRGKYALADASLKMADPNRFRGKDLPV
LDQLLEVPVWKEATTTLFCASDAKAYDTEVHNVWATHACVPTDPNPQEV
ALENVTENFNMWKNNMVEQMHEDIISLWDQSLKPCVKLTPLCVTLNCTD
LRNATSRNVTNTTSSSRGMVGGGEMKNCSFNITTGIRGKVQKEYALFYE
LDIVPIDNKIDRYRLISCNTSVITQACPKVSFEPIPIHYCAPAGFAILK
CKDKKFNGKGPCSNVSTVQCTHGIRPVVSTQLLLNGSLAEEEVVIRSEN
FTNNAKTIIVQLNESVEINCTRPNNNTRKSINIGPGRAFYTTGEIIGDI
RQAHCNLSRAKWNDTLNKIVIKLREQFGNKTIVFKHSSGGDPEIVTHSF
NCGGEFFYCNSTQLFNSTWNVTEESNNTVENNTITLPCRIKQIINMWQE
VGRAMYAPPIRGQIRCSSNITGLLLTRDGGPEDNKTEVFRPGGGDMRDN
WRSELYKYKVVKIEPLGVAPTKAKRRVVQRE

gD signal sequence is underlined; mature N-terminal gD purification tag is italicized; linker sequence is in bold.

gD_BAL-rgp120; UCSC 1375; codon optimized
(SEQ ID NO: 19)
ATGGGGGGGGCTGCCGCCAGGTTGGGGGCCGTGATTTTGTTTGTCGTCAT
AGTGGGCCTCCATGGGGTCCGCGGCAAATATGCCTTGGCGGATGCCTCTC
TCAAGATGGCCGACCCCAATCGATTTCGCGGCAAAGACCTTCCGGTCCTG
GACCAGCTGCTGGAGGTACCTGTGTGGAAAGAGGCCACCACCACACTGTT
CTGTGCCTCCGATGCCAAGGCCTACGATACCGAGGTGCACAACGTGTGGG
CCACTCATGCCTGCGTGCCCACCGATCCTAATCCTCAAGAAGTGGCCCTG
GAAAACGTGACCGAGAACTTCAACATGTGGAAGAACAACATGGTCGAGCA
GATGCACGAGGACATCATCAGCCTGTGGGACCAGAGCCTGAAGCCTTGCG
TGAAGCTGACCCCTCTGTGCGTGACCCTGAACTGCACCGACCTGAGAAAC
GCCACCAGCCGGAACGTGACCAATACCACCTCTAGCAGCAGAGGCATGGT
TGGAGGCGGCGAGATGAAGAACTGCAGCTTCAACATCACCACCGGCATCA
GAGGCAAGGTGCAGAAAGAGTACGCCCTGTTCTACGAGCTGGACATCGTG
CCCATCGACAACAAGATCGACCGGTACAGACTGATCAGCTGCAACACCAG
CGTGATCACCCAGGCCTGTCCTAAGGTGTCCTTCGAGCCCATTCCTATCC
ACTACTGTGCCCCTGCCGGCTTCGCCATCCTGAAGTGCAAGGACAAGAAG
TTCAACGGCAAGGGCCCCTGCAGCAACGTGTCCACAGTGCAGTGTACACA
CGGCATCAGGCCCGTGGTGTCTACACAGCTGCTGCTGAATGGCAGCCTGG
CCGAGGAAGAGGTGGTCATCAGAAGCGAGAATTTCACCAACAACGCCAAG
ACCATCATCGTGCAGCTGAACGAGAGCGTGGAAATCAACTGCACCCGGCC
TAACAACAACACCCGGAAGTCCATCAACATCGGCCCTGGCAGAGCCTTCT
ACACAACCGGCGAGATCATCGGCGACATCAGACAGGCCCACTGCAACCTG
TCTCGGGCCAAGTGGAACGACACCCTGAACAAGATTGTGATCAAGCTGAG
AGAGCAGTTCGGCAACAAGACGATCGTGTTCAAGCACAGCTCTGGCGGCG
ACCCTGAGATCGTGACCCACAGCTTTAATTGTGGCGGCGAGTTCTTCTAC
TGCAACAGCACCCAGCTGTTCAACTCCACCTGGAATGTGACCGAGGAAAG
CAACAATACCGTCGAGAACAACACCATCACACTGCCCTGCCGGATCAAGC
AGATCATCAATATGTGGCAAGAAGTCGGCAGGGCTATGTACGCCCCTCCT
ATCAGAGGCCAGATCCGGTGCAGCAGCAATATCACAGGCCTGCTGCTCAC
CAGAGATGGCGGCCCTGAGGATAACAAGACCGAGGTGTTCAGACCCGGCG
GAGGCGACATGAGAGACAATTGGAGAAGCGAGCTGTACAAGTACAAGGTG
GTCAAGATCGAGCCCCTGGGCGTCGCCCCTACAAAGGCTAAGAGAAGAGT
GGTGCAGCGGGAA

TZ97008-rgp120; UCSC 1374; codon optimized
(SEQ ID NO: 23)
MGGAAARLGAVILFVVIVGLHGVRGKYALADASLKMADPNRFRGKDLP
VLDQLLEVPVWKEAKTTLFCASEAKGYEKEVHNVWATHACVPTDPSPH
ELVLENVTENFNMWENDMVDQMHEDIISLWDQSLKPCVKLTPLCVTLN
CTNVTGTNVTGNDMKGEMTNCSFNATTEIKDRKKNVYALFYKLDVVQL
EGNSSNSTYSTYRLINCNTSVITQACPKVSFDPIPIHYCAPAGYAILK
CNNKTFNGTGPCNNVSTVQCTHGIKPVVSTQLLLNGSLAEKEIVIRSK

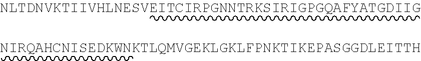

SFNCRGEFFYCNTTKLFNSTYRPNANANSSSSNNTITLQCKIKQIINM
WQEVGRAMYAPPIAGNITCTSNITGLLLVRDGGNNSTEEEIFRPGGGN

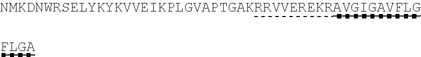

gD signal sequence is underlined; mature N-terminal gD purification tag is italicized; linker sequence is in bold. Dotted line ( [symbol] ) Location of basic residues that are targets for furin and trypsin like enzymes. Translational stop codons for C-terminal purification tags can be incorporated at the beginning to this sequence. If a C-terminal purification tag may be included, then stop codon can be inserted at either the beginning or end of the sequence. Broken line ( [symbol] ): C-terminal or 3′ sequences not required for expression. V1/V2 domain is double underlined and V3 domain is indicated with a wavy line.

TZ97008-rgp120; UCSC1374; codon optimized
(SEQ ID NO: 24)
ATGGGGGGGGCTGCCGCCAGGTTGGGGGCCGTGATTTTGTTTGTCGTC
ATAGTGGGCCTCCATGGGGTCCGCGGCAAATATGCCTTGGCGGATGCC
TCTCTCAAGATGGCCGACCCCAATCGATTTCGCGGCAAAGACCTTCCG
GTCCTGGACCAGCTGCTGGAGGTACCAGTGTGGAAAGAGGCCAAGACC
ACACTGTTCTGTGCCAGCGAGGCCAAGGGCTACGAGAAAGAGGTGCAC
AACGTCTGGGCCACACACGCCTGTGTGCCTACCGATCCTTCTCCTCAC
GAACTGGTGCTGGAAAACGTGACCGAGAACTTCAACATGTGGGAGAAC
GACATGGTGGACCAGATGCACGAGGACATCATCAGCCTGTGGGACCAG
AGCCTGAAGCCTTGCGTGAAGCTGACCCCTCTGTGCGTGACCCTGAAC
TGCACCAATGTGACCGGCACCAACGTGACAGGGAACGATATGAAGGGC
GAGATGACCAACTGCAGCTTCAACGCCACCACCGAGATCAAGGACCGG
AAGAAAAACGTGTACGCCCTGTTCTACAAGCTGGACGTGGTGCAGCTG
GAAGGCAACAGCAGCAACTCCACCTACAGCACCTACCGGCTGATCAAC
TGCAACACCAGCGTGATCACCCAGGCCTGTCCTAAGGTGTCCTTCGAT
CCCATTCCTATCCACTACTGTGCCCCTGCCGGCTACGCCATCCTGAAG
TGCAACAACAAGACCTTCAACGGCACAGGCCCCTGCAACAACGTGTCC
ACCGTGCAGTGTACCCACGGCATCAAGCCAGTGGTGTCCACACAGCTG
CTGCTGAATGGAAGCCTGGCCGAGAAAGAAATCGTGATCAGAAGCAAG
AACCTGACCGACAACGTCAAGACCATCATCGTGCACCTGAACGAGAGC
GTGGAAATCACCTGTATCAGACCCGGCAACAACACCAGAAAGAGCATC
AGAATCGGCCCAGGCCAGGCCTTTTATGCCACCGGCGATATCATCGGC
AACATCAGACAGGCCCACTGTAACATCAGCGAGGACAAGTGGAACAAG
ACCCTGCAGATGGTCGGAGAGAAGCTGGGCAAGCTGTTCCCCAACAAG
ACAATCAAGTTCGAGCCCGCCTCTGGCGGCGACCTGGAAATTACCACA
CACAGCTTCAATTGTCGGGGCGAGTTCTTCTACTGCAATACCACCAAG
CTGTTTAATAGCACCTACAGGCCCAACGCCAATGCCAACAGCTCCAGC
TCCAACAACACTATCACCCTGCAGTGCAAGATCAAGCAGATCATCAAT
ATGTGGCAAGAAGTCGGCAGGGCTATGTACGCCCCTCCTATCGCCGGC
AACATTACCTGCACCAGCAACATCACAGGCCTGCTGCTCGTTAGAGAT
GGCGGCAACAATAGCACCGAGGAAGAGATCTTCAGACCTGGCGGCGGA
AACATGAAGGACAACTGGCGGAGCGAGCTGTACAAGTACAAGGTGGTC
GAGATTAAGCCCCTGGGCGTTGCACCTACTGGCGCCAAGAGAAGAGTG

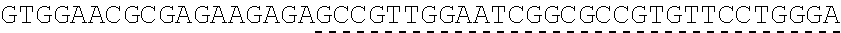

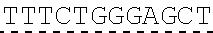

gD signal sequence encoding sequence is underlined; mature N-terminal gD purification tag is italicized; linker sequence is in bold. Dotted line ( [symbol] ): C-terminal or 3′ sequences not required for expression.

CN97001_D179N-rgp120 codon optimized
(SEQ ID NO: 25)
VPVWKEATTTLFCASDAKAYDTEVRNVWATHACVPADPNPQEMVLENVT
ENFNMWKNEMVNQMQEDVISLWDQSLKPCVKLTPLCVTLECRNVSSNSN
GAHNETYHESMKEMKNCSFNATTVVRDRKQTVYALFYRLNIVPLTKKNS
SENSSEYYRLINCNTSAITQACPKVTFDPIPIHYCTPAGYAILKCNDKI
FNGTGPCHNVSTVQCTHGIKPVVSTQLLLNGSLAEGEIIIRSENLTNNV
KTIIVHLNQSVEIVCTRPGNNTRKSIRIGPGQTFYATGDIIGDIRQAHC
NISEDKWNETLQRVSKKLAEHFQNKTIKFASSSGGDLEITTHSFNCRGE
FFYCNTSGLFNGTYTPNGTKSNSSSIITIPCRIKQIINMWQEVGRAMYA
PPIEGNITCKSNITGLLLVRDGGTEPNDTETFRPGGGDMRNNWRSELYK
YKVVEIKPLGVAPTTA

V1/V2 domain is double underlined and V3 domain is underlined.

CN97001_ D179N-rgp120; UCSC199; codon optimized
(SEQ ID NO: 26)
MGGAAARLGAVILFVVIVGLHGVRGKYALADASLKMADPNRFRGKDLPV
LDQLLEVPVWKEATTTLFCASDAKAYDTEVRNVWATHACVPADPNPQEM
VLENVTENFNMWKNEMVNQMQEDVISLWDQSLKPCVKLTPLCVTLECRN
VSSNSNGAHNETYHESMKEMKNCSFNATTVVRDRKQTVYALFYRLNIVP
LTKKNSSENSSEYYRLINCNTSAITQACPKVTFDPIPIHYCTPAGYAIL
KCNDKIFNGTGPCHNVSTVQCTHGIKPVVSTQLLLNGSLAEGEIIIRSE
NLTNNVKTIIVHLNQSVEIVCTRPGNNTRKSIRIGPGQTFYATGDIIGD
IRQAHCNISEDKWNETLQRVSKKLAEHFQNKTIKFASSSGGDLEITTHS
FNCRGEFFYCNTSGLFNGTYTPNGTKSNSSSIITIPCRIKQIINMWQEV
GRAMYAPPIEGNITCKSNITGLLLVRDGGTEPNDTETFRPGGGDMRNNW

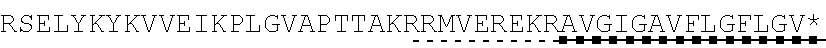

gD signal sequence is underlined; mature N-terminal gD purification tag is italicized; linker sequence is in bold. Dotted line ( [symbol] ): Location of basic residues that are targets for furin and trypsin like enzymes. Translational stop codons for C-terminal purification tags can be incorporated at the beginning to this sequence. If a C-terminal purification tag may be included, then stop codon can be inserted at either the beginning or end of the sequence. Broken line ( [symbol] ): C-terminal or 3′ sequences not required for expression. * indicates location to insert translational stop codon or C-terminal purification Tag.

CN97001_D179N-rgp120; UCSC 199; codon optimized
(SEQ ID NO: 27):
ATGGGGGGGGCTGCCGCCAGGTTGGGGGCCGTGATTTTGTTTGTCGTCA
TAGTGGGCCTCCATGGGGTCCGCGGCAAATATGCCTTGGCGGATGCCTC
TCTCAAGATGGCCGACCCCAATCGATTTCGCGGCAAAGACCTTCCGGTC
CTGGACCAGCTGCTGGAGGTACCAGTGTGGAAGGAAGCCACCACAACCC
TCTTCTGCGCCAGCGATGCCAAGGCCTACGACACGGAGGTCCGCAATGT
GTGGGCCACCCATGCCTGTGTGCCCGCCGACCCCAACCCCCAGGAGATG
GTCCTGGAGAATGTCACGGAGAACTTCAACATGTGGAAGAACGAGATGG
TGAACCAGATGCAGGAGGACGTCATCTCCCTGTGGGACCAGAGCCTGAA
ACCCTGCGTCAAACTGACACCCCTCTGTGTGACCCTGGAGTGCAGGAAC
GTGTCCTCCAACAGCAACGGCGCCCACAACGAGACCTACCACGAAAGCA
TGAAAGAGATGAAGAACTGCAGCTTCAATGCCACAACCGTGGTGCGGGA
CCGGAAGCAGACGGTGTACGCGTTGTTCTACCGGCTGAATATCGTCCCC
CTCACGAAGAAAAATTCCAGCGAGAACTCCTCCGAGTATTATCGCCTGA
TCAACTGCAACACCAGCGCCATCACGCAGGCCTGCCCCAAAGTGACCTT
CGACCCCATCCCCATCCACTACTGCACCCCAGCTGGGTACGCCATCCTG
AAGTGCAATGACAAAATCTTCAACGGCACAGGCCCCTGCCACAATGTGA
GCACCGTCCAGTGCACCCACGGCATCAAGCCAGTGGTCTCCACCCAGCT
CCTCCTGAATGGGAGCCTGGCAGAGGGCGAGATCATCATCCGCTCCGAG
AACCTGACCAACAATGTCAAGACCATCATCGTCCACCTGAATCAGTCCG
TGGAGATCGTCTGCACCAGACCCGGCAACAACACGCGGAAAAGCATCCG
CATCGGCCCAGGGCAGACCTTCTATGCCACGGGGGACATCATTGGGGAC
ATCAGGCAGGCCCACTGCAACATCAGCGAAGACAAGTGGAACGAAACCC
TGCAGCGGGTGTCCAAAAAACTCGCCGAGCACTTCCAGAACAAGACGAT
CAAGTTCGCATCCTCCAGCGGGGGGGACCTGGAGATCACCACGCACAGC
TTCAACTGCCGGGGGGAATTTTTCTACTGCAACACCTCCGGGCTGTTCA
ACGGGACCTACACCCCCAACGGCACCAAGTCCAACTCCAGCAGCATCAT
CACCATCCCATGCAGGATCAAGCAGATCATCAACATGTGGCAGGAGGTG
GGCCGGGCCATGTACGCCCCCCCCATCGAGGGCAATATCACCTGCAAGT
CCAACATCACGGGGCTGCTGCTGGTGCGGGATGGGGGGACCGAGCCCAA
CGACACCGAGACCTTCAGGCCAGGGGGGGGGGATATGCGGAACAACTGG
CGCAGCGAGCTCTACAAGTACAAAGTGGTGGAGATCAAACCCCTGGGGG
TGGCCCCCACCACAGCCAAACGCAGGATGGTGGAGCGGGAGAAGCGGGC
AGTGGGCATTGGGGCCGTGTTCTTGGGCTTCCTtGGCGtG

gD signal sequence encoding sequence is underlined; mature N-terminal gD purification tag encoding sequence is italicized; linker sequence encoding sequence is in bold.

A244_N334-rgp140; codon optimized
(SEQ ID NO: 5)
MRVKETQMNWPNLWKWGTLILGLVIICSASDNLWVTVYYGVPVWKEADT
TLFCASDAKAHETEVHNVWATHACVPTDPNPQEIDLENVTENFNMWKNN
MVEQMQEDVISLWDQSLKPCVKLTPLCVTLHCTNANLTKANLTNVNNRT
NVSNIIGNITDEVRNCSFNMTTELRDKKQKVHALFYKLDIVPIEDNNDS
SEYRLINCNTSVIKQACPKISFDPIPIHYCTPAGYAILKCNDKNFNGTG
PCKNVSSVQCTHGIKPVVSTQLLLNGSLAEEEIIIRSDNLTNNAKTIIV
HLNKSVVINCTRPSNNTRTSITIGPGQVFYRTGDIIGDIRKAYCEINGT
EWNKALKQVTEKLKEHFNNKPIIFQPPSGGDLEITMHHFNCRGEFFYCN
TTRLFNNTCIANGTIEGCNGNITLPCKIKQIINMWQGAGQAMYAPPISG
TINCVSNITGILLTRDGGATNNTNNETIRPGGGNIKDNWRNELYKYKVV
QIEPLGVAPTRAKRRVVEREKRAVGIGAMIFGFLGAAGSTMGAASITLT
VQARQLLSGIVQQQSNLLRAIEAQQHLLQLTVWGIKQLQARVLAVERYL
KDQKFLGLWGCSGKIICTTAVPWNSTWSNKSLEEIWSNMTWIEWEREIS
NYTNQIYEILTKSQDQQDRNEKDLLELDKWASLWTWFDITNWLWYIK

Wild type HIV signal sequence is underlined. Mature N-terminal HIV envelope sequences for gp140 trimers is italicized.

A244_N334-rgp140; codon optimized
(SEQ ID NO: 6)
ATGAGAGTGAAGGAGACACAGATGAATTGGCCAAACTTGTGGAAATGGG
GGACTTTGATCCTTGGGTTGGTGATAATTTGTAGTGCCTCAGACAACTT
GTGGGTTACAGTTTATTATGGGGTACCAGTGTGGAAGGAAGCCGACACA
ACCCTCTTCTGCGCCAGCGATGCCAAGGCCCACGAGACGGAGGTCCACA
ATGTGTGGGCCACCCATGCCTGTGTGCCCACGGACCCCAACCCCCAGGA
GATTGACCTGGAGAATGTCACGGAGAACTTCAACATGTGGAAGAACAAC
ATGGTGGAGCAGATGCAGGAGGACGTCATCTCCCTGTGGGACCAGAGCC
TGAAACCCTGCGTCAAACTGACACCCCCCTGTGTGACCCTGCACTGCAC
GAACGCCAACCTGACCAAGGCCAACCTCACCAACGTGAACAATCGGACC
AACGTGTCCAACATCATCGGGAACATCACAGATGAGGTGAGGAACTGCA
GCTTCAATATGACAACCGAGCTCCGGGACAAAAAGCAGAAGGTGCACGC
GTTGTTCTACAAACTGGATATCGTCCCCATCGAGGACAATAATGACAGC
TCCGAGTATCGCCTGATCAACTGCAACACCAGCGTCATCAAACAGGCCT
GCCCCAAAATTTCCTTCGACCCCATCCCCATCCACTACTGCACCCCAGC
TGGGTACGCCATCCTGAAGTGCAATGACAAGAACTTCAACGGCACAGGG
CCCTGCAAGAATGTGAGCTCCGTCCAGTGCACCCACGGCATCAAGCCAG
TGGTCTCCACCCAGCTCCTCCTGAATGGGAGCCTGGCAGAGGAAGAGAT
CATCATCCGCTCCGAGAACCTGACCAACAATGCCAAGACCATCATCGTC
CACCTGAATAAGTCCGTGGTCATCAACTGCACCAGACCCAGCAACAACA
CGCGGACCAGCATCACCATCGGCCCAGGGCAGGTCTTCTATAGGACGGG
GGACATCATTGGGGACATCAGGAAGGCCTACTGCGAAATCAATGGGACC
GAGTGGAACAAAGCCCTGAAACAGGTGACCGAAAAACTCAAGGAGCACT
TCAACAACAAGCCAATCATCTTCCAGCCCCCCAGCGGGGGGGACCTGGA
GATCACCATGCACCATTTCAACTGCCGGGGGGAATTCTTCTACTGCAAC
ACCACCCGCCTGTTCAACAACACCTGCATCGCCAACGGCACCATCGAGG
GCTGCAATGGCAACATCACCCTCCCATGCAAAATCAAGCAGATCATCAA
CATGTGGCAGGGGGCAGGCCAGGCCATGTACGCCCCCCCCATCTCCGGC
ACGATCAACTGCGTGTCCAACATCACGGGGATCCTGCTGACCCGGGATG
GGGGGGCTACCAACAATACGAACAATGAGACCTTCAGGCCAGGGGGGGG
GAACATCAAAGACAACTGGCGCAATGAGCTCTACAAGTACAAAGTGGTG
CAGATCGAGCCCCTGGGGGTGGCCCCCACCCGGGCCAAACGCAGGGTGG
TGGAGCGGGAGAAGCGGGCAGTGGGCATTGGGGCCATGATCTTCGGCTT
TCTGGGAGCCGCCGGATCTACAATGGGAGCTGCCAGCATCACCCTGACC
GTGCAGGCTAGACAACTGCTGTCTGGCATCGTGCAGCAGCAGAGCAATC
TGCTGAGAGCCATTGAGGCCCAGCAGCATCTGCTGCAGCTGACAGTGTG
GGGCATCAAACAGCTGCAGGCCAGAGTGCTGGCCGTGGAAAGATACCTG
AAGGACCAGAAATTCCTCGGCCTGTGGGGCTGCAGCGGCAAGATCATCT
GTACAACAGCCGTGCCTTGGAACAGCACCTGGTCCAACAAGAGCCTGGA
AGAGATCTGGTCCAATATGACCTGGATCGAGTGGGAGAGAGAGATCAGC
AACTACACCAACCAGATCTACGAGATCCTGACCAAGAGCCAGGACCAGC
AGGACCGGAACGAGAAGGATCTGCTGGAACTGGACAAGTGGGCCAGCCT
GTGGACTTGGTTTGACATCACCAACTGGCTGTGGTACATCAAG

Wild type HIV signal sequence encoding nucleic acid sequence is underlined. Mature N-terminal HIV envelope sequences encoding nucleic acid sequence for gp140 trimers is italicized.

A244_N332-rgp140
(SEQ ID NO: 7)
MRVKETQMNWPNLWKWGTLILGLVIICSASDNLWVTVYYGVPVWKEADT
TLFCASDAKAHETEVHNVWATHACVPTDPNPQEIDLENVTENFNMWKNN
MVEQMQEDVISLWDQSLKPCVKLTPLCVTLHCTNANLTKANLTNVNNRT
NVSNIIGNITDEVRNCSFNMTTELRDKKQKVHALFYKLDIVPIEDNNDS
SEYRLINCNTSVIKQACPKISFDPIPIHYCTPAGYAILKCNDKNFNGTG
PCKNVSSVQCTHGIKPVVSTQLLLNGSLAEEEIIIRSDNLTNNAKTIIV
HLNKSVVINCTRPSNNTRTSITIGPGQVFYRTGDIIGDIRKAYCNISGT
EWNKALKQVTEKLKEHFNNKPIIFQPPSGGDLEITMHHFNCRGEFFYCN
TTRLFNNTCIANGTIEGCNGNITLPCKIKQIINMWQGAGQAMYAPPISG
TINCVSNITGILLTRDGGATNNTNNETPRPGGGNIKDNWRNELYKYKVV
QIEPLGVAPTRAKRRVVEREKRAVGIGAMIFGFLGAAGSTMGAASITLT
VQARQLLSGIVQQQSNLLRAIEAQQHLLQLTVWGIKQLQARVLAVERYL
KDQKFLGLWGCSGKIICTTAVPWNSTWSNKSLEEIWSNMTWIEWEREIS
NYTNQIYEILTKSQDQQDRNEKDLLELDKWASLWTWFDITNWLWYIK

Wild type HIV signal sequence is underlined. Mature N-terminal HIV envelope sequences for gp140 trimers is italicized.

A244_N332-rgp140; codon optimized
(SEQ ID NO: 8)
ATGAGAGTGAAGGAGACACAGATGAATTGGCCAAACTTGTGGAAATGGG
GGACTTTGATCCTTGGGTTGGTGATAATTTGTAGTGCCTCAGACAACTT
GTGGGTTACAGTTTATTATGGGGTACCAGTGTGGAAGGAAGCCGACACA
ACCCTCTTCTGCGCCAGCGATGCCAAGGCCCACGAGACGGAGGTCCACA
ATGTGTGGGCCACCCATGCCTGTGTGCCCACGGACCCCAACCCCCAGGA
GATTGACCTGGAGAATGTCACGGAGAACTTCAACATGTGGAAGAACAAC
ATGGTGGAGCAGATGCAGGAGGACGTCATCTCCCTGTGGGACCAGAGCC
TGAAACCCTGCGTCAAACTGACACCCCCCTGTGTGACCCTGCACTGCAC
GAACGCCAACCTGACCAAGGCCAACCTCACCAACGTGAACAATCGGACC
AACGTGTCCAACATCATCGGGAACATCACAGATGAGGTGAGGAACTGCA
GCTTCAATATGACAACCGAGCTCCGGGACAAAAAGCAGAAGGTGCACGC
GTTGTTCTACAAACTGGATATCGTCCCCATCGAGGACAATAATGACAGc
TCCGAGTATCGCCTGATCAACTGCAACACCAGcGTCATCAAACAGGCCT
GCCCCAAAATTTCCTTCGACCCCATCCCCATCCACTACTGCACCCCAGC
TGGGTACGCCATCCTGAAGTGCAATGACAAGAACTTCAACGGCACAGGG
CCCTGCAAGAATGTGAGCTCCGTCCAGTGCACCCACGGCATCAAGCCAG
TGGTCTCCACCCAGCTCCTCCTGAATGGGAGCCTGGCAGAGGAAGAGAT
CATCATCCGCTCCGAGAACCTGACCAACAATGCCAAGACCATCATCGTC
CACCTGAATAAGTCCGTGGTCATCAACTGCACCAGACCCAGCAACAACA
CGCGGACCAGCATCACCATCGGCCCAGGGCAGGTCTTCTATAGGACGGG
GGACATCATTGGGGACATCAGGAAGGCCTACTGCAACATCAGTGGGACC
GAGTGGAACAAAGCCCTGAAACAGGTGACCGAAAAACTCAAGGAGCACT
TCAACAACAAGCCAATCATCTTCCAGCCCCCCAGCGGGGGGGACCTGGA
GATCACCATGCACCATTTCAACTGCCGGGGGGAATTCTTCTACTGCAAC
ACCACCCGCCTGTTCAACAACACCTGCATCGCCAACGGCACCATCGAGG
GCTGCAATGGCAACATCACCCTCCCATGCAAAATCAAGCAGATCATCAA
CATGTGGCAGGGGGCAGGCCAGGCCATGTACGCCCCCCCCATCTCCGGC
ACGATCAACTGCGTGTCCAACATCACGGGGATCCTGCTGACCCGGGATG
GGGGGGCTACCAACAATACGAACAATGAGACCTTCAGGCCAGGGGGGGG
GAACATCAAAGACAACTGGCGCAATGAGCTCTACAAGTACAAAGTGGTG
CAGATCGAGCCCCTGGGGGTGGCCCCCACCCGGGCCAAACGCAGGGTGG
TGGAGCGGGAGAAGCGGGCAGTGGGCATTGGGGCCATGATCTTCGGCTT
TCTGGGAGCCGCCGGATCTACAATGGGAGCTGCCAGCATCACCCTGACC
GTGCAGGCTAGACAACTGCTGTCTGGCATCGTGCAGCAGCAGAGCAATC
TGCTGAGAGCCATTGAGGCCCAGCAGCATCTGCTGCAGCTGACAGTGTG
GGGCATCAAACAGCTGCAGGCCAGAGTGCTGGCCGTGGAAAGATACCTG
AAGGACCAGAAATTCCTCGGCCTGTGGGGCTGCAGCGGCAAGATCATCT
GTACAACAGCCGTGCCTTGGAACAGCACCTGGTCCAACAAGAGCCTGGA
AGAGATCTGGTCCAATATGACCTGGATCGAGTGGGAGAGAGAGATCAGC
AACTACACCAACCAGATCTACGAGATCCTGACCAAGAGCCAGGACCAGC
AGGACCGGAACGAGAAGGATCTGCTGGAACTGGACAAGTGGGCCAGCCT
GTGGACTTGGTTTGACATCACCAACTGGCTGTGGTACATCAAG

Wild type HIV signal sequence encoding nucleic acid sequence is underlined. Mature N-terminal HIV envelope sequences encoding nucleic acid sequence for gp140 trimers is italicized.

MN-rgp140-N301_N332;
(SEQ ID NO: 15)
MRVKGIRRNYQHWWGWGTMLLGLLMICSATEKLWVTVYYGVPVWKEATT
TLFCASDAKAYDTEAHNVWATHACVPTDPNPQEVELVNVTENFNMWKNN
MVEQMHEDIISLWDQSLKPCVKLTPLCVTLNCTDLRNTTNTNNSTDNNN
SKSEGTIKGGEMKNCSFNITTSIGDKMQKEYALLYKLDIEPIDNDSTSY
RLISCNTSVITQACPKISFEPIPIHYCAPAGFAILKCNDKKFSGKGSCK
NVSTVQCTHGIRPVVSTQLLLNGSLAEEEVVIRSEDFTDNAKTIIVHLK
ESVQINCTRPNNNTRKRIHIGPGRAFYTTKNIKGTIRQAHCNISRAKWN
DTLRQIVSKLKEQFKNKTIVFNPSSGGDPEIVMHSFNCGGEFFYCNTSP
LFNSIWNGNNTWNNTTGSNNNITLQCKIKQIINMWQKVGKAMYAPPIEG
QIRCSSNITGLLLTRDGGEDTDTNDTEIFRPGGGDMRDNWRSELYKYKV
VTIEPLGVAPTKAKRRVVQREKRAAIGALFLGFLGAAGSTMGAASVTLT
VQARLLLSGIVQQQNNLLRAIEAQQHMLQLTVWGIKQLQARVLAVERYL
KDQQLLGFWGCSGKLICTTTVPWNASWSNKSLDDIWNNMTWMQWEREID
NYTSLIYSLLEKSQTQQEKNEQELLELDKWASLWNWFDITNWLWYIK

Wild type HIV signal sequence is underlined. Mature N-terminal HIV envelope sequences for gp140 trimers is italicized.

MN-rgp140_N301_N332;
(SEQ ID NO: 16)
ATGAGAGTGAAGGGGATCAGGAGGAATTATCAGCACTGGTGGGGATGGG
GCACGATGCTCCTTGGGTTATTAATGATCTGTAGTGCTACAGAAAAATT
GTGGGTCACAGTCTATTATGGGGTACCTGTGTGGAAAGAAGCAACCACC
ACTCTATTTTGTGCATCAGATGCTAAAGCATATGATACAGAGGCACATA
ATGTTTGGGCCACACATGCCTGTGTACCCACAGACCCCAACCCACAAGA
AGTAGAATTGGTAAATGTGACAGAAAATTTTAACATGTGGAAAAATAAC
ATGGTAGAACAGATGCATGAGGATATAATCAGTTTATGGGATCAAAGCC
TAAAGCCATGTGTAAAATTAACCCCACTCTGTGTTACTTTAAATTGCAC
TGATTTGAGGAATACTACTAATACCAATAATAGTACTGATAATAACAAT
AGTAAAAGCGAGGGAACAATAAAGGGAGGAGAAATGAAAAACTGCTCTT
TCAATATCACCACAAGCATAGGAGATAAGATGCAGAAAGAATATGCACT
TCTTTATAAACTTGATATAGAACCAATAGATAATGATAGTACCAGCTAT
AGGTTGATAAGTTGTAATACCTCAGTCATTACACAAGCTTGTCCAAAGA
TATCCTTTGAGCCAATTCCCATACACTATTGTGCCCCGGCTGGTTTTGC
GATTCTAAAGTGTAACGATAAAAAGTTCAGTGGAAAAGGATCATGTAAA
AATGTCAGCACAGTACAATGTACACATGGAATTAGGCCAGTAGTATCAA
CTCAACTGCTGTTAAATGGCAGTCTAGCAGAAGAAGAGGTAGTAATTAG
ATCTGAGGATTTCACTGATAATGCTAAAACCATCATAGTACATCTGAAA
GAATCTGTACAAATTAATTGTACAAGACCCAACAACAATACCAGAAAAA
GGATACATATAGGACCAGGGAGAGCATTTTATACAACAAAAAATATAAA
AGGAACTATAAGACAAGCACATTGTAACATTAGTAGAGCAAAATGGAAT
GACACTTTAAGACAGATAGTTAGCAAGTTAAAAGAACAATTTAAGAATA
AAACAATAGTCTTTAATCCATCCTCAGGAGGGGACCCAGAAATTGTAAT
GCACAGTTTTAATTGTGGAGGGGAATTTTTCTACTGTAATACATCACCA
CTGTTTAATAGTATTTGGAATGGTAATAATACTTGGAATAATACTACAG
GGTCAAATAACAATATCACACTTCAATGCAAAATAAAACAAATTATAAA
CATGTGGCAGAAAGTAGGAAAAGCAATGTATGCCCCTCCCATTGAAGGA
CAAATTAGATGTTCATCAAATATTACAGGGCTACTATTAACAAGAGATG
GTGGTGAGGACACGGACACGAACGACACCGAGATCTTCAGACCTGGAGG
AGGAGATATGAGGGACAATTGGAGAAGTGAATTATATAAATATAAAGTA
GTAACAATTGAACCATTAGGAGTAGCACCCACCAAGGCAAAGAGAAGAG
TGGTGCAGAGAGAAAAAAGAGCAGCGATAGGAGCTCTGTTCCTTGGGTT
CTTAGGAGCAGCAGGAAGCACTATGGGCGCAGCGTCAGTGACGCTGACG
GTACAGGCCAGACTATTATTGTCTGGTATAGTGCAACAGCAGAACAATT
TGCTGAGGGCCATTGAGGCGCAACAGCATATGTTGCAACTCACAGTCTG
GGGCATCAAGCAGCTCCAGGCAAGAGTCCTGGCTGTGGAAAGATACCTA
AAGGATCAACAGCTCCTGGGGTTTTGGGGTTGCTCTGGAAAACTCATTT
GCACCACTACTGTGCCTTGGAATGCTAGTTGGAGTAATAAATCTCTGGA
TGATATTTGGAATAACATGACCTGGATGCAGTGGGAAAGAGAAATTGAC
AATTACACAAGCTTAATATACTCATTACTAGAAAAATCGCAAACCCAAC
AAGAAAAGAATGAACAAGAATTATTGGAATTGGATAAATGGGCAAGTTT
GTGGAATTGGTTTGACATAACAAATTGGCTGTGGTATATAAAA

Wild type HIV signal sequence encoding nucleic acid sequence is underlined. Mature N-terminal HIV envelope sequences encoding nucleic acid sequence for gp140 trimers is italicized.

BAL-rgp140
(SEQ ID NO: 20)
MRVTEIRKSYQHWWRWGIMLLGILMICNAEEKLWVTVYYGVPVWKEATT
TLFCASDAKAYDTEVHNVWATHACVPTDPNPQEVALENVTENFNMWKNN
MVEQMHEDIISLWDQSLKPCVKLTPLCVTLNCTDLRNATSRNVTNTTSS
SRGMVGGGEMKNCSFNITTGIRGKVQKEYALFYELDIVPIDNKIDRYRL
ISCNTSVITQACPKVSFEPIPIHYCAPAGFAILKCKDKKFNGKGPCSNV
STVQCTHGIRPVVSTQLLLNGSLAEEEVVIRSENFTNNAKTIIVQLNES
VEINCTRPNNNTRKSINIGPGRAFYTTGEIIGDIRQAHCNLSRAKWNDT
LNKIVIKLREQFGNKTIVFKHSSGGDPEIVTHSFNCGGEFFYCNSTQLF
NSTWNVTEESNNTVENNTITLPCRIKQIINMWQEVGRAMYAPPIRGQIR
CSSNITGLLLTRDGGPEDNKTEVFRPGGGDMRDNWRSELYKYKVVKIEP
LGVAPTKAKRRVVQREKRAVGIGAVFLGFLGAAGSTMGAASMTLTVQAR
LLLSGIVQQQNNLLRAIEAQQHLLQLTVWGIKQLQARVLAVERYLRDQQ
LLGIWGCSGKLICTTAVPWNASWSNKSLNKIWDNMTWMEWDREINNYTS
IIYSLIEESQNQQEKNEQELLELDKWASLWNWFDITKWLWYIK

Wild type HIV signal sequence is underlined. Mature N-terminal HIV envelope sequences for gp140 trimers is italicized.

BAL-rgp140
(SEQ ID NO: 21)
ATGAGAGTGACGGAGATCAGGAAGAGTTATCAGCACTGGTGGAGATGGG
GCATCATGCTCCTTGGGATATTAATGATCTGTAATGCTGAAGAAAAATT
GTGGGTCACAGTCTATTATGGGGTACCTGTGTGGAAAGAGGCCACCACC
ACACTGTTCTGTGCCTCCGATGCCAAGGCCTACGATACCGAGGTGCACA
ACGTGTGGGCCACTCATGCCTGCGTGCCCACCGATCCTAATCCTCAAGA
AGTGGCCCTGGAAAACGTGACCGAGAACTTCAACATGTGGAAGAACAAC
ATGGTCGAGCAGATGCACGAGGACATCATCAGCCTGTGGGACCAGAGCC
TGAAGCCTTGCGTGAAGCTGACCCCTCTGTGCGTGACCCTGAACTGCAC
CGACCTGAGAAACGCCACCAGCCGGAACGTGACCAATACCACCTCTAGC
AGCAGAGGCATGGTTGGAGGCGGCGAGATGAAGAACTGCAGCTTCAACA
TCACCACCGGCATCAGAGGCAAGGTGCAGAAAGAGTACGCCCTGTTCTA
CGAGCTGGACATCGTGCCCATCGACAACAAGATCGACCGGTACAGACTG
ATCAGCTGCAACACCAGCGTGATCACCCAGGCCTGTCCTAAGGTGTCCT
TCGAGCCCATTCCTATCCACTACTGTGCCCCTGCCGGCTTCGCCATCCT
GAAGTGCAAGGACAAGAAGTTCAACGGCAAGGGCCCCTGCAGCAACGTG
TCCACAGTGCAGTGTACACACGGCATCAGGCCCGTGGTGTCTACACAGC
TGCTGCTGAATGGCAGCCTGGCCGAGGAAGAGGTGGTCATCAGAAGCGA
GAATTTCACCAACAACGCCAAGACCATCATCGTGCAGCTGAACGAGAGC
GTGGAAATCAACTGCACCCGGCCTAACAACAACACCCGGAAGTCCATCA
ACATCGGCCCTGGCAGAGCCTTCTACACAACCGGCGAGATCATCGGCGA
CATCAGACAGGCCCACTGCAACCTGTCTCGGGCCAAGTGGAACGACACC
CTGAACAAGATTGTGATCAAGCTGAGAGAGCAGTTCGGCAACAAGACGA
TCGTGTTCAAGCACAGCTCTGGCGGCGACCCTGAGATCGTGACCCACAG
CTTTAATTGTGGCGGCGAGTTCTTCTACTGCAACAGCACCCAGCTGTTC
AACTCCACCTGGAATGTGACCGAGGAAAGCAACAATACCGTCGAGAACA
ACACCATCACACTGCCCTGCCGGATCAAGCAGATCATCAATATGTGGCA
AGAAGTCGGCAGGGCTATGTACGCCCCTCCTATCAGAGGCCAGATCCGG
TGCAGCAGCAATATCACAGGCCTGCTGCTCACCAGAGATGGCGGCCCTG
AGGATAACAAGACCGAGGTGTTCAGACCCGGCGGAGGCGACATGAGAGA
CAATTGGAGAAGCGAGCTGTACAAGTACAAGGTGGTCAAGATCGAGCCC
CTGGGCGTCGCCCCTACCAAGGCAAAGAGAAGAGTGGTGCAGAGAGAAA
AAAGAGCAGTGGGAATAGGAGCTGTGTTCCTTGGGTTCTTGGGAGCAGC
AGGAAGCACTATGGGCGCAGCATCAATGACGCTGACGGTACAGGCCAGA
CTATTATTGTCTGGTATAGTGCAACAGCAGAACAATCTGCTGAGAGCTA
TTGAGGCGCAACAGCATCTGTTGCAACTCACAGTCTGGGGCATTAAGCA
GCTCCAGGCAAGAGTCCTGGCTGTGGAAAGATACCTAAGGGATCAACAG
CTCCTGGGGATTTGGGGTTGCTCTGGAAAACTCATCTGCACCACTGCCG
TGCCTTGGAATGCTAGTTGGAGTAATAAATCTCTGAATAAGATTTGGGA
TAACATGACCTGGATGGAGTGGGACAGAGAAATTAACAATTACACAAGC
ATAATATACAGCTTAATTGAAGAATCGCAGAACCAACAAGAAAAGAATG
AACAAGAATTATTAGAATTAGATAAATGGGCAAGTTTGTGGAATTGGTT
TGACATAACAAAATGGCTGTGGTATATAAAA

Wild type HIV signal sequence encoding sequence is underlined. Mature N-terminal HIV envelope sequence encoding sequence for gp140 trimers is italicized.

Clade C: TZ97008-rgp120; UCSC 1374; codon
optimized
(SEQ ID NO: 22)
VPVWKEAKTTLFCASEAKGYEKEVHNVWATHACVPTDPSPHELVLENVTE
NFNMWENDMVDQMHEDIISLWDQSLKPCVKLTPLCVTLNCTNVTGTNVTG
NDMKGEMTNCSFNATTEIKDRKKNVYALFYKLDVVQLEGNSSNSTYSTYR
LINCNTSVITQACPKVSFDPIPIHYCAPAGYAILKCNNKTFNGTGPCNNV
STVQCTHGIKPVVSTQLLLNGSLAEKEIVIRSKNLTDNVKTIIVHLNESV
EITCIRPGNNTRKSIRIGPGQAFYATGDIIGNIRQAHCNISEDKWNKTLQ
MVGEKLGKLFPNKTIKEPASGGDLEITTHSFNCRGEFFYCNTTKLFNSTY
RPNANANSSSSNNTITLQCKIKQIINMWQEVGRAMYAPPIAGNITCTSNI
TGLLLVRDGGNNSTEEEIFRPGGGNMKDNWRSELYKYKVVEIKPLGVAPT
GAK
BG505-rgp120. L111A-rgp120; codon optimized
(SEQ ID NO: 28)
MPMGSLQPLATLYLLGMLVASVLAAENLWVTVYYGVPVWKDAETTLFCAS
DAKAYETEKHNVWATHACVPTDPNPQEIHLENVTEEFNMWKNNMVEQMHT
DIISAWDQSLKPCVKLTPLCVTLQCTNVTNNITDDMRGELKNCSFNMTTE
LRDKKQKVYSLFYRLDVVQINENQGNRSNNSNKEYRLINCNTSAITQACP
KVSFEPIPIHYCAPAGFAILKCKDKKFNGTGPCPSVSTVQCTHGIKPVVS
TQLLLNGSLAEEEVMIRSENITNNAKNILVQFNTPVQINCTRPNNNTRKS
IRIGPGQAFYATGDIIGDIRQAHCNVSKATWNETLGKVVKQLRKHFGNNT
IIRFANSSGGDLEVTTHSFNCGGEFFYCNTSGLENSTWISNTSVQGSNST
GSNDSITLPCRIKQIINMWQRIGQAMYAPPIQGVIRCVSNITGLILTRDG
GSTNSTTETFRPGGGDMRDNWRSELYKYKVVKIEPLGVAPTRAKSSVVGS
EKSG

Wild type HIV signal sequence is underlined. Mature N-terminal HIV envelope sequences for gp140 trimers is italicized.

BG505-rgp120.L111A-rgp120
(SEQ ID NO: 29)
ATGCCTATGGGCAGCCTGCAGCCTCTGGCCACACTGTACCTGCTGGGCAT
GCTGGTGGCCTCTGTGCTGGCCGCCGAGAACCTGTGGGTGACAGTGTACT
ACGGCGTGCCCGTGTGGAAGGACGCCGAGACAACCCTGTTCTGCGCCAGC
GACGCCAAGGCCTACGAGACAGAGAAGCACAACGTGTGGGCCACCCACGC
CTGCGTGCCAACCGACCCTAACCCCCAGGAAATCCACCTGGAAAACGTGA
CCGAAGAGTTCAACATGTGGAAGAACAACATGGTGGAACAGATGCACACC
GACATCATCAGCGCCTGGGACCAGAGCCTGAAGCCCTGCGTGAAGCTGAC
CCCCCTGTGCGTGACCCTGCAGTGCACCAACGTGACCAACAACATCACCG
ACGACATGCGGGGCGAGCTGAAGAACTGCAGCTTCAACATGACCACCGAG
CTGCGGGACAAGAAACAGAAGGTGTACAGCCTGTTCTACCGGCTGGACGT
GGTGCAGATCAACGAGAACCAGGGCAACAGAAGCAACAACAGCAACAAAG
AGTACCGGCTGATCAACTGCAACACCAGCGCCATCACCCAGGCCTGCCCC
AAGGTGTCCTTCGAGCCCATCCCCATCCACTACTGCGCCCCTGCCGGCTT
CGCCATCCTGAAGTGCAAGGACAAGAAGTTCAACGGCACCGGCCCCTGCC
CCAGCGTGTCCACAGTGCAGTGTACCCACGGCATCAAGCCCGTGGTGTCC
ACCCAGCTGCTGCTGAACGGCAGCCTGGCCGAAGAGGAAGTGATGATCAG
AAGCGAGAACATCACCAACAACGCCAAGAACATCCTGGTGCAGTTCAACA
CCCCCGTGCAGATTAACTGCACCCGGCCCAACAACAACACCAGAAAGAGC
ATCCGGATCGGCCCAGGCCAGGCCTTCTACGCCACCGGCGACATCATCGG
CGACATCCGGCAGGCCCACTGCAACGTGTCCAAGGCCACCTGGAACGAGA
CACTGGGCAAGGTGGTGAAACAGCTGCGGAAGCACTTCGGGAACAACACC
ATCATCCGCTTCGCCAACAGCTCTGGCGGCGACCTGGAAGTGACCACCCA
CAGCTTCAACTGTGGCGGCGAGTTCTTCTACTGCAATACCTCCGGCCTGT
TCAACAGCACCTGGATCAGCAATACCAGCGTGCAGGGCAGCAACAGCACC
GGCAGCAACGACAGCATCACCCTGCCCTGCCGGATCAAGCAGATCATCAA
TATGTGGCAGCGGATTGGCCAGGCTATGTACGCCCCACCCATCCAGGGCG
TGATCAGATGCGTGTCCAATATCACCGGCCTGATCCTGACCCGGGACGGC
GGCTCTACCAACAGCACCACCGAAACCTTCAGACCCGGCGGAGGCGACAT
GAGAGACAACTGGCGGAGCGAGCTGTACAAGTACAAAGTGGTGAAAATCG
AGCCCCTGGGCGTGGCCCCCACCAGAGCCAAGAGCAGCGTGGTCGGAAGC
GAGAAGTCCGGC

Wild type HIV signal sequence encoding sequence is underlined. Mature N-terminal HIV envelope sequence encoding sequence for gp140 trimers is italicized.

BG505-rgp140; not codon optimized
(SEQ ID NO: 30)
MRVMGIQRNCQHLFRWGTMILGMIIICSAAENLWVTVYYGVPVWKDAETT
LFCASDAKAYETEKHNVWATHACVPTDPNPQEIHLENVTEEFNMWKNNMV
EQMHTDIISAWDQSLKPCVKLTPLCVTLQCTNVTNNITDDMRGELKNCSF
NMTTELRDKKQKVYSLFYRLDVVQINENQGNRSNNSNKEYRLINCNTSAI
TQACPKVSFEPIPIHYCAPAGFAILKCKDKKFNGTGPCPSVSTVQCTHGI
KPVVSTQLLLNGSLAEEEVMIRSENITNNAKNILVQFNTPVQINCTRPNN
NTRKSIRIGPGQAFYATGDIIGDIRQAHCNVSKATWNETLGKVVKQLRKH
FGNNTIIRFANSSGGDLEVTTHSFNCGGEFFYCNTSGLFNSTWISNTSVQ
GSNSTGSNDSITLPCRIKQIINMWQRIGQAMYAPPIQGVIRCVSNITGLI
LTRDGGSTNSTTETFRPGGGDMRDNWRSELYKYKVVKIEPLGVAPTRAKR
RVVGREKRAVGIGAVFLGFLGAAGSTMGAASMTLTVQARNLLSGIVQQQS
NLLRAIEAQQHLLKLTVWGIKQLQARVLAVERYLRDQQLLGIWGCSGKLI
CTTNVPWNSSWSNRNLSEIWDNMTWLQWDKEISNYTQIIYGLLEESQNQQ
EKNEQDLLALDKWASLWNWFDISNWLWYIK

Wild type HIV signal sequence is underlined. Mature N-terminal HIV envelope sequences for gp140 trimers is italicized.

BG505-rgp140; not codon optimized
(SEQ ID NO: 31)
ATGAGAGTGATGGGGATACAGAGGAATTGTCAGCACTTATTCAGATGGGG
AACTATGATCTTGGGGATGATAATAATCTGTAGTGCAGCAGAAAACTTGT
GGGTCACTGTCTACTATGGGGTGCCCGTGTGGAAGGACGCCGAGACAACC
CTGTTCTGCGCCAGCGACGCCAAGGCCTACGAGACAGAGAAGCACAACGT
GTGGGCCACCCACGCCTGCGTGCCAACCGACCCTAACCCCCAGGAAATCC
ACCTGGAAAACGTGACCGAAGAGTTCAACATGTGGAAGAACAACATGGTG
GAACAGATGCACACCGACATCATCAGCGCCTGGGACCAGAGCCTGAAGCC
CTGCGTGAAGCTGACCCCCCTGTGCGTGACCCTGCAGTGCACCAACGTGA
CCAACAACATCACCGACGACATGCGGGGCGAGCTGAAGAACTGCAGCTTC
AACATGACCACCGAGCTGCGGGACAAGAAACAGAAGGTGTACAGCCTGTT
CTACCGGCTGGACGTGGTGCAGATCAACGAGAACCAGGGCAACAGAAGCA
ACAACAGCAACAAAGAGTACCGGCTGATCAACTGCAACACCAGCGCCATC
ACCCAGGCCTGCCCCAAGGTGTCCTTCGAGCCCATCCCCATCCACTACTG
CGCCCCTGCCGGCTTCGCCATCCTGAAGTGCAAGGACAAGAAGTTCAACG
GCACCGGCCCCTGCCCCAGCGTGTCCACAGTGCAGTGTACCCACGGCATC
AAGCCCGTGGTGTCCACCCAGCTGCTGCTGAACGGCAGCCTGGCCGAAGA
GGAAGTGATGATCAGAAGCGAGAACATCACCAACAACGCCAAGAACATCC
TGGTGCAGTTCAACACCCCCGTGCAGATTAACTGCACCCGGCCCAACAAC
AACACCAGAAAGAGCATCCGGATCGGCCCAGGCCAGGCCTTCTACGCCAC
CGGCGACATCATCGGCGACATCCGGCAGGCCCACTGCAACGTGTCCAAGG
CCACCTGGAACGAGACACTGGGCAAGGTGGTGAAACAGCTGCGGAAGCAC
TTCGGGAACAACACCATCATCCGCTTCGCCAACAGCTCTGGCGGCGACCT
GGAAGTGACCACCCACAGCTTCAACTGTGGCGGCGAGTTCTTCTACTGCA
ATACCTCCGGCCTGTTCAACAGCACCTGGATCAGCAATACCAGCGTGCAG
GGCAGCAACAGCACCGGCAGCAACGACAGCATCACCCTGCCCTGCCGGAT
CAAGCAGATCATCAATATGTGGCAGCGGATTGGCCAGGCTATGTACGCCC
CACCCATCCAGGGCGTGATCAGATGCGTGTCCAATATCACCGGCCTGATC
CTGACCCGGGACGGCGGCTCTACCAACAGCACCACCGAAACCTTCAGACC
CGGCGGAGGCGACATGAGAGACAACTGGCGGAGCGAGCTGTACAAGTACA
AAGTGGTGAAAATCGAGCCCCTGGGCGTGGCCCCCACCAGAGCCAAGAGA
AGAGTGGTGGGGAGAGAAAAAAGAGCAGTTGGAATAGGAGCTGTCTTCCT
TGGGTTCTTAGGAGCAGCAGGAAGCACTATGGGCGCGGCGTCAATGACGC
TGACGGTACAGGCCAGAAATTTATTATCTGGCATAGTGCAACAGCAAAGC
AATTTGCTGAGGGCTATAGAGGCTCAACAACATCTGTTGAAACTCACGGT
CTGGGGCATTAAACAGCTCCAGGCAAGGGTCCTGGCTGTGGAAAGATACC
TAAGGGATCAACAGCTTCTAGGAATTTGGGGCTGCTCTGGAAAACTCATC
TGCACCACTAATGTGCCCTGGAACTCTAGTTGGAGTAATAGAAACCTGAG
TGAGATATGGGACAACATGACCTGGCTGCAATGGGATAAAGAAATTAGCA
ATTACACACAGATAATATATGGGCTACTTGAAGAATCGCAGAACCAGCAG
GAAAAGAATGAACAAGACTTATTGGCATTGGATAAGTGGGCAAGTCTGTG
GAATTGGTTTGACATATCAAACTGGCTGTGGTATATAAAA

Wild type HIV signal sequence encoding sequence is underlined. Mature N-terminal HIV envelope sequence encoding sequence for gp140 trimers is italicized.

TV1.21-rgp120
(SEQ ID NO: 32)
MRVMGTQKNCQQWWIWGILGFWMLMICNTKDLWVTVYYGVPVWREAK
TTLFCASDAKAYETEVHNVWATHACVPTDPNPQEIVLGNVTENFNMW
KNDMADQMHEDIISLWDQSLKPCVKLTPLCVTLNCTETNVTGNRTVI
GNTNDTNIANATYKYEEMKNCSFNVTTELRNKKHKEYALFYRLDIVP
LNENGDNSKYRLINCNTSAITQACPKVSFDPIPIHYCAPAGYAILKC
NNKTFNGTGPCYNVSTVQCTHGIKPVVSTQLLLNGSLAEEGMIIRSE
NLTENTKTIIVHLNESVEINCTRPNNNTRKSVRIGPGQAFYATNDVI
GDIRQAHCNISTDRWNKTLQQVMKKLGEHFPNKTIQFKPHAGGDIE
ITMHSFNCRGEFFYCNTSNLFNSTYHSNNGTYKYNGNSSSPITLQCK
IKQIVRMWQGVGQAMYAPPIAGNITCRSNITGILLTRDGGFNTTNNT

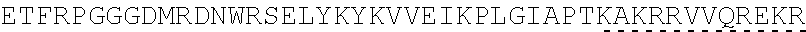

Wild type HIV signal sequence is underlined. Mature N-terminal gD purification Tag is italicized. Dotted line ( [symbol] ): indicates location of basic residues that are targets for furin and trypsin like enzymes. Translational stop codons for C-terminal purification tags can be incorporated at the beginning to this sequence. If a C-terminal purification tag is not included, then stop codon can be inserted at either the beginning or end of this sequence.

TV1.21-rgp120; not codon optimized
(SEQ ID NO: 33)
ATGAGAGTGATGGGGACACAGAAGAATTGTCAACAATGGTGGATATGGGG
CATCTTAGGCTTCTGGATGCTAATGATTTGTAATACAAAGGACTTGTGGG
TCACAGTCTATTATGGGGTACCTGTGTGGAGAGAAGCAAAAACTACCCTA
TTCTGTGCATCAGATGCTAAAGCATATGAGACAGAAGTGCATAATGTCTG
GGCTACACATGCCTGTGTGCCCACAGACCCCAACCCACAAGAAATAGTTT
TGGGAAATGTAACAGAAAATTTTAATATGTGGAAAAATGACATGGCAGAT
CAGATGCATGAGGATATAATCAGTTTATGGGATCAAAGCCTAAAGCCATG
TGTAAAGTTGACCCCACTCTGTGTCACTTTAAACTGTACAGAGACAAATG
TTACAGGTAATAGAACTGTTATAGGTAATACAAATGATACCAATATTGCA
AATGCTACATATAAGTATGAAGAAATGAAAAATTGCTCTTTCAATGTAAC
CACAGAACTAAGAAATAAGAAACATAAGGAGTATGCACTCTTTTATAGAC
TTGACATAGTACCACTTAATGAGAATGGTGACAACTCTAAATATAGATTG
ATAAATTGCAATACCTCAGCCATAACACAAGCCTGTCCAAAGGTCTCTTT
TGACCCGATTCCTATACATTACTGTGCTCCAGCTGGTTATGCGATTCTAA
AGTGTAATAATAAGACATTCAATGGGACAGGACCATGTTATAATGTCAGC
ACAGTACAATGTACACATGGAATTAAGCCAGTGGTATCAACTCAACTACT
GTTAAATGGTAGCCTAGCAGAAGAAGGGATGATAATTAGATCTGAAAATT
TGACAGAAAATACCAAAACAATAATAGTACATCTTAATGAATCTGTAGAG
ATTAATTGTACAAGACCCAACAATAATACAAGAAAAAGTGTAAGGATAGG
ACCAGGACAAGCCTTCTATGCAACAAATGATGTAATAGGAGACATAAGAC
AAGCACATTGTAACATTAGTACAGATAGATGGAACAAAACTCTACAACAG
GTAATGAAAAAACTAGGAGAGCATTTCCCTAATAAAACAATACAATTTAA
ACCACATGCAGGAGGGGATATAGAAATTACAATGCATAGCTTTAATTGTA
GAGGAGAATTTTTCTATTGCAATACATCAAACCTGTTTAATAGTACATAC
CACTCTAATAATGGTACATACAAATATAATGGTAATTCAAGCTCACCCAT
CACACTCCAATGCAAAATAAAACAAATTGTACGCATGTGGCAAGGGGTAG
GACAAGCAATGTATGCCCCTCCCATTGCAGGAAACATAACATGTAGATCA
AACATCACAGGAATACTATTGACACGCGATGGAGGATTTAACACCACAAA
CAACACAGAGACATTCAGACCTGGAGGAGGAGATATGAGGGATAACTGGA
GAAGTGAACTATATAAATATAAAGTAGTAGAAATTAAGCCATTGGGAATA
GCACCCACTAAGGCAAAAAGAAGAGTGGTGCAGAGAGAAAAAAGA

Wild type HIV signal sequence encoding sequence is underlined. Mature N-terminal gD purification Tag encoding sequence is italicized.

TV1.21-rgp140; not codon optimized
(SEQ ID NO: 34)
MRVMGTQKNCQQWWIWGILGFWMLMICNTKDLWVTVYYGVPVWREAKTTL
FCASDAKAYETEVHNVWATHACVPTDPNPQEIVLGNVTENFNMWKNDMAD
QMHEDIISLWDQSLKPCVKLTPLCVTLNCTETNVTGNRTVIGNTNDTNIA
NATYKYEEMKNCSFNVTTELRNKKHKEYALFYRLDIVPLNENGDNSKYRL
INCNTSAITQACPKVSFDPIPIHYCAPAGYAILKCNNKTFNGTGPCYNVS
TVQCTHGIKPVVSTQLLLNGSLAEEGMIIRSENLTENTKTIIVHLNESVE
INCTRPNNNTRKSVRIGPGQAFYATNDVIGDIRQAHCNISTDRWNKTLQQ
VMKKLGEHFPNKTIQFKPHAGGDIEITMHSFNCRGEFFYCNTSNLFNSTY
HSNNGTYKYNGNSSSPITLQCKIKQIVRMWQGVGQAMYAPPIAGNITCRS
NITGILLTRDGGFNTTNNTETFRPGGGDMRDNWRSELYKYKVVEIKPLGI
APTKAKRRVVQREKRAVGIGAVFLGFLGAAGSTMGAASITLTVQARQLLS
GIVQQQSNLLKAIEAQQHMLQLTVWGIKQLQARVLAIERYLKDQQLLGIW
GCSGRLICTTAVPWNSSWSNKSEADIWDNMTWMQWDREINNYTEAIFRLL
EDSQNQQEKNEKDLLELDKWNSLWNWFNISNWLWYIK

Wild type HIV signal sequence is underlined. Mature N-terminal gD purification Tag is italicized.

TV1.21-rgp140; not codon optimized
(SEQ ID NO: 35)
ATGAGAGTGATGGGGACACAGAAGAATTGTCAACAATGGTGGATATGGGG
CATCTTAGGCTTCTGGATGCTAATGATTTGTAATACAAAGGACTTGTGGG
TCACAGTCTATTATGGGGTACCTGTGTGGAGAGAAGCAAAAACTACCCTA
TTCTGTGCATCAGATGCTAAAGCATATGAGACAGAAGTGCATAATGTCTG
GGCTACACATGCCTGTGTGCCCACAGACCCCAACCCACAAGAAATAGTTT
TGGGAAATGTAACAGAAAATTTTAATATGTGGAAAAATGACATGGCAGAT
CAGATGCATGAGGATATAATCAGTTTATGGGATCAAAGCCTAAAGCCATG
TGTAAAGTTGACCCCACTCTGTGTCACTTTAAACTGTACAGAGACAAATG
TTACAGGTAATAGAACTGTTATAGGTAATACAAATGATACCAATATTGCA
AATGCTACATATAAGTATGAAGAAATGAAAAATTGCTCTTTCAATGTAAC
CACAGAACTAAGAAATAAGAAACATAAGGAGTATGCACTCTTTTATAGAC
TTGACATAGTACCACTTAATGAGAATGGTGACAACTCTAAATATAGATTG
ATAAATTGCAATACCTCAGCCATAACACAAGCCTGTCCAAAGGTCTCTTT
TGACCCGATTCCTATACATTACTGTGCTCCAGCTGGTTATGCGATTCTAA
AGTGTAATAATAAGACATTCAATGGGACAGGACCATGTTATAATGTCAGC
ACAGTACAATGTACACATGGAATTAAGCCAGTGGTATCAACTCAACTACT
GTTAAATGGTAGCCTAGCAGAAGAAGGGATGATAATTAGATCTGAAAATT
TGACAGAAAATACCAAAACAATAATAGTACATCTTAATGAATCTGTAGAG
ATTAATTGTACAAGACCCAACAATAATACAAGAAAAAGTGTAAGGATAGG
ACCAGGACAAGCCTTCTATGCAACAAATGATGTAATAGGAGACATAAGAC
AAGCACATTGTAACATTAGTACAGATAGATGGAACAAAACTCTACAACAG
GTAATGAAAAAACTAGGAGAGCATTTCCCTAATAAAACAATACAATTTAA
ACCACATGCAGGAGGGGATATAGAAATTACAATGCATAGCTTTAATTGTA
GAGGAGAATTTTTCTATTGCAATACATCAAACCTGTTTAATAGTACATAC
CACTCTAATAATGGTACATACAAATATAATGGTAATTCAAGCTCACCCAT
CACACTCCAATGCAAAATAAAACAAATTGTACGCATGTGGCAAGGGGTAG
GACAAGCAATGTATGCCCCTCCCATTGCAGGAAACATAACATGTAGATCA
AACATCACAGGAATACTATTGACACGCGATGGAGGATTTAACACCACAAA
CAACACAGAGACATTCAGACCTGGAGGAGGAGATATGAGGGATAACTGGA
GAAGTGAACTATATAAATATAAAGTAGTAGAAATTAAGCCATTGGGAATA
GCACCCACTAAGGCAAAAAGAAGAGTGGTGCAGAGAGAAAAAAGAGCAGT
GGGAATAGGAGCTGTGTTCCTTGGGTTCTTGGGAGCAGCAGGAAGCACTA
TGGGCGCAGCGTCAATAACGCTGACGGTACAGGCCAGACAACTGTTGTCT
GGTATAGTGCAACAGCAAAGCAATTTGCTGAAGGCTATAGAGGCGCAACA
GCATATGTTGCAACTCACAGTCTGGGGCATTAAGCAGCTCCAGGCGAGAG
TCCTGGCTATAGAAAGATACCTAAAGGATCAACAGCTCCTAGGGATTTGG
GGCTGCTCTGGAAGACTCATCTGCACCACTGCTGTGCCTTGGAACTCCAG
TTGGAGTAATAAATCTGAAGCAGATATTTGGGATAACATGACTTGGATGC
AGTGGGATAGAGAAATTAATAATTACACAGAAGCAATATTCAGGTTGCTT
GAAGACTCGCAAAACCAGCAGGAAAAGAATGAAAAAGATTTATTAGAATT
GGACAAGTGGAACAGTCTGTGGAATTGGTTTAACATATCAAACTGGCTGT
GGTATATAAAA

Wild type HIV signal sequence encoding sequence is underlined. Mature N-terminal gD purification Tag encoding sequence is italicized.

1086C-rgp120; not codon optimized
(SEQ ID NO: 36)
MRVRGIWKNWPQWLIWSILGFWIGNMEGSWVTVYYGVPVWKEAKTTLFCA
SDAKAYEKEVHNVWATHACVPTDPNPQEMVLANVTENFNMWKNDMVEQMH
EDIISLWDESLKPCVKLTPLCVTLNCTNVKGNESDTSEVMKNCSFKATTE
LKDKKHKVHALFYKLDVVPLNGNSSSSGEYRLINCNTSAITQACPKVSFD
PIPLHYCAPAGFAILKCNNKTFNGTGPCRNVSTVQCTHGIKPVVSTQLLL
NGSLAEEEIIIRSENLTNNAKTIIVHLNESVNIVCTRPNNNTRKSIRIGP
GQTFYATGDIIGNIRQAHCNINESKWNNTLQKVGEELAKHFPSKTIKFEP
SSGGDLEITTHSFNCRGEFFYCNTSDLFNGTYRNGTYNHTGRSSNGTITL
QCKIKQIINMWQEVGRAIYAPPIEGEITCNSNITGLLLLRDGGQSNETND
TETFRPGGGDMRDNWRSELYKYKVVEIKPLGVAPTEAK

Wild type HIV signal sequence is underlined. Mature N-terminal gD purification Tag is italicized.

1086C-rgp120; not codon optimized
(SEQ ID NO: 37)
ATGAGAGTGAGGGGGATATGGAAGAATTGGCCACAATGGTTGATATGGAG
CATCTTAGGCTTTTGGATAGGTAATATGGAGGGCTCGTGGGTCACAGTTT
ACTATGGAGTGCCTGTGTGGAAAGAAGCAAAAACTACTCTATTCTGTGCA
TCAGATGCTAAAGCATATGAGAAAGAAGTGCATAATGTCTGGGCTACACA
TGCCTGTGTGCCCACAGATCCCAACCCACAAGAAATGGTTTTGGCAAATG
TAACAGAAAATTTTAACATGTGGAAAAATGATATGGTAGAGCAGATGCAT
GAGGATATAATTAGTTTGTGGGATGAAAGCCTGAAGCCATGTGTGAAGTT
GACCCCACTCTGTGTCACTTTAAATTGTACAAATGTTAAAGGGAATGAGA
GTGACACCAGTGAAGTAATGAAAAATTGCTCTTTCAAGGCAACCACGGAA
CTAAAGGATAAAAAACATAAGGTGCATGCGCTTTTTTATAAACTTGATGT
AGTACCACTTAATGGAAACAGCAGCAGCTCTGGAGAGTATAGATTAATAA
ATTGCAATACCTCAGCCATAACACAAGCCTGTCCAAAGGTCTCTTTTGAC
CCAATTCCTTTACATTACTGTGCACCAGCTGGTTTTGCGATTCTAAAGTG
TAATAATAAGACATTCAATGGGACAGGACCATGTCGTAATGTCAGCACAG
TACAATGTACACATGGAATTAAGCCAGTGGTATCAACTCAACTACTGTTA
AATGGTAGCCTAGCAGAAGAAGAGATAATAATTAGATCTGAAAATCTGAC
AAACAATGCCAAAACAATAATAGTACACCTCAATGAATCTGTAAACATTG
TGTGTACAAGACCCAATAATAATACAAGAAAAAGTATAAGGATAGGACCA
GGACAAACATTCTATGCAACAGGTGACATAATAGGAAACATAAGACAGGC
ACATTGTAACATTAATGAAAGTAAATGGAACAACACTTTACAAAAGGTAG
GAGAAGAATTAGCAAAACACTTCCCTAGTAAAACAATAAAGTTTGAACCA
TCCTCAGGAGGGGATCTAGAAATTACAACACATAGCTTTAATTGTAGAGG
AGAGTTTTTCTATTGCAATACATCAGACCTGTTTAATGGTACATACAGAA
ATGGTACATACAATCATACAGGAAGAAGTTCAAATGGAACCATCACCCTC
CAATGCAAAATAAAACAAATTATAAACATGTGGCAGGAGGTAGGAAGAGC
AATATATGCCCCTCCCATTGAAGGAGAAATAACATGTAACTCAAATATCA
CAGGACTACTATTGCTACGTGATGGAGGTCAATCAAATGAAACAAATGAC
ACAGAGACATTCAGACCTGGAGGAGGAGATATGAGGGACAATTGGAGAAG
TGAATTATATAAATATAAAGTAGTAGAAATTAAACCATTGGGAGTAGCAC
CCACTGAGGCAAAA
1086C-rgp140
(SEQ ID NO: 38)
MRVRGIWKNWPQWLIWSILGFWIGNMEGSWVTVYYGVPVWKEAKTTLFCA
SDAKAYEKEVHNVWATHACVPTDPNPQEMVLANVTENFNMWKNDMVEQMH
EDIISLWDESLKPCVKLTPLCVTLNCTNVKGNESDTSEVMKNCSFKATTE
LKDKKHKVHALFYKLDVVPLNGNSSSSGEYRLINCNTSAITQACPKVSFD
PIPLHYCAPAGFAILKCNNKTFNGTGPCRNVSTVQCTHGIKPVVSTQLLL
NGSLAEEEIIIRSENLTNNAKTIIVHLNESVNIVCTRPNNNTRKSIRIGP
GQTFYATGDIIGNIRQAHCNINESKWNNTLQKVGEELAKHFPSKTIKFEP
SSGGDLEITTHSFNCRGEFFYCNTSDLFNGTYRNGTYNHTGRSSNGTITL
QCKIKQIINMWQEVGRAIYAPPIEGEITCNSNITGLLLLRDGGQSNETND
TETFRPGGGDMRDNWRSELYKYKVVEIKPLGVAPTEAKRRVVEREKRAVG
IGAVFLGFLGAAGSTMGAASMTLTVQARQLLSGIVQQQSNLLRAIEAQQH
MLQLTVWGIKQLQARVLAIERYLKDQQLLGMWGCSGKLICTTAVPWNSSW
SNKSQNEIWGNMTWMQWDREINNYTNTIYRLLEDSQNQQEKNEKDLLALD
SWKNLWNWFDISKWLWYIK

Wild type HIV signal sequence is underlined. Mature N-terminal gD purification Tag is italicized.

1086C-rgp140
(SEQ ID NO: 39)
ATGAGAGTGAGGGGGATATGGAAGAATTGGCCACAATGGTTGATATGGAG
CATCTTAGGCTTTTGGATAGGTAATATGGAGGGCTCGTGGGTCACAGTTT
ACTATGGAGTGCCTGTGTGGAAAGAAGCAAAAACTACTCTATTCTGTGCA
TCAGATGCTAAAGCATATGAGAAAGAAGTGCATAATGTCTGGGCTACACA
TGCCTGTGTGCCCACAGATCCCAACCCACAAGAAATGGTTTTGGCAAATG
TAACAGAAAATTTTAACATGTGGAAAAATGATATGGTAGAGCAGATGCAT
GAGGATATAATTAGTTTGTGGGATGAAAGCCTGAAGCCATGTGTGAAGTT
GACCCCACTCTGTGTCACTTTAAATTGTACAAATGTTAAAGGGAATGAGA
GTGACACCAGTGAAGTAATGAAAAATTGCTCTTTCAAGGCAACCACGGAA
CTAAAGGATAAAAAACATAAGGTGCATGCGCTTTTTTATAAACTTGATGT
AGTACCACTTAATGGAAACAGCAGCAGCTCTGGAGAGTATAGATTAATAA
ATTGCAATACCTCAGCCATAACACAAGCCTGTCCAAAGGTCTCTTTTGAC
CCAATTCCTTTACATTACTGTGCACCAGCTGGTTTTGCGATTCTAAAGTG
TAATAATAAGACATTCAATGGGACAGGACCATGTCGTAATGTCAGCACAG
TACAATGTACACATGGAATTAAGCCAGTGGTATCAACTCAACTACTGTTA
AATGGTAGCCTAGCAGAAGAAGAGATAATAATTAGATCTGAAAATCTGAC
AAACAATGCCAAAACAATAATAGTACACCTCAATGAATCTGTAAACATTG
TGTGTACAAGACCCAATAATAATACAAGAAAAAGTATAAGGATAGGACCA
GGACAAACATTCTATGCAACAGGTGACATAATAGGAAACATAAGACAGGC
ACATTGTAACATTAATGAAAGTAAATGGAACAACACTTTACAAAAGGTAG
GAGAAGAATTAGCAAAACACTTCCCTAGTAAAACAATAAAGTTTGAACCA
TCCTCAGGAGGGGATCTAGAAATTACAACACATAGCTTTAATTGTAGAGG
AGAGTTTTTCTATTGCAATACATCAGACCTGTTTAATGGTACATACAGAA
ATGGTACATACAATCATACAGGAAGAAGTTCAAATGGAACCATCACCCTC
CAATGCAAAATAAAACAAATTATAAACATGTGGCAGGAGGTAGGAAGAGC
AATATATGCCCCTCCCATTGAAGGAGAAATAACATGTAACTCAAATATCA
CAGGACTACTATTGCTACGTGATGGAGGTCAATCAAATGAAACAAATGAC
ACAGAGACATTCAGACCTGGAGGAGGAGATATGAGGGACAATTGGAGAAG
TGAATTATATAAATATAAAGTAGTAGAAATTAAACCATTGGGAGTAGCAC
CCACTGAGGCAAAAAGGAGAGTGGTGGAGAGAGAAAAAAGAGCAGTGGGA
ATAGGAGCTGTGTTCCTTGGGTTCTTGGGAGCAGCCGGAAGCACTATGGG
CGCAGCATCAATGACGCTGACGGTACAGGCCAGGCAATTATTGTCTGGTA
TAGTGCAACAGCAAAGCAATTTGCTGAGGGCTATAGAGGCGCAACAGCAT
ATGTTGCAACTCACGGTCTGGGGCATTAAACAGCTCCAGGCAAGAGTCCT
GGCTATAGAAAGATACCTAAAGGATCAACAGCTCCTAGGGATGTGGGGCT
GCTCTGGAAAACTCATCTGCACCACTGCTGTGCCTTGGAACTCCAGTTGG
AGTAACAAATCTCAAAATGAAATTTGGGGGAACATGACCTGGATGCAGTG
GGACAGAGAAATTAATAATTACACAAACACAATATATAGGTTACTTGAAG
ACTCACAAAACCAGCAGGAAAAAAATGAGAAAGATTTGTTAGCATTGGAC
AGTTGGAAAAATCTGTGGAATTGGTTTGACATATCAAAGTGGCTGTGGTA
TATAAAA

Wild type HIV signal sequence encoding sequence is underlined. Mature N-terminal gD purification Tag encoding sequence is italicized.

CAP45.2.00.G3-rgp120; not codon optimized
(SEQ ID NO: 40)
MRVRGILRNWPQWWIWSILGFWMLIICRVMGNLWVTVYYGVPVWKEAKAT
LFCASDARAYEKEVHNVWATHACVPTDPNPQEIYLGNVTENFNMWKNDMV
DQMHEDIISLWDQSLKPCVKLTPLCVTLRCTNATINGSLTEEVKNCSFNI
TTELRDKKQKAYALFYRPDVVPLNKNSPSGNSSEYILINCNTSTITQACP
KVSFDPIPIHYCAPAGYAILKCNNKTENGTGPCNNVSTVQCTHGIKPVVS
TQLLLNGSLAEEDIIIKSENLTNNIKTIIVHLNKSVEIVCRRPNNNTRKS
IRIGPGQAFYATNDIIGDIRQAHCNINNSTWNRTLEQIKKKLREHFLNRT
IEFEPPSGGDLEVTTHSFNCGGEFFYCNTTRLFKWSSNVTNDTITIPCRI
KQFINMWQGAGRAMYAPPIEGNITCNSSITGLLLTRDGGKTDRNDTEIFR
PGGGNMKDNWRNELYKYKVVEIKPLGVAPTEARRRVVEREKR

Wild type HIV signal sequence is underlined. Mature N-terminal gD purification Tag is italicized.

CAP45.2.00.G3-rgp120
(SEQ ID NO: 41)
ATGAGAGTGAGGGGGATACTGAGGAATTGGCCACAATGGTGGATATGGAG
CATCTTAGGCTTTTGGATGCTAATAATTTGTAGGGTGATGGGGAACTTGT
GGGTCACAGTCTATTATGGGGTACCTGTGTGGAAAGAAGCAAAAGCTACT
CTATTCTGTGCATCAGATGCTAGAGCATATGAGAAAGAAGTGCATAATGT
CTGGGCTACACATGCCTGTGTACCCACAGACCCCAACCCACAAGAAATAT
ACTTGGGAAATGTAACAGAAAATTTTAACATGTGGAAAAATGACATGGTG
GATCAGATGCATGAGGATATAATCAGTTTATGGGATCAAAGTCTAAAGCC
ATGTGTAAAGTTGACCCCACTCTGTGTCACTTTAAGGTGTACAAATGCTA
CTATTAATGGTAGCCTGACGGAAGAAGTAAAAAATTGCTCTTTCAATATA
ACCACAGAGCTAAGAGATAAGAAACAGAAAGCGTATGCACTTTTTTATAG
ACCTGATGTAGTACCACTTAATAAGAATAGCCCTAGTGGGAATTCTAGTG
AGTATATATTAATAAATTGCAATACCTCAACCATAACACAAGCCTGTCCA
AAGGTCTCTTTTGACCCAATTCCTATACATTATTGTGCTCCAGCTGGTTA
TGCGATTCTAAAGTGTAATAATAAGACATTTAATGGGACAGGACCATGCA
ATAATGTCAGCACAGTACAATGTACACATGGAATTAAACCAGTGGTATCA
ACTCAACTACTGTTAAATGGTAGCTTAGCAGAAGAAGATATCATAATTAA
ATCTGAAAATCTGACAAACAATATCAAAACAATAATAGTACACCTTAATA
AATCTGTAGAAATTGTGTGTAGAAGACCCAACAATAATACAAGGAAAAGT
ATAAGGATAGGACCAGGACAGGCTTTCTATGCAACAAATGACATAATAGG
AGACATAAGACAAGCACATTGTAATATTAATAATTCTACATGGAACAGAA
CTTTAGAACAGATAAAGAAAAAATTAAGAGAACACTTCCTTAATAGAACA
ATAGAATTTGAACCACCCTCAGGGGGGGATCTAGAAGTTACAACACATAG
CTTTAATTGTGGAGGAGAATTTTTCTATTGCAATACAACACGACTGTTTA
AGTGGTCTAGTAATGTCACAAACGACACAATCACAATCCCATGCAGAATA
AAACAATTTATAAACATGTGGCAAGGGGCAGGACGAGCAATGTATGCCCC
TCCCATTGAAGGAAACATAACATGTAACTCAAGTATCACAGGACTCCTAT
TGACACGTGATGGAGGGAAAACAGACAGGAATGACACAGAGATATTCAGA
CCTGGAGGAGGAAATATGAAGGACAATTGGAGAAATGAATTATATAAATA
TAAAGTGGTAGAAATTAAGCCATTGGGAGTAGCACCCACTGAGGCAAGAA
GGAGAGTGGTGGAGAGAGAAAAAAGA

Wild type HIV signal sequence is underlined. Mature N-terminal gD purification Tag is italicized.

CAP45.2.00.G3-_rgp140
(SEQ ID NO: 42)
MRVRGILRNWPQWWIWSILGFWMLIICRVMGNLWVTVYYGVPVWKEAKAT
LFCASDARAYEKEVHNVWATHACVPTDPNPQEIYLGNVTENFNMWKNDMV
DQMHEDIISLWDQSLKPCVKLTPLCVTLRCTNATINGSLTEEVKNCSFNI
TTELRDKKQKAYALFYRPDVVPLNKNSPSGNSSEYILINCNTSTITQACP
KVSFDPIPIHYCAPAGYAILKCNNKTENGTGPCNNVSTVQCTHGIKPVVS
TQLLLNGSLAEEDIIIKSENLTNNIKTIIVHLNKSVEIVCRRPNNNTRKS
IRIGPGQAFYATNDIIGDIRQAHCNINNSTWNRTLEQIKKKLREHFLNRT
IEFEPPSGGDLEVTTHSFNCGGEFFYCNTTRLFKWSSNVTNDTITIPCRI
KQFINMWQGAGRAMYAPPIEGNITCNSSITGLLLTRDGGKTDRNDTEIFR
PGGGNMKDNWRNELYKYKVVEIKPLGVAPTEARRRVVEREKRAVGIGAVL
LGFLGAAGSTMGAASITLTVQARQLLSGIVQQQSNLLRAIEAQQHMLQLT
VWGIKQLQTRVLAIERYLKDQQLLGLWGCSGKLICTTNVPWNSSWSNKSQ
TDIWDNMTWIQWDREISNYSNTIYKLLEGSQNQQEQNEKDLLALDSWNNL
WNWFNITNWLWYIK

Wild type HIV signal sequence is underlined. Mature N-terminal gD purification Tag is italicized.

CAP45.2.00.G3-rgp140
(SEQ ID NO: 43)
ATGAGAGTGAGGGGGATACTGAGGAATTGGCCACAATGGTGGATATGGAG
CATCTTAGGCTTTTGGATGCTAATAATTTGTAGGGTGATGGGGAACTTGT
GGGTCACAGTCTATTATGGGGTACCTGTGTGGAAAGAAGCAAAAGCTACT
CTATTCTGTGCATCAGATGCTAGAGCATATGAGAAAGAAGTGCATAATGT
CTGGGCTACACATGCCTGTGTACCCACAGACCCCAACCCACAAGAAATAT
ACTTGGGAAATGTAACAGAAAATTTTAACATGTGGAAAAATGACATGGTG
GATCAGATGCATGAGGATATAATCAGTTTATGGGATCAAAGTCTAAAGCC
ATGTGTAAAGTTGACCCCACTCTGTGTCACTTTAAGGTGTACAAATGCTA
CTATTAATGGTAGCCTGACGGAAGAAGTAAAAAATTGCTCTTTCAATATA
ACCACAGAGCTAAGAGATAAGAAACAGAAAGCGTATGCACTTTTTTATAG
ACCTGATGTAGTACCACTTAATAAGAATAGCCCTAGTGGGAATTCTAGTG
AGTATATATTAATAAATTGCAATACCTCAACCATAACACAAGCCTGTCCA
AAGGTCTCTTTTGACCCAATTCCTATACATTATTGTGCTCCAGCTGGTTA
TGCGATTCTAAAGTGTAATAATAAGACATTTAATGGGACAGGACCATGCA
ATAATGTCAGCACAGTACAATGTACACATGGAATTAAACCAGTGGTATCA
ACTCAACTACTGTTAAATGGTAGCTTAGCAGAAGAAGATATCATAATTAA
ATCTGAAAATCTGACAAACAATATCAAAACAATAATAGTACACCTTAATA
AATCTGTAGAAATTGTGTGTAGAAGACCCAACAATAATACAAGGAAAAGT
ATAAGGATAGGACCAGGACAGGCTTTCTATGCAACAAATGACATAATAGG
AGACATAAGACAAGCACATTGTAATATTAATAATTCTACATGGAACAGAA
CTTTAGAACAGATAAAGAAAAAATTAAGAGAACACTTCCTTAATAGAACA
ATAGAATTTGAACCACCCTCAGGGGGGGATCTAGAAGTTACAACACATAG
CTTTAATTGTGGAGGAGAATTTTTCTATTGCAATACAACACGACTGTTTA
AGTGGTCTAGTAATGTCACAAACGACACAATCACAATCCCATGCAGAATA
AAACAATTTATAAACATGTGGCAAGGGGCAGGACGAGCAATGTATGCCCC
TCCCATTGAAGGAAACATAACATGTAACTCAAGTATCACAGGACTCCTAT
TGACACGTGATGGAGGGAAAACAGACAGGAATGACACAGAGATATTCAGA
CCTGGAGGAGGAAATATGAAGGACAATTGGAGAAATGAATTATATAAATA
TAAAGTGGTAGAAATTAAGCCATTGGGAGTAGCACCCACTGAGGCAAGAA
GGAGAGTGGTGGAGAGAGAAAAAAGAGCAGTGGGAATAGGAGCTGTACTC
CTTGGGTTCTTGGGAGCAGCAGGAAGCACTATGGGCGCGGCGTCAATAAC
GCTGACGGTACAGGCCAGGCAACTGTTGTCTGGTATAGTGCAACAGCAAA
GCAATTTGCTGAGAGCTATAGAGGCGCAACAGCACATGTTGCAACTCACG
GTCTGGGGCATTAAGCAGCTCCAGACAAGAGTCCTGGCTATAGAAAGGTA
CCTAAAGGATCAACAGCTCCTAGGGCTTTGGGGCTGCTCTGGAAAACTCA
TCTGCACCACTAATGTGCCTTGGAACTCCAGTTGGAGTAATAAATCTCAA
ACAGATATTTGGGATAACATGACCTGGATACAGTGGGATAGAGAAATTAG
TAATTACTCAAACACAATATACAAGTTGCTTGAAGGCTCGCAAAATCAGC
AGGAGCAAAATGAAAAAGACTTATTAGCATTGGACAGTTGGAATAATCTG
TGGAATTGGTTCAACATAACAAATTGGCTGTGGTATATAAAA

Wild type HIV signal sequence encoding sequence is underlined. Mature N-terminal gD purification Tag encoding sequence is italicized.

As noted herein, the HIV envelope gp may be expressed with a tag at the N-terminus and/or the C-terminus. Sequences of exemplary tags are provided:

Herpes simplex virus I glycoprotein D ss (gD-1 ss)
(SEQ ID NO: 44)
MGGAAARLGAVILFVVIVGLHGVRG.
Fruit bat herpes simplex virus glycoprotein D ss
(FBgD-1 ss)
(SEQ ID NO: 45)
MAYPAVIVLVCGLFWVPATQG.
Intracellular adhesion molecule ss (ICAM-1 ss)
(SEQ ID NO: 46)
MAPSSPRPALPALLVLLGALFPGPGNA.
Tissue plasminogen activator ss (TPA ss)
(SEQ ID NO: 47)
MDAMKRGLCCVLLLCGAVFVSPSQEIHARFRRGARW.
gD-1 tag
(SEQ ID NO: 48)
KYALADASLKMADPNRFRGKDLPVLDQ
1-14D-1 tag
(SEQ ID NO: 49)
YVRADPSLSMVNPNRFRGGHLPPLVQQ
HIVgp120 tag
(SEQ ID NO: 50)
TDNLWVTVYYG
6X His tag
(SEQ ID NO: 51)
HHHHHH
Avi tag
(SEQ ID NO: 52)
GLNDIFEAQKIEWHE
Strep-Tactin (Strep) tag
(SEQ ID NO: 53)
WSHPQFEK
His-Strep tag
(SEQ ID NO: 54)
HHHHHHSSWSHPQ1-BK
His-Strep-6X His tag (C-terminus)
(SEQ ID NO: 55)
HHHHHHSSWSHPQFEKSSHHHHHH
His-Strep-His (HSH) tag (N-terminus)
(SEQ ID NO: 56)
HHHHHHSHPQFEKHHHHHHQSG

As noted herein, HIV env gp can be expressed with or without the following sequence at the C-terminus. [symbol] (SEQ ID NO:57). This sequence includes location of basic residues that are targets for furin and trypsin like enzymes. Translational stop codons for C-terminal purification tags can be incorporated at the beginning to this sequence. If a C-terminal purification tag is included, then stop codon can be inserted at either the beginning or end of the sequence.

As noted herein, HIV env gp can be expressed with or without the following sequence at the C-terminus:

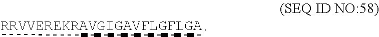

Dotted line ( [symbol] ): This sequence includes location of basic residues that are targets for furin and trypsin like enzymes. Translational stop codons for C-terminal purification tags can be incorporated at the beginning to this sequence. If a C-terminal purification tag is included, then stop codon can be inserted at either the beginning or end of the sequence. Broken line ( [symbol] ): C-terminal or 3′ sequences not required for expression.

### EXAMPLES

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

Generation of Mgat1⁻ CHO-S Cell Line

This report describes the use of the CRISPR/Cas9 gene editing system to inactivate the Mannosyl (Alpha-1,3-)-Glycoprotein Beta-1,2-N Acetylglucosaminyltransferase (Mgat1) gene in CHO cells for the purpose of creating a stable cell line, with growth properties suitable for biopharmaceutical production, for the purpose of producing HIV envelope proteins for use as vaccine immunogens.

It is widely believed that for an HIV vaccine to be successful, it needs stimulate the formation of broadly neutralizing antibodies (bNAbs). After more than 30 years of research none to the candidate vaccines developed to date are able to elicit these types of antibodies. For many years the specificity of bNAbs was unknown. Over the last few years advancements in B-cell cloning technology have allowed the isolation of broadly neutralizing monoclonal antibodies (bN-mAbs) from HIV infected humans. Surprisingly, many of these were found to recognize glycan dependent epitopes that required specific types of N-linked glycosylation for binding. The N-linked glycans required are high-mannose forms, primarily mannose-5 and mannose-9 that normally are early intermediates in the N-linked glycosylation pathway. These glycans differ from the normal complex, sialic acid containing carbohydrates found in mature membrane bound and secreted proteins. The fact that virtually all previous HIV vaccines possessed the normal type of complex glycosylation may explain their inability to elicit glycan dependent bNAbs. Genetic techniques were used to create cell lines that incorporate these early intermediate glycoforms (mannnose-5, mannose-8, and mannose-9) at N-linked glycosylation sites in all cellular proteins as well as heterologous proteins such as HIV envelope proteins. Disclosed herein is the use of the CRISPR/Cas9 gene editing system to knockout the Mannosyl (Alpha-1,3-)-Glycoprotein Beta-1,2-N-Acetylglucosaminyltransferase (Mgat1) gene of the Chinese hamster Ovary (CHO) cells to produce CHO cells suitable for biopharmaceutical production. This mutation prevents the processing of N-linked glycans beyond the mannnose-5 (Man₅) form, enabling the production of envelope proteins with high level of glycosylation with mannose N-glycans. Monomeric gp120 produced by transient transfection of this cell line binds the prototypic glycan dependent bNAb PG9. Taking advantage of the robust productivity of CHO cells, this line has been established for the development of HIV-1 vaccine antigens as well as other vaccines, diagnostic, and therapeutic products requiring the incorporation of mostly mannose glycans.

Materials and Methods

Cells and Antibodies.

Suspension adapted CHO-S and 293 HEK Freestyle cells were obtained from Thermo Fisher (Thermo Fisher, Life technologies, Carlsbad, Calif.). HEK GNT1⁻ cells were obtained from ATCC (ATCC, Manassas, Va.). Broadly neutralizing monoclonal antibody PG9 was produced from synthetic genes created on the basis of published sequence data (available from the NIH AIDS Reagent Program, Germantown, Md.). The antibody genes were expressed in 293 HEK cells using standard techniques. Polyclonal rabbit sera was from rabbits immunized using a Complete Freund's Adjuvant/Incomplete Freund's Adjuvant (CFA/IFA) protocol (Pocono Rabbit Farms, AAALAC #926, Canadensis, Pa.) with A244rgp120 produced in GNT1-HEK293 cells. Fluorescently conjugated anti-Human, anti-Rabbit, and anti-Murine antibodies were obtained from Invitrogen (Invitrogen, Thermo Fisher, Carlsbad, Calif.)

Cell Culture Conditions.

Stocks of suspension adapted CHO-S, 293 HEK, and GNT1⁻ cells were maintained in shake flasks (Corning, Corning N.Y.) using a Kuhner ISF1-X shaker incubator (Kuhner, Birsfelden, Switzerland). For normal cell propagation shake flasks cultures were maintained at 37° C., 8% CO₂, and 125 rpm. Static cultures were maintained in 96 or 24 well cell culture dishes and grown in a Sanyo incubator (Sanyo, Moriguchi, Osaka, Japan) at 37° C. and 8% CO₂.

Cell Culture Media.

For normal CHO cell growth, cells were maintained in CD-CHO medium supplemented with 0.1% pluronic acid, 8 mM GlutaMax and 1× Hypoxanthine/Thymidine (Thermo Fisher, Life Technologies, Carlsbad, Calif.). 293 HEK (Freestyle) and GNT1⁻ 293 HEK cells were maintained in Freestyle 293 cell culture media (Life Technologies, Carlsbad, Calif.). For CHO cell protein production the cells were maintained in OptiCHO medium supplemented with 0.1% pluronic acid, 8 mM GlutaMax and 1×H/T (Thermo Fisher, Life Technologies, Carlsbad, Calif.). For protein production experiments the growth medium was supplemented with MaxCyte CHO A Feed (0.5% Yeastolate, BD, Franklin Lakes, N.J.; 2.5% CHO-CD Efficient Feed A; and 0.25 mM GlutaMAX, 2 g/L Glucose (Sigma-Aldrich St. Louis, Mo.).

Cell Counts and Growth Calculation.

All cell counts were performed using a TC20™ automated cell counter (BioRad, Hercules, Calif.) with viability determined by trypan blue (Thermo Fisher, Life Technologies, Carlsbad, Calif.) exclusion. Cell-doubling time in hours was calculated using the formula: (((time2−time1)×24)×log(2))/(log(density2)−log(density1)).

Gene Sequencing.

The CHO Mgat1 gene sequence was confirmed using predicted mRNA transcript XM_007644560.1 to design primers. Genomic DNA was extracted using Qiagen AllPrep kit (Qiagen, Germantown, Md.). The Mgat1 gene was PCR amplified using the primers F_CAGGCAAGCCAAAGGCAGCCTTG (SEQ ID NO: 59) and R_CTCAGGGACTGCAGGCCTGTCTC (SEQ ID NO: 60) (Eurofins Genomics, Louisville, Ky.) with Taq and dNTPs supplied by New England BioLabs (Ipswich, Mass.). The PCR product was gel purified using a Zymoclean kit (Zymo Research, Irvine, Calif.), then sequenced by Sanger method at the UC Berkeley Sequencing Center (UC Berkeley, Berkeley, Calif.). Mgat1 knockouts were sequenced in the same manner.

CRISPR/Cas9 Target Design and Plasmid Preparation.

Target sequences to knock out CHO Mgat1 were designed using an online CRISPR RNA Configurator tool (GE Dharmacon, Lafayette, Colo.). Target 1: CCCTGGAACTTGCGGTGGTC (SEQ ID NO: 61), target 2: GGGCATTCCAGCCCACAAAG (SEQ ID NO: 62), and target 3: GGCGGAACACCTCACGGGTG (SEQ ID NO: 63). Each sequence was run in NCBI's BLAST tool for homologies with off-target sites in the CHO genome. Single stranded DNA oligonucleotides and their complement strands were synthesized (Eurofins Genomics, Louisville, Ky.) with extra bases on the 3′ ends for ligation into GeneArt CRISPR nuclease vector (Thermo Fisher, GeneArt). The strands were ligated and annealed into GeneArt CRISPR vector using the protocol and reagents supplied with the kit. One Shot® TOP10 Chemically Competent E. coli were transformed and plated following the Invitrogen protocol (Thermo Fisher, Invitrogen, Carlsbad, Calif.) Five colonies from each target plate were picked the following day. These were incubated in 5 mL LB broth at 37° C. and 225 rpm overnight. Minipreps were performed using according to manufactures instructions (Qiagen, Germantown, Md.) and sent to UC Berkeley DNA Sequencing Facility (Berkeley, Calif.) with U6 primers included in GeneArt® CRISPR kit to confirm successful integration of guide sequences via Sanger sequencing. A single 500 mL MaxiPrep was performed for each of the three target sequences using PureLink™ MaxiPrep kit (Thermo Fisher, Invitrogen, Carlsbad, Calif.).

Electroporation.

Electroporation was performed using a MaxCyte STX scalable transfection system (MaxCyte Inc., Gaithersburg, Md.) according to the manufacturer's instructions. Briefly, CHO-S cells were maintained at >95% viability prior to transfection. All steps were performed using aseptic technique. Cells were pelleted at 250 g for 10 minutes, and then re-suspended in MaxCyte EP buffer (MaxCyte Inc., Gaithersburg, Md.) at a density of 2×10⁸ cells/mL. Transfections were carried out in the OC-400 processing assembly (MaxCyte Inc., Gaithersburg, Md.) with a total volume of 400 μL and 8×10⁷ total cells. Crispr/Cas9 exonuclease with guide sequence plasmid DNA, in endotoxin-free water was added to the cells in EP buffer for a final concentration of 300 μg of DNA/mL. The processing assemblies were then transferred to the MaxCyte STX electroporation device and appropriate conditions (CHO protocol) were selected using the MaxCyte STX software. Following completion of electroporation, the cells in Electroporation buffer were removed from the processing assembly and placed in 125 mL Erlenmeyer cell culture shake flasks (Corning, Corning N.Y.). The flasks were placed into 37° C. incubators with no agitation for 40 minutes. Following the rest period pre-warmed OPTI-CHO media was added to the flasks for a final cell density of 4×10⁶ cells/mL. Flasks were then moved the Kuhner shaker and agitated at 125 rpm.

Plating, Expansion, and Culture of CRISPR Transfected CHO-S Cells.

14 hours post transfection a 100 μL aliquot was taken from each of the transfected pools for cell viability counts and to check for orange fluorescent protein expression using a light microscope (Zeiss Axioskop 2, Zeiss, Jena, Germany). 96 well flat bottom cell culture plates (Corning, Corning, N.Y.) were filled with 50 μL of conditioned CD-CHO media. Each of the three transfected pools were serially diluted with warmed media to 10 cells/mL and added to five plates per pool in 50 μL volumes. Final calculated cell density was 0.5 cells/well in 100 μL of media. Once any single-colony well reached ≈20% confluency, the contents were moved to a 24 well cell culture plate (Corning, Corning, N.Y.) in 500 uL of media. When confluency reached 50%, a 200 μL aliquot was removed for testing via a GNA lectin-binding assay. Following successful lectin binding, cells were moved to a 6 well cell culture plate (Corning, Corning, N.Y.) with 2 mL of media per well. After 5 days of growth in 6 well plates, GNA assay was repeated. Those colonies that still showed uniform lectin binding to all cells were moved to 125 mL shake flasks with an initial 6 mL of media. Daily counts were taken and cell culture expanded to maintain 0.3×10⁶ to 1.0×10⁶ cells/mL density.

Lectin Binding Assay.

Fluorescein labeled Galanthus nivalis lectin, GNA (Vector Laboratories, Burlingame, Calif.), was used to probe for the expression of Man₅ glycoforms on the cell surfaces. 200 μL samples from 24 well plate wells were spun down at 3000 rpm for 3 minutes. The supernatant was discarded and the cell pellet washed three successive times with 500 μL of ice-cold 10 μM EDTA (Boston BioProducts, Ashland, Mass.) PBS (Thermo Fisher, Gibco, Carlsbad, Calif.). Following the final wash, the cell pellet was re-suspended in 200 μL ice cold 10 μM EDTA PBS with 5 μg/mL of GNA-fluorescein. Samples were incubated with GNA in dark, on ice, for 30 minutes. Following incubation, samples were washed three times and re-suspended to a volume of 50 μl in 10 μM EDTA PBS. Samples were then examined under light microscope (Zeiss Axioskop 2, Zeiss, Jena, Germany) with 495 nm excitation. Wild type CHO-S cells were used as a negative control and HEK Gnt1 were used as a positive. Representative images were taken on a Leica DM5500 B Widefield Microscope (Leica Microsystems, Buffalo Grove, Ill.) at the UC Santa Cruz microscopy center.

Small Scale Gp120 Test Transfection.

4×10⁵ cells of each candidate line were placed in 450 μl of media in a 24 well cell culture plate. In 1.7 μl of Fugene (Promega, Madison, Wis.) was pre-incubated at room temperature for 30 minutes with 550 ng of DNA in a total volume of 50 μL of media. Following an incubation period, 50 μL of the Fugene/DNA mixture was added to each well, for a final transfected volume of 500 μL. Aliquots of supernatant were removed for testing 72 hours post transfection.

Experimental Protein Production.

Cells were electroporated following the above method. 24 hours post electroporation, the culture was supplemented a single time with 1 mM sodium butyrate (Thermo Fisher, Life Technologies, Carlsbad, Calif.) and the temperature lowered to 34° C. Production culture was fed daily equivalent to 3.5% of the original volume with MaxCyte CHO A Feed. Cultures were run until viability dropped below 50%. Supernatant was harvested by pelleting the cells at 250 g for 30 minutes followed by pre-filtration through Nalgene™ Glass Pre-filters (Thermo Scientific, Waltham, Mass.) and 0.45 micron SFCA filtration Nalgene (Thermo Scientific, Waltham, Mass.), then stored frozen at −20° C. before purification.

Protein Purification.

Proteins were purified using an N-terminal affinity tag as previously described (Yu, B. et al., 2012).

Glycosidase Digestion and SDS-PAGE.

Endo H and PNGase F (New England BioLabs, Ipswich, Mass.) digests were performed per the manufacturer's protocol on 5 μg of purified protein using on unit of glycosidase. Digested samples were run on NuPAGE (Thermo Fisher, Invitrogen, Carlsbad Calif.) 4-12% BisTris precast gels in MES running buffer then stained with SimplyBlue stain (Thermo Fisher, Invitrogen, Carlsbad, Calif.). Western blot analysis primary antibody was in-house 34.1 anti-gD flag mAb and secondary was HRP conjugated goat anti-mouse IgG (American Qualex, San Clemente, Calif.). Substrate was WesternBright ECL (Advansta, Menlo Park, Calif.).

Isoelectric Focusing.

Isoelectric focusing was performed using ReadyPrep™ 2-D kit (Bio-Rd Laboratories, Hercules, Calif.). 50 μg of proteins were mixed with 150 uL IEF sample buffer. 4 μl of two internal weight standards were added: carbonic anhydrase isozyme (pI=5.9, 29kDA) and Amyloglucosidase (pI=3.6, 97 kDa) (Sigma-Aldrich, St. Louis, Mo.). The protein mixture was loaded onto a ReadyStrip™ IPG strip (pH 3-10, 11 cm) and separated by a preset protocol on a Protean® IEF Cell. Following first dimensional separation, the strips were loaded, along with a molecular weight marker (Novex® Sharp prestained standard, Invitrogen) onto a 4-20% polyacrylamide TRIS HCL gel (BIO-RAD, Hercules, Calif.) and run for 1 hour at 225 V. The gels were then stained with SimplyBlue™ SafeStain (Invitrogen).

Fluorescence Intensity Assays (FIA).

A semi-automated fluorescence immunoassay (FIA) was used to measure the binding of polyclonal or monoclonal antibodies to recombinant envelope proteins. For antibody binding to purified proteins, Greiner Fluortrac 600 microtiter plates (Greiner Bio-one, Germany) were coated with 2 μg/mL of peptide overnight in PBS with shaking. Plates were blocked in PBS+2.5% BSA (blocking buffer for 90 min, then washed 4 times with PBS containing 0.05% Tween-20 (Sigma). Serial dilutions of PG9 were added in a range from 10 ug/mL to 0.0001 ug/mL, then incubated at 25° C. for 90 min with shaking. After incubation and washing, fluorescently conjugated anti-Hu or anti-Mu (Invitrogen, CA) was added at a 1:3000 dilution. Plates were incubated for 90 minutes with shaking then washed three times with 0.05% tween PBS using an automated plate washer. Plates were then imaged in a plate spectrophotometer (Envision System, Perkin Elmer) at excitation (ex395 nm) and emission (em490 nm).

For antibody binding to unpurified culture supernatant, Greiner Fluortrac 600 microtiter plates (Greiner Bio-one, Germany) were coated with 2 μg/mL of purified monoclonal antibody (Berman lab, anti gD tag 34.1 or anti V2 peptide 10C10) overnight in PBS with shaking. Plates were blocked in PBS+2.5% BSA (blocking buffer for 90 min, then washed 4 times with PBS containing 0.05% Tween-20 (Sigma). 150 μl of 40× diluted supernatant were then added to each well or 10 μg/mL of purified protein in control lanes, then incubated at 25° C. for 90 min with shaking. After incubation and washing, PG9 was added in a range from 10 μg/mL to 0.0001 μg/mL, then incubated at 25° C. for 90 min with shaking. After incubation and washing, fluorescently conjugated anti-Hu or anti-Mu (Invitrogen, CA) was added at a 1:3000 dilution. Plates were incubated for 90 minutes with shaking then washed three times with 0.05% TWEEN® PBS using an automated plate washer. Plates were then imaged in a plate spectrophotometer (Envision System, Perkin Elmer, Waltham, Mass.) at excitation (ex395 nm) and emission (em490 nm).

All steps except coating were carried out at room temperature on a shaking platform; incubation steps were 90 min on a shaking platform. All dilutions were done in blocking buffer (1% BSA in PBS with 0.05% Normal Goat Serum). Polyclonal rabbit sera was from rabbits immunized using a CFA/IF protocol (Pocono Rabbit Farms) with A244rgp120 produced in GNT1−/− HEK293 cells.

Glycan Composition Analysis by MALDI-TOF-MS.

Glycan analysis by mass spectrometry was performed by the Complex Carbohydrate Research Center at the University of Georgia (Athens, Ga.). Glycans were released from HIV-1 envelope proteins with PNGase F, permethylated, than analyzed by MALDI-TOF-MS.

MVM Infectivity Assay.

MVM infectivity assay was performed by IDEXX BioResearch (Columbia, Mo.). Cells were cultured at 4×10⁵ cells/mL, in 100 mL total volume under conditions described above in a spinner flask for five days. Wild type CHO-S and MGAT1 cells were infected with 1 MOI of MVMp or MVMi and evaluated in triplicate. 5 mL aliquots were removed on days 1, 3, and 5, and cells were pelleted by centrifugation and stored at −20° C. Day 5 samples were evaluated by PCR for MVM and 18S using proprietary primers. qPCR crossing point (CP) values were reported and copies based upon standard curves.

Results

Target Design and Cleavage of CHO-S MGAT1.

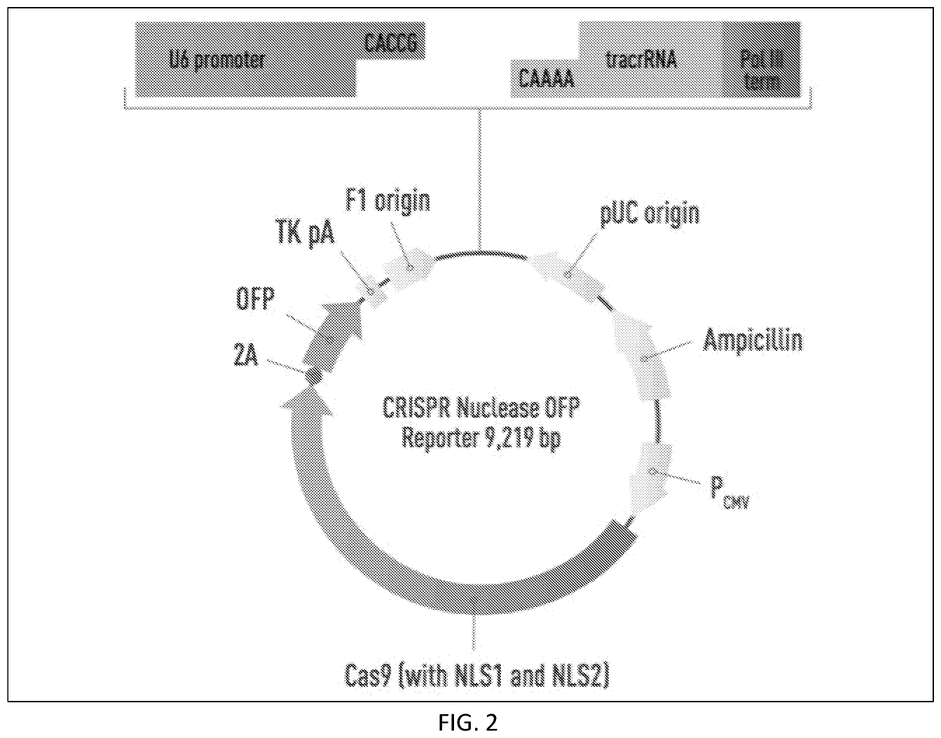

CRISPR/Cas9 allows for specific targeting of genes for knockout or modification by introducing double stranded breaks (DSB) followed by non-homologous end joining (NHEJ) or homology directed repair (HDR). The details of CRISPR/Cas9, NHEJ, and HDR have been covered in a number of review articles (Hsu, P. D. et al., Cell. 157(6): p. 1262-1278; Sander, J. D. and J. K. Joung, Nat Biotech, 2014. 32(4): p. 347-355). GeneArt® CRISPR Nuclease Vector with OFP Reporter allows contains all the elements needed for gene knockout given a well-designed target sequence. A target specific double stranded guide sequence is ligated into the vector between a U6 promoter and a tracrRNA sequence. The same plasmid encodes the Cas9 endonuclease and an orange fluorescent protein reporter separated by a self-cleaving 2A peptide linker (FIG. 2). Following ligation of these guide sequences into the vector they were transfected into CHO-S cells using the MaxCyte electroporation system. This electroporation allows near 100% transfection, even with large plasmids, increasing the odds of finding successful knockouts in a given population. Targets 1 and 2 were introduced individually, and target 3 plasmid was mixed and added together in equal ratio with target 2, creating separate pools of transfected cells. Twenty-four hours post transfection samples from each of the three conditions were serially diluted and spread across five 96-well flat-bottoms plates at a calculated density of 0.5 cells per well. The plates were examined daily, any well with more than a single colony was discarded. Across the fifteen total plates, between fifteen and thirty wells per plate contained single viable colonies. Upon reaching approximately 20% confluency, those were expanded to 24-well plate wells in 500 μL of media, taking between twelve and fifteen days to pass. Those that did not have at least several dozen cells by day fifteen were discarded. A total of 166 colonies were expanded to 24 well plates: 55 from target 1 pool, 67 from target 2 pool, and 44 from combined target ⅔ pool.

Lectin Binding Assay.

If Mgat1 was successfully knocked out then any N-linked glycoprotein expressed by the cell should have exclusively high mannose glycans with a preponderance of Man₅ isoforms. To determine successful knockout of Mgat1 at a phenotypic level, a fluorescein-conjugated Galanthus nivalis lectin (GNA—also known as GNL, Vector Laboratories, Burlingame, Calif.) was used. GNA is an unusual lectin in that it does not require a Ca²⁺ or Mg²⁺ cofactor to bind, allowing the use of 10 μM EDTA to ameliorate cell clumping during repeated centrifugation and wash steps.

A total of 20 candidate lines from the original 166 showed uniformly high GNA binding and were chosen for expansion and further analysis. This represents a potentially successful knockout rate of 12%, though many colonies were rejected early due to slow growth in the 96 well plates, so the overall rate may have been higher. Three days following initial GNA selection, the cell line candidates were re-examined and six were rejected for lack of uniform lectin binding across the sample population, leaving 14 candidates.

Cell Growth and Expression of Full Length Gp120 and V1/V2 Fragments.

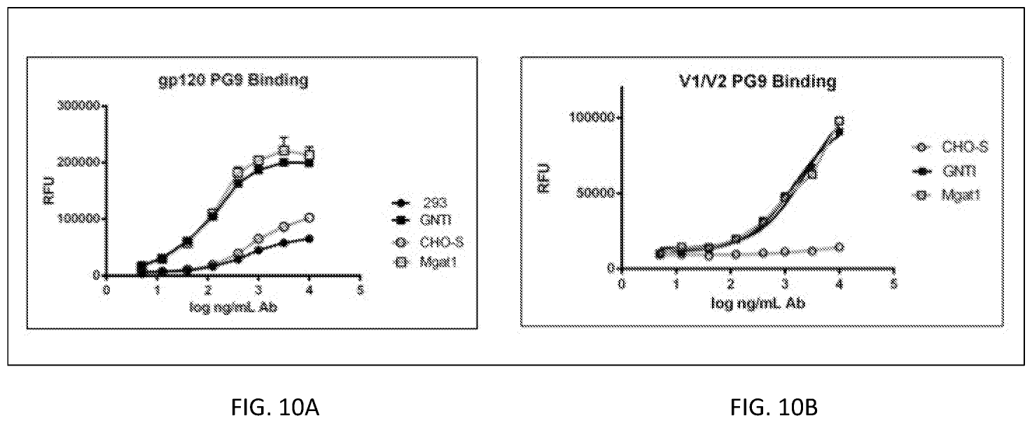

The fourteen candidate cell lines were grown in 125 mL shaker flasks for two weeks with cell counts taken daily. At the end of this period the four lines with the shortest average population doubling time were transiently transfected with a full-length gp120 gene (A244) (SEQ ID NO:4) and a V1/V2 Env fragment also from A244 protein via electroporation. Five days post transfection, the proteins were purified by affinity chromatography and were tested via FIA for their ability to bind the PG9 bNAb that requires mannoses for binding (FIGS. 10A and 10B). This assay identified the highest protein producer of the four lines, and confirmed the cell lines could produce envelope proteins with the correct glycans required to bind PG9. Material produced using wild type CHO-S and HEK Gnt1 was used as a comparator for both quantification and a PG9 high mannose binding baseline. From this analysis, a single Mgat1-CHO cell line, designated 3.4F10, was selected for further characterization and analysis.

Identification of CRISPR/Cas9 Induced Genetic Alteration.

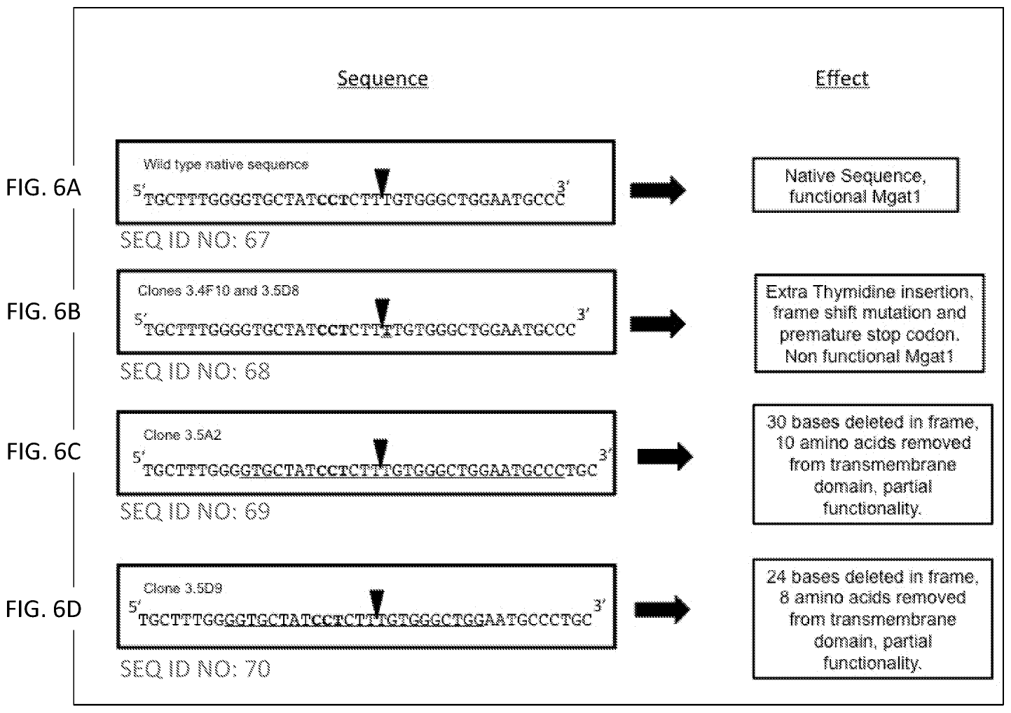

Up until this point all the analysis on the putative Mgat1⁻ cell lines had been phenotypic. To confirm that Mgat1 had been altered to the point of non-functionality on a genetic level, the Mgat1 gene from the 3.4F10 line as well as the Mgat1 gene from the next three best candidates were sequenced. In 3.4F10, an extra thymidine had been inserted at the cleavage site, introducing a frame shift mutation, leading to 23 altered codons and a premature stop (FIG. 6B). 3.5D8 has the same mutation, while 3.5D9 and 3.5A2 both had in frame deletions of 24 and 30 nucleotides respectively. The deleted codons of 3.5D9 and 3.5A2 corresponded to the transmembrane domain of the Gnt1 protein, leaving the active domain intact. This may explain why the envelope protein produced in these lines did not bind PG9 while 3.4F10 produced envelope did.

Characterization of CHO-S Mgat1⁻ Gp120 Glycosylation.

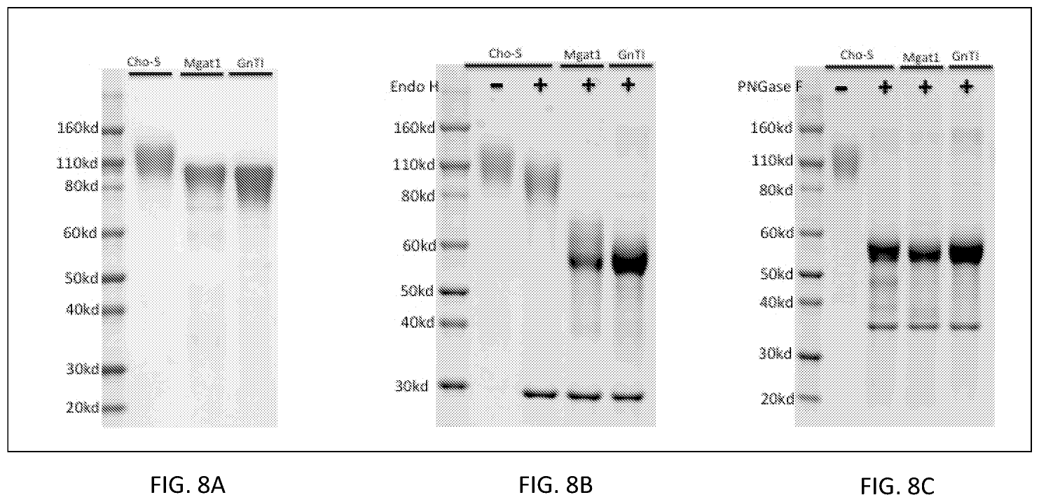

To fully characterize the lead CHO-S Mgat1⁻ cell line (hereafter, simply referred to as Mgat1) glycosylation as high mannose the following assays were performed: Glycosidase digestion, 2-D isoelectric focusing, and mass spec analysis. Affinity purified, monomeric A244 gp120 produced in CHO-S, GnT1-, 293 HEK, and Mgat1⁻ cells. These were digested overnight by PnGase F and Endo H that removes only high mannose glycans. The digest products were then separated on an SDS-PAGE gel and stained with Coomassie blue (FIGS. 8A-8C). As expected, the proteins expressed in normal CHO and 293 cells were only partially sensitive to Endo H, whereas the proteins produced in the GNT1-293 and Mgat1-CHO cells were about 20 kD smaller than the CHO-S material, due to the lower mass of Man₅ glycan structures. Endo H cleaves N-linked high-mannose glycan structures, while complex glycans are insensitive to it. Following Endo H digestion the CHO-S material is largely unaltered, but both the Mgat1 and Gnt1 products are reduced to ≈60 kd in size. This is consistent with the observation that approximately half the mass of a given gp120 molecule is from glycosylation (Binley, J. M., et al., Journal of Virology, 2010. 84(11): p. 5637-5655; Zhu, X., et al., Biochemistry, 2000. 39(37): p. 11194-11204; Go, E. P., et al., Journal of proteome research, 2013. 12(3): p. 1223-1234). The complete sensitivity to Endo H, consistent with that of Gnt1, indicates that the glycosylation of the Mgat1 line is exclusively high mannose. When digested with PNGase F, all samples dropped to the same size, confirming undigested gp120 size variances were due to glycosylation size differences and not an under laying amino acid diversity.

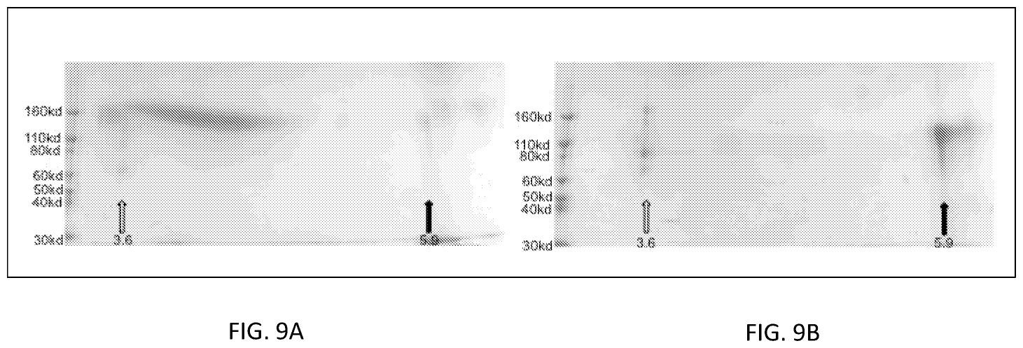

The CHO-S and Mgat1 material were resolved on 11 cm IPG strips followed by fractionation in the second dimension (FIGS. 9A and 9B). The CHO-S material had broad pI spread and was heterogeneous of both charge and mass, due to the varying levels and type of glycosylation. As expected, the charge of the Mgat1 material was highly homogenous and collapsed to a single spot.

Beyond the strong indicators above that the selected Mgat1 line was producing glycoproteins with purely high-mannose residues, the precise glycan composition of the A244 rgp120 envelope proteins was then determined. MALDI-TOFF-MS was used on CHO-S and Mgat1⁻ produced material, confirming that the Mgat1 line produced only high-mannose material with that least 70% of that being the Man₅ isoform. Thus at least 70% of the glycosylation could be attributed to mannose 5 glycans and as much as 30% could be attributed to earlier glycan precursors such as mannose 8 and mannose 9.

Binding to PG9.

To confirm whether Mgat1⁻ cell line could produce monomeric, full-length rgp120 capable of binding PG9, an FIA with both A244 rgp120 and A244 V1/V2 fragment proteins was performed (FIGS. 10A and 10B). Envelope proteins produced by HEK 293, HEK Gnt1, CHO-S, and Mgat1⁻ cells were all compared. Both the 293 and CHO-S material bound poorly, while the Gnt1 and Mgat1 material showed significant improvement over their glycan wild type counterparts, containing the necessary Man₅ epitope component.

Discussion

The overwhelming majority of HIV-1 vaccine research over the better part of three decades has focused on designing an antigen capable of eliciting a safe and effective protective immune response. While this goal has not yet been realized, there is hope. The RV144 trial demonstrated for the first time that some level of protection could be achieved through the use of a subunit vaccine (Rerks-Ngarm, S., et al., New England Journal of Medicine, 2009. 361(23): p. 2209-2220; Karasavvas, N., et al., AIDS Res Hum Retroviruses, 2012. 28(11): p. 1444-57; Kim, J. H. et al., Annu Rev Med, 2015. 66: p. 423-37). Since that time much has been learned about both the envelope protein itself and the panoply of new bNAbs that bind to it. Two general concepts have clarified the requirements for an envelope protein based manufacturing scheme. First, the glycan topography became better understood, as well as the critical role of high-mannose glycans for the binding of bNAbs; something generally avoided in bio-therapeutic production (Doores, K. J., et al., Proceedings of the National Academy of Sciences, 2010. 107(31): p. 13800-13805; Bonomelli, C., et al., PLOS ONE, 2011. 6(8): p. e23521; Go, E. P., et al., J Virol, 2011. 85(16): p. 8270-84; Pritchard, L. K., et al., Nat Commun, 2015. 6: p. 7479; Cao, L., et al., 2017. 8: p. 14954). Second, a new class of potently neutralizing bNAbs were discovered that specifically required interaction with these high-mannose structures (McLellan, J. S., et al., Nature, 2011. 480(7377): p. 336-43; Pejchal, R., et al., Science, 2011. 334(6059): p. 1097-103; Lavine, C. L., et al., Journal of Virology, 2012. 86(4): p. 2153-2164; Kong, L., et al., Nat Struct Mol Biol, 2013. 20(7): p. 796-803). The gp120 used in the RV144 trial used cell lines and methods in keeping with the best understanding of both HIV-1 and biopharmaceutical production of the time. This meant CHO production of recombinant gp120 with as much sialic acid as possible to increase stability and improve pharmacokinetic/pharmacodynamic properties. As the understanding of HIV-1 and its interaction with the immune system has matured, it became clear that high sialic acid content and complex glycosylation was likely a hindrance to the development of neutralizing antibodies. These new understandings are guiding the current development of what a HIV-1 vaccine may look like.

This creates the need for a cell platform capable of producing large amounts of recombinant high-mannose proteins. Disclosed herein is a cell line specifically for the scalable production high-mannose HIV-1 vaccine antigen. A CHO-S Mgat1 knock out line limited to Man₅₋₉N-linked glycoforms was established using the CRISPR/Cas9 gene editing system.

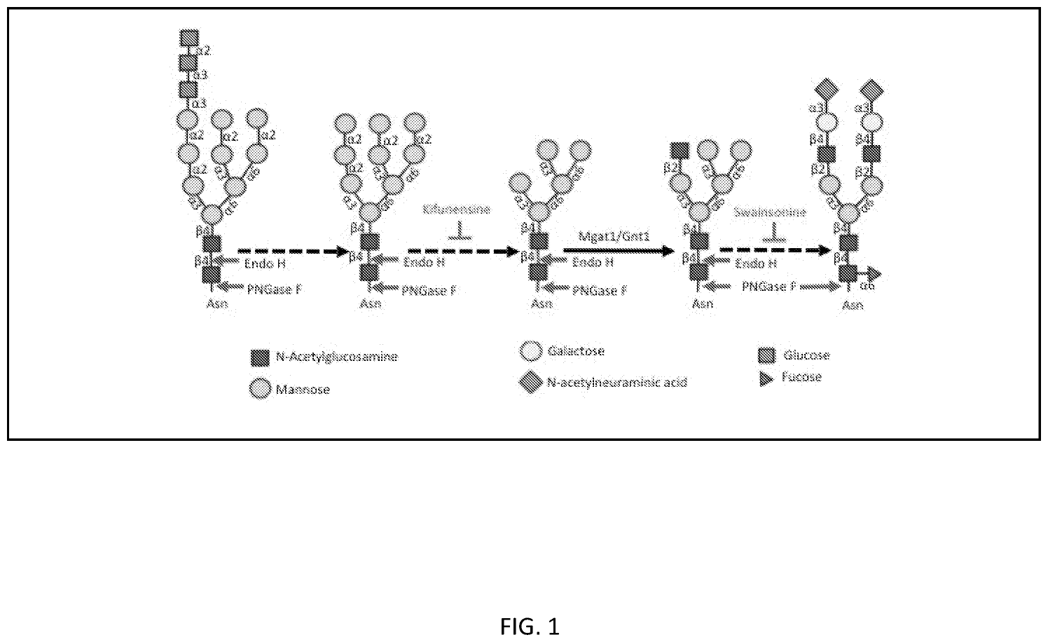

With the recent sequencing of the CHO genome (Wurm, F. M. and D. Hacker, Nat Biotech, 2011. 29(8): p. 718-720; Xu, X., et al., Nat Biotech, 2011. 29(8): p. 735-741) and the advent of CRISPR gene technology, these were used as tools to efficiently knock out Mgat1. This particular glycosyltransferase is something of a standout in the N-linked glycosylation pathway in that its action is one of the few bottlenecks (FIG. 1). While enzymes before and after this point in processing have their preferred substrates, there is some minor overlapping and branch points (Bieberich, E., Advances in Neurobiology, 2014. 9: p. 47-70; Moremen, K. W., M. Tiemeyer, and A. V. Nairn, Nat Rev Mol Cell Biol, 2012. 13(7): p. 448-62). This means that there are multiple potential paths to arrive at the same glycoform or diverge to create different structures. If the expression of Mgat1 is silenced, then N-glycan processing essentially stops at Man₅ (though α1,6 fucosylation of the primary GlcNAc by Fut8 may still occur independent of Mgat1 (Chang, V. T., et al., Structure, 2007. 15(3): p. 267-73), preventing the formation of hybrid or complex type glycans. Though the maturation process cannot proceed beyond Man5, upstream high-mannose glycoforms such as Man₈ and Man₉, required for 2G12 binding, are not precluded and may still be present on completed proteins.

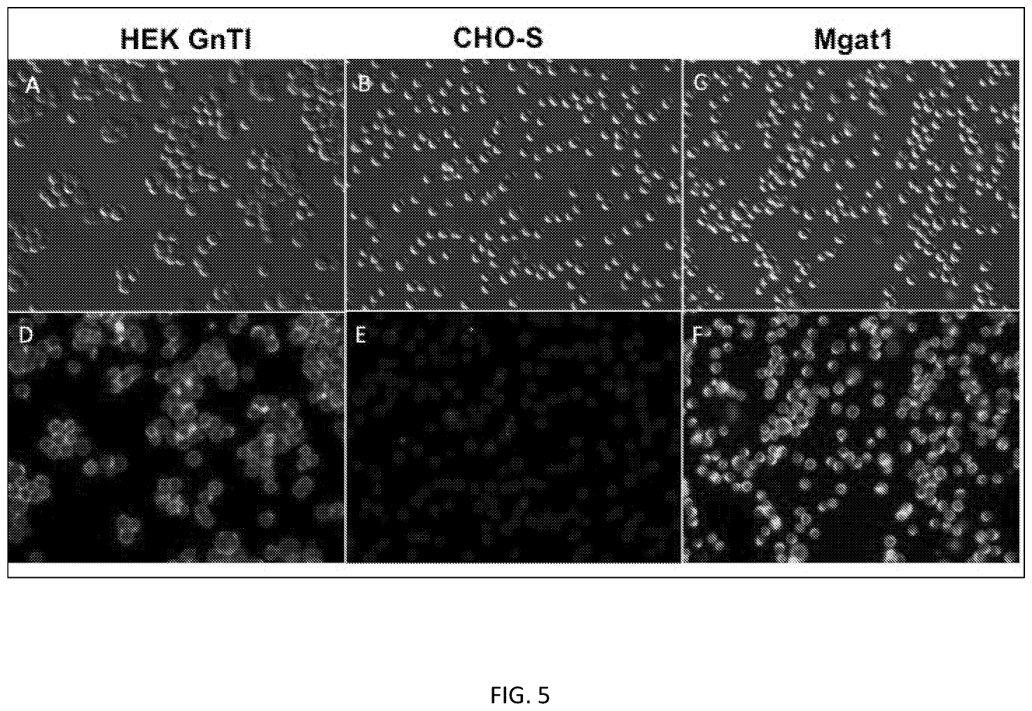

When creating this cell line an initial screening was performed by a positive selection test using GNA lectin, a mannose binding lectin with a preference for α1,3 linked mannose residues. This is in contrast to previously isolated Mgat1/Gnt1 lines, generated by mutagenesis and zinc-finger nucleases, which have relied upon negative selection through ricin lectins, such as Ricinus communis agglutinin-I and II (RCA-I, RCA-II) (Sealover, N. R., et al., Journal of Biotechnology, 2013. 167(1): p. 24-32; Patnaik, S. K. and P. Stanley, Methods in Enzymology, 2006. 416: p. 159-182; Lee, J., et al., Biochemistry, 2003. 42(42): p. 12349-12357). Unlike complex and hybrid glycans, high-mannose glycans are rare in high concentrations on healthy cell surface glycoproteins (Christiansen, M. N., et al., Proteomics, 2014. 14(4-5): p. 525-46; Hamouda, H., et al., Journal of Proteome Research, 2014. 13(12): p. 6144-6151). Positive binding of GNA to surface high-mannose glycans would be strongly indicative of successful knockout of Mgat1. Initial tests comparing the GNA-fluorescein surface staining of HEK Gnt1⁻ and CHO-S cells confirmed this with a clear difference in staining intensity (FIG. 5).

In order to be useful for viable for large-scale production, the cells have to have a reasonable growth rate. One of the features that have made CHO the dominant substrate for bio-manufacturing production is their robust growth; CHO-S cells have an average doubling time of 24.3 hours when split daily to 0.35e⁶ cells/mL. When seeded at the same densities, the four best candidate lines doubled between 24.0 and 38.3 hours. While rapid growth is one goal, the overall protein production level and quality is paramount. The candidate lines still had to demonstrate they could produce sufficient gp120 with the correct glycosylation to bind glycan dependent bNAbs. To show this, a small-scale transient transfection was performed using an A244 gp120 then performed a HA with the purified material. This told us whether the candidate lines could produce monomeric gp120 with the correct glycosylation to bind PG9. Affinity purified HEK Gnt1 produced A244 gp120 was used as a positive comparator. While all the candidate lines material bound PG9, the cell line candidate that grew the most slowly, 3.4F10 (38.3 hr doubling time), had the highest level of PG9 binding, equal to that of the HEK Gnt1 material. As expected, the WT CHO-S material, with complex and hybrid glycosylation, bound poorly. When the Mgat1-gene was sequenced in the knockouts, the two lines with the lowest relative amount of PG9 binding showed only a partial knockout of the Mgat1 gene. They each had multiple-codon in-frame deletions, corresponding to the transmembrane domain of the Mgat1 protein (FIG. 6A-6D). With the catalytic domain intact, it appears that Mgat1 mannosidase functionality in these two lines was curtailed, but not eliminated.

A single cell line 3.4F10 was selected from the initial growth characteristics and PG9 binding HA data to advance as a high-mannose HIV-1 antigen production line. A 1.3 L transient transfection of A244 gp120 was performed and affinity purified the material for further glycan analysis and bNAb affinity binding. Digestion with Endo H confirmed the uniformly high-mannose glycosylation of the gp120 produced (FIG. 8 WT CHO-S and Mgat1-produced A244 gp120 were then compared through 2D isoelectric focusing (FIG. 9). The CHO-S material, similar to what was used in the RV144 trial (Rerks-Ngarm, S., et al., New England Journal of Medicine, 2009. 361(23): p. 2209-2220; Berman, P. W., AIDS Res Hum Retroviruses, 1998. 14 Suppl 3: p. S277-89; Berman, P. W., et al., Virology, 1999. 265(1): p. 1-9), showed broad heterogeneity of charge caused by varying levels of sialylation. The Mgat1 material, devoid of sialic acid and complex glycosylation, collapsed to a single discrete point. All the tests performed up to this point (lectin biding, size shifts, glycosidase digests, 2D electrophoresis) had been secondary indicators that the Mgat1 line was producing solely high-mannose material. As a final confirmation the Mgat1-A244 gp120 material was analyzed via MALDI-TOF mass spectrometry. This definitively showed the Mgat1-line is limited to high-mannose glycoprotein production, with the preponderance of species being Man₅.

It was then determined that the Mgat1 line was an improved substrate for the production of HIV-1 vaccines. The PG9 epitope is frequently described as quaternary, requiring a gp120 native-like trimer for binding (Burton, D. R. and L. Hangartner, Annu Rev Immunol, 2016. 34: p. 635-59; Davenport, T. M., et al., Journal of Virology, 2011. 85(14): p. 7095-7107). The requisite high-mannose glycans are thought to result from the high degree of glycosylation, large size, and complex nature of the trimeric gp120 molecule preventing glycosidases and glycotransferases from effectively maturing the initial high mannose structures (Doores, K. J., et al., Proceedings of the National Academy of Sciences, 2010. 107(31): p. 13800-13805; Bonomelli, C., et al., PLOS ONE, 2011. 6(8): p. e23521; Go, E. P., et al., J Virol, 2011. 85(16): p. 8270-84). When these pathways are controlled, the same high-mannose structures can be generated on monomeric gp120, enabling PG9 binding. When comparing A244 gp120 produced by WT CHO-S cells, the Mgat1-material demonstrated a high level of binding (FIG. 10A).

At large-scale manufacturing facilities a viral contamination can be devastating, effectively shutting down production and only cleared with great effort and expense (Henzler, H.-J. and K. Kaiser, Nat Biotech, 1998. 16(11): p. 1077-1079; Moody, M., et al., PDA J Pharm Sci Technol, 2011. 65(6): p. 580-8). One of the principle causes for failed fermentation of CHO cells is infection by Minute Virus of Mice (MVM), a tiny (20 nM) non-enveloped single stranded DNA parvovirus (Moody, M., et al., PDA J Pharm Sci Technol, 2011. 65(6): p. 580-8). Because the receptor for MVM is thought to be sialic acid MVM virus infectivity assays were carried out. These studies showed that Mgat1⁻ CHO cells were resistant to infection by the strain MVMc, but sensitive to two other strains. While a full resistance to all MVM strains would be preferable, this removes on source of potential manufacturing contamination and factory shut-down.

FIG. 1. Simplified view of N-linked glycosylation pathway. N-linked glycosylation begins in the endoplasmic reticulum with the en-block transfer of a highly conserved Gluc₃Man₉GlcNac₂ structure (left) to asparagine residues within the N-X-S/T motif of nascent proteins. This initial structure is sequentially trimmed down to Man₅GlucNac₂ (center) by a number of glycosidases as the protein moves from the ER to the Golgi apparatus. Various glycosyltransferases then add monosaccharides creating hybrid (second from right) and complex (right) glycoforms. Kifunensine and Swainsonine are both inhibitors that halt further processing at the points shown above. EndoH and PNGase F remove the glycan structures where indicated by the arrows, with hybrid and complex glycans being insensitive to Endo H.

FIG. 2. GeneArt® CRISPR Nuclease vector. The orange fluorescent protein (OFP) reporter and Cas9 is expressed as a single unit, driven by a CMV promoter sequence, and joined by a self-cleaving 2A peptide linker. Nuclear localization signals NLS1 and NLS2 usher Cas9 to the nucleus. The target sequence specific double stranded DNA oligo that will generate the crRNA is inserted into the pre-linearized vector via 5 base pair overhangs. The tracrRNA sequence is located 3′ of the crRNA DNA oligo insert and is followed to by a DNA polymerase III termination sequence to ensure correct RNA folding for loading in the Cas9 complex. A U6 promoter drives expression of the crRNA and tracrRNA, which together will form the mature gRNA. Figure adapted from GeneArt® technical manual.

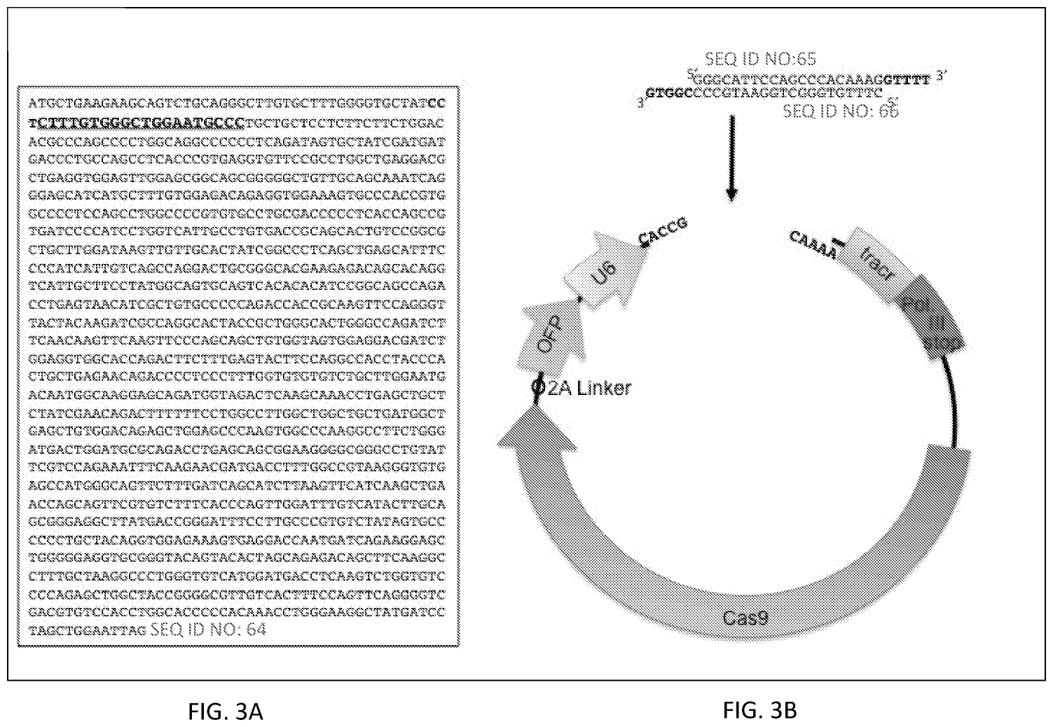

FIGS. 3A and 3B. Vector to Edit CHO Mgat1 gene. The CHO Mgat1 gene (FIG. 3A) is a single exon gene. Three gRNA sequences were designed to correspond with three target sequences in the 5′ region of the gene. One target is shown underlined above with the requisite protospacer adjacent motif (PAM) in bold. Since Cas9 causes a double stranded break, either the template or non-template strand may be targeted. In this case the guide RNA was designed to be complementary to the template strand. FIG. 3B: Following design of the gRNA, a complementary oligonucleotide was ligated to the gRNA with sticky ends complementary to the GeneArt CRISPR nuclease vector (Thermo Fisher) to ensure correct directionality following ligation into the vector. This vector includes an orange fluorescent protein (OFP) reporter attached by a self-cleaving 2A linker to the Cas9 exonuclease enzyme. Three separate gRNA sequences were created, each targeting the 5′ end of the gene. The crRNA sequence shown was used for creation of the GB Mgat1 line.

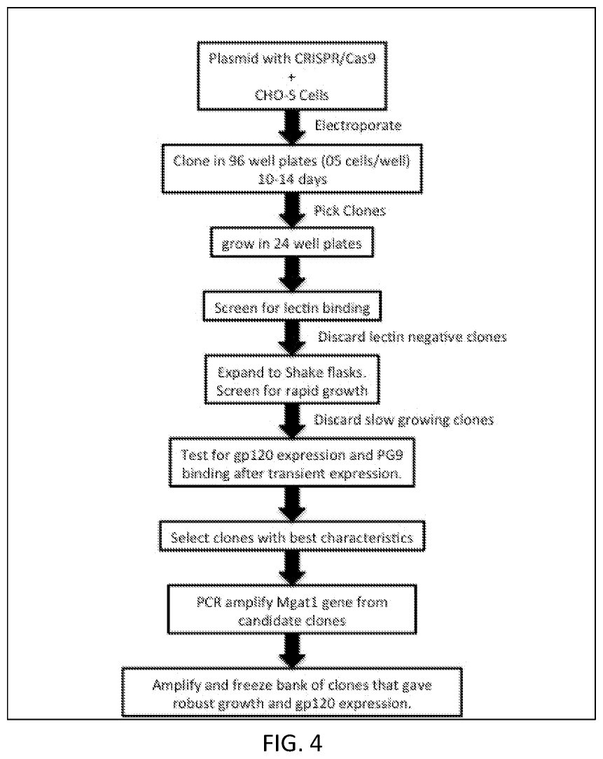

FIG. 4. Flow chart of Mgat1 gene editing and cell line selection strategy. The Cas9 nuclease vector with gRNA sequence inserted was electroporated into suspension adapted CHO-S cells. The transfected cells were re-suspended in conditioned media and cloned in 96 well plates at a calculated density of 0.5 cells/well. Those single cell derived colonies that grew well after 10-14 days were moved to 24 well plates. Aliquots were removed from each 24 plate well and screened for GNA lectin binding. Those that did not demonstrate uniform lectin binding were discarded. Candidate lines were expanded to shake flasks and screened for rapid growth, discarding slow growers. A test transient transfection was performed with A244 gp120 (SEQ ID NO:4) to determine relative expression levels and PG9 binding properties of gp120 produced by candidate lines. Those with the best growth and PG9 binding were moved forward. The Mgat1 gene was PCR amplified from the remaining candidates and sequenced. The clones with the most robust growth and gp120 expression were expanded and frozen banks created. Two of these cell lines are deposited at ATCC (PTA-124141; or PTA-124142).

FIG. 5. A GNA lectin Binding assay was used to find cells with high mannose surface glycoproteins following CRISPR/Cas9 targeted cleavage of Mgat1. As a first step to determine successful knockout of the Mgat1 gene, the candidate cells were examined for fluorescein conjugated GNA lectin binding to surface glycoproteins. GNA binds exposed mannose residues with a preference for terminal α1,3 mannose residues, such as those found on the Man₅ glycoforms. Cells were removed from culture, washed of media three times in ice cold 10 μM EDTA PBS, then re-suspended in same wash buffer with 5 μg/mL fluorescein conjugated GNA and kept on ice for 30 minute incubation. Following incubation all cells were washed three times again to remove unbound GNA. Wild type CHO-S cells should have predominantly complex and hybrid glycans on surface glycoproteins and demonstrated very little binding to GNA (E) serving as a negative control. HEK Gnt1⁻ is limited to Man₅ glycans and demonstrated positive GNA binding (D). A representative sample of transfected CHO-S cells that showed uniform GNA binding is shown in F and C. Those wells that demonstrate uniform GNA binding were advanced for growth, productivity, and genetic characterization. All images are at 20×. A, B, and C are shown in differential interference contrast (DIC), D, E, F, are shown under 495 nm excitation.

FIGS. 6A-6D. NHEJR induced changes to Mgat1 gene. Following initially promising phenotypic analysis, the four leading candidate lines Mgat1 genes were sequenced via Sanger sequencing to confirm silencing of the gene. The guide RNA was designed to be complementary with the template strand, using the PAM 'AGG. Show above is the coding strands with the PAM complement, CTT, in bold and the putative double stranded cut site indicated by the black triangle. Changes from the native sequence are underlined. A: The native sequence. B: Clones 3.4F10 and 3.5D8, each had the same mutation. C: Clone 3.5A2. D: Clone 3.5D9.

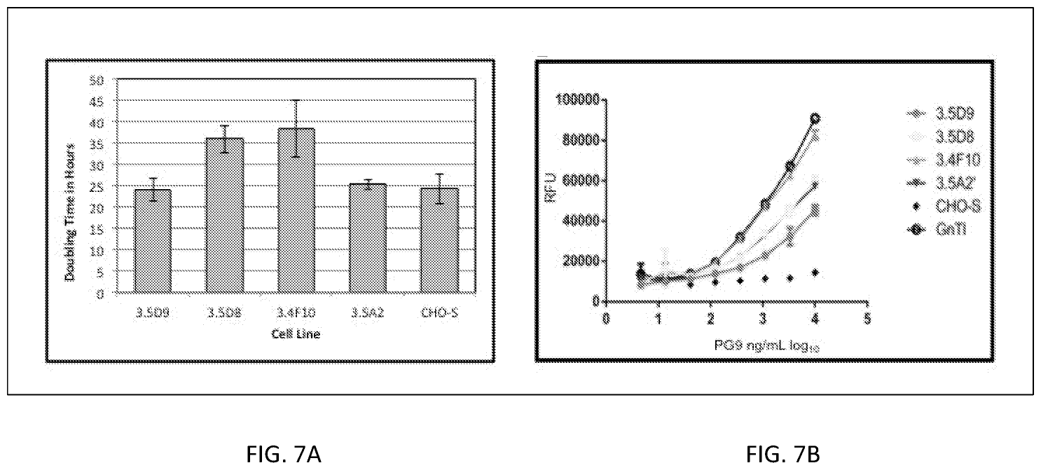

FIGS. 7A and 7B. Cell doubling time and transient expression of gp120 in Mgat1-CHO cell lines. FIG. 7A: Candidate cell lines were placed in 125 mL shake flasks at 20 mL volumes. Cell counts were taken daily for 14 days and cells were back split to 3.5×10⁵ cells/mL daily. FIG. 7B: Transient transfections were performed using Fugene in 24 well plates. Five days post transfection unpurified supernatant was tested via FIA using a gD flag epitope capture and detection with PG9. Purified gp120 from a HEK Gnt1 cell line was used as a comparator high-mannose line.

FIGS. 8A-8C. Expression of gp120 in GB Mgat1⁻ CHO cell line. FIG. 8A: Purified A244 produced by WT CHO-S, GB Mgat1 CHO, and 293 HEK Gnt1⁻ cells, reduced and denatured then run on pre-cast 4-12% tris-glycine SDS Page gel (NuPage, ThermoFisher) and stained with Simply Blue Safe Stain (ThermoFisher) Samples of the same proteins were than then digested with glycosidases Endo (New England BioLabs) H or PNGase F (New England BioLabs) for 16 hours at 37° C. FIG. 8B: Endo H digest. FIG. 8C: PNGase F digest.

FIGS. 9A and 9B. Isoelectric focusing of CHO-S and Mgat1⁻gp120. Purified CHO-S (FIG. 9A) and Mgat1 (FIG. 9B) produced gp120 was fractionated in the first dimension by isoelectric focusing on 11 cm IPG (pI 3-10) strips. Second dimension fractionation was performed using a 4-20% Tris-HCL SDS PAGE pre-cast gel. Two internal pH standards were included, pI 5.6 carbonic anhydrase isozyme II (solid arrow) and pI 3.9 amyloglucosidase (open arrow).

MALDI-TOFF analysis of glycans present on gp120 produced by CHO-S and Mgat1 cell lines. The glycosylation on A244 gp120 produced by CHO-S and Mgat1 cells was stripped by PNGase F digestion and examined by MALDI-TOFF MS. The CHO-S glycosylation is heterogeneous with 72% being complex and 25% high mannose. The Mgat1 material was almost exclusively high mannose (99.47%). This analysis was performed by the Complex Carbohydrate Research Center at the University of Georgia.

FIGS. 10A and 10B. PG9 binding to monomeric gp120 and V1/V2 scaffold improved by Mgat1 knockout in CHO cells. Purified A244 gp120 (FIG. 10A) and V1/V2 fragment (FIG. 10B) protein produced by WT CHO-S, 293 (gp120 only), HEK Gnt1, and GB Mgat1⁻ cell lines was compared for binding affinity to the canonical glycan dependent bNAb PG9.

### Example 2

Construction of Plasmid for the Expression of A244_N332-Rgp120 HIV-1 Vaccine Immunogen in CHO Cell Lines

A244-rgp120 produced in Mgat1⁻ CHO-S cell lines showed increased binding to broadly neutralizing antibody (bNAb) PG9.

This report describes the construction of a plasmid (UCSC1331) for the expression of a mutated HIV-1 envelope gene A244-N332-rgp120 in stable CHO cell lines.

The A244-N332-rgp120 gene encodes a recombinant protein that differs from the parental A244-rgp120 gene product in its ability to bind multiple broadly neutralizing antibodies (bNAbs) that depend on the presence of an N-linked glycosylation site at asparagine residue, N332. The A244-rgp120 immunogen is significant since it was a major component of a prime/boost immunization regimen used in the RV144 clinical trial. This 16,000 person study carried out in Thailand (2003-2009) is the only vaccine trial to demonstrate vaccine induced protection in humans. It is thought that the N332 mutation will improve the A244-rgp120 vaccine immunogen by adding multiple epitopes recognized by broadly neutralizing antibodies (bNAbs). The use of vaccine immunogen that contains multiple epitopes recognized by glycan dependent bNAbs has the potential to improve the level of vaccine efficacy from ˜31% observed in the RV144 trial to a level of 50% or more required for regulatory approval and clinical deployment.

Materials and Methods

The starting plasmid for the construction of UCSC1331 was the PCF1 expression developed in the Berman lab at UCSC. Standard genetic engineering methods, including PCR based mutagenesis, were used to splice and mutate specific gene fragments. A synthetic, codon optimized gene encoding the A244-rgp120 was mutagenized using standard methods to alter the location of N-linked glycosylation sites. Plasmids were propagated in the DH5a strain of E. coli, and plasmids were purified using the endotoxin free QiaGen Gigprep purification kit (cat No. 12391) DNA sequencing was carried out at the University of California at Berkeley Core Sequencing facility using Sanger chain termination sequencing.

Results

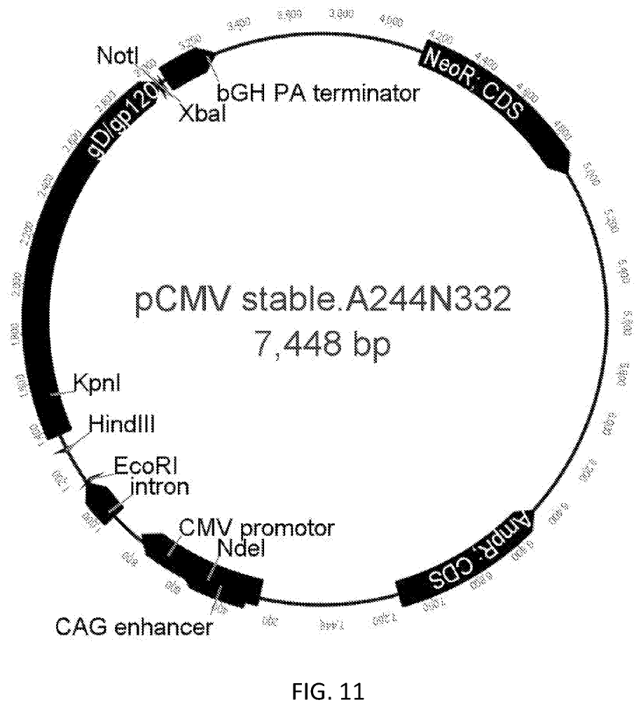

The UCSC1331 plasmid (FIG. 11) was engineered to contain three principal elements: 1) a bacterial plasmid backbone originally derived from PBR322 containing a bacterial origin of replication and a bacterial transcription unit enabling the expression of a gene (β-lactamase) conferring resistance to ampicillin when expressed in bacterial cells, 2) a chimeric DNA fragment containing an transcription unit where an SV40 promoter and origin of replication that enables plasmid replication and the expression of neomycin phosphotransferase and confers resistance to the antibiotic G418 when expressed in mammalian cells; and 3) a second transcription unit with cloning sites for the expression of any transgene (e.g. HIV envelope protein) with a 3′ stop codon for expression in mammalian cells. The second transcription unit includes a partial CMV promoter sequence, and a polyA adenylation sequence from bovine growth hormone (BGH).

Design of Promoter and 5′ Untranslated Sequences.

The core CMV promoter in UCSC1331 differs from the CMV promoter found in many commercially available vectors (e.g. pCDNA3.1) that are useful for transient transfection, but unsuitable for the production of stable cell lines because of gradual inactivation of the CMV promoter by mammalian cell methyltransferases. To allow stable expression in mammalian cells, by avoiding inactivation of the CMV promoter by CHO methyl-transferases, the CMV promoter was mutated to remove two CpG sites at positions C41G and C179G, as described by Moritz and Gopfert (Moritz, B. et al., Scientific Reports, 2015. 5: p. 16952). Other features designed to improve expression levels compared to those achieved by commercial expression vectors was the insertion of a chimeric intron downstream of the CMV promoter (Bothwell, A. L., et al., Cell, 1981. 24(3): p. 625-37; Senapathy, P. et al., Methods Enzymol, 1990. 183: p. 252-78) and a 5′ UTR spacer, upstream from the translational start codon. The precise arrangement of the CMV promoter, the intron, the A244_N332-rgp120 transgene and the bovine growth hormone (BGH) poly A tail expression cassette is diagrammed in FIG. 11.

Design Heterologous Signal Sequence and N-Terminal Purification Tag.

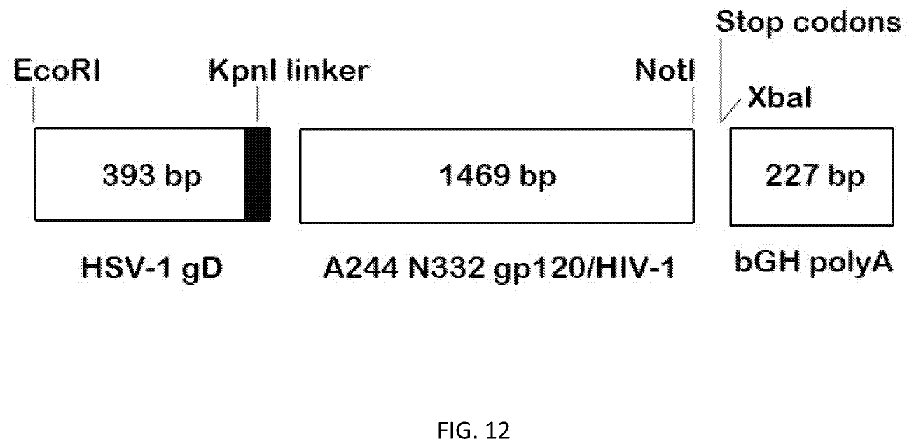

The A244_N332-rgp120 protein produced in these studies was expressed as a fusion protein (FIG. 12) with the N-terminal signal sequences and a 27 amino acid purification tag from Herpes Simplex Virus Type 1 glycoprotein D (gD).

Mutagenesis of N332 and N334 N-Linked Glycosylation Sites.

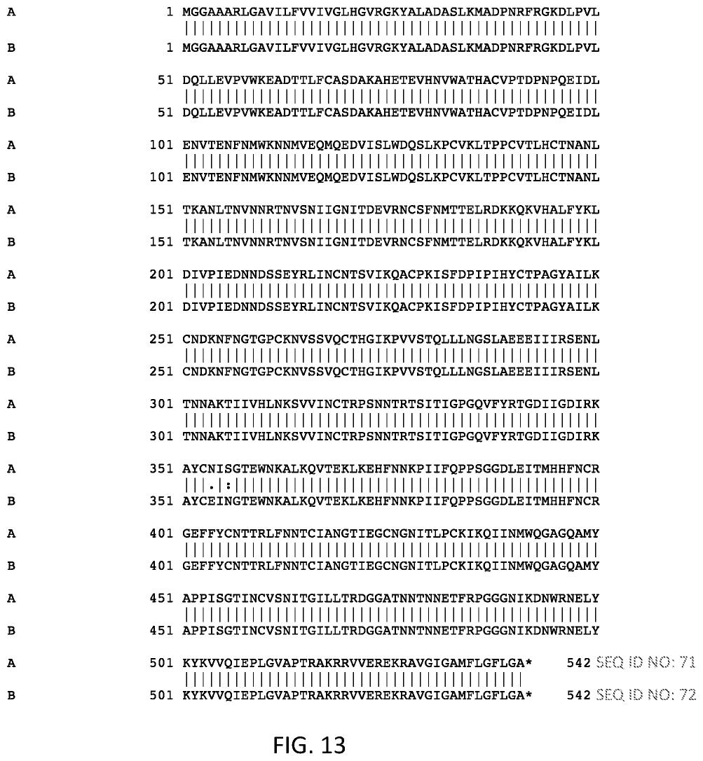

A major functional difference between the wild type and A244-N332 gene products is the location of a critical predicted N-linked glycosylation site (PNGS) in the base of the V3 loop (V3/C3 domain). Thus the N334 PNGS in the wildtype A244-rgp120 gene was deleted and replaced with an alternative PNGS site added at position N332 (Doran et al. 2017, manuscript in preparation). The change is known to facilitate the binding of a major class of broadly neutralizing monoclonal antibodies (bN-mAbs) such as PGT121, PGT128 and 1010-74 that require a glycosylation site at N332. A comparison of the A244-rgp120 and A244-N332-rgp120 protein sequences is provided in a pairwise alignment (FIG. 13).

Assembly of the Chimeric A244 N332-Rgp120 Coding Sequence.

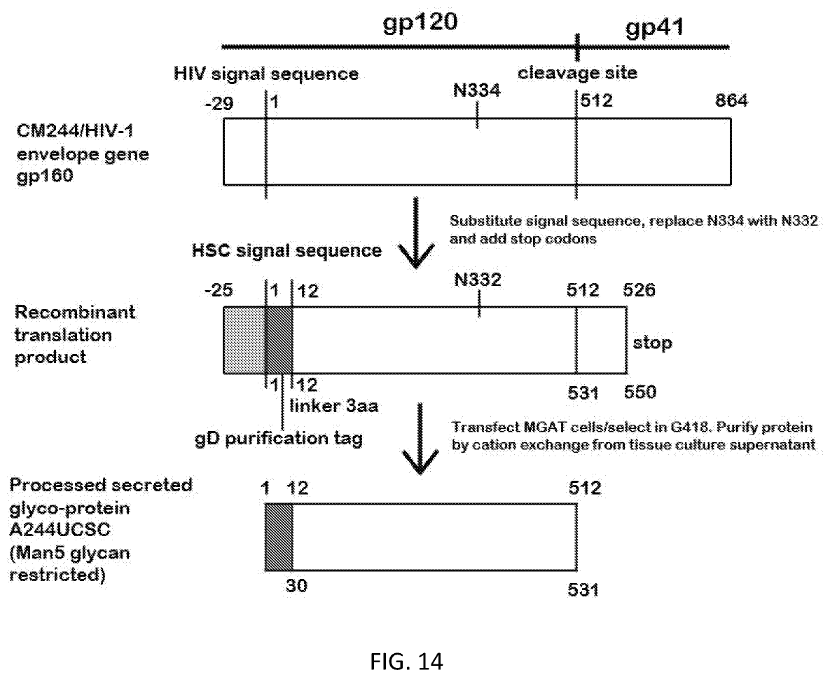

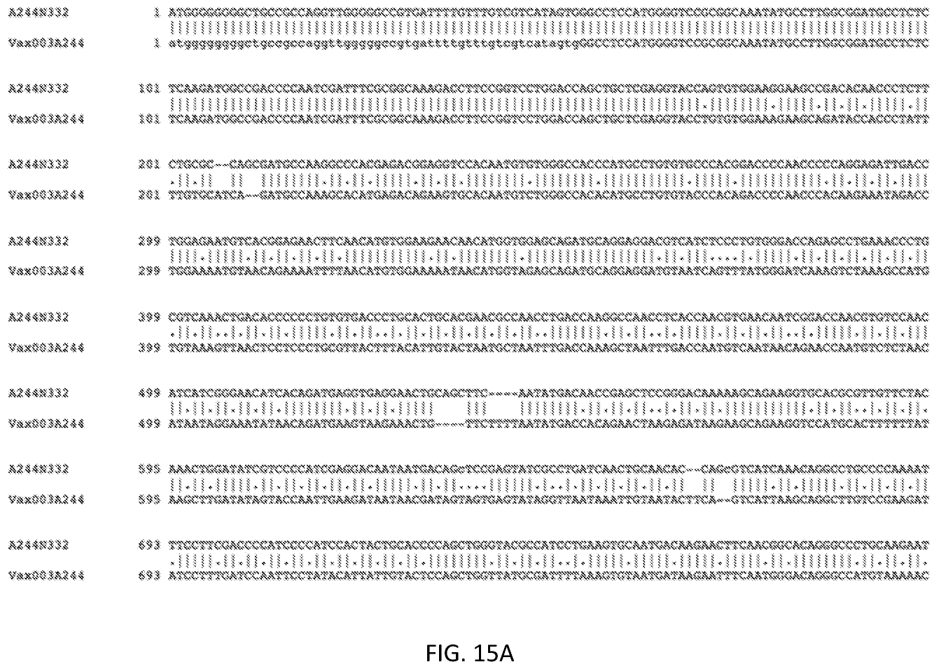

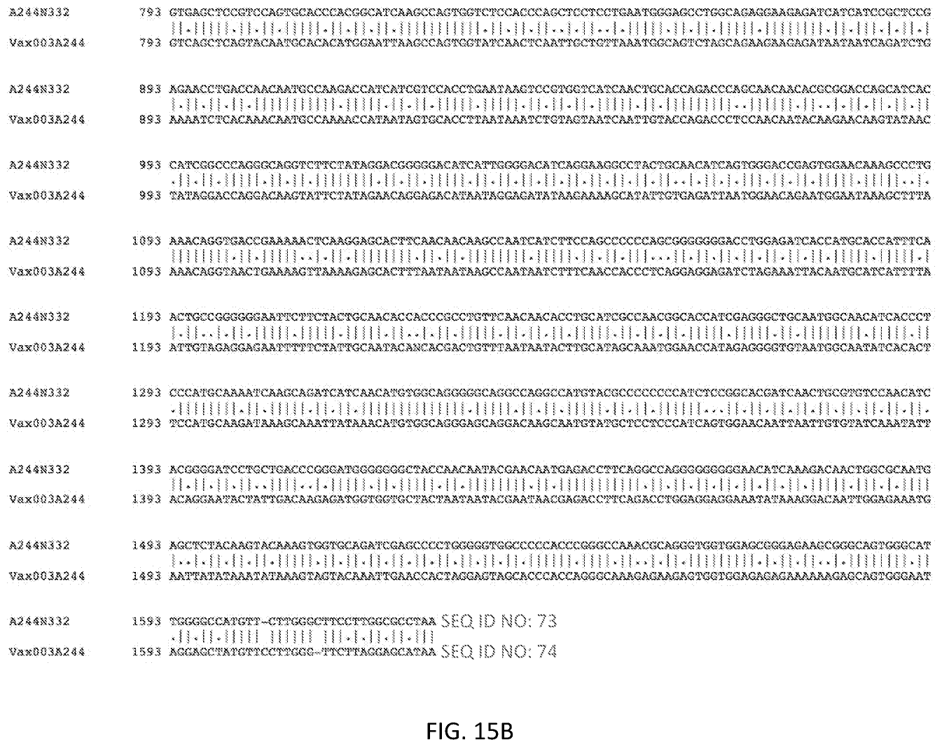

High level expression of multiple rgp120 genes was previously achieved (Lasky, L. A., et al., Science, 1986. 233(4760): p. 209-12) by codon optimization and replacing the signal sequence and 5′ UTR of HIV-1 with that of the Herpes Simplex virus Type 1 glycoprotein D (HSV-1 gD) gene. In addition a 27 amino acid purification tag and a 3 amino acid linker sequence (LEE) was fused to amino acid 12 of the mature fully processed sequence of gp120. A diagram showing this structure is provided in FIG. 14. To construct A244_N332-rgp120 gene, a synthetic DNA sequence encoding a modified CMV core promoter, a chimeric intron, a 5′ UTR spacer, the HSV-1gD signal sequence, and a 27 amino acid HSV-1 gD flag epitope with a three amino acid (LLE) linker sites was purchased from Thermo Fisher (Waltham, Mass., USA). The fragment was designed to include unique restriction sites after the 5′UTR (EcoR1) and at the gD-flag linker (Kpn1) and a Not1 site for convenient cloning of gp120 sequences missing the first 11 amino acids, and is flanked by Hind III and XBa1 restriction sites, which are compatible with the HindIII-XBa1 digested pCF1 vector fragment. In addition, multiple stop codons are encoded between the Not1 the XBa1 site. Assembly of the expression construct was a two-step process: an intermediate was assembled by ligation of the Hind III-XBa1 restricted synthetic sequence to the HindIII-Xba1 fragment of pCF1 (+) to produce the “empty” expression cassette (UCSC1324). The resultant vector was then digested with Kpn1 and Not1, and ligated to a Kpn1-Not1 fragment from plasmid UCSC1250 that encodes a codon optimized A244_{UCSC} gene sequence, and the resulting plasmid (UCSC_CHO.A244N332) was sequenced. A schematic of the fully ligated, codon optimized, chimeric expression gene chimeric gene used to express A244_N332-rgp120 compared to the wildtype A244-rgp120 sequence use in shown in FIGS. 15A and 15B. Chimeric protein expressed by the UCSC_CHO.A244N332 plasmid can be affinity purified using antibody to the gD flag. This vector can be used for transient, or for stable expression by selecting transfected cells with the antibiotic G-418 (Southern, P. J. and P. Berg, J Mol Appl Genet, 1982. 1(4): p. 327-41).

FIG. 11. Diagram of UCSC1331 plasmid used to express A244_N332-rgp120.

FIG. 12. Diagram of the chimeric gene used for the expression of A244_N332-rgp120.

FIG. 13. Emboss Needle pairwise sequence alignment of the amino acid sequence of the A244_N332-rgp120 transcription product with the A244-rgp120 transcription product used to produce rgp120 for the RV144 clinical trial. A is A244_{UCSC} rgp120. B is A244_{GNE} rgp120.

FIG. 14. Comparison of the wild-type A244-rgp120 transcription product with the A244-N332-rgp120 transcription product and the mature processed form of the 244_N332-rgp120 protein.

FIGS. 15A and 15B. Emboss Needle pairwise sequence alignment of the nucleotide sequence of the codon optimized A244_N332-rgp120 gene and the A244-rgp120 gene used to produce A244-rgp120 for the RV144 clinical trial.

### Example 3

Preparation of Goat Polyclonal Antibody Required for Selection Stable Cell Lines Expressing HIV Envelope Proteins Using the ClonePix 2 Robot

The production of affinity purified polyclonal antibodies reactive with HIV envelope protein, gp120, derived from clade B (MN), clade C (CN97001) and clade CRF01_AE (TH023) strains of HIV-1 is described. These antibodies represent an essential reagent for use in the robotic selection of stable cell lines expressing high levels of recombinant HIV envelope proteins.

The ClonePix 2 robotic cell line selection technology requires a fluorescently labeled antibody mixture to a specific secreted gene product that is capable of forming a precipitin band around colonies of cells suspended in a semisolid matrix (e.g. methylcellulose or soft agar). The size of the precipitin band, and the intensity of antibody staining, is proportional to the amount of gene product secreted and serves as the basis for identifying and ranking cell colonies in order of the amount of protein being secreted. Based on this ranking the ClonePix robot is able to sort through tens of thousands of individual cell colonies and identify the small percentage of unusual variants capable of secreting extraordinarily large amounts of proteins. A typical ClonePix 2 experiment might involve screening 40-50,000 individual colonies and selecting 20-40 for further growth and analysis. Before the availability of this instrument, investigators had to manually pick, culture, and assay thousands of individual cell colonies (clones) in order to identify a rare cell line producing high levels of a secreted transgene gene product suitable for biopharmaceutical production. This process was extremely time and labor intensive, usually requiring a team of researchers to pick, culture, and assay the thousands of clones in order to find a high producer cell line. Some proteins such as immunoglobulins are easy to express and high producing cell lines can readily be identified by manual selection in 6 months. However, other proteins, such as HIV envelope proteins, are difficult to express and the identification of high producing cell lines by manual selection typically takes 12-24 months using selective conditions requiring repeated cycles of gene amplification and selection targeting selectable markers such as dihydrofolate reductase and glutamine synthetase.

The ClonePix2 instrument automates the selection of cell lines producing large amounts of secreted gene products, providing a significant reduction in the time and cost of selecting a high producing cell line that can be used for biopharmaceutical production. Commercial antibody reagents are available for the isolation of cell lines producing monoclonal antibodies, but are not available for other proteins such as HIV envelope proteins. Therefore reagents that could be used for the identification of cells expressing levels of recombinant HIV envelope proteins >50 mg/L in transfected CHO cells were created. Initial experiments based on the suggestions of the ClonePix2 manufacturer (Molecular Devices, Mountain View, Calif.) and other HIV vaccine researchers (Lu, S. 2015. HIV Env. Manufacturing Workshop, NIAID, Bethesda, Md. Jun. 11, 2015) involved the growth and production mixtures of fluorescently labeled monoclonal antibodies. The formation of precipitin bands requires an antigen with at least three different epitopes and antibodies in approximately equal concentrations to each of these epitopes. However, after spending ˜18 months trying cocktails of three or more monoclonal antibodies to different gp120 epitopes precipitin bands around colonies of cells known to express gp120 could not be observed using this technique. It was therefore concluded that the same approach used in selecting cell lines producing monoclonal antibodies was unlikely to work for selecting cell lines producing gp120, and that a different strategy was needed. Protein A or protein G purified polyclonal rabbit and goat antibodies to recombinant gp120 were then used to label cell lines secreting HIV envelope proteins. This approach was similarly unsuccessful. Finally, it was reasoned that the background fluorescence in purified polyclonal sera might obscure the visualization of the minute precipitin bands surrounding each cell colony.

Materials and Methods

Ethics Statement.

Animal experiments were performed according to the guidelines of the Animal Welfare Act. Pocono Rabbit Farm and Laboratory, Inc. has an Animal Welfare Assurance on file with The Office of Laboratory Animal Welfare (OLAW). The Animal Welfare Assurance number is A3886-01 effective Jan. 29, 2013 through Jan. 31, 2017.

Gp120 Immunogens.

Purified gp120s from three clades of HIV (CRF01_AE, B, and C) were expressed by large scale transient expression in 293 cells. Each protein was expressed as a fusion protein containing an N-terminal 27 amino acid purification tag from Herpes Simplex Virus type 1 glycoprotein D (gD). Growth conditioned cell culture medium was harvested, filtered, and the gp120 proteins were purified by immunoaffinity chromatography using a monoclonal antibody to gD coupled to an insoluble matrix. The proteins recovered consisted of gp120s from the A244, MN, CN97001 isolates of HIV-1. SDS-PAGE gels of the proteins used for immunization are provided in FIG. 16). The lots of the three antigens used were: 1) CN97001-rgp120, produced in 293HEK cells, 2) MN468-rgp120 (lot 456; produced in Gnt1-293 cells); and 3) A244_{GNE}-rgp120 (lots 368, 329, and 338, produced in Gnt1-293 cells).

Goat Immunization.

A single male goat (557) weighing approximately 56 kg was immunized with a mixture of three gp120 antigens at Pocono Laboratories, Canadensis, Pa. Immunization began on day 0 with a mixture of all three immunogens (100 μg each) and booster immunizations on days 7, 14, and 35, 49 and 63. The primary immunization on day 0 was via intradermal injection using Complete Freund's Adjuvant (CFA). The boosts at days 7, 14, and 35 were intra muscular and used Incomplete Freund's Adjuvant with MightyQuick Stimulator (PRF&L's proprietary immune stimulator). Bleeds were taken on days 0 (prebleed), 21, 28, 35, 42, 56, 63, 70, and a final exsanguination bleed at day 77. 2.5 L of 557 serum is stored at −20° C. at UCSC.

Verification of Antibody Levels in Goal Serum.

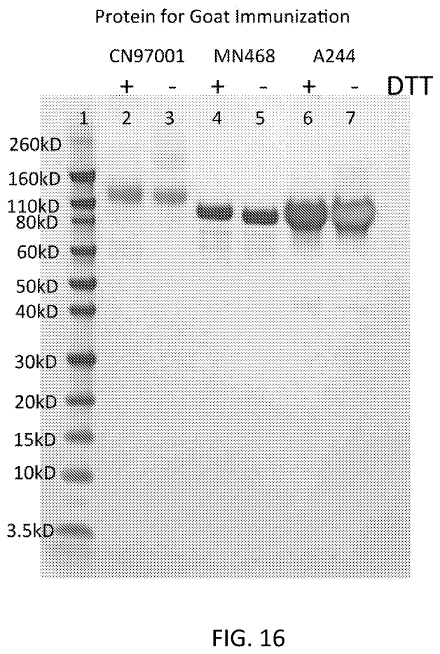

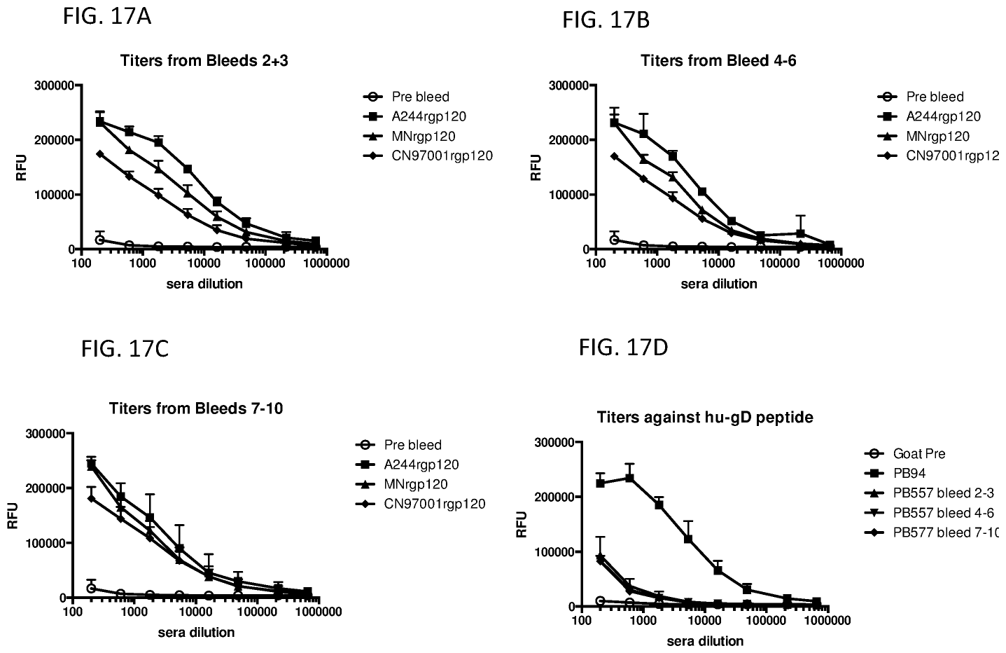

The goat serum was assayed by direct FIA assay using 96-well plates (Fluortrac 600, Greiner) coated with 2 ng/ml of protein overnight in PBS. Bound antibody was detected using a polyclonal donkey anti-goat antibody at a dilution of 1/5000 (Life Technologies, Carlsbad, Calif.), and plates read on an Envision plate reader (Perkin Elmer, Waltham, Mass.). Results are shown in FIG. 17.

Purification of Antibodies.

Total IgG was purified from goat serum by affinity chromatography using a HiTrap Protein G column (GE Healthcare, Little Chalfont, United Kingdom), following the manufacturer's instructions. The purified antibodies were stored at 20 mg/ml in PBS at −20° C. Immunoaffinity columns were prepared by coupling MNgp120-rgp120 and A244-rgp120 to cyanogen bromide activated sepharose (GE Healthcare, Little Chalfont, United Kingdom). An aliquot of serum was purified by successive purification on two affinity columns created with TH023-rgp120, MN-rgp120, respectively. Columns were washed with 10 column volumes of 50 mM Tris, 0.5 M NaCl, 0.1 M TMAC (tetramethyl ammonium chloride) buffer (pH 7.4), and eluted with 0.1 M sodium acetate buffer, pH 3.0. The pH of the buffer was neutralized by the addition of 1.0 M Tris (1:10 ratio) and the resulting solution was concentrated using an AMICON molecular weight cutoff centrifuge tube (Millipore, Billericia, Mass.). The purified protein was adjusted to a final concentration of 1-2 mg/mL in PBS buffer. Protein concentrations were determined using the bicinchoninic acid assay (BCA) method.

Alexa 488 Antibody Labeling.

Two aliquots of goat 557 polyclonal antibody were labeled with Alexa 488 (Thermo Fisher Scientific, Waltham, Mass.). The first batch was protein G purified and the second, immunoaffinity purified. Conjugate labeling was performed using an Alexa Fluor labeling kit (Thermo Fisher Scientific, Waltham, Mass.) as per instructions excepting that the labeled antibody was separated from unlabeled dye using a 30K cutoff Amicon Ultra spin column centrifuging three times 10 min at in a 3750 rpm/2750 rcf washing with 10 ml of PBS each time until no dye was detected in the filtrate. The Alexa 488-conjugated antibody was concentrated to 1.8-2 mg/ml, and the amount of dye coupled to antibody, was calculated using a Nanodrop spectrophotometer (Thermo Fisher Scientific, Waltham, Mass.). It was determined to be to be four moles and six moles per mole respectively, for the protein G and immunoaffinity batches. Anywhere between 4-9 moles per mole is deemed acceptable by the manufacturers protocol. Labeled antibody filter sterilized through a 0.2 micron (will use 0.1 in future) filter, and was stored at 4° C. in the dark in a refrigerator in room 288 Baskin labs, UCSC.

Results

Recombinant gp120s for Immunization Studies.

Recombinant gp120s from the A244, MN, and CN97001 isolates of HIV-1 were expressed 293 HEK cells by transient transfection as described previously (Nakamura, G. R., et al., J Virol, 1993. 67(10): p. 6179-91; Smith, D. H., et al., PLoS One, 2010. 5(8): p. e12076). Growth conditioned cell culture supernatants were collected, filtered, and applied to an immuno-affinity column prepared with a purified monoclonal antibody reactive with the N-terminal 27 amino acids of Herpes Simplex Virus Type 1 glycoprotein D (gD). The column was eluted at pH 3, and the eluted gp120 was purified by immune-affinity and size exclusion chromatography. The purified proteins were analyzed for purity and quality (e.g. proteolytic degradation) by SDS_PAGE. Visualization after Coomassie blue staining (FIG. 16) showed that all of the protein ran as a single band and that there was little if any evidence of dimerization or proteolysis upon reduction with dithiothreitol.

Immunization of Goat 577 with Purified Gp120.

A healthy goat with a documented record of veterinary care was immunized five times with a mixture of gp120s from 3 different clades using a protocol compliant with USDA guidelines and the Animal Welfare Act. Adjuvants were provided by the contract research organization, Pocono Laboratories, Canadensis, Pa. Samples of antisera were collected after each immunization and pooled sera from pairs of immunization were monitored for the presence of antibodies to all three gp120s used for immunization as well as antibodies to the HSV-1 gD purification tag present on all three immunogens. Antibodies to all three antibodies were detected in the pooled sera analyzed (FIG. 17), however the titers to CN97001-rgp120 were lower than the titers to the other two antigens until after bleeds 7-10. Serum was collected and stored as described above (Materials and Methods).

Comparison of Protein G and Antigen Specific Affinity-Purified Antibody in ClonePix Assay.

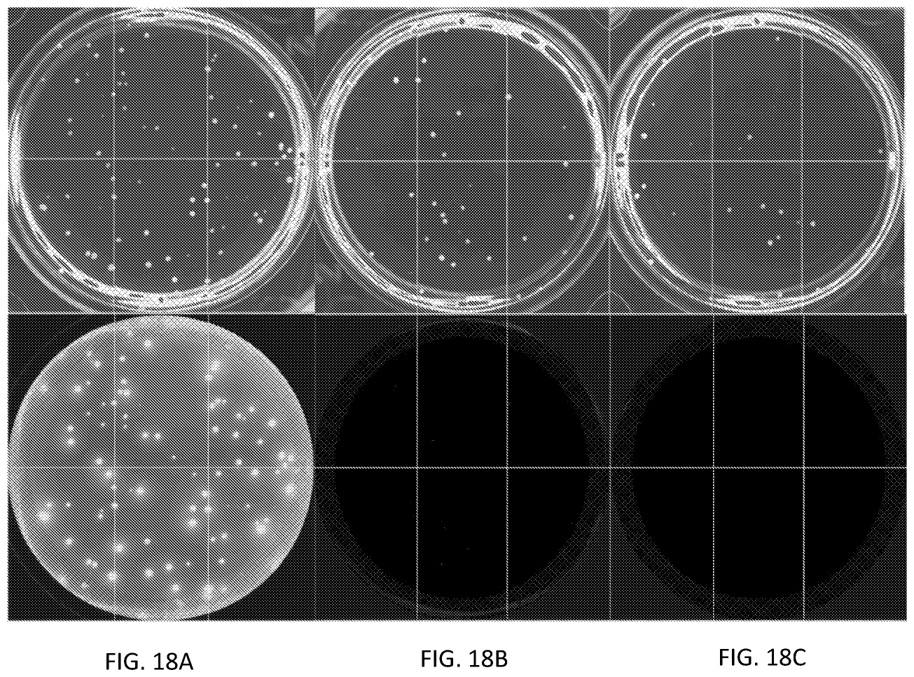

An Mgat1⁻CHO cell line expressing A244_N332-rgp120 (clone 5F) were diluted to 25 cells/ml in CHO-A matrix (Molecular Devices, Sunnyvale, Calif.) containing 10 μg/ml of either Alexa 488, protein G purified, IgG from goat 577 or immuno-affinity purified, Alexa 488 labeled, IgG antibody purified goat 557. Colonies were imaged using the ClonePix 2 after 14 days in culture. Halos were visible around clones that had been incubated in the presence of Immuno-affinity purified, Alexa 488 antibody, but were absent in the protein-G purified Alexa 488 test wells (FIG. 18). These results demonstrate that polyclonal antibodies to gp120 can be used to visualize colonies of cells secreting recombinant HIV envelope proteins provided that they are immunoaffinity purified prior to labeling with an appropriate fluorophore (e.g. Alexa 488).

FIG. 16. All three proteins were boiled with LDS sample loading buffer (Invitrogen) with or without reducing reagent DTT addition for 2 minutes. Then it was run in 4-12% Bis-Tris gel with MES running buffer (Thermo Fisher, Life Technologies, Carlsbad, Calif.), stained by SimplyBlue Safe stain (Thermo Fisher, Life Technologies, Carlsbad, Calif.) for a hour and destained overnight in distilled water. SDS-Gel image was captured by Fluorchem Q system (Alpha Innotech, Genetic Technologies, Grover, Mo.). Lane 1: Molecular Weight standard (Thermo Fisher, Life Technologies, Carlsbad, Calif.) Lane 2 and 3. Clade C gp120: CN97001. It was produced from 293 cells in Genetech with/without reducing reagent DTT addition respectively. Lane 4 and 5. Clade B gp120: MN468, a glycosylation mutation of MN strain. It was produced from Gnt1-cells, purified by affinity and gel filtration chromatograph in UCSC, with/without reducing reagent DTT addition respectively. Lane 6 and 7. Clade AE gp120: A244. It was produced from Gnt1-cells, purified by affinity and gel filtration chromatograph in UCSC, with/without reducing reagent DTT addition respectively.

FIGS. 17A-17D. Measurement of antibodies to A244-, MN-, and CN97001 gp120s and to the HSV1 glycoprotein purification tag during the course of immunization of Goat 577. Protein lots #647, #648 and #15 of gp120 and a synthetic peptide corresponding to the gD purification tag were used to in a direct coat HA assay. Titer data is grouped for production lots that were combined for purification purposes. Bleeds 2 and 3 were protein G purified and affinity purified for use in the ClonePix2 cell line selection experiments.

FIGS. 18A-18C. Comparison of ClonePix2 images obtained with protein G purified, Alexa 488 labeled goat IgG and with affinity-purified, Alexa 488 labeled IgG. Mgat1-CHO cells were transfected with the UCSC1331 plasmid by electroporation and the resulting cells were suspended in semi-solid CHO-A growth media (Molecular Devices, Sunnyvale, Calif.) containing Alexa488-labelled IgG elicited against a mixture recombinant gp120s from the MN-, A244-, and CN97001-strains of HIV-1. The cells were cultured for 14 days at 37° C. in 8% CO₂ and then visualized in the ClonePix 2 robotic selection system. FIG. 18A, images of cells after a 14 day incubation of Mgat1-cells expressing A244-N332-rgp120 with polyclonal immuno-affinity purified Alexa 488 labeled goat IgG (goat557). Top row, white light; bottom row, fluorescent light (535 nM). FIG. 18B, images of cells after a 14 day incubation of Mgat1-cells expressing A244_N332-rgp120 with 10 ug/ml of Alexa 488 labeled, protein G purified, goat IgG. Top row, white light; bottom row, fluorescent light (535 nM). FIG. 18C, images of cells from a control experiment where of Mgat1-cells expressing A244_N332-rgp120 were incubated for 14 days without added antibody. Top row, white light; bottom row, fluorescent light (535 nM).

### Example 4

Method for the Selection of Stable CHO Cell Lines Producing Recombinant HIV Envelope Proteins for Use as Vaccine Immunogens

This report describes a novel method for the rapid development of a stable CHO cell lines producing recombinant forms of the HIV-1 envelope proteins, gp120, where N-linked glycosylation is limited to mannose-5 glycans and earlier structures in the N-linked glycosylation pathway. This method provides major economic advantages in the HIV vaccine manufacturing process, and provides major biologic advantages in pharmacokinetics and antigenic structure. These improvements derive from improved method for creating novel cell lines with extraordinarily high gp120 production capacity, as well the use of a novel cell line Mgat1 CHO that limits N-linked glycosylation primarily to mannose-5 glycans. Because the final product incorporates multiple glycan dependent epitopes recognized by broadly neutralizing antibodies, the new molecule (A244_N332-rgp120) described in this report should be more effective than previous gp120 vaccines in eliciting protective immunity than and can be manufactured more efficiently at a substantially reduced cost.

The development of a safe, effective, and affordable HIV vaccine is a global public health priority. After more than 30 years of vaccine development, a vaccine with these properties has yet to be described. To date, the only clinical study to show that vaccination can prevent HIV infection is the 16,000 RV144 trial carried out in Thailand between 2003 and 2009 (Rerks-Ngarm, S., et al., N Engl J Med, 2009. 361(23): p. 2209-20). This study involved immunization with a recombinant canarypox virus to induce cellular immunity and a bivalent recombinant gp120 vaccine designed to elicit protective antibody responses (Berman, P. W., et al., Virology, 1999. 265(1): p. 1-9; Berman, P. W., AIDS Res Hum Retroviruses, 1998. 14 Suppl 3: p. S277-89). Unfortunately only a modest level protection (31%) was achieved in this study, resulting in an urgent need to find a way to improve the level of protection. Improving the efficacy of gp120 vaccines from 31% seen in RV144 to the level of protection of 50% or more (thought to be required for regulatory approval), is likely faster and more cost effective than developing a new vaccine concept from scratch. Several correlates of protection studies have suggested that the protection achieved in the RV144 trial can be attributed to antibodies to gp120 (Haynes, B. F., et al., N Engl J Med, 2012. 366(14): p. 1275-86; Montefiori, D. C., et al., J Infect Dis, 2012. 206(3): p. 431-41; O'Connell, R. J. et al., Expert Rev Vaccines, 2014. 13(12): p. 1489-500). A roadmap to improve the gp120 vaccine used in the RV144 trial has been provided by the recent identification of multiple broadly neutralizing monoclonal antibodies (bN-mAbs) to gp120. Surprisingly, many of these were found to recognize unusual glycan dependent epitopes that were dependent on mannose-5 or mannose-9 structures (Walker, L. M., et al., Science, 2009. 326(5950): p. 285-9; Walker, L. M., et al., Nature, 2011. 477(7365): p. 466-70). Since the gp120 vaccine used in the RV144 trials lacked these structures, they also lacked multiple epitopes with the potential to stimulate protective virus neutralizing antibodies. The work described in this report represents the results of a focused effort to find a practical and economical way to produce an improved gp120 vaccine antigens possessing the glycan structures required to bind bNAbs.

Previous experience showed that the production of recombinant HIV envelope proteins (gp120 and gp140) for clinical research and commercial deployment was extremely challenging. Not only was it difficult to isolate stable cell lines producing commercially acceptable yields (e.g. >50 mg/mL), but it was also difficult to consistently manufacture a high quality, well defined product with uniform glycosylation, free of proteolytic clipping and aggregated species. Key breakthroughs in improving the yields of HIV envelope expression came with the discovery that the native HIV envelope glycoprotein signal sequence often limited expression and that replacement with other signal sequences such as Herpes Simplex Virus glycoprotein D (gD) or the prepro signal sequence of tissue plasminogen activator enhanced expression (Lasky, L. A., et al., Science, 1986. 233(4760): p. 209-12). Additional progress was achieved when it was recognized that codon optimization could enhance HIV envelope glycoprotein expression (Haas, J. et al., Curr Biol, 1996. 6(3): p. 315-24). However, even with these improvements it was often difficult to create stable CHO cell lines, suitable for vaccine production that expressed more than 2-20 mg/L. These low levels of expression necessitated production of candidate HIV vaccine antigens at large scale (up to 10,000 L) in order to produce sufficient material for large scale vaccine trials such as the 16,000 person RV144 HIV vaccine trial (Rerks-Ngarm, S., et al., N Engl J Med, 2009. 361(23): p. 2209-20). Vaccine production at this scale is very expensive and required the use of manufacturing facilities costing in excess of S500 million for production. In principle, a major way to reduce the cost of manufacturing and production is to develop high producing cell lines yielding 200-2000 mg/L such as those used to produce therapeutic monoclonal antibodies. Because the required dosage of subunit vaccines is typically less than 1 mg the size of the manufacturing facility required for the commercial production of gp120 vaccines from a high producing cell line is proportionally less (e.g. 1,000 L) as is the cost of materials and supplies required to recover the recombinant proteins from the smaller size fermentation cultures. This report describes a rapid method to produce a high yielding CHO cell line producing gp120 that should result in a 10-fold or more reduction in the cost of manufacturing and production compared to HIV vaccines described previously. Moreover the disclosed method for producing gp120 cell lines requires only 2-3 months compared to previous efforts which have taken 12-24 months.

Another challenge in the development of recombinant gp120 derives from the fact that it is highly glycosylated and typically possess 25-26 predicted N-linked glycosylation sites. Because each glycosylation site can be occupied by as many as 40 different glycans (Go, E. P., et al., J Virol, 2011. 85(16): p. 8270-84; Go, E. P., et al., Journal of proteome research, 2013. 12(3): p. 1223-1234), some with as many as 4 sialic acid residues, the net charge and biophysical properties of recombinant gp120 are highly variable. The variability in glycosylation makes it difficult to purify and difficult to define the chemical structure of the recombinant protein. Moreover since the pharmacokinetic and pharmacodynamic properties of glycoproteins such as gp120 that are in large part determined by the sialic acid content, glycan variability represents a potential source of product variability (Sinclair, A. M. and S. Elliott, J Pharm Sci, 2005. 94(8): p. 1626-35). Disclosed herein is a solution to the problems in glycosylation heterogeneity. The solution involves the production of gp120 in a novel CHO cell line with a mutation in the Mgat1 gene (see Example 1). Production of recombinant gp120 in this cell line limits glycosylation primarily to mannose-5 and earlier structures in N-linked glycosylation pathway. This approach considerably improves the homogeneity of the recombinant gp120 and simplifies the recovery process required to manufacture the protein. It also reduces “lot to lot” variation and should improves the consistency and biological activity of the protein. Finally, as described above, mannose-5 glycans are an essential feature of many epitopes recognized by broadly neutralizing antibodies. Thus the novel method for producing gp120 described in this report substantially improves the quality and biologic activity of recombinant gp120 while at the same time lowering the manufacturing costs compared to previous methods.

Materials and Methods

Broadly Neutralizing Human Monoclonal Antibodies.

The following reagents were obtained through the NIH AIDS Reagent Program, Division of AIDS, NIAID, NIH: PG9 (Walker, L. M., et al., Science, 2009. 326(5950): p. 285-9), VRC01 (Wu, X., et al., Science, 2010. 329(5993): p. 856-861), PGT121, PGT128 (Walker, L. M., et al., Nature, 2011. 477(7365): p. 466-70), and 101074 (Shingai, M., et al., Nature, 2013. 503(7475): p. 277-280). PG16 was purchased from and Polymun A.G. (Vienna, Austria). The antiviral compound CD4-IgG has been described previously (Ashkenazi, A., et al., Proc Natl Acad Sci USA, 1991. 88(16): p. 7056-60; Capon, D. J., et al., Nature, 1989. 337(6207): p. 525-31) and was provided by GSID. All secondary polyclonal antibody conjugates were purchased from Jackson ImmunoResearch Laboratories, West Grove).

Transfection of Gp120 Genes by Electroporation.

Mgat1⁻ CHO is a novel cell line derived from the commercially available CHO-S cell line (Thermo Fisher, Life Technologies, Carlsbad, Calif.). The cell line possess mutation that inactivate both copies of the Mannosyl (Alpha-1,3-)-Glycoprotein Beta-1,2-N Acetylglucosaminyltransferase 1 gene (Mgat1). Cells with this produce proteins where N-linked glycosylation is limited primarily to Man(5) GlcNAc(2) glycans with a small percentage of glycans possessing earlier structures in the N-linked glycosylation pathway (e.g. Mannose-8 and mannose-9) (Byrne et al 2017, manuscript in preparation). Cell cultures of Mgat1 were maintained in CD-CHO (Thermo Fisher Life Carlsbad Calif.) 8 mM Glutamax, 1×HT (Thermo Fisher Life Carlsbad Calif.) culturing at 37° C., with 8% CO₂ and 85% humidity, rotating at 135 rpm in a Climo 1SF1× shaker (Kuhner, San Carlos, Calif.). Mgat1 cells were transfected with a linearized plasmid expression vector (UCSC1331) containing a chimeric gene directing the synthesis of a variant of the gp120 gene from the A244 isolate of HIV-1. The protein synthesized by this gene is termed A244_{UCSC}rgp120. This plasmid contains a gene encoding the neomycin resistance allowing selection in the antibiotic G418 (Southern, P. J. and P. Berg, J Mol Appl Genet, 1982. 1(4): p. 327-41). Also transfected, was a plasmid directing the expression of dihydrofolate reductase (DHFR) that could be used as a selectable marker. Transfection of Mgat1-cell was accomplished using electroporation using the MaxCyte Scalable transfection system (MaxCyte Inc., Gaithersburg, Md.) according to the manufacturer's protocol. Briefly, 120 μg of plasmid was mixed with 8×E107 cells in C400 cuvette in MaxCyte transfection buffer. After electroporation the cells were cultured for 24 hrs in 15 mL of non-selective CD opti-CHO (Thermo Fisher Life, Carlsbad Calif.) media supplemented with 2 mM glutamax (Thermo Fisher Life Technologies, Carlsbad Calif.), 0.1% Pluronic (Thermo Fisher Life Technologies, Carlsbad Calif.) 1× hypoxanthine/thymidine (Thermo Fisher Life Technologies, Carlsbad Calif.) in a 125 ml Corningflask (Thermo Fisher Life Technologies, Carlsbad Calif.), at 8% Co2 370C, rotating at 135 rpm, with 85% humidity.

Seeding of Transfected Cells in Semi Solid Media.

Twenty four hours after electroporation, cells were counted and diluted to a concentration o5×10³/ml in 50 ml of semi-solid CHO-Growth A with L-glutamine (Molecular Devices, Sunnyvale, Calif.) containing 500 μg/ml of Geneticin (G418) (Thermo Fisher, Life Technologies, Carlsbad, Calif.), 2.5% New Zealand Fetal Bovine Serum (Thermo Fisher, Life Technologies, Carlsbad, Calif.) 100 ng/ml Methotrexoate (Sigma-Aldrich, St. Louis, Mo.) and 10 μg/ml Alexa 488 labeled affinity-purified polyclonal antibody in 6 well plates (Greiner, Kremsmünster, Austria). The plates were incubated in static culture at CO₂ 37° C. with 8% and 85% humidity. Distinct colonies with a fluorescent halo were visible after 6 days, but robotic selection was performed after 16 days to allow for additional antibody selection.

Isolation of Single High Producing Clones.

The ClonePix2 system (Molecular Devices, Sunnyvale, Calif.) was used to image colonies secreting A244_{ucsc}rgp120 into the semi-solid media. Colonies were imaged under white light and fluorescence (Lee, C., et al., Bioprocess International, 2006. 4(sup 3): p. 32-35). Both images were superimposed, and the colonies sorted according to mean exterior fluorescent intensity. The top 0.1% were aspirated with micro-pins controlled by the ClonePix 2 system, and dispersed automatically in a 96-well plate containing 100 μl of rescue media XP CHO (Genetix Mol Devices, Sunnyvale, Calif.) conditioned 0.2 micron filtered CD-CHO (Thermo Fisher, Life Technologies, Carlsbad, Calif.) at 50:50 ratio, with 1× hypoxanthine and thymidine (HT) supplement (Thermo Fisher, Life Technologies, Carlsbad, Calif.), 1× Insulin/Transferrin/Selenium supplement (Thermo Fisher, Life Technologies, Carlsbad, Calif.) with a final concentration of 500 μg/ml Geneticin/G418 (Thermo Fisher, Life Technologies, Carlsbad, Calif.), and cultured at 37° C., with 8% CO₂ 85% humidity. After 5 days in culture, a further 100 μl of rescue media was added to each well. Cultures were assayed at day 9 to confirm rgp120 production, and positive colonies transferred to 2 ml wells (37° C., 8% CO₂ and 85% humidity). Supernatants from 2 ml wells were assayed for protein production by capture ELISA and western blot. Cells simultaneously at a viable density and positive for A244_{ucsc} rgp120 expression were transferred to 50 ml shaker tubes, then 125 ml shaker flasks, culturing at 37° C., with 8% CO₂ and 85% humidity, rotating at 135 rpm in a Climo 1SF1× shaker (Kuhner, San Carlos, Calif.).

Batch Fed Culture Expression.

The ClonePix2 system (Molecular Devices, Sunnyvale, Calif.) was used to image colonies secreting A244_{UCSC}rgp120 into the semi-solid media. Colonies were imaged under white light and fluorescence (Lee, C., et al., Bioprocess International, 2006. 4(sup 3): p. 32-35). Both images were superimposed, and the colonies sorted according to mean exterior fluorescent intensity. The top 0.1% were aspirated with micro-pins controlled by the ClonePix 2 system, and dispersed automatically in a 96-well plate containing 100 μl of rescue media XP CHO (Genetix Mol Devices, Sunnyvale, Calif.) conditioned 0.2 micron filtered CD-CHO (Thermo Fisher, Life Technologies, Carlsbad, Calif.) at 50:50 ratio, with 1× hypoxanthine and thymidine (HT) supplement (Thermo Fisher, Life Technologies, Carlsbad, Calif.), 1× Insulin/Transferrin/Selenium supplement (Thermo Fisher, Life Technologies, Carlsbad, Calif.) with a final concentration of 500 μg/ml Geneticin/G418 (Thermo Fisher, Life Technologies, Carlsbad, Calif.), and cultured at 37° C., with 8% CO₂ and 85% humidity. After 5 days in culture, a further 100 μl of rescue media was added to each well. Cultures were assayed at day 9 to confirm rgp120 production, and positive colonies transferred to 2 ml wells (37° C., 8% CO₂ and 85% humidity). Supernatants from 2 ml wells were assayed for protein production by capture ELISA and western blot. Cells simultaneously at a viable density and positive for A244_{UCSC}rgp120 expression were transferred to 50 ml shaker tubes, then 125 ml shaker flasks, culturing at 37° C., with 8% CO₂ and 85% humidity, rotating at 135 rpm in a Climo 1SF1X shaker (Kuhner, San Carlos, Calif.).

Batch Fed Culture Expression.

At day 56, clones selected for a larger scale batch fed protein production experiment were cultured in production media, that is CD-OptiCHO (Thermo Fisher, Life Technologies, Carlsbad, Calif.) at 32° C., supplemented with 1 mM sodium butyrate, 2 mM Glutamax, X1HT, 0.1% Pluronic® at 8% CO₂85% humidity, and a rotation speed of 135 rpm at a starting density of 1×10⁷ cells/ml, until the viability dropped below 50%. Cultures were fed daily with MaxCyte CHO A Feed, 0.5% Yeastolate (BD, Franklin Lakes, N.J.), 2.5% CHO-CD Efficient Feed A, 0.25 mM GlutaMAX, 2 g/L Glucose (Sigma-Aldrich, St. Louis, Mo.). Supernatant was harvested by pelleting the cells at 250 g for 30 min followed by pre-filtration through Nalgene™ Glass Pre-filters (Thermo Scientific, Waltham, Mass.) and 0.45 micron SFCA filtration Nalgene (Thermo Scientific, Waltham, Mass.), then stored frozen at −20° C. before purification.

ELISA to Measure A244_{UCSC}-Rgp120 Production.

An indirect capture ELISA was carried out as follows: 96-well Nunc MaxiSorb flat bottom plates (Thermo Fisher Scientific, Waltham, Mass.) were coated with 2 μg/ml of anti-gD flag antibody 34.1 in PBS. After blocking for 1 hr with 5% milk/PBS, recombinant protein from tissue culture supernatant was captured by overnight incubation at 4° C. The plates were washed four times with 0.05% Tween/PBS, and bound protein was detected using antigen specific anti-CRF01AE/MN rabbit polyclonal antibody (PB94) or the bNAb PG9 followed by either goat anti-rabbit or goat anti-human H and L chain affinity purified secondary Horse Radish Peroxidase (HRP) conjugated antibodies at a 1/5000 dilution in 5% milk/PBS, as appropriate (Jackson ImmunoResearch Laboratories, West Grove, Pa.). Control standards included three-fold serial dilutions of purified recombinant r-gD-gp120 proteins starting at 10 μg/ml. HRP was detected using o-Phenylenediamine dihydrochloride substrate (Thermo Fisher Scientific, Waltham, Mass.) following the manufacturer's instructions. Assays were stopped after 10 min development with 3 M H₂SO₄, and read on a microtiter plate reader at a wavelength of 490 nm. Protein yield was quantified by serial dilution and interpolation from a standard curve prepared by serial dilution of purified A244_{UCSC}rgp120 HIV-1 produced by transient transfection of MGAT1 cells, and assayed at the same time.

ELISA to Measure the Binding of bNAbs.

A direct ELISA format was used to measure the binding of monoclonal antibodies to A244_{UCSC}-rgp120 Purified protein was carried out on 96-well Nunc MaxiSorb flat bottom plates (Thermo Fisher Scientific, Waltham, Mass.) coated with 2 μg/ml PBS of A244_{UCSC}rgp120 HIV-1 or A244_{GNE} rgp120 protein overnight. Plates were blocked with 5% milk/PBS for 1 hr, washed four times with 0.05% Tween/PBS, and bNAbs three-fold serially diluted in blocking buffer, added for 1 hr. The plates were washed four times with 0.05% Tween/PBS, and specific bNAb binding detected using a goat anti-human L and H chain HRP conjugated secondary antibody as previously described. Data was plotted and analyzed using Prism version 6.00 for Mac (GraphPad Software, La Jolla, Calif., www.graphpad.com).

Western Blot to Detect Antibody Binding to Gp120 Produced by Different Clones.

Growth conditioned cell culture supernatants (1-10 ul) or 50 ng of purified proteins were aliquoted and treated with SDS-PAGE sample buffer with and/or without reduction by dithiothreitol (DTT). The specimens were fractionated on a 4-12% NuPage PAGE SDS gel in MES buffer (Thermo Scientific, Waltham, Mass.). Protein was transferred to a PDVF membrane using the iBlot 2® Dry Blotting System (Thermo Fisher, Life Technologies, Carlsbad, Calif.). The membrane was blocked for 1 hr in 5% milk/PBS, then probed with polyclonal rabbit anti-A244/MN_{GNE} antibody at 1 μg/ml overnight at 4° C., washed three times for 10 min with each wash using 100 ml of 0.05% Tween/PBS, then probed with an affinity purified secondary HRP conjugated goat anti-rabbit H+L chain antibody (Jackson ImmunoResearch Laboratories, West Grove, Pa., ImmunoResearch, West Grove, Pa.) for 1 hr at room temperature. After a final (×3) wash with 0.05% Tween/PBS the membrane was developed using WesternBright ECL kit (Advanta, Menlo Park, Calif.) and visualized using an Innotech FluoChem2 system (Genetic Technologies, Grover, Mo.).

Immunoaffinity Purification of A244_{UCSC} rgp120.

The A244_{UCSC}-rgp120 proteins from individual clones were immunoaffinity purified using the gD purification tag as described previously (Lasky, L. A., et al., Science, 1986. 233(4760): p. 209-12; Smith, D. H., et al., PLoS One, 2010. 5(8): p. e12076). Briefly, 5 ml of cell culture medium was applied to an anti-gD flag monoclonal antibody coupled controlled poured glass column. The column was washed with 10 column volumes of 50 mM Tris, 0.5 M NaCl, 0.1 M TMAC (tetramethylammonium chloride) buffer (pH 7.4), and eluted with 0.1 M sodium acetate buffer, pH 3.0. The pH of the buffer was neutralized by the addition of 1.0 M Tris (1:10 ratio) and the resulting solution was concentrated using an AMICON molecular weight cutoff centrifuge tube (Millipore, Billericia, Mass.). The purified protein was adjusted to a final concentration of 1-2 mg/mL in PBS buffer. Protein concentrations were determined using the bicinchoninic acid assay (BCA) method.

bNAb Binding to gp120s.

The binding of bNAbs to gp120 proteins to was assayed using a capture Fluorescence Immunoassay (FIA) assay. Briefly, 2 μg/mL of anti-gD tag monoclonal antibody, 34.1, was diluted into PBS and incubated at 4° C. overnight in 96 well black-microtiter plates (Greiner, Bio-One, USA). Plates were blocked in PBS containing 1% BSA+0.05% normal goat serum in 0.01% thimerosal for two hours at room temperature. Wells were incubated with 60 uL of blocking solution containing 6 ug/mL of purified rgp120 overnight at 4° C. Three-fold serial dilutions of primary antibody were added starting at 10 ug/mL, followed by incubation with a 1:3,000 dilution of goat-anti-human or donkey-anti-goat AlexaFluor 488 conjugated polyclonal (Jackson ImmunoResearch Laboratories, West Grove, Pa., Life Technologies, Carlsbad, Calif.). All dilutions were performed in solution of PBS containing 1% BSA with 0.05% normal goat serum and 0.01% thimerosal, and incubations were carried out for 90 min at room temperature followed by a 4× wash in PBST buffer unless otherwise noted. Absorbance was read using an EnVision Multilabel Plate Reader (PerkinElmer, Inc Waltham, Mass.) with a FITC 353 emission filter and a FITC 485 excitation filter. Each assay was performed in duplicate and results were reported as half maximal effective concentration, (EC50), or the concentration of antibody required for half of the maximal binding readout. Polyclonal goat sera against the full-length gp120 and human isotype control were used as coating and negative controls, respectively.

Results

Colony Selection.

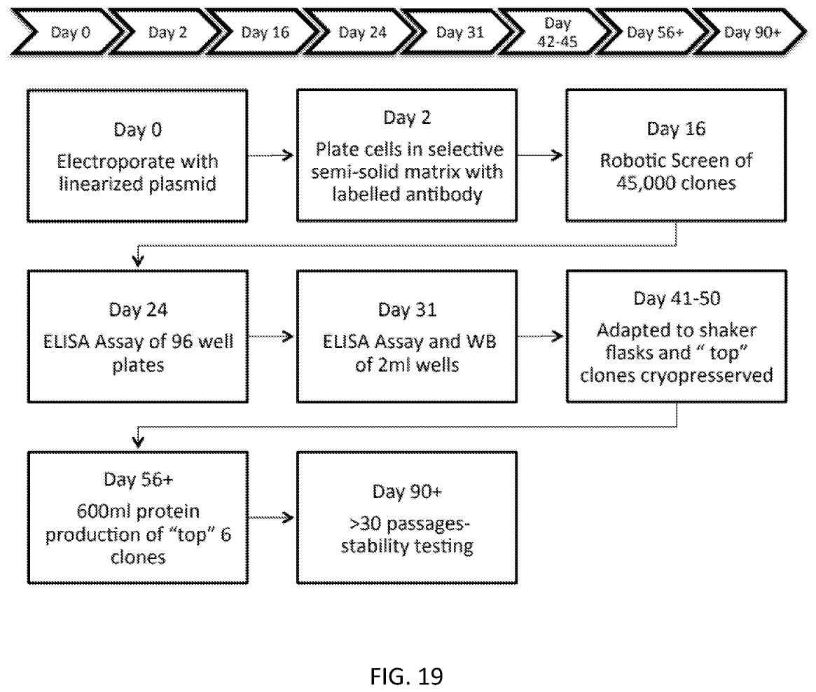

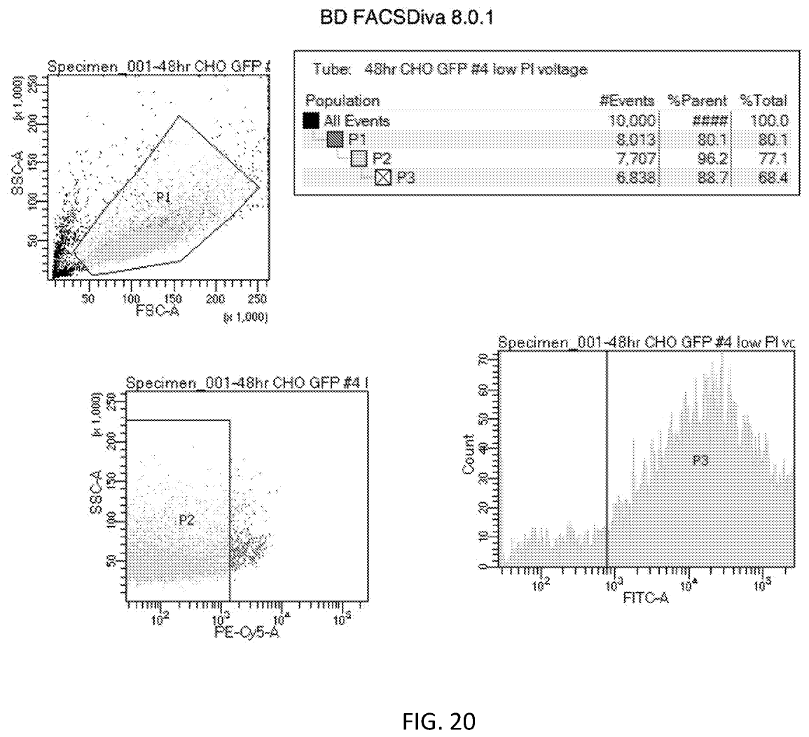

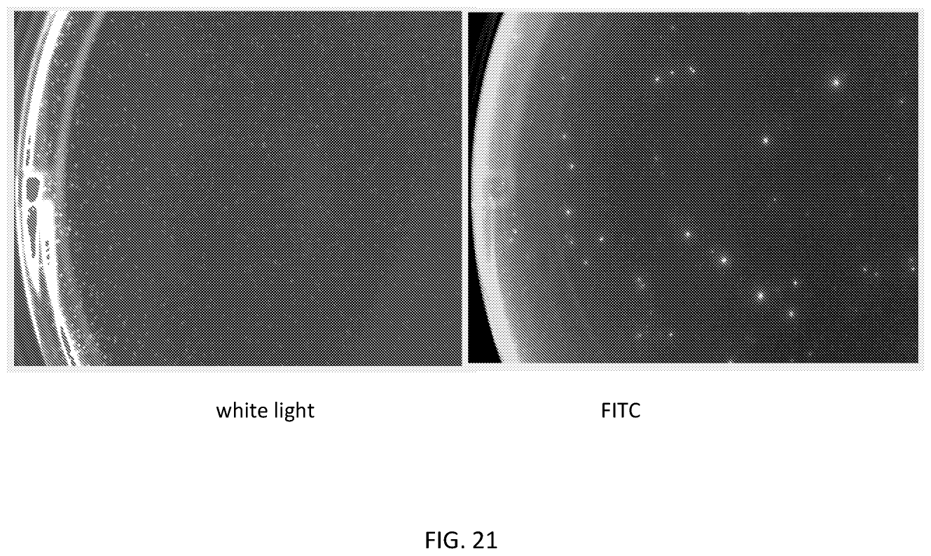

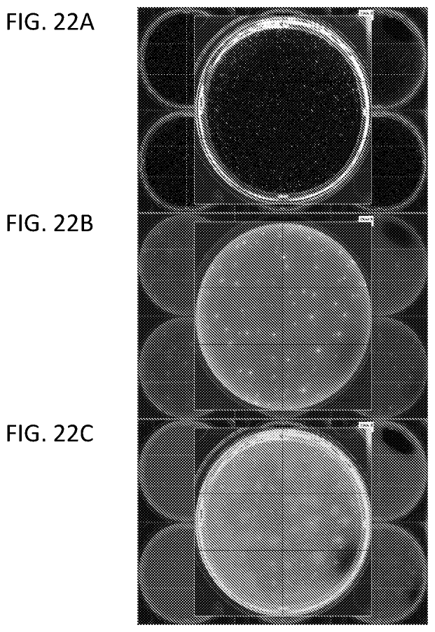

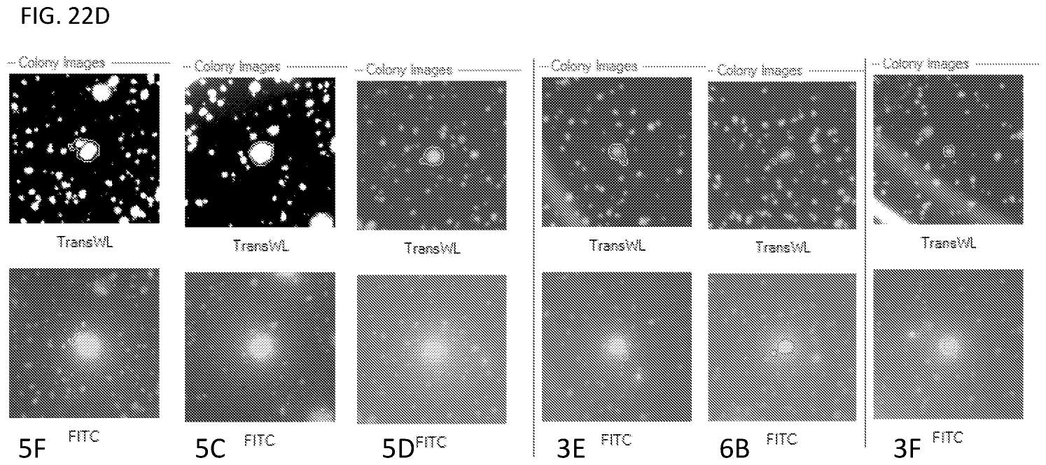

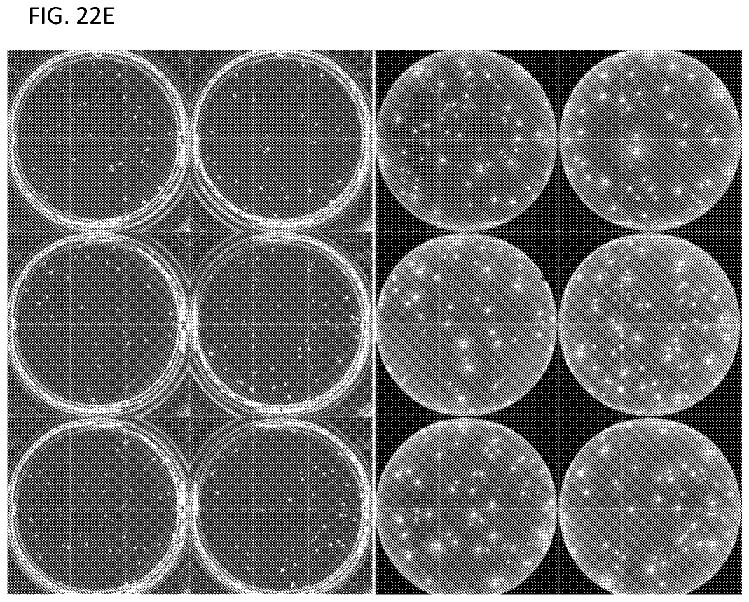

The timeline for production of clones expressing A244_{ucsc}rgp120/HIV-1 is shown in FIG. 19. A total of 8×10⁷ Mgat1 cells were transfected with the expression plasmid UCSC1331 (UCSC_CHO.A244N332) by electroporation. Transfection of CHO-S cells using the MaxCyte electroporation system is highly efficient (>88% expression of GFP by FACS at 48 hr) (FIG. 20). Just six days after setting up Mgat1/UCSC1331 electroporated cells with gp120 specific, immune-affinity purified, Alexa 488 labeled polyclonal antibody, precipitin halos were visible under fluorescent light around a small percentage of colonies in each 6-well plate (FIG. 21). After 16 days in selective media, cells transiently expressing protein had died or were dying off, and thriving colonies of cells expressing antibiotic resistance are clearly visible by white light (FIGS. 22A-22E). 45,000 colonies from four 6-well plates were screened using the ClonePix 2, and of these, approximately 0.1% were picked and transferred into 96 well plates.

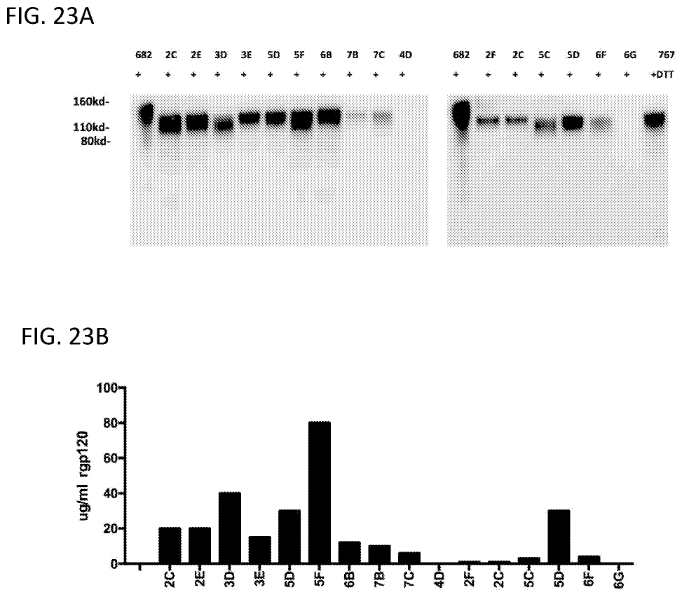

Forty-three of the selected colonies grew and actively secreted A244rgp120 HIV-1 highest mean external fluorescent intensity and final clone selection did not completely correlate. Only fifteen out of the forty-three positive clones selected secreted a protein that bound both polyclonal anti-gD gp120 (PB94) and the bNAb PG9 in ELISA. In general, with the exception of a single clone (5C), clones with the highest level of mean external intensity at pick did not bind PG9, and did not survive transfer from 96-well plate to 2 ml wells. After 31 days in culture, 14/15 of PG9/PB94 positive clones were secreting A244_{ucsc} rgp120 HIV-1 was confirmed by western blot (FIGS. 23A and 23B) with an antigen specific polyclonal serum. Individual A244_{ucsc} rgp120 HIV-1 clones had slightly different growth characteristics, but some had a particular tendency to form large clumps in suspension. Clones were cryo-preserved and the most promising ones carried forward.

Batch Fed Culture Expression.

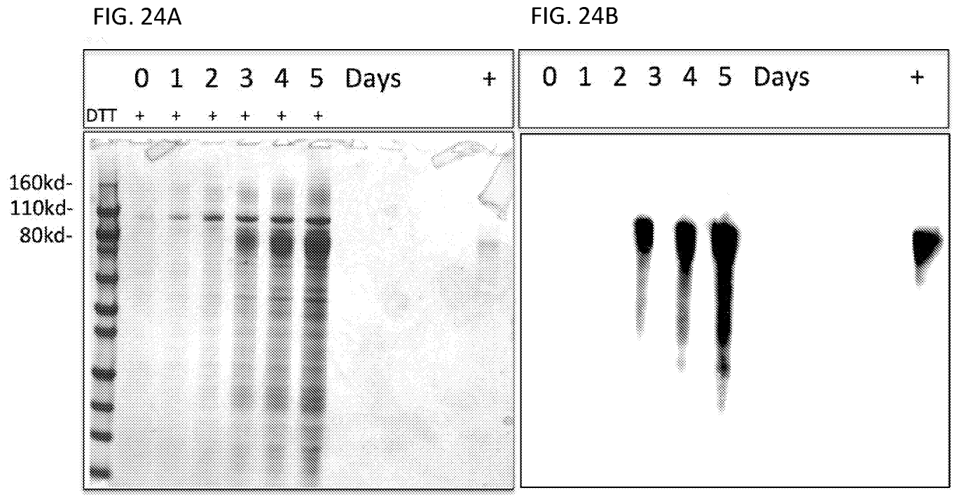

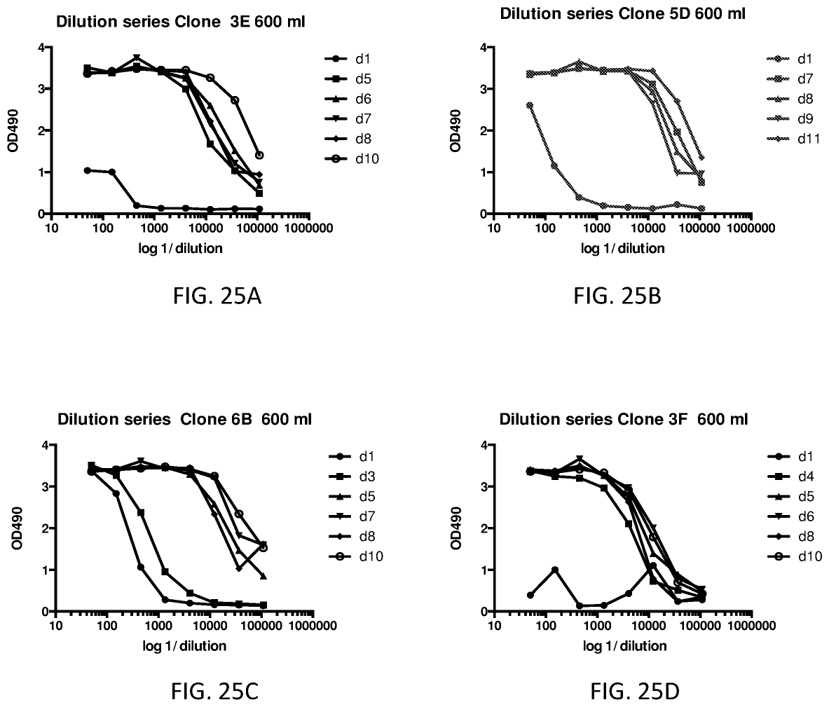

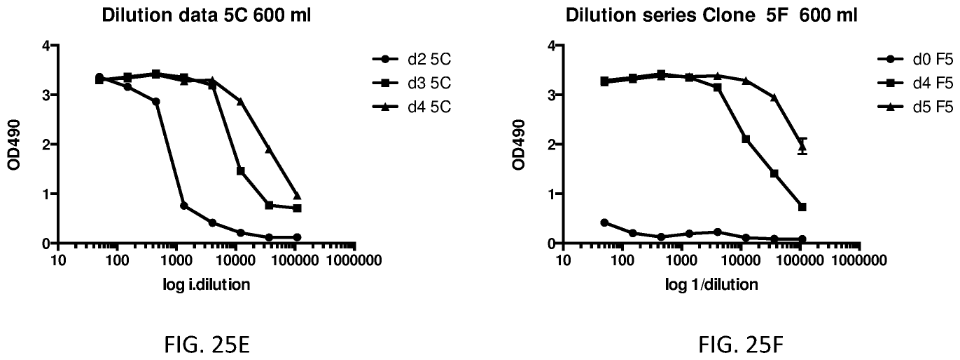

Two months after the initial transfection, six Mgat1 A244_N332-rgp120 clones selected for optimal protein expression were assayed for protein production. Each clone expanded to 600 ml fed batch culture with a 1×10⁷ cells/ml seed. Flasks were cultured in the presence of 1 mM sodium butyrate at 32° C., 135 rpm with 8% CO₂ and 85% humidity until the viability dropped below 50%. Protein accumulation was detectable in daily 10 μl samples of cell supernatant by SDS/PAGE (FIGS. 24A and 24B). By day 5, recombinant gp120 was the principle protein in the tissue culture supernatant. Protein production by indirect ELISA of supernatant, and raw three-fold dilution data for the six clones demonstrated rapid protein accumulation (FIGS. 25A-25F). Clone 5C only survived 3 days, clone 5F, 5 days. All of the other clones were stable for 10-11 days in culture with daily feeding.

Protein Recovery and bNAb Binding.

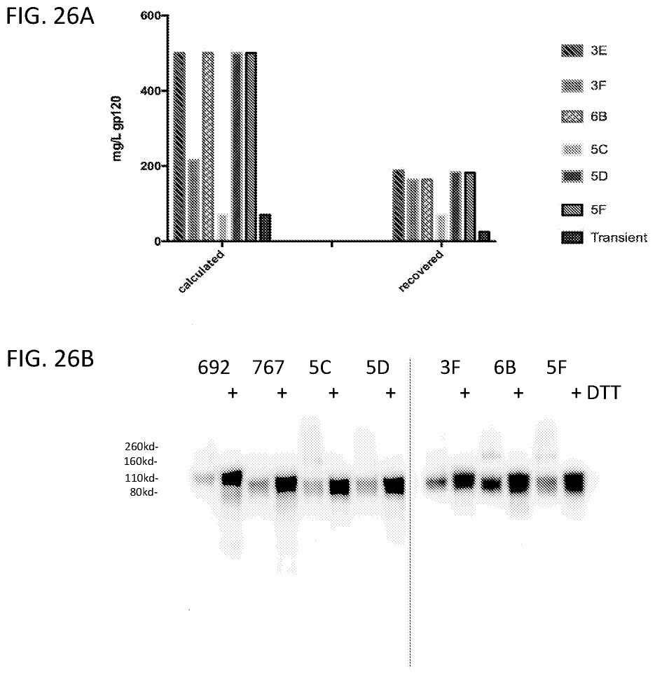

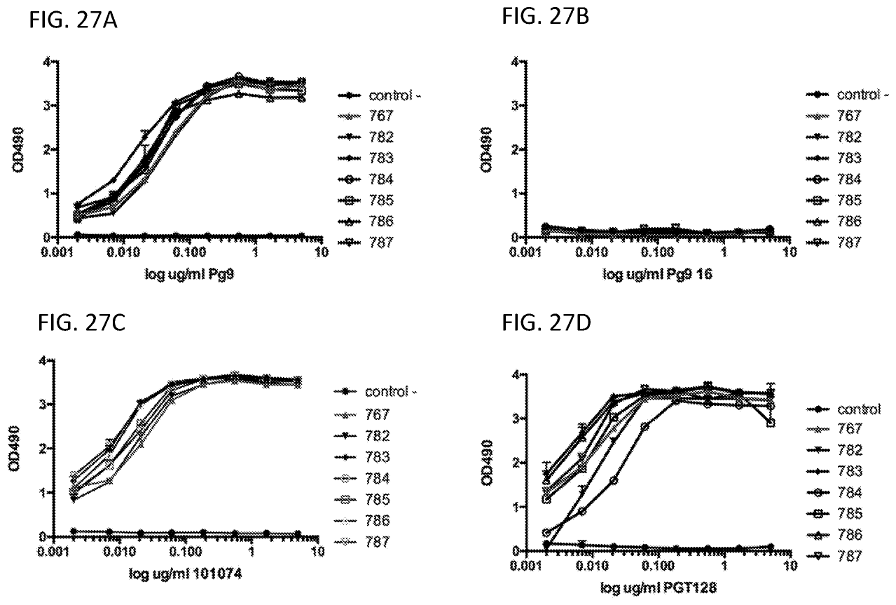

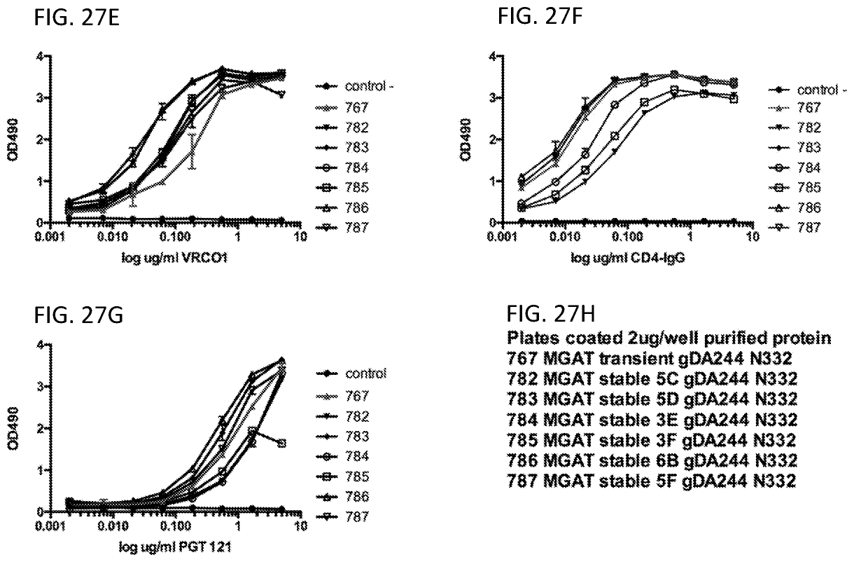

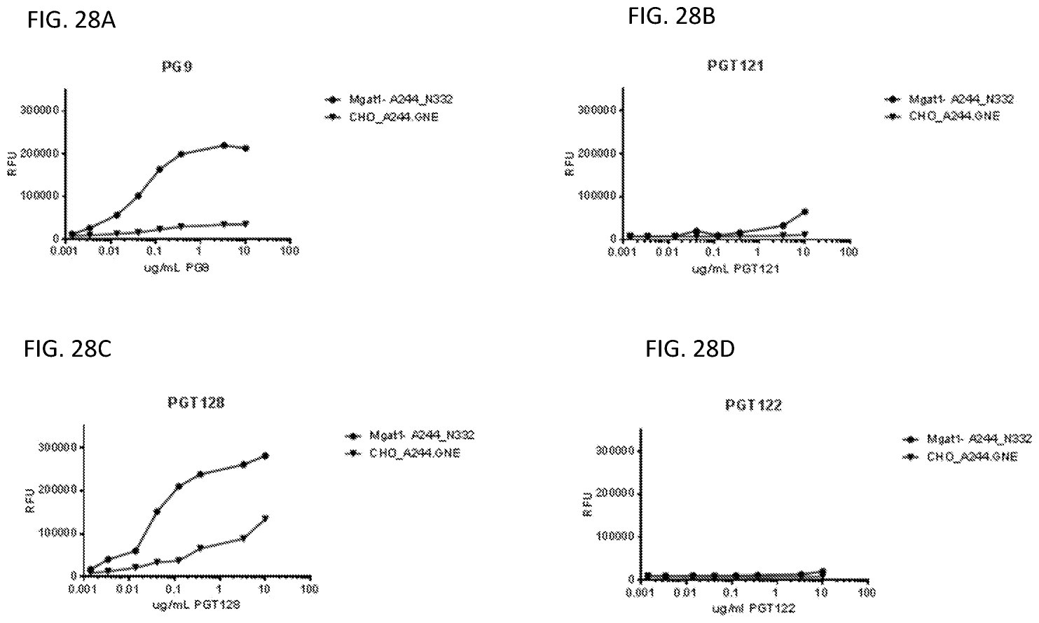

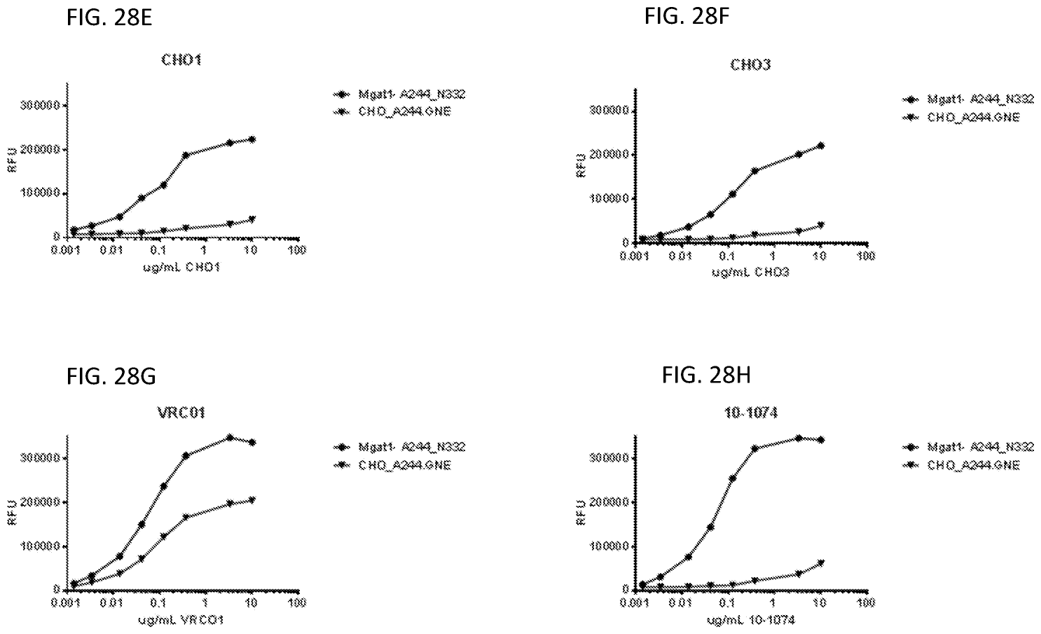

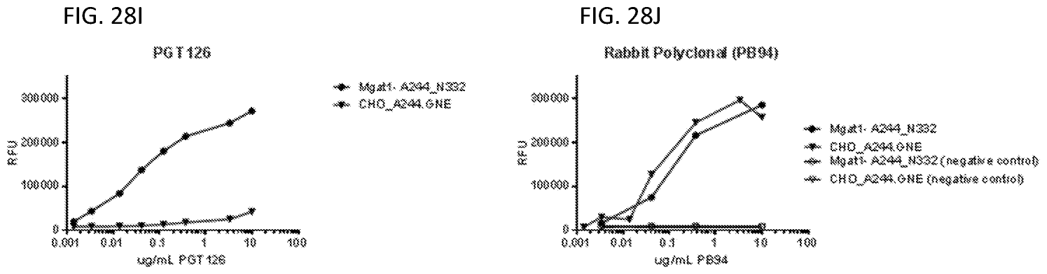

The A244 _{UCSC}-rgp120 proteins from different clones were immunoaffinity purified using the gD purification tag as described. A western blot using polyclonal anti-gp120 sera determined that there was minimal proteolysis or aggregation of the affinity purified proteins FIGS. 26A and 26B. At least three clones produced at more than 200 mg/L of affinity purified protein (clones 3E, 3D, 5F). The protein produced by individual clones was assayed by binding to glycan dependent- and glycan independent-bNAbs (FIG. 27A-27H). There was little or no difference in bNAb binding by gp120s recovered from different Mgat1-A244_{UCSC}-rgp120 clones, or protein isolated following transient protein production. The proteins all behaved in a similar manner by ELISA, all bound to the bNAbs: PG9, PGT128, VRC01 and CD4-IgG, but not to PG16. In additional experiments, (FIG. 28A-28J) the antigenicity of A244_N332-rgp120-rgp120 produced in the Mgat1-cell line was compared to A244-rgp120 produced in normal DG44 CHO cells and used in the RV144 clinical trial. These studies showed markedly enhanced binding of glycan dependent bNAbs (PG9, PGT128, CH01, PGT126, CHO3 and 10-107410-1074) to A244_N332-rgp120 expressed in Mgat1⁻ CHO cells compared A244-rgp120 to expressed in normal DG44 CHO cells. Neither gp120 was able to bind the glycan dependent antibodies PGT121, and PGT122. Surprisingly the protein produced in Mgat1-cells also exhibited enhanced binding of VRCO1, an antibody that recognizes a glycan independent epitope that overlaps the CD4 binding site. Thus the incorporation of smaller high mannose structures appear to enhance the binding of antibodies to glycan dependent epitopes, perhaps by minimizing steric hindrance.

Cryopreservation of Cells and Pathogen Testing.

A master cell bank of cryopreserved cells was created from the 5F clone that secreted the highest levels of A244-N332-rgp120. Vials containing 1×10⁷ cells were transferred to the ATCC for archival storage and distribution. Cells from this bank were also transferred to the IDEXX commercial cell line testing facility in Columbia, Mo. These were tested for contamination by other cell lines (e.g. HeLA and 293), mycoplasma, and a large panel of human and animal viruses such as minute virus of mice (MVM). The results of these assays are provided in Berman Lab Technical Report TR-01-17.

Preliminary data indicates that clone 5F is stable for at least 90 as clones were cells were still expressing >200 mg/L protein as measured by ELISA/FIA assay.

Discussion

This report describes the development of an improved method for the construction of stable CHO cell lines producing an improved variant of recombinant gp120 for use as a candidate HIV vaccine immunogen. The improved method of stable cell lines depended the development of methods, reagent, and procedures allow selection of rare high producing cell lines by robotic selection using the ClonePix2 robot (Molecular Devices, Sunnyvale, Calif.). This protocol and the MaxCyte electroporation device allow the screening of at least 45,000 transfected CHO cells in a single day—a task that would take many months if cell lines were picked by conventional approaches such as of manual selection. A major unexpected finding from these experiments was that it was not necessary to employ standard methods of gene amplification based on co-expression of dihihydrofolate (dhfr) or glutamine synthetase (GS) transgenes. The elimination of this approach further saves months if not years of time in the identification of a high producing cell line. The results suggest that the disclosed screening method involving ClonePix 2 can identify extremely rare high producing cell lines with protein yields in excess of 200 mg/L. These yields are comparable to those in cells selected using conventional techniques that can be performed in a fraction of the time (i.e. 2-3 months compared to 12-24 months).

Besides improving protein yield, another major goal of this project was to improve the antigenic structure of the A244-rgp120 protein thought to be the principal immunogen responsible for protection in the RV144 clinical trials (Rerks-Ngarm, S., et al., N Engl J Med, 2009. 361(23): p. 2209-20). The A244_{UCSC} rgp120 described in this report appears to represent an improved form of the original immunogen. Several studies have shown that the type and location of N-linked glycosylation sites are major determinants of antigenic structure and the binding of bNAbs. The A244_{UCSC} rgp120 produced in the Mgat1⁻ cell line is the first gp120 produced under conditions suitable for biopharmaceutical production able to take advantage of both aspects of envelope structure. Although A244-rgp120 is unusual in its ability to bind several bNAbs such as PG9, PGT128, and 10-1074, the binding to these sites is enhanced by production in the Mgat1 cell line that restricts glycosylation primarily to mannose-5 structures. The fact that glycans are not completely limited to mannose-5 and approximately 20% of the glycans are mannose-9 is an unexpected benefit in that mannose-9 is preferred by PGT128. A further enhancement in binding is attributable to relocating the predicted N-linked glycosylation site at N334 in the wild-type A244-rgp120 protein to N332 in the A244_N332-rgp1 protein. The N322 glycan has been reported to be essential for a number of bNAbs (Walker, L. M., et al., Science, 2009. 326(5950): p. 285-9; Shingai, M., et al., Nature, 2013. 503(7475): p. 277-280).

Finally, an unanticipated benefit of gp120 production in the Mgat1⁻ cell line is that the protein is far more homogenous in glycan content and net charge compared to gp120s produced in normal cell lines. Historically, controlling glycosylation is difficult in commercial manufacturing, and different fermentations often yield proteins with different glycan content. Differences in glycan content can affect recovery yields and affect pharmacokinetic half-life, biodistribution, and product immunogenicity (Sinclair, A. M. and S. Elliott, J Pharm Sci, 2005. 94(8): p. 1626-35; Solá, R. J. and K. Griebenow, BioDrugs, 2010. 24(1): p. 9-21). Variation in any one of these properties can alter the potency and biologic efficacy of protein biopharmaceuticals and regulatory approval. The improvement in glycan homogeneity in the A244-N332-rgp120 produced in the Mgat1-cell line allows for a simpler, more productive purification process provides for improved manufacturing reproducibility and more consistent biologic activity. It is anticipated that these improvements in the location and structure of N-linked glycosylation sites will enhance the efficacy of the gp120 vaccine used in the RV144 trial from a level of ˜31% to a vaccine efficacy of 50% or greater thought to be required for regulatory approval and clinical deployment.

FIG. 19. Diagram of method for rapid production of cell lines expressing recombinant gp120.

FIG. 20. GFP expression after MaxCyte STX electroporation of CHO-S cells. At 48 hr, 88.7% of live cells were expressing GFP. Gate P1, all cells, Gate P2 live cells, Gate P3, live and expressing GFP.

FIG. 21. White and fluorescent images from a single well of UCSC_CHO.A244N332 transfected cells on the ClonePix 2. Images were captured 6 days (16-32 cells per colony) after plating in semi solid selective matrix, in the presence of Alexa488 labeled affinity-purified polyclonal antibody. Antigen specific precipitin rings (or halos) are visible around a proportion of colonies under selection G418 selection.

FIGS. 22A-22E. ClonePix 2 Clone images at Day 16. FIG. 22A Day 16, a single 35 mm well of UCSC_CHO.A244N332 transfected colonies illuminated by white light alone; FIG. 22B the same well as in FIG. 22A but FITC imaged: FIG. 22C the superimposition of white and FITC images reveals the “halo’ area outside the colony where secreted antigen interacts with FITC labeled antibody in the matrix. Mean fluorescence intensity is calculated from these images by the ClonePix 2. FIG. 22D Six colonies picked on Day 16 and expanded. Expression was tested at Day 24, 31 Day 56 and Day 90. Top row colonies visualized with white light, bottom row, with FITC. FIG. 22E Clone 5F recloned (from early passage cryopreserved cells) at 25 cells/ml. Left panel white light, right panel FITC.

FIGS. 23A-23B. Expression of proteins in 2 ml wells (Day 31). FIG. 23A Western blot of tissue culture supernatant from 2 ml wells (not controlled for cell density or viability). 10 μl of supernatant, 4-5 days growth (<5E+05 cells/ml), reduced in DTT and electrophoresed on a 4-12% SDS/PAGE gel was transferred to a PVDF membrane and probed with an antigen specific polyclonal rabbit serum. Bound antibody was detected with a goat anti-rabbit HRP conjugate. Size markers for rgp120 A244_{GNE} produced from transient transfection of CHO-S cells (682) and transient A244_{GNE} expression (lot 767) are included as size markers. FIG. 23B Indirect ELISA quantification of rgp120 A244N332. Supernatants were captured by anti-gD (34.1 A64 2 μg/ml). Bound antigen was detected with 1 μg/ml polyclonal rabbit sera followed by a goat anti-rabbit HRP at a 1/5000 dilution. Protein concentration was determined by serial dilution of cell supernatant then interpolation from a standard curve using GraphPad Prism version 6.00 for Mac, GraphPad Software, La Jolla Calif. USA.

FIGS. 24A and 24B. Batch Fed Culture Expression of Clone 5F: accumulation of rgp120 during 600 ml protein expression trial culture. FIG. 24A. 10 μl DTT reduced tissue culture supernatant (days 0-5) loaded per lane of a 4-12% Bis-Tris/MED buffer SDS/PAGE gel stained with Coomassie blue. FIG. 24B. 1 μl DTT reduced tissue culture supernatant (days 0-5) loaded per lane of a 4-12% Bis-Tris/MES buffer SDS/PAGE gel western blotted with an antigen specific polyclonal rabbit serum. Bound antibody was detected with a goat anti-rabbit HRP conjugate. 100 ng of DTT treated purified MGAT CHO-S gDA244 N332 is included as a control on each gel.

FIGS. 25A-25F. Batch Fed Culture Expression. Indirect ELISA showing raw dilution data of tissue culture supernatant collected during the course of a 600 ml batch fed protein expression assay. Wells were coated with 34.1 (A64) at 2 μg/ml for indirect capture of serial dilutions of supernatant containing gp120. Bound protein was detected using an antigen specific polyclonal rabbit serum (PB94) and an anti-rabbit HRP conjugate (Jackson ImmunoResearch West Grove Pa.).

FIGS. 26A and 26B. Protein yield. FIG. 26A Yield from 600 ml batch fed cultures pre and post purification by immuno-affinity capture. Pre-purification yield was determined by indirect ELISA (anti-gD, 34.1 A64, 2 μg/ml) capture followed by detection of bound antigen by polyclonal rabbit anti-gp120 (PB94) and goat anti-rabbit HRP. FIG. 26B Western blot of protein purified by affinity chromatography from 600 ml batch fed cultures. 50 ng of each non or DTT reduced protein, was loaded per lane (with the exception of 3E) of a 4-12% PAGE/SDS MES buffer gel. Protein 692, A244rgp120 produced in DG44 cells, and protein lot 767, a transiently produced A244_{UCSC}, were included as controls. Recombinant gp120 was detected using an antigen specific polyclonal rabbit serum (PB94) and an anti-rabbit HRP conjugate.

FIG. 27A-27H. Direct binding of purified MGAT g120 HIV-1 proteins to bNAbs. Nunc Immulon 96 well plates were coated with 2 μg/ml of affinity purified protein from the stable lines (UCSC protein batches 782-787) and a transiently produced protein (767) overnight in PBS. After blocking for 1 hr at room temperature, 3-fold serial dilutions of antibody were made in 5% milk/PBS and incubated directly with the protein coated wells for 1 hr at room temperature. Bound antibody was detected by incubation with a 1/5000 dilution of rabbit anti-human HRP conjugate (Jackson Immuno, West Grove, Pa.) and developed with o-phenyldiamine-dichloride (OPD) Thermo-Fisher Waltham Mass.) according to the manufacturers protocol. The reaction was stopped after 10 minutes using H₂SO₄ and the plates read on a Maxisorb plate reader at 490 nm.

FIGS. 28A-28J. Comparison of bNAb binding to CHO A244_{GNE}-rgp120 produced in normal CHO cells and used in the RV144 trial, and improved A244-N332-rgp120 produced in Mgat1⁻ cells. Recombinant gp120s were captured onto the surface of microtiter plates coated with a monoclonal antibody (34.1) to the gD purification tag present at the N-terminus of both proteins. Wells were incubated with an Alexa 488-labeled Three-fold serial dilutions of primary antibody were added starting at 10 ug/mL, followed by incubation with a 1:3,000 dilution of goat-anti-human or donkey-anti-goat AlexaFluor 488 conjugated polyclonal (Jackson ImmunoResearch Laboratories, West Grove, Pa., Life Technologies, Carlsbad, Calif.).

### Example 5

Purification of Recombinant Gp120 Produced in an Mgat1⁻ Cell Line

It is disclosed herein that recombinant gp120 (A244-N332_rgp120) produced in Mgat1⁻ cells, incorporating primarily the mannose-5 glycans, is highly homogeneous in net charge and can be purified by conventional, cost-effective, ion-exchange and size exclusion column chromatography. It is known that gp120 expressed in normal CHO cells incorporated highly heterogeneous, sialic acid containing glycans and cannot be efficiently purified using conventional column chromatography. The variation in sialic acid content in gp120 produced in normal CHO cell lines resulted in heterogeneity in net change and other biophysical properties that prevented efficient purification by standard methods without experiencing a substantial loss of in yield (e.g. 30-60%). As a consequence most commercial scale recovery processes, designed to purify gp120, involved the use of expensive affinity resins prepared from monoclonal antibodies or lectins (e.g. GNA) to recover the gp120 containing complex glycosylation. This affinity purification step added considerable time and expense related to the production need to manufacture antibodies and lectins by processes compliant with current Good Manufacturing Practices (cGMP). It is disclosed herein that conventional methods of protein purification, suitable for biopharmaceutical manufacturing, can be used to efficiently purify A244-N332-rgp120 and results in a final product with high yields (>90%) yields and high product purity.

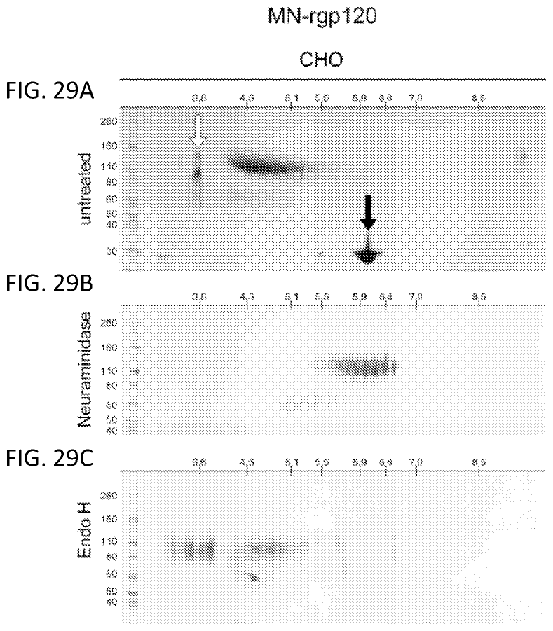

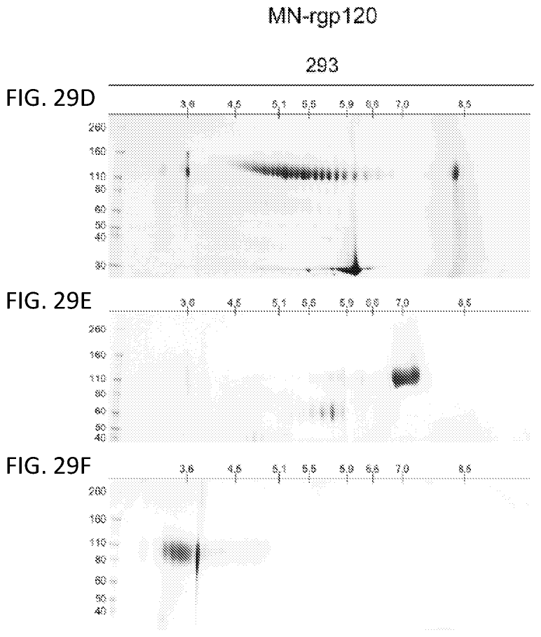

Historically, the development of HIV envelope proteins (e.g. gp120 and gp140) for use as vaccines has been limited by the fact that they are poorly expressed in conventional mammalian cell culture expression systems. Thus many investigators have reported expression levels in the 2-20 mg/L range whereas yields for other recombinant proteins often exceed 50 mg/L and often, as in the case of antibodies, can be produced in the 0.5 to 5 g/L range. Moreover recombinant gp120s are difficult to purify due to the fact that they are highly heterogeneous due to the presence of approximately 26 N-linked glycosylation sites (Leonard, C. K., et al., J Biol Chem, 1990. 265(18): p. 10373-82). Many of these contain anywhere from one to four residues of sialic acid, leading to unusually large variation in net charge. When expressed in normal mammalian cell lines (e.g. CHO or 293HEK), as many as 40 different glycan structures described for a single site (Go, E. P., et al., Journal of proteome research, 2013. 12(3): p. 1223-1234). The heterogeneity in glycosylation results in considerable heterogeneity in net charge (FIG. 29) with 20-40 discrete bands typically visible on 2-dimensional isoelectric focusing gels (Yu, B., et al., PLoS One, 2012. 7(8): p. e43903). A consequence of the heterogeneity in net charge is it has been difficult to purify recombinant HIV glycoproteins by standard, cost-effective, chromatographic methods that can be used for biopharmaceutical production. To circumvent the dual problems of low yields and high heterogeneity in molecular mass and net charge, most approaches to purify recombinant HIV envelope proteins make use of an affinity chromatography step that makes use of monoclonal antibody (Yu, B., et al., PLoS One, 2012. 7(8): p. e43903; Lasky, L. A., et al., Science, 1986. 233(4760): p. 209-12) or a lectin (Srivastava, I. K., et al., J Virol, 2002. 76(6): p. 2835-47; Sellhorn, G., et al., Journal of virology, 2012. 86(1): p. 128-142; Arthos, J., et al., Nat Immunol, 2008. 9(3): p. 301-9). Either type of affinity column adds additional steps to the purification process and requires expensive custom reagents that must be produced and tested under validated current Good Manufacturing Practices (cGMPs). For example the preparation of an antibody or lectin affinity columns for the large scale (2,000-10,000 L) production of gp120 can easily cost hundreds of thousands to millions of dollars and requires extensive quality control and validation to define its ligand binding capacity, cleaning and elution procedures, antibody leaching into the final product, and the number of times it can be used before it needs to be replaced. Moreover, the proteins recovered from the affinity purification step are still heterogeneous with respect to glycosylation and need additional purification (polishing) and virus inactivation steps by standard chromatographic methods such as ion-exchange chromatography (IEX), size exclusion chromatography (SEC), and tangential flow filtration (TFF) before they can be vialed and used as a vaccine. As a consequence of this multi-step process, there is typically considerable loss of material, often 30-50%. Additionally, the heterogeneous glycosylation in the conventionally purified proteins results in heterogeneity at critical epitopes recognized by glycan dependent monoclonal antibodies. Many of the most potent and broadly neutralizing antibodies to HIV-1 (e.g. PG9, PGT128, PGT121, and 10-1074) recognize glycan dependent epitopes. Uniformity in epitopes recognized by bNAbs may be a key factor in defining vaccine potency and efficacy. Production of vaccines in the Mgat1⁻ cell line described in this report is currently the only scalable method to produce recombinant envelope proteins that primarily contain mannose-5 glycans required for the binding on multiple bNAbs.

Materials and Methods

Growth Conditioned Cell Culture Medium Containing A244 N332-Rgp120.

The stable 5F clone of the Mgat1⁻ CHO cell line transfected with the gene encoding A244-N332-rgp120 was grown in a 1.6 L shake flask in serum free CD-OptiCHO growth medium (Gibco, Thermofisher) at 37° C. After achieving a density of 1×10⁷ cells/mL, sodium butyrate was added (1 mM) and the temperature was shifted to 32° C. Once cell viability dropped to 50% (day 5). The growth conditioned cell culture was harvested by centrifugation and vacuum filtered through a 0.45 um SCFS membrane and stored frozen at −20° C.

Purification of Gp120 by Column Chromatography.

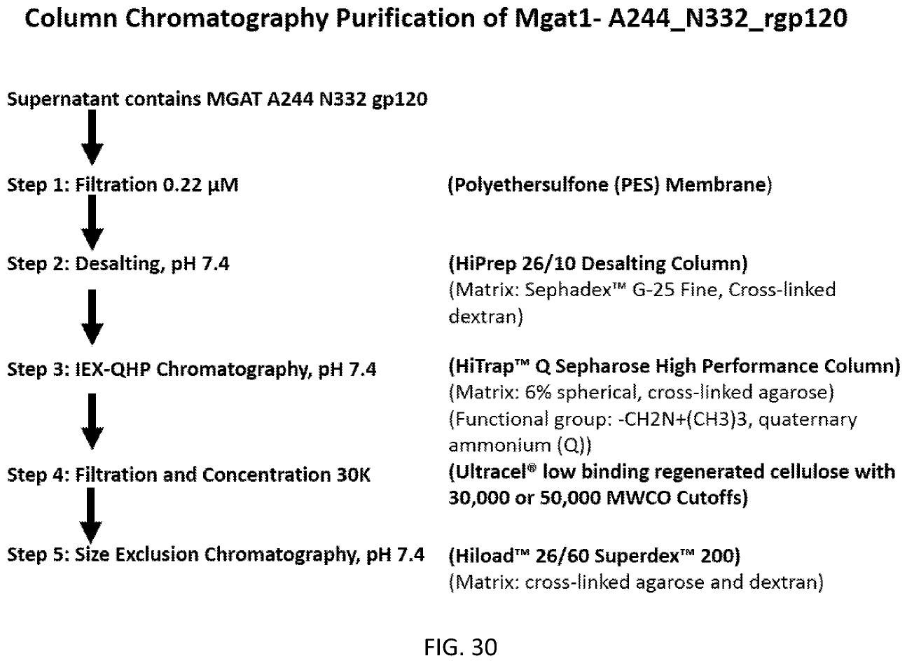

After thawing, the gp120 was recovered by column chromatography according to the process described in FIG. 30.

Purification by Affinity Chromatography.

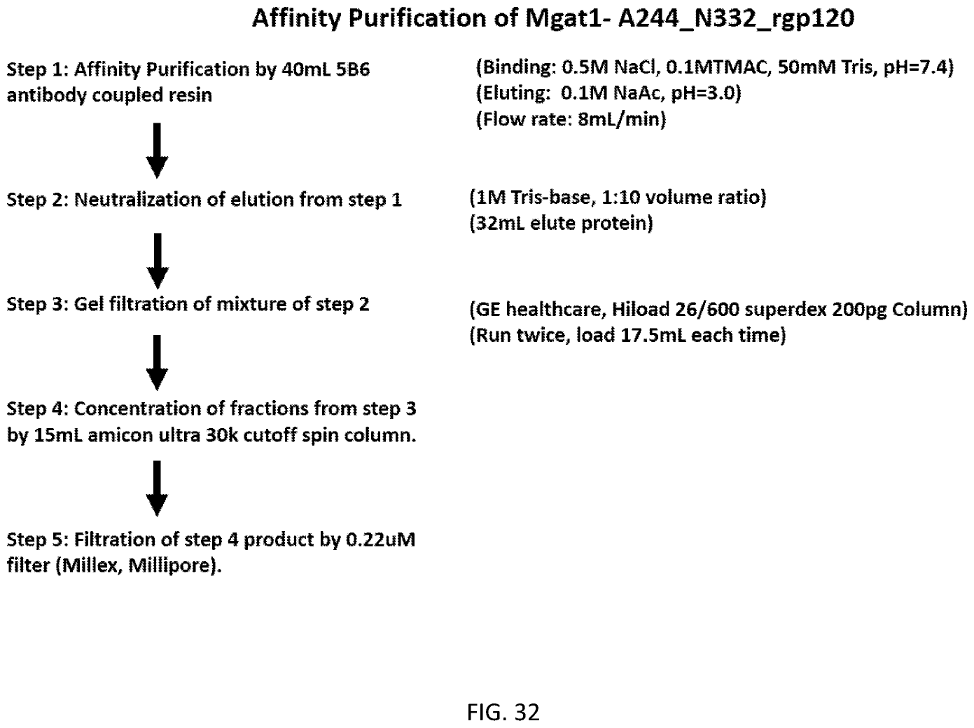

After thawing, the gp120 was recovered by column chromatography according to the process described in FIG. 32.

Carbohydrate Content.

After purification the carbohydrate content of A244_N332-rgp120 was determined by MALDI-TOF mass spectroscopy by Dr. Parastoo Azadi of the Complex Carbohydrate Research Center (university of Georgia, Athens, Ga.).

Results

Comparison of A244_N332-Rgp120 Purified by Immunoaffinity Chromatography and by Conventional Ion Exchange Chromatography.

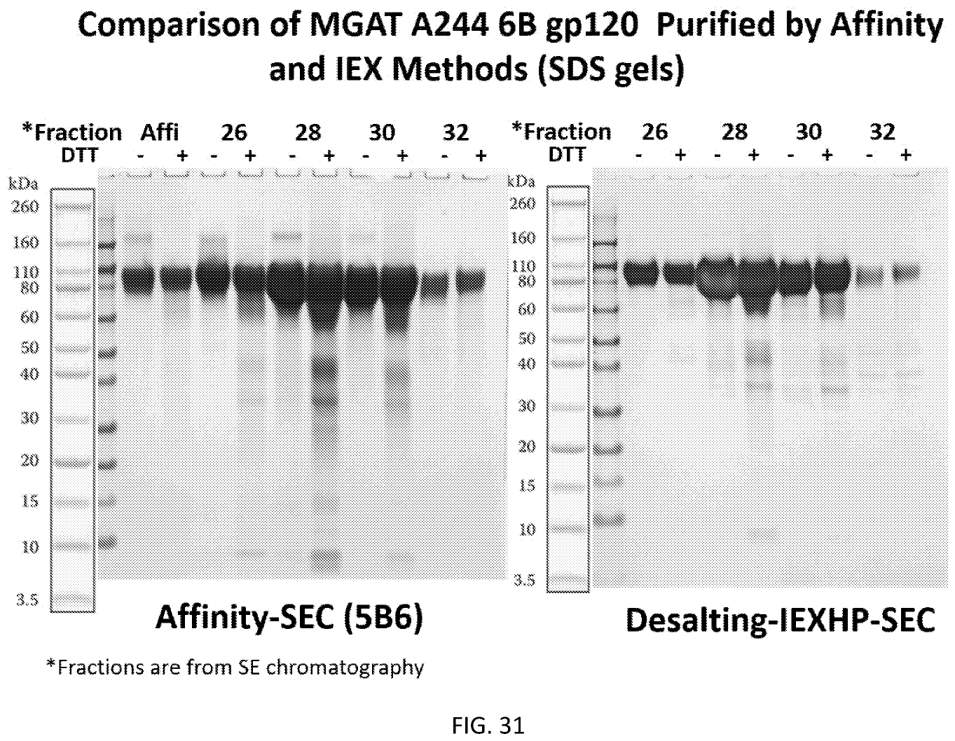

Experiments were carried out to determine whether A244_N332-rgp120 produced in the Mgat1⁻ cell line could be purified by a practical, high yielding recovery process suitable for biopharmaceutical production. These experiments involved screening different chromatography resins and different conditions for adsorption and elution (data not shown). The recovery process described in FIG. 30 represents the final method developed in this study. When analyzed by SDS-PAGE the resulting gp120 (FIG. 31) possessed physical properties closely resembling A244-N322-rgp120 protein purified by a process requiring immunoaffinity chromatography developed at Genentech in the early 1990s (FIG. 32). This immunoaffinity process (FIG. 32) was similar to that used in the large scale production of HIV vaccine for multiple clinical trials including the 16,000 person RV144 trial (Rerks-Ngarm, S., et al., N Engl J Med, 2009. 361(23): p. 2209-20).

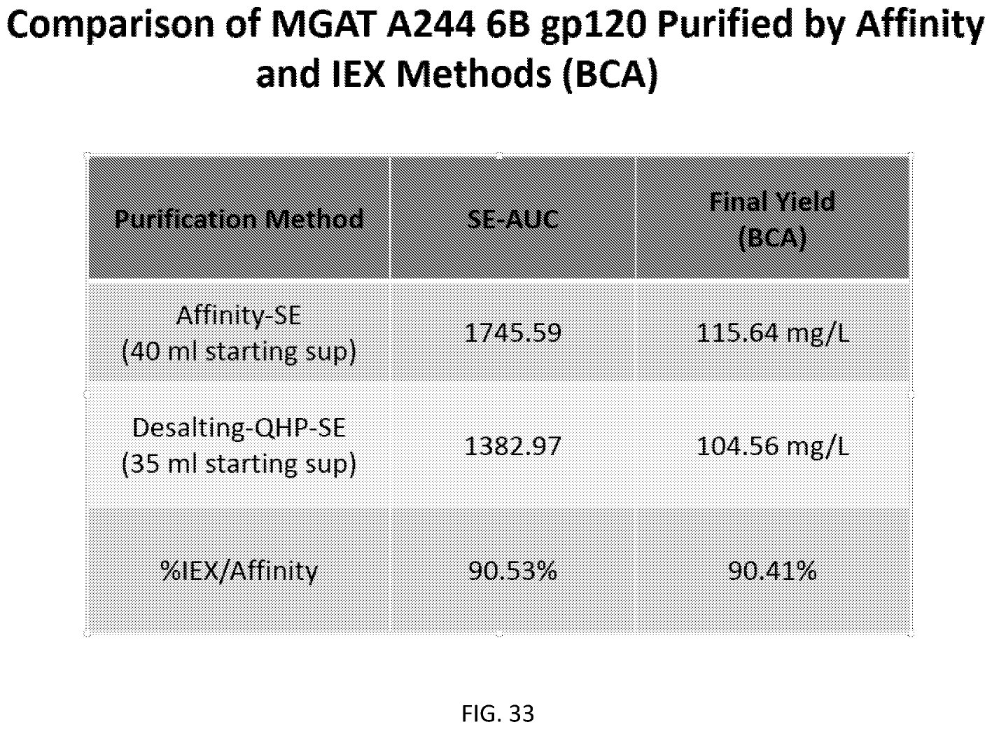

To compare the efficiency of purification by both processes side by side purifications were carried out with the same staring material. The results of this study (FIG. 33) showed that both recovery processes resulted in protein of comparable purity with yields of approximately 90%. Thus comparable results could be obtained by both processes, however the conventional process is more economical to run and doesn't involve the use of custom made monoclonal antibodies. Another potential advantage of the conventional process is that eliminate the low pH elution step required for the affinity process. Although there is no direct evidence from these studies that the low pH step harms protein structure, low pH treatment often results in conformational changes that lower the potency of treated proteins and hence are usually avoided.

Carbohydrate Analysis.

Finally, the glycosylation on the A244-4gp120 protein was characterized by mass spectrometry and compared to the glycosylation present of two gp120 proteins (TV1 and 1086) currently being tested in clinical trials in Africa. It can be seen that the N-linked glycosylation present on the gp120 made in the Mgat1⁻ cell line is predominately mannose 5, with small amounts of mannose-8 and mannose-9, whereas the glycans present on the gp120s made in normal CHO cells consist of a broad spectrum of high mannose and sialic acid containing glycans.

In summary, these results confirm that recombinant HIV-1 envelope proteins (e.g. A244-rgp120N332) produced in the Mgat1-cell line are homogeneous and can be purified by conventional column chromatography without significant loss of material during recovery.

Diagram of gp120 from the IIIB strain of HIV-1 showing the location of N-linked glycosylation sites is published in Leonard et al 1990 (Leonard, C. K., et al., Assignment of intrachain disulfide bonds and characterization of potential glycosylation sites of the type 1 recombinant human immunodeficiency virus envelope glycoprotein (gp120) expressed in Chinese hamster ovary cells. J Biol Chem, 1990. 265(18): p. 10373-82).

FIGS. 29A-29F. Data from 2-dimensional isoelectric focusing gel analysis of MN-rgp120 produced in CHO and 293 HEK cells. Data shows showing the heterogeneity of net charge of in proteins purified by immuno-affinity chromatography (FIGS. 29A and 29D). The sensitivity of gp120s to digestion of the glycosidases, neuraminidase (FIGS. 29B and 29E) and endoglycosidase H (FIGS. 29C and 29F) was also measured. Digestion with neuraminidase, specific for sialic acid, shows that much of the heterogeneity in isoelectric point and net charge can be attributed to the incorporation of sialic acid. Digestion with Endo H shows that glycans lacking sialic acid are present in the two gp120 preparations and account for more heterogeneity in CHO cells than 293 cells. Data taken from Yu, et al 2012 (Yu, B., et al., Glycoform and Net Charge Heterogeneity in gp120 Immunogens Used in HIV Vaccine Trials. PLoS One, 2012. 7(8): p. e43903).

FIG. 30. Purification of A244_N332-rgp120 by column chromatography.

FIG. 31. Comparison of A244_N332-rgp120 recovered by an immunoaffinity recovery process dependent of the 5B6 monoclonal antibody and column chromatography (Desalting-IEXHP-SEC) recovery process. Data shows the proteins in fraction from the size exclusion step common to both recovery processes. Pluses (+) and minuses (−) indicate the presence or absence of the reducing agent dithiothreitol (DTT).

FIG. 32. Purification of A244_N332-rgp120 by immunoaffinity chromatography and size exclusion chromatography.

FIG. 33. Comparison of the recovered yields of A244_N332-rgp120 obtained from the recovery process containing an immunoaffinity step and the recovery process depending only on column chromatography. AUC indicates area under the curve. BCA indicates data from modified Bradford assay to measure protein concentration.

Mass spectroscopy analysis of glycans present in A244_N332-rgp120 recovered from the stable Mgat1-CHO cell line expressing A244_N332-rgp120 and gp120s from the TV1.0 and 1086.0 strains of HIV1 produced in normal CHO cell lines shows that the glycosylation was 99.47% high mannose. Data on A244_N332-rgp120 was kindly provided by Dr. Parastoo Asadi (Complex Carbohydrate Research Center, University of Georgia, Athens, Ga.). Data showing the glycan analysis of the TV1 and 1086 gp120 protein was taken from Wang et al. (Wang, Z., et al., Vaccines, 2016. 4(2): p. 17).

Although preferred embodiments of the subject invention have been described in some detail, it is understood that obvious variations can be made without departing from the spirit and the scope of the invention as defined herein.

What is claimed is:

1. A genetically modified Chinese hamster ovary (CHO) cell line comprising:
a heterologous nucleic acid comprising a nucleotide sequence encoding a human immunodeficiency virus (HIV) envelope glycoprotein polypeptide or fragment thereof comprising an N-linked glycosylation site; and
a mutation of an endogenous gene encoding mannosyl (alpha-1,3)-glycoprotein beta-1,2-N-Acetylglucosaminyltransferase (Mgat1),
wherein the mutation prevents Mgat1-mediated addition of a N-acetylglucosamine moiety to a terminal mannose residue present at the N-linked glycosylation site of the HIV envelope glycoprotein polypeptide such that at least 75% of the HIV envelope glycoprotein polypeptides produced by the genetically modified cell line comprise terminal mannose-5, mannose-8, or mannose-9 glycans at the N-linked glycosylation site.

2. The genetically modified cell line of claim 1, wherein the polypeptide is gp120 or an N-linked glycosylation site containing fragment thereof.

3. The genetically modified cell line of claim 2, wherein the fragment comprises variable regions 1 and 2 (V1/V2) or V3 domain comprising N-linked glycosylation sites N301 and N332.

4. The genetically modified cell line of claim 3, wherein the fragment comprising variable regions 1 and 2 is a monomer.

5. The genetically modified cell line of claim 1, wherein the polypeptide or fragment thereof is gp140.

6. The genetically modified cell line of claim 5, wherein the polypeptide or fragment thereof is expressed as a trimer.

7. The genetically modified cell line of any one of the preceding claims, wherein the polypeptide is fused to a heterologous signal sequence.

8. The genetically modified cell line of claim 7, wherein the heterologous signal sequence comprises the amino acid sequence set forth in one of SEQ ID NOs: 44-47.

9. The genetically modified cell line of any one of the preceding claims, wherein the polypeptide comprises a purification tag.

10. The genetically modified cell line of claim 9, wherein the purification tag comprises the amino acid sequence set forth in one of SEQ ID NOs: 48-56.

11. The genetically modified cell line of claim 1, wherein the polypeptide comprises the amino acid sequence set forth in SEQ ID NO: 1, 2, 3, 5, 7, 9, 10, 12, 13, 15, 17, 18, 20, 22, 23, 25, 26, 28, 30, 32, 34, 36, 38, 40, or 42.

12. The genetically modified cell line of any one of the preceding claims, wherein the cell line produces the polypeptide at a concentration of at least 50 mg/L after 5 days of culturing.

13. The genetically modified cell line of any one of the preceding claims, wherein the cell line is of CHO K1 lineage.

14. The genetically modified cell line of any one of the preceding claims, wherein the cell line is of CHO-S lineage.

15. The genetically modified cell line of any one of the preceding claims, wherein the cell line comprises an endogenous gene encoding glutamine synthetase (GS).

16. The genetically modified cell line of any one of the preceding claims, wherein the cell line comprises an endogenous gene encoding dihydrofolate reductase (DHFR).

17. A genetically modified Chinese hamster ovary (CHO) cell line comprising a mutation of gene encoding mannosyl (alpha-1,3)-glycoprotein beta-1,2-N-Acetylglucosaminyltransferase (Mgat1), wherein the genetically modified cell line is deposited with American Type Culture Collection (ATCC) as:
i) PTA-124141; or
ii) PTA-124142.

18. A method of producing a human immunodeficiency virus (HIV) envelope glycoprotein polypeptide or fragment thereof, the fragment comprising an N-linked glycosylation site, the polypeptide or fragment thereof comprising terminal mannose-5 glycans, the method comprising:
a) introducing a nucleic acid comprising a nucleotide sequence encoding the HIV envelope glycoprotein polypeptide into a genetically modified Chinese hamster ovary (CHO) cell line comprising a mutation of an endogenous gene encoding mannosyl (alpha-1,3)-glycoprotein beta-1,2-N-Acetylglucosaminyltransferase (Mgat1),
wherein the mutation prevents Mgat1 mediated addition of a N-acetylglucosamine moiety to a terminal mannose residue such that at least 75% of the HIV envelope glycoprotein polypeptide produced by the genetically modified cell line comprises terminal mannose-5, mannose-8, or mannose-9 glycans; and
b) culturing the cell line in a liquid culture medium under conditions sufficient for production of the HIV envelope glycoprotein polypeptide comprising terminal mannose-5, mannose-8, or mannose-9 glycans.

19. The method of claim 18, wherein the envelope glycoprotein fragment comprises variable region 3 (V3) and optionally, C3 domain.

20. The method of claim 18, wherein the envelope glycoprotein is gp120 or a fragment thereof.

21. The method of claim 18, wherein the fragment comprises variable regions 1 and 2 (V1/V2).

22. The method of claim 21, wherein the fragment comprising variable regions 1 and 2 is a monomer.

23. The method of claim 18, wherein the polypeptide is gp140 or a fragment thereof.

24. The method of claim 23, wherein polypeptide is expressed as a trimer.

25. The method of any one claims 18-24, wherein the polypeptide is fused to a heterologous signal sequence.

26. The method of claim 25, wherein the heterologous signal sequence comprises the amino acid sequence set forth in one of SEQ ID NOs: 44-47.

27. The method of any one of claims 18-26, wherein the polypeptide comprises a purification tag.

28. The method of claim 27, wherein the purification tag comprises the amino acid sequence set forth in one of SEQ ID NOs: 48-56.

29. The method of claim 18, wherein the polypeptide comprises the amino acid sequence set forth in SEQ ID NO: 1, 2, 3, 5, 7, 9, 10, 12, 13, 15, 17, 18, 20, 22, 23, 25, 26, 28, 30, 32, 34, 36, 38, 40, or 42.

30. The method of claim 18, wherein the nucleic acid comprises a nucleotide sequence set forth in SEQ ID NO:4, 6, 8, 11, 14, 16, 19, 21, 24, 27, 29, 31, 33, 35, 37, 39, 41, or 43.

31. The method of any one of claims 18-30, comprising screening individual clones of the cell line to identify clones expressing the highest amounts of the polypeptide, the screening comprising plating the clones in a semisolid matrix and contacting the clones with a detectably labeled antibody that binds to the polypeptide.

32. The method of claim 31, wherein the antibodies are fluorescently labeled antibodies that bind to the polypeptide and form a precipitate around the clones, wherein the precipitate is visible under fluorescent light.

33. The method of claim 32, further comprising identifying clones surrounded by precipitate meeting a selection threshold and isolating the identified clones.

34. The method of any one of claims 31-33, wherein the antibodies are polyclonal antibodies.

35. The method of claim 34, wherein the polyclonal antibodies are affinity purified antibodies that bind to the polypeptide.

36. The method of any one of claims 32-35, wherein the fluorescent label is Alexa dye.

37. The method of any one of claims 18-36, further comprising recovering the HIV envelope glycoprotein polypeptide comprising terminal mannose-5, mannose-8, or mannose-9 glycans from the culture medium.

38. A recombinant HIV envelope glycoprotein polypeptide or a fragment thereof comprising at least one N-linked glycosylation site, wherein the polypeptide or the fragment comprises terminal mannose-5, mannose-8, or mannose-9 glycans at the N-linked glycosylation site.

39. The recombinant HIV envelope glycoprotein polypeptide or a fragment thereof of claim 38 comprising a plurality of N-linked glycosylation sites, wherein the polypeptide or the fragment comprises terminal mannose-5, mannose-8, or mannose-9 glycans at the plurality of N-linked glycosylation sites.

40. The recombinant HIV envelope glycoprotein polypeptide or a fragment thereof of claim 38, wherein at least 75% of the N-linked glycosylation sites of the polypeptide or the fragment comprise terminal mannose-5, mannose-8, or mannose-9 glycans.

41. The recombinant HIV envelope glycoprotein polypeptide or a fragment thereof of any one of claims 38-40, wherein the polypeptide is gp120 or a fragment thereof.

42. The recombinant HIV envelope glycoprotein polypeptide or a fragment thereof of any one of claims 38-41, wherein the fragment comprises variable regions 1 and 2 (V1/V2) or V3 domain comprising N-linked glycosylation sites N301 and N332.

43. The recombinant HIV envelope glycoprotein polypeptide or a fragment thereof of claim 42, wherein the fragment comprising variable regions 1 and 2 is a monomer.

44. The recombinant HIV envelope glycoprotein polypeptide or a fragment thereof of any one of claims 38-41, wherein the polypeptide or fragment thereof is gp140.

45. The recombinant HIV envelope glycoprotein polypeptide or a fragment thereof of claim 44, wherein the polypeptide or the fragment is expressed as a trimer.

46. The recombinant HIV envelope glycoprotein polypeptide or a fragment thereof of any one of claims 38-45, wherein the polypeptide or the fragment is fused to a heterologous signal sequence.

47. The recombinant HIV envelope glycoprotein polypeptide or a fragment thereof of claim 46, wherein the heterologous signal sequence comprises the amino acid sequence set forth in one of SEQ ID NOs: 44-47.

48. The recombinant HIV envelope glycoprotein polypeptide or a fragment thereof of any one of claims 38-47, wherein the polypeptide or the fragment comprises a purification tag.

49. The recombinant HIV envelope glycoprotein polypeptide or a fragment thereof of claim 48, wherein the purification tag comprises the amino acid sequence set forth in one of SEQ ID NOs: 48-56.

50. The recombinant HIV envelope glycoprotein polypeptide or a fragment thereof of any one of claims 38-40, wherein the polypeptide or the fragment comprises the amino acid sequence set forth in SEQ ID NO: 1, 2, 3, 5, 7, 9, 10, 12, 13, 15, 17, 18, 20, 22, 23, 25, 26, 28, 30, 32, 34, 36, 38, 40, or 42 or comprises an amino acid sequence at least 85% identical to the amino acid sequence set forth in SEQ ID NO: 1, 2, 3, 5, 7, 9, 10, 12, 13, 15, 17, 18, 20, 22, 23, 25, 26, 28, 30, 32, 34, 36, 38, 40, or 42.

51. A composition comprising the polypeptide or the fragment of any one of claims 38-50 and a pharmaceutically acceptable excipient.

52. A method for inducing an immune response to HIV in a mammal, the method comprising administering to the mammal the composition of claim 51.


SEQUENCE LISTING (72 sequences)

SEQ ID NO: 1 - protein, 470 aa, Artificial Sequence
  synthetic polypeptide
VPVWKEADTTLFCASDAKAHETEVHNVWATHACVPTDPNPQEIDLENVTENFNMWKMVEQMQEDVISLWDQSLKPCVKLTPPCVTLHCTNANLTKANLTNVNNRTNVSNIIGNITDEVRNCSFNMTTELRDKKQKVHALFYKLDIVPIEDNNDSSEYRLINCNTSVIKQACPKISFDPIPIHYCTPAGYAILKCNDKNFNGTGPCKNVSSVQCTHGIKPVVSTQLLLNGSLAEEEIIIRSENLTNNAKTIIVHLNKSVVINCTRPSNNTRTSITIGPGQVFYRTGDIIGDIRKAYCNISGTEWNKALKQVTEKLKEHFNNKPIIFQPPSGGDLEITMHHFNCRGEFFYCNTTRLFNNTCIANGTIEGCNGNITLPCKIKQIINMWQGAGQAMYAPPISGTINCVSNITGILLTRDGGATNNTNNETFRPGGGNIKDNWRNELYKYKVVQIEPLGVAPTRAKRRVVEREKR

SEQ ID NO: 2 - protein, 462 aa, Artificial sequence
  synthetic polypeptide
VPVWKEADTTLFCASDAKAHETEVHNVWATHACVPTDPNPQEIDLENVTENFNMWKNNMVEQMQEDVISLWDQSLKPCVKLTPPCVTLHCTNANLTKANLTNVNNRTNVSNIIGNITDEVRNCSFNMTTELRDKKQKVHALFYKLDIVPIEDNNDSSEYRLINCNTSVIKQACPKISFDPIPIHYCTPAGYAILKCNDKNFNGTGPCKNVSSVQCTHGIKPVVSTQLLLNGSLAEEEIIIRSENLTNNAKTIIVHLNKSVVINCTRPSNNTRTSITIGPGQVFYRTGDIIGDIRKAYCNISGTEWNKALKQVTEKLKEHFNNKPIIFQPPSGGDLEITMHHFNCRGEFFYCNTTRLFNNTCIANGTIEGCNGNITLPCKIKQIINMWQGAGQAMYAPPISGTINCVSNITGILLTRDGGATNNTNNETFRPGGGNIKDNWRNELYKYKVVQIEPLGVAPTRA

SEQ ID NO: 3 - protein, 517 aa, Artificial sequence
  synthetic polypeptide
MGGAAARLGAVILFVVIVGLHGVRGKYALADASLKMADPNRFRGKDLPVLDQLLEVPVWKEADTTLFCASDAKAHETEVHNVWATHACVPTDPNPQEIDLENVTENFNMWKNNMVEQMQEDVISLWDQSLKPCVKLTPPCVTLHCTNANLTKANLTNVNNRTNVSNIIGNITDEVRNCSFNMTTELRDKKQKVHALFYKLDIVPIEDNNDSSEYRLINCNTSVIKQACPKISFDPIPIHYCTPAGYAILKCNDKNFNGTGPCKNVSSVQCTHGIKPVVSTQLLLNGSLAEEEIIIRSENLTNNAKTIIVHLNKSVVINCTRPSNNTRTSITIGPGQVFYRTGDIIGDIRKAYCNISGTEWNKALKQVTEKLKEHFNNKPIIFQPPSGGDLEITMHHFNCRGEFFYCNTTRLFNNTCIANGTIEGCNGNITLPCKIKQIINMWQGAGQAMYAPPISGTINCVSNITGILLTRDGGATNNTNNETFRPGGGNIKDNWRNELYKYKVVQIEPLGVAPTRA

SEQ ID NO: 4 - DNA, 1581 bp, Artificial sequence
  synthetic nucleic acid
atggggggggctgccgccaggttgggggccgtgattttgtttgtcgtcatagtgggcctccatggggtccgcggcaaatatgccttggcggatgcctctctcaagatggccgaccccaatcgatttcgcggcaaagaccttccggtcctggaccagctgctcgaggtaccagtgtggaaggaagccgacacaaccctcttctgcgccagcgatgccaaggcccacgagacggaggtccacaatgtgtgggccacccatgcctgtgtgcccacggaccccaacccccaggagattgacctggagaatgtcacggagaacttcaacatgtggaagaacaacatggtggagcagatgcaggaggacgtcatctccctgtgggaccagagcctgaaaccctgcgtcaaactgacacccccctgtgtgaccctgcactgcacgaacgccaacctgaccaaggccaacctcaccaacgtgaacaatcggaccaacgtgtccaacatcatcgggaacatcacagatgaggtgaggaactgcagcttcaatatgacaaccgagctccgggacaaaaagcagaaggtgcacgcgttgttctacaaactggatatcgtccccatcgaggacaataatgacagctccgagtatcgcctgatcaactgcaacaccagcgtcatcaaacaggcctgccccaaaatttccttcgaccccatccccatccactactgcaccccagctgggtacgccatcctgaagtgcaatgacaagaacttcaacggcacagggccctgcaagaatgtgagctccgtccagtgcacccacggcatcaagccagtggtctccacccagctcctcctgaatgggagcctggcagaggaagagatcatcatccgctccgagaacctgaccaacaatgccaagaccatcatcgtccacctgaataagtccgtggtcatcaactgcaccagacccagcaacaacacgcggaccagcatcaccatcggcccagggcaggtcttctataggacgggggacatcattggggacatcaggaaggcctactgcaacatcagtgggaccgagtggaacaaagccctgaaacaggtgaccgaaaaactcaaggagcacttcaacaacaagccaatcatcttccagccccccagcgggggggacctggagatcaccatgcaccatttcaactgccggggggaattcttctactgcaacaccacccgcctgttcaacaacacctgcatcgccaacggcaccatcgagggctgcaatggcaacatcaccctcccatgcaaaatcaagcagatcatcaacatgtggcagggggcaggccaggccatgtacgccccccccatctccggcacgatcaactgcgtgtccaacatcacggggatcctgctgacccgggatgggggggctaccaacaatacgaacaatgagaccttcaggccagggggggggaacatcaaagacaactggcgcaatgagctctacaagtacaaagtggtgcagatcgagcccctgggggtggcccccacccgggccaaacgcagggtggtggagcgggagaagcgg

SEQ ID NO: 5 - protein, 684 aa, Artificial sequence
  synthetic polypeptide
MRVKETQMNWPNLWKWGTLILGLVIICSASDNLWVTVYYGVPVWKEADTTLFCASDAKAHETEVHNVWATHACVPTDPNPQEIDLENVTENFNMWKNNMVEQMQEDVISLWDQSLKPCVKLTPLCVTLHCTNANLTKANLTNVNNRTNVSNIIGNITDEVRNCSFNMTTELRDKKQKVHALFYKLDIVPIEDNNDSSEYRLINCNTSVIKQACPKISFDPIPIHYCTPAGYAILKCNDKNFNGTGPCKNVSSVQCTHGIKPVVSTQLLLNGSLAEEEIIIRSDNLTNNAKTIIVHLNKSVVINCTRPSNNTRTSITIGPGQVFYRTGDIIGDIRKAYCEINGTEWNKALKQVTEKLKEHFNNKPIIFQPPSGGDLEITMHHFNCRGEFFYCNTTRLFNNTCIANGTIEGCNGNITLPCKIKQIINMWQGAGQAMYAPPISGTINCVSNITGILLTRDGGATNNTNNETFRPGGGNIKDNWRNELYKYKVVQIEPLGVAPTRAKRRVVEREKRAVGIGAMIFGFLGAAGSTMGAASITLTVQARQLLSGIVQQQSNLLRAIEAQQHLLQLTVWGIKQLQARVLAVERYLKDQKFLGLWGCSGKIICTTAVPWNSTWSNKSLEEIWSNMTWIEWEREISNYTNQIYEILTKSQDQQDRNEKDLLELDKWASLWTWFDITNWLWYIK

SEQ ID NO: 6 - DNA, 2052 bp, Artificial sequence
  synthetic nucleic acid
atgagagtgaaggagacacagatgaattggccaaacttgtggaaatgggggactttgatccttgggttggtgataatttgtagtgcctcagacaacttgtgggttacagtttattatggggtaccagtgtggaaggaagccgacacaaccctcttctgcgccagcgatgccaaggcccacgagacggaggtccacaatgtgtgggccacccatgcctgtgtgcccacggaccccaacccccaggagattgacctggagaatgtcacggagaacttcaacatgtggaagaacaacatggtggagcagatgcaggaggacgtcatctccctgtgggaccagagcctgaaaccctgcgtcaaactgacacccccctgtgtgaccctgcactgcacgaacgccaacctgaccaaggccaacctcaccaacgtgaacaatcggaccaacgtgtccaacatcatcgggaacatcacagatgaggtgaggaactgcagcttcaatatgacaaccgagctccgggacaaaaagcagaaggtgcacgcgttgttctacaaactggatatcgtccccatcgaggacaataatgacagctccgagtatcgcctgatcaactgcaacaccagcgtcatcaaacaggcctgccccaaaatttccttcgaccccatccccatccactactgcaccccagctgggtacgccatcctgaagtgcaatgacaagaacttcaacggcacagggccctgcaagaatgtgagctccgtccagtgcacccacggcatcaagccagtggtctccacccagctcctcctgaatgggagcctggcagaggaagagatcatcatccgctccgagaacctgaccaacaatgccaagaccatcatcgtccacctgaataagtccgtggtcatcaactgcaccagacccagcaacaacacgcggaccagcatcaccatcggcccagggcaggtcttctataggacgggggacatcattggggacatcaggaaggcctactgcgaaatcaatgggaccgagtggaacaaagccctgaaacaggtgaccgaaaaactcaaggagcacttcaacaacaagccaatcatcttccagccccccagcgggggggacctggagatcaccatgcaccatttcaactgccggggggaattcttctactgcaacaccacccgcctgttcaacaacacctgcatcgccaacggcaccatcgagggctgcaatggcaacatcaccctcccatgcaaaatcaagcagatcatcaacatgtggcagggggcaggccaggccatgtacgccccccccatctccggcacgatcaactgcgtgtccaacatcacggggatcctgctgacccgggatgggggggctaccaacaatacgaacaatgagaccttcaggccagggggggggaacatcaaagacaactggcgcaatgagctctacaagtacaaagtggtgcagatcgagcccctgggggtggcccccacccgggccaaacgcagggtggtggagcgggagaagcgggcagtgggcattggggccatgatcttcggctttctgggagccgccggatctacaatgggagctgccagcatcaccctgaccgtgcaggctagacaactgctgtctggcatcgtgcagcagcagagcaatctgctgagagccattgaggcccagcagcatctgctgcagctgacagtgtggggcatcaaacagctgcaggccagagtgctggccgtggaaagatacctgaaggaccagaaattcctcggcctgtggggctgcagcggcaagatcatctgtacaacagccgtgccttggaacagcacctggtccaacaagagcctggaagagatctggtccaatatgacctggatcgagtgggagagagagatcagcaactacaccaaccagatctacgagatcctgaccaagagccaggaccagcaggaccggaacgagaaggatctgctggaactggacaagtgggccagcctgtggacttggtttgacatcaccaactggctgtggtacatcaag

SEQ ID NO: 7 - protein, 684 aa, Artificial sequence
  synthetic polypeptide
MRVKETQMNWPNLWKWGTLILGLVIICSASDNLWVTVYYGVPVWKEADTTLFCASDAKAHETEVHNVWATHACVPTDPNPQEIDLENVTENFNMWKNNMVEQMQEDVISLWDQSLKPCVKLTPLCVTLHCTNANLTKANLTNVNNRTNVSNIIGNITDEVRNCSFNMTTELRDKKQKVHALFYKLDIVPIEDNNDSSEYRLINCNTSVIKQACPKISFDPIPIHYCTPAGYAILKCNDKNFNGTGPCKNVSSVQCTHGIKPVVSTQLLLNGSLAEEEIIIRSDNLTNNAKTIIVHLNKSVVINCTRPSNNTRTSITIGPGQVFYRTGDIIGDIRKAYCNISGTEWNKALKQVTEKLKEHFNNKPIIFQPPSGGDLEITMHHFNCRGEFFYCNTTRLFNNTCIANGTIEGCNGNITLPCKIKQIINMWQGAGQAMYAPPISGTINCVSNITGILLTRDGGATNNTNNETFRPGGGNIKDNWRNELYKYKVVQIEPLGVAPTRAKRRVVEREKRAVGIGAMIFGFLGAAGSTMGAASITLTVQARQLLSGIVQQQSNLLRAIEAQQHLLQLTVWGIKQLQARVLAVERYLKDQKFLGLWGCSGKIICTTAVPWNSTWSNKSLEEIWSNMTWIEWEREISNYTNQIYEILTKSQDQQDRNEKDLLELDKWASLWTWFDITNWLWYIK

SEQ ID NO: 8 - DNA, 2052 bp, Artificial sequence
  synthetic nucleic acid
atgagagtgaaggagacacagatgaattggccaaacttgtggaaatgggggactttgatccttgggttggtgataatttgtagtgcctcagacaacttgtgggttacagtttattatggggtaccagtgtggaaggaagccgacacaaccctcttctgcgccagcgatgccaaggcccacgagacggaggtccacaatgtgtgggccacccatgcctgtgtgcccacggaccccaacccccaggagattgacctggagaatgtcacggagaacttcaacatgtggaagaacaacatggtggagcagatgcaggaggacgtcatctccctgtgggaccagagcctgaaaccctgcgtcaaactgacacccccctgtgtgaccctgcactgcacgaacgccaacctgaccaaggccaacctcaccaacgtgaacaatcggaccaacgtgtccaacatcatcgggaacatcacagatgaggtgaggaactgcagcttcaatatgacaaccgagctccgggacaaaaagcagaaggtgcacgcgttgttctacaaactggatatcgtccccatcgaggacaataatgacagctccgagtatcgcctgatcaactgcaacaccagcgtcatcaaacaggcctgccccaaaatttccttcgaccccatccccatccactactgcaccccagctgggtacgccatcctgaagtgcaatgacaagaacttcaacggcacagggccctgcaagaatgtgagctccgtccagtgcacccacggcatcaagccagtggtctccacccagctcctcctgaatgggagcctggcagaggaagagatcatcatccgctccgagaacctgaccaacaatgccaagaccatcatcgtccacctgaataagtccgtggtcatcaactgcaccagacccagcaacaacacgcggaccagcatcaccatcggcccagggcaggtcttctataggacgggggacatcattggggacatcaggaaggcctactgcaacatcagtgggaccgagtggaacaaagccctgaaacaggtgaccgaaaaactcaaggagcacttcaacaacaagccaatcatcttccagccccccagcgggggggacctggagatcaccatgcaccatttcaactgccggggggaattcttctactgcaacaccacccgcctgttcaacaacacctgcatcgccaacggcaccatcgagggctgcaatggcaacatcaccctcccatgcaaaatcaagcagatcatcaacatgtggcagggggcaggccaggccatgtacgccccccccatctccggcacgatcaactgcgtgtccaacatcacggggatcctgctgacccgggatgggggggctaccaacaatacgaacaatgagaccttcaggccagggggggggaacatcaaagacaactggcgcaatgagctctacaagtacaaagtggtgcagatcgagcccctgggggtggcccccacccgggccaaacgcagggtggtggagcgggagaagcgggcagtgggcattggggccatgatcttcggctttctgggagccgccggatctacaatgggagctgccagcatcaccctgaccgtgcaggctagacaactgctgtctggcatcgtgcagcagcagagcaatctgctgagagccattgaggcccagcagcatctgctgcagctgacagtgtggggcatcaaacagctgcaggccagagtgctggccgtggaaagatacctgaaggaccagaaattcctcggcctgtggggctgcagcggcaagatcatctgtacaacagccgtgccttggaacagcacctggtccaacaagagcctggaagagatctggtccaatatgacctggatcgagtgggagagagagatcagcaactacaccaaccagatctacgagatcctgaccaagagccaggaccagcaggaccggaacgagaaggatctgctggaactggacaagtgggccagcctgtggacttggtttgacatcaccaactggctgtggtacatcaag

SEQ ID NO: 9 - protein, 463 aa, Artificial sequence
  synthetic polypeptide
  misc_feature (191)..(191): Xaa can be any naturally occurring amino acid
VPVWKEATTTLFCASDAKAYDTEAHNVWATHACVPTDPNPQEVELVNVTENFNMWKNNMVEQMHEDIISLWDQSLKPCVKLTPLCVTLNCTDLRNTTNTNNSTDNNNSKSEGTIKGGEMKNCSFNITTSIGDKMQKEYALLYKLDIEPIDNDSTSYRLISCNTSVITQACPKISFEPIPIHYCAPAGFAIXKCNDKKFSGKGSCKNVSTVQCTHGIRPVVSTQLLLNGSLAEEEVVIRSEDFTDNAKTIIVHLNESVQINCTRPNNNTRKRIHIGPGRAFYTTKNIKGTIRQAHCNISRAKWNDTLRQIVSKLKEQFKNKTIVFNPSSGGDPEIVMHSFNCGGEFFYCNTSPLFNSIWNGNNTWNNTTGSNNNITLQCKIKQIINMWQKVGKAMYAPPIEGQIRCSSNITGLLLTRDGGEDTDTNDTEIFRPGGGDMRDNWRSELYKYKVVTIEPLGVAPTKA

SEQ ID NO: 10 - protein, 526 aa, Artificial sequence
  synthetic polypeptide
  misc_feature (246)..(246): Xaa can be any naturally occurring amino acid
MGGAAARLGAVILFVVIVGLHGVRGKYALADASLKMADPNRFRGKDLPVLDQLLEVPVWKEATTTLFCASDAKAYDTEAHNVWATHACVPTDPNPQEVELVNVTENFNMWKNNMVEQMHEDIISLWDQSLKPCVKLTPLCVTLNCTDLRNTTNTNNSTDNNNSKSEGTIKGGEMKNCSFNITTSIGDKMQKEYALLYKLDIEPIDNDSTSYRLISCNTSVITQACPKISFEPIPIHYCAPAGFAIXKCNDKKFSGKGSCKNVSTVQCTHGIRPVVSTQLLLNGSLAEEEVVIRSEDFTDNAKTIIVHLNESVQINCTRPNNNTRKRIHIGPGRAFYTTKNIKGTIRQAHCNISRAKWNDTLRQIVSKLKEQFKNKTIVFNPSSGGDPEIVMHSFNCGGEFFYCNTSPLFNSIWNGNNTWNNTTGSNNNITLQCKIKQIINMWQKVGKAMYAPPIEGQIRCSSNITGLLLTRDGGEDTDTNDTEIFRPGGGDMRDNWRSELYKYKVVTIEPLGVAPTKAKRRVVQRE

SEQ ID NO: 11 - DNA, 1578 bp, Artificial sequence
  synthetic nucleic acid
  misc_feature (736)..(736): n is a, c, g, or t
atggggggggctgccgccaggttgggggccgtgattttgtttgtcgtcatagtgggcctccatggggtccgcggcaaatatgccttggcggatgcctctctcaagatggccgaccccaatcgatttcgcggcaaagaccttccggtcctggaccagctgctcgaggtacctgtgtggaaagaagcaaccaccactctattttgtgcatcagatgctaaagcatatgatacagaggcacataatgtttgggccacacatgcctgtgtacccacagaccccaacccacaagaagtagaattggtaaatgtgacagaaaattttaacatgtggaaaaataacatggtagaacagatgcatgaggatataatcagtttatgggatcaaagcctaaagccatgtgtaaaattaaccccactctgtgttactttaaattgcactgatttgaggaatactactaataccaataatagtactgataataacaatagtaaaagcgagggaacaataaagggaggagaaatgaaaaactgctctttcaatatcaccacaagcataggagataagatgcagaaagaatatgcacttctttataaacttgatatagaaccaatagataatgatagtaccagctataggttgataagttgtaatacctcagtcattacacaagcttgtccaaagatatcctttgagccaattcccatacactattgtgccccggctggttttgcgattntaaagtgtaacgataaaaagttcagtggaaaaggatcatgtaaaaatgtcagcacagtacaatgtacacatggaattaggccagtagtatcaactcaactgctgttaaatggcagtctagcagaagaagaggtagtaattagatctgaggatttcactgataatgctaaaaccatcatagtacatctgaacgaatctgtacaaattaattgtacaagacccaacaacaataccagaaaaaggatacatataggaccagggagagcattttatacaacaaaaaatataaaaggaactataagacaagcacattgtaacattagtagagcaaaatggaatgacactttaagacagatagttagcaagttaaaagaacaatttaagaataaaacaatagtctttaatccatcctcaggaggggacccagaaattgtaatgcacagttttaattgtggaggggaatttttctactgtaatacatcaccactgtttaatagtatttggaatggtaataatacttggaataatactacagggtcaaataacaatatcacacttcaatgcaaaataaaacaaattataaacatgtggcagaaagtaggaaaagcaatgtatgcccctcccattgaaggacaaattagatgttcatcaaatattacagggctactattaacaagagatggtggtgaggacacggacacgaacgacaccgagatcttcagacctggaggaggagatatgagggacaattggagaagtgaattatataaatataaagtagtaacaattgaaccattaggagtagcacccaccaaggcaaagagaagagtggtgcagagagaa

SEQ ID NO: 12 - protein, 461 aa, Artificial sequence
  synthetic polypeptide
VPVWKEATTTLFCASDAKAYDTEAHNVWATHACVPTDPNPQEVELVNVTENFNMWKNNMVEQMHEDIISLWDQSLKPCVKLTPLCVTLNCTDLRNTTNTNNSTDNNNSKSEGTIKGGEMKNCSFNITTSIGDKMQKEYALLYKLDIEPIDNDSTSYRLISCNTSVITQACPKISFEPIPIHYCAPAGFAILKCNDKKFSGKGSCKNVSTVQCTHGIRPVVSTQLLLNGSLAEEEVVIRSEDFTDNAKTIIVHLKESVQINCTRPNNNTRKRIHIGPGRAFYTTKNIKGTIRQAHCNISRAKWNDTLRQIVSKLKEQFKNKTIVFNPSSGGDPEIVMHSFNCGGEFFYCNTSPLFNSIWNGNNTWNNTTGSNNNITLQCKIKQIINMWQKVGKAMYAPPIEGQIRCSSNITGLLLTRDGGEDTDTNDTEIFRPGGGDMRDNWRSELYKYKVVTIEPLGVAPT

SEQ ID NO: 13 - protein, 526 aa, Artificial sequence
  synthetic polypeptide
MGGAAARLGAVILFVVIVGLHGVRGKYALADASLKMADPNRFRGKDLPVLDQLLEVPVWKEATTTLFCASDAKAYDTEAHNVWATHACVPTDPNPQEVELVNVTENFNMWKNNMVEQMHEDIISLWDQSLKPCVKLTPLCVTLNCTDLRNTTNTNNSTDNNNSKSEGTIKGGEMKNCSFNITTSIGDKMQKEYALLYKLDIEPIDNDSTSYRLISCNTSVITQACPKISFEPIPIHYCAPAGFAILKCNDKKFSGKGSCKNVSTVQCTHGIRPVVSTQLLLNGSLAEEEVVIRSEDFTDNAKTIIVHLKESVQINCTRPNNNTRKRIHIGPGRAFYTTKNIKGTIRQAHCNISRAKWNDTLRQIVSKLKEQFKNKTIVFNPSSGGDPEIVMHSFNCGGEFFYCNTSPLFNSIWNGNNTWNNTTGSNNNITLQCKIKQIINMWQKVGKAMYAPPIEGQIRCSSNITGLLLTRDGGEDTDTNDTEIFRPGGGDMRDNWRSELYKYKVVTIEPLGVAPTKAKRRVVQRE

SEQ ID NO: 14 - DNA, 1578 bp, Artificial sequence
  synthetic nucleic acid
atggggggggctgccgccaggttgggggccgtgattttgtttgtcgtcatagtgggcctccatggggtccgcggcaaatatgccttggcggatgcctctctcaagatggccgaccccaatcgatttcgcggcaaagaccttccggtcctggaccagctgctcgaggtacctgtgtggaaagaagcaaccaccactctattttgtgcatcagatgctaaagcatatgatacagaggcacataatgtttgggccacacatgcctgtgtacccacagaccccaacccacaagaagtagaattggtaaatgtgacagaaaattttaacatgtggaaaaataacatggtagaacagatgcatgaggatataatcagtttatgggatcaaagcctaaagccatgtgtaaaattaaccccactctgtgttactttaaattgcactgatttgaggaatactactaataccaataatagtactgataataacaatagtaaaagcgagggaacaataaagggaggagaaatgaaaaactgctctttcaatatcaccacaagcataggagataagatgcagaaagaatatgcacttctttataaacttgatatagaaccaatagataatgatagtaccagctataggttgataagttgtaatacctcagtcattacacaagcttgtccaaagatatcctttgagccaattcccatacactattgtgccccggctggttttgcgattctaaagtgtaacgataaaaagttcagtggaaaaggatcatgtaaaaatgtcagcacagtacaatgtacacatggaattaggccagtagtatcaactcaactgctgttaaatggcagtctagcagaagaagaggtagtaattagatctgaggatttcactgataatgctaaaaccatcatagtacatctgaaagaatctgtacaaattaattgtacaagacccaacaacaataccagaaaaaggatacatataggaccagggagagcattttatacaacaaaaaatataaaaggaactataagacaagcacattgtaacattagtagagcaaaatggaatgacactttaagacagatagttagcaagttaaaagaacaatttaagaataaaacaatagtctttaatccatcctcaggaggggacccagaaattgtaatgcacagttttaattgtggaggggaatttttctactgtaatacatcaccactgtttaatagtatttggaatggtaataatacttggaataatactacagggtcaaataacaatatcacacttcaatgcaaaataaaacaaattataaacatgtggcagaaagtaggaaaagcaatgtatgcccctcccattgaaggacaaattagatgttcatcaaatattacagggctactattaacaagagatggtggtgaggacacggacacgaacgacaccgagatcttcagacctggaggaggagatatgagggacaattggagaagtgaattatataaatataaagtagtaacaattgaaccattaggagtagcacccaccaaggcaaagagaagagtggtgcagagagaa

SEQ ID NO: 15 - protein, 684 aa, Artificial sequence
  synthetic polypeptide
MRVKGIRRNYQHWWGWGTMLLGLLMICSATEKLWVTVYYGVPVWKEATTTLFCASDAKAYDTEAHNVWATHACVPTDPNPQEVELVNVTENFNMWKNNMVEQMHEDIISLWDQSLKPCVKLTPLCVTLNCTDLRNTTNTNNSTDNNNSKSEGTIKGGEMKNCSFNITTSIGDKMQKEYALLYKLDIEPIDNDSTSYRLISCNTSVITQACPKISFEPIPIHYCAPAGFAILKCNDKKFSGKGSCKNVSTVQCTHGIRPVVSTQLLLNGSLAEEEVVIRSEDFTDNAKTIIVHLKESVQINCTRPNNNTRKRIHIGPGRAFYTTKNIKGTIRQAHCNISRAKWNDTLRQIVSKLKEQFKNKTIVFNPSSGGDPEIVMHSFNCGGEFFYCNTSPLFNSIWNGNNTWNNTTGSNNNITLQCKIKQIINMWQKVGKAMYAPPIEGQIRCSSNITGLLLTRDGGEDTDTNDTEIFRPGGGDMRDNWRSELYKYKVVTIEPLGVAPTKAKRRVVQREKRAAIGALFLGFLGAAGSTMGAASVTLTVQARLLLSGIVQQQNNLLRAIEAQQHMLQLTVWGIKQLQARVLAVERYLKDQQLLGFWGCSGKLICTTTVPWNASWSNKSLDDIWNNMTWMQWEREIDNYTSLIYSLLEKSQTQQEKNEQELLELDKWASLWNWFDITNWLWYIK

SEQ ID NO: 16 - DNA, 2052 bp, Artificial sequence
  synthetic nucleic acid
atgagagtgaaggggatcaggaggaattatcagcactggtggggatggggcacgatgctccttgggttattaatgatctgtagtgctacagaaaaattgtgggtcacagtctattatggggtacctgtgtggaaagaagcaaccaccactctattttgtgcatcagatgctaaagcatatgatacagaggcacataatgtttgggccacacatgcctgtgtacccacagaccccaacccacaagaagtagaattggtaaatgtgacagaaaattttaacatgtggaaaaataacatggtagaacagatgcatgaggatataatcagtttatgggatcaaagcctaaagccatgtgtaaaattaaccccactctgtgttactttaaattgcactgatttgaggaatactactaataccaataatagtactgataataacaatagtaaaagcgagggaacaataaagggaggagaaatgaaaaactgctctttcaatatcaccacaagcataggagataagatgcagaaagaatatgcacttctttataaacttgatatagaaccaatagataatgatagtaccagctataggttgataagttgtaatacctcagtcattacacaagcttgtccaaagatatcctttgagccaattcccatacactattgtgccccggctggttttgcgattctaaagtgtaacgataaaaagttcagtggaaaaggatcatgtaaaaatgtcagcacagtacaatgtacacatggaattaggccagtagtatcaactcaactgctgttaaatggcagtctagcagaagaagaggtagtaattagatctgaggatttcactgataatgctaaaaccatcatagtacatctgaaagaatctgtacaaattaattgtacaagacccaacaacaataccagaaaaaggatacatataggaccagggagagcattttatacaacaaaaaatataaaaggaactataagacaagcacattgtaacattagtagagcaaaatggaatgacactttaagacagatagttagcaagttaaaagaacaatttaagaataaaacaatagtctttaatccatcctcaggaggggacccagaaattgtaatgcacagttttaattgtggaggggaatttttctactgtaatacatcaccactgtttaatagtatttggaatggtaataatacttggaataatactacagggtcaaataacaatatcacacttcaatgcaaaataaaacaaattataaacatgtggcagaaagtaggaaaagcaatgtatgcccctcccattgaaggacaaattagatgttcatcaaatattacagggctactattaacaagagatggtggtgaggacacggacacgaacgacaccgagatcttcagacctggaggaggagatatgagggacaattggagaagtgaattatataaatataaagtagtaacaattgaaccattaggagtagcacccaccaaggcaaagagaagagtggtgcagagagaaaaaagagcagcgataggagctctgttccttgggttcttaggagcagcaggaagcactatgggcgcagcgtcagtgacgctgacggtacaggccagactattattgtctggtatagtgcaacagcagaacaatttgctgagggccattgaggcgcaacagcatatgttgcaactcacagtctggggcatcaagcagctccaggcaagagtcctggctgtggaaagatacctaaaggatcaacagctcctggggttttggggttgctctggaaaactcatttgcaccactactgtgccttggaatgctagttggagtaataaatctctggatgatatttggaataacatgacctggatgcagtgggaaagagaaattgacaattacacaagcttaatatactcattactagaaaaatcgcaaacccaacaagaaaagaatgaacaagaattattggaattggataaatgggcaagtttgtggaattggtttgacataacaaattggctgtggtatataaaa

SEQ ID NO: 17 - protein, 466 aa, Artificial sequence
  synthetic polypeptide
VPVWKEATTTLFCASDAKAYDTEVHNVWATHACVPTDPNPQEVALENVTENFNMWKNNMVEQMHEDIISLWDQSLKPCVKLTPLCVTLNCTDLRNATSRNVTNTTSSSRGMVGGGEMKNCSFNITTGIRGKVQKEYALFYELDIVPIDNKIDRYRLISCNTSVITQACPKVSFEPIPIHYCAPAGFAILKCKDKKFNGKGPCSNVSTVQCTHGIRPVVSTQLLLNGSLAEEEVVIRSENFTNNAKTIIVQLNESVEINCTRPNNNTRKSINIGPGRAFYTTGEIIGDIRQAHCNLSRAKWNDTLNKIVIKLREQFGNKTIVFKHSSGGDPEIVTHSFNCGGEFFYCNSTQLFNSTWNVTEESNNTVENNTITLPCRIKQIINMWQEVGRAMYAPPIRGQIRCSSNITGLLLTRDGGPEDNKTEVFRPGGGDMRDNWRSELYKYKVVKIEPLGVAPTKAKRRVVQRE

SEQ ID NO: 18 - protein, 521 aa, Artificial sequence
  synthetic polypeptide
MGGAAARLGAVILFVVIVGLHGVRGKYALADASLKMADPNRFRGKDLPVLDQLLEVPVWKEATTTLFCASDAKAYDTEVHNVWATHACVPTDPNPQEVALENVTENFNMWKNNMVEQMHEDIISLWDQSLKPCVKLTPLCVTLNCTDLRNATSRNVTNTTSSSRGMVGGGEMKNCSFNITTGIRGKVQKEYALFYELDIVPIDNKIDRYRLISCNTSVITQACPKVSFEPIPIHYCAPAGFAILKCKDKKFNGKGPCSNVSTVQCTHGIRPVVSTQLLLNGSLAEEEVVIRSENFTNNAKTIIVQLNESVEINCTRPNNNTRKSINIGPGRAFYTTGEIIGDIRQAHCNLSRAKWNDTLNKIVIKLREQFGNKTIVFKHSSGGDPEIVTHSFNCGGEFFYCNSTQLFNSTWNVTEESNNTVENNTITLPCRIKQIINMWQEVGRAMYAPPIRGQIRCSSNITGLLLTRDGGPEDNKTEVFRPGGGDMRDNWRSELYKYKVVKIEPLGVAPTKAKRRVVQRE

SEQ ID NO: 19 - DNA, 1563 bp, Artificial sequence
  synthetic nucleic acid
atggggggggctgccgccaggttgggggccgtgattttgtttgtcgtcatagtgggcctccatggggtccgcggcaaatatgccttggcggatgcctctctcaagatggccgaccccaatcgatttcgcggcaaagaccttccggtcctggaccagctgctggaggtacctgtgtggaaagaggccaccaccacactgttctgtgcctccgatgccaaggcctacgataccgaggtgcacaacgtgtgggccactcatgcctgcgtgcccaccgatcctaatcctcaagaagtggccctggaaaacgtgaccgagaacttcaacatgtggaagaacaacatggtcgagcagatgcacgaggacatcatcagcctgtgggaccagagcctgaagccttgcgtgaagctgacccctctgtgcgtgaccctgaactgcaccgacctgagaaacgccaccagccggaacgtgaccaataccacctctagcagcagaggcatggttggaggcggcgagatgaagaactgcagcttcaacatcaccaccggcatcagaggcaaggtgcagaaagagtacgccctgttctacgagctggacatcgtgcccatcgacaacaagatcgaccggtacagactgatcagctgcaacaccagcgtgatcacccaggcctgtcctaaggtgtccttcgagcccattcctatccactactgtgcccctgccggcttcgccatcctgaagtgcaaggacaagaagttcaacggcaagggcccctgcagcaacgtgtccacagtgcagtgtacacacggcatcaggcccgtggtgtctacacagctgctgctgaatggcagcctggccgaggaagaggtggtcatcagaagcgagaatttcaccaacaacgccaagaccatcatcgtgcagctgaacgagagcgtggaaatcaactgcacccggcctaacaacaacacccggaagtccatcaacatcggccctggcagagccttctacacaaccggcgagatcatcggcgacatcagacaggcccactgcaacctgtctcgggccaagtggaacgacaccctgaacaagattgtgatcaagctgagagagcagttcggcaacaagacgatcgtgttcaagcacagctctggcggcgaccctgagatcgtgacccacagctttaattgtggcggcgagttcttctactgcaacagcacccagctgttcaactccacctggaatgtgaccgaggaaagcaacaataccgtcgagaacaacaccatcacactgccctgccggatcaagcagatcatcaatatgtggcaagaagtcggcagggctatgtacgcccctcctatcagaggccagatccggtgcagcagcaatatcacaggcctgctgctcaccagagatggcggccctgaggataacaagaccgaggtgttcagacccggcggaggcgacatgagagacaattggagaagcgagctgtacaagtacaaggtggtcaagatcgagcccctgggcgtcgcccctacaaaggctaagagaagagtggtgcagcgggaa

SEQ ID NO: 20 - protein, 680 aa, Artificial sequence
  synthetic polypeptide
MRVTEIRKSYQHWWRWGIMLLGILMICNAEEKLWVTVYYGVPVWKEATTTLFCASDAKAYDTEVHNVWATHACVPTDPNPQEVALENVTENFNMWKNNMVEQMHEDIISLWDQSLKPCVKLTPLCVTLNCTDLRNATSRNVTNTTSSSRGMVGGGEMKNCSFNITTGIRGKVQKEYALFYELDIVPIDNKIDRYRLISCNTSVITQACPKVSFEPIPIHYCAPAGFAILKCKDKKFNGKGPCSNVSTVQCTHGIRPVVSTQLLLNGSLAEEEVVIRSENFTNNAKTIIVQLNESVEINCTRPNNNTRKSINIGPGRAFYTTGEIIGDIRQAHCNLSRAKWNDTLNKIVIKLREQFGNKTIVFKHSSGGDPEIVTHSFNCGGEFFYCNSTQLFNSTWNVTEESNNTVENNTITLPCRIKQIINMWQEVGRAMYAPPIRGQIRCSSNITGLLLTRDGGPEDNKTEVFRPGGGDMRDNWRSELYKYKVVKIEPLGVAPTKAKRRVVQREKRAVGIGAVFLGFLGAAGSTMGAASMTLTVQARLLLSGIVQQQNNLLRAIEAQQHLLQLTVWGIKQLQARVLAVERYLRDQQLLGIWGCSGKLICTTAVPWNASWSNKSLNKIWDNMTWMEWDREINNYTSIIYSLIEESQNQQEKNEQELLELDKWASLWNWFDITKWLWYIK

SEQ ID NO: 21 - DNA, 2040 bp, Artificial sequence
  synthetic nucleic acid
atgagagtgacggagatcaggaagagttatcagcactggtggagatggggcatcatgctccttgggatattaatgatctgtaatgctgaagaaaaattgtgggtcacagtctattatggggtacctgtgtggaaagaggccaccaccacactgttctgtgcctccgatgccaaggcctacgataccgaggtgcacaacgtgtgggccactcatgcctgcgtgcccaccgatcctaatcctcaagaagtggccctggaaaacgtgaccgagaacttcaacatgtggaagaacaacatggtcgagcagatgcacgaggacatcatcagcctgtgggaccagagcctgaagccttgcgtgaagctgacccctctgtgcgtgaccctgaactgcaccgacctgagaaacgccaccagccggaacgtgaccaataccacctctagcagcagaggcatggttggaggcggcgagatgaagaactgcagcttcaacatcaccaccggcatcagaggcaaggtgcagaaagagtacgccctgttctacgagctggacatcgtgcccatcgacaacaagatcgaccggtacagactgatcagctgcaacaccagcgtgatcacccaggcctgtcctaaggtgtccttcgagcccattcctatccactactgtgcccctgccggcttcgccatcctgaagtgcaaggacaagaagttcaacggcaagggcccctgcagcaacgtgtccacagtgcagtgtacacacggcatcaggcccgtggtgtctacacagctgctgctgaatggcagcctggccgaggaagaggtggtcatcagaagcgagaatttcaccaacaacgccaagaccatcatcgtgcagctgaacgagagcgtggaaatcaactgcacccggcctaacaacaacacccggaagtccatcaacatcggccctggcagagccttctacacaaccggcgagatcatcggcgacatcagacaggcccactgcaacctgtctcgggccaagtggaacgacaccctgaacaagattgtgatcaagctgagagagcagttcggcaacaagacgatcgtgttcaagcacagctctggcggcgaccctgagatcgtgacccacagctttaattgtggcggcgagttcttctactgcaacagcacccagctgttcaactccacctggaatgtgaccgaggaaagcaacaataccgtcgagaacaacaccatcacactgccctgccggatcaagcagatcatcaatatgtggcaagaagtcggcagggctatgtacgcccctcctatcagaggccagatccggtgcagcagcaatatcacaggcctgctgctcaccagagatggcggccctgaggataacaagaccgaggtgttcagacccggcggaggcgacatgagagacaattggagaagcgagctgtacaagtacaaggtggtcaagatcgagcccctgggcgtcgcccctaccaaggcaaagagaagagtggtgcagagagaaaaaagagcagtgggaataggagctgtgttccttgggttcttgggagcagcaggaagcactatgggcgcagcatcaatgacgctgacggtacaggccagactattattgtctggtatagtgcaacagcagaacaatctgctgagagctattgaggcgcaacagcatctgttgcaactcacagtctggggcattaagcagctccaggcaagagtcctggctgtggaaagatacctaagggatcaacagctcctggggatttggggttgctctggaaaactcatctgcaccactgccgtgccttggaatgctagttggagtaataaatctctgaataagatttgggataacatgacctggatggagtgggacagagaaattaacaattacacaagcataatatacagcttaattgaagaatcgcagaaccaacaagaaaagaatgaacaagaattattagaattagataaatgggcaagtttgtggaattggtttgacataacaaaatggctgtggtatataaaa

SEQ ID NO: 22 - protein, 453 aa, Artificial sequence
  synthetic polypeptide
VPVWKEAKTTLFCASEAKGYEKEVHNVWATHACVPTDPSPHELVLENVTENFNMWENDMVDQMHEDIISLWDQSLKPCVKLTPLCVTLNCTNVTGTNVTGNDMKGEMTNCSFNATTEIKDRKKNVYALFYKLDVVQLEGNSSNSTYSTYRLINCNTSVITQACPKVSFDPIPIHYCAPAGYAILKCNNKTFNGTGPCNNVSTVQCTHGIKPVVSTQLLLNGSLAEKEIVIRSKNLTDNVKTIIVHLNESVEITCIRPGNNTRKSIRIGPGQAFYATGDIIGNIRQAHCNISEDKWNKTLQMVGEKLGKLFPNKTIKEPASGGDLEITTHSFNCRGEFFYCNTTKLFNSTYRPNANANSSSSNNTITLQCKIKQIINMWQEVGRAMYAPPIAGNITCTSNITGLLLVRDGGNNSTEEEIFRPGGGNMKDNWRSELYKYKVVEIKPLGVAPTGAK

SEQ ID NO: 23 - protein, 531 aa, Artificial sequence
  synthetic polypeptide
MGGAAARLGAVILFVVIVGLHGVRGKYALADASLKMADPNRFRGKDLPVLDQLLEVPVWKEAKTTLFCASEAKGYEKEVHNVWATHACVPTDPSPHELVLENVTENFNMWENDMVDQMHEDIISLWDQSLKPCVKLTPLCVTLNCTNVTGTNVTGNDMKGEMTNCSFNATTEIKDRKKNVYALFYKLDVVQLEGNSSNSTYSTYRLINCNTSVITQACPKVSFDPIPIHYCAPAGYAILKCNNKTFNGTGPCNNVSTVQCTHGIKPVVSTQLLLNGSLAEKEIVIRSKNLTDNVKTIIVHLNESVEITCIRPGNNTRKSIRIGPGQAFYATGDIIGNIRQAHCNISEDKWNKTLQMVGEKLGKLFPNKTIKEPASGGDLEITTHSFNCRGEFFYCNTTKLFNSTYRPNANANSSSSNNTITLQCKIKQIINMWQEVGRAMYAPPIAGNITCTSNITGLLLVRDGGNNSTEEEIFRPGGGNMKDNWRSELYKYKVVEIKPLGVAPTGAKRRVVEREKRAVGIGAVFLGFLGA

SEQ ID NO: 24 - DNA, 1596 bp, Artificial sequence
  synthetic nucleic acid
atggggggggctgccgccaggttgggggccgtgattttgtttgtcgtcatagtgggcctccatggggtccgcggcaaatatgccttggcggatgcctctctcaagatggccgaccccaatcgatttcgcggcaaagaccttccggtcctggaccagctgctggaggtaccagtgtggaaagaggccaagaccacactgttctgtgccagcgaggccaagggctacgagaaagaggtgcacaacgtctgggccacacacgcctgtgtgcctaccgatccttctcctcacgaactggtgctggaaaacgtgaccgagaacttcaacatgtgggagaacgacatggtggaccagatgcacgaggacatcatcagcctgtgggaccagagcctgaagccttgcgtgaagctgacccctctgtgcgtgaccctgaactgcaccaatgtgaccggcaccaacgtgacagggaacgatatgaagggcgagatgaccaactgcagcttcaacgccaccaccgagatcaaggaccggaagaaaaacgtgtacgccctgttctacaagctggacgtggtgcagctggaaggcaacagcagcaactccacctacagcacctaccggctgatcaactgcaacaccagcgtgatcacccaggcctgtcctaaggtgtccttcgatcccattcctatccactactgtgcccctgccggctacgccatcctgaagtgcaacaacaagaccttcaacggcacaggcccctgcaacaacgtgtccaccgtgcagtgtacccacggcatcaagccagtggtgtccacacagctgctgctgaatggaagcctggccgagaaagaaatcgtgatcagaagcaagaacctgaccgacaacgtcaagaccatcatcgtgcacctgaacgagagcgtggaaatcacctgtatcagacccggcaacaacaccagaaagagcatcagaatcggcccaggccaggccttttatgccaccggcgatatcatcggcaacatcagacaggcccactgtaacatcagcgaggacaagtggaacaagaccctgcagatggtcggagagaagctgggcaagctgttccccaacaagacaatcaagttcgagcccgcctctggcggcgacctggaaattaccacacacagcttcaattgtcggggcgagttcttctactgcaataccaccaagctgtttaatagcacctacaggcccaacgccaatgccaacagctccagctccaacaacactatcaccctgcagtgcaagatcaagcagatcatcaatatgtggcaagaagtcggcagggctatgtacgcccctcctatcgccggcaacattacctgcaccagcaacatcacaggcctgctgctcgttagagatggcggcaacaatagcaccgaggaagagatcttcagacctggcggcggaaacatgaaggacaactggcggagcgagctgtacaagtacaaggtggtcgagattaagcccctgggcgttgcacctactggcgccaagagaagagtggtggaacgcgagaagagagccgttggaatcggcgccgtgttcctgggatttctgggagct

SEQ ID NO: 25 - protein, 457 aa, Artificial sequence
  synthetic polypeptide
VPVWKEATTTLFCASDAKAYDTEVRNVWATHACVPADPNPQEMVLENVTENFNMWKNEMVNQMQEDVISLWDQSLKPCVKLTPLCVTLECRNVSSNSNGAHNETYHESMKEMKNCSFNATTVVRDRKQTVYALFYRLNIVPLTKKNSSENSSEYYRLINCNTSAITQACPKVTFDPIPIHYCTPAGYAILKCNDKIFNGTGPCHNVSTVQCTHGIKPVVSTQLLLNGSLAEGEIIIRSENLTNNVKTIIVHLNQSVEIVCTRPGNNTRKSIRIGPGQTFYATGDIIGDIRQAHCNISEDKWNETLQRVSKKLAEHFQNKTIKFASSSGGDLEITTHSFNCRGEFFYCNTSGLFNGTYTPNGTKSNSSSIITIPCRIKQIINMWQEVGRAMYAPPIEGNITCKSNITGLLLVRDGGTEPNDTETFRPGGGDMRNNWRSELYKYKVVEIKPLGVAPTTA

SEQ ID NO: 26 - protein, 536 aa, Artificial sequence
  synthetic polypeptide
MGGAAARLGAVILFVVIVGLHGVRGKYALADASLKMADPNRFRGKDLPVLDQLLEVPVWKEATTTLFCASDAKAYDTEVRNVWATHACVPADPNPQEMVLENVTENFNMWKNEMVNQMQEDVISLWDQSLKPCVKLTPLCVTLECRNVSSNSNGAHNETYHESMKEMKNCSFNATTVVRDRKQTVYALFYRLNIVPLTKKNSSENSSEYYRLINCNTSAITQACPKVTFDPIPIHYCTPAGYAILKCNDKIFNGTGPCHNVSTVQCTHGIKPVVSTQLLLNGSLAEGEIIIRSENLTNNVKTIIVHLNQSVEIVCTRPGNNTRKSIRIGPGQTFYATGDIIGDIRQAHCNISEDKWNETLQRVSKKLAEHFQNKTIKFASSSGGDLEITTHSFNCRGEFFYCNTSGLFNGTYTPNGTKSNSSSIITIPCRIKQIINMWQEVGRAMYAPPIEGNITCKSNITGLLLVRDGGTEPNDTETFRPGGGDMRNNWRSELYKYKVVEIKPLGVAPTTAKRRMVEREKRAVGIGAVFLGFLGV

SEQ ID NO: 27 - DNA, 1608 bp, Artificial sequence
  synthetic nucleic acid
atggggggggctgccgccaggttgggggccgtgattttgtttgtcgtcatagtgggcctccatggggtccgcggcaaatatgccttggcggatgcctctctcaagatggccgaccccaatcgatttcgcggcaaagaccttccggtcctggaccagctgctggaggtaccagtgtggaaggaagccaccacaaccctcttctgcgccagcgatgccaaggcctacgacacggaggtccgcaatgtgtgggccacccatgcctgtgtgcccgccgaccccaacccccaggagatggtcctggagaatgtcacggagaacttcaacatgtggaagaacgagatggtgaaccagatgcaggaggacgtcatctccctgtgggaccagagcctgaaaccctgcgtcaaactgacacccctctgtgtgaccctggagtgcaggaacgtgtcctccaacagcaacggcgcccacaacgagacctaccacgaaagcatgaaagagatgaagaactgcagcttcaatgccacaaccgtggtgcgggaccggaagcagacggtgtacgcgttgttctaccggctgaatatcgtccccctcacgaagaaaaattccagcgagaactcctccgagtattatcgcctgatcaactgcaacaccagcgccatcacgcaggcctgccccaaagtgaccttcgaccccatccccatccactactgcaccccagctgggtacgccatcctgaagtgcaatgacaaaatcttcaacggcacaggcccctgccacaatgtgagcaccgtccagtgcacccacggcatcaagccagtggtctccacccagctcctcctgaatgggagcctggcagagggcgagatcatcatccgctccgagaacctgaccaacaatgtcaagaccatcatcgtccacctgaatcagtccgtggagatcgtctgcaccagacccggcaacaacacgcggaaaagcatccgcatcggcccagggcagaccttctatgccacgggggacatcattggggacatcaggcaggcccactgcaacatcagcgaagacaagtggaacgaaaccctgcagcgggtgtccaaaaaactcgccgagcacttccagaacaagacgatcaagttcgcatcctccagcgggggggacctggagatcaccacgcacagcttcaactgccggggggaatttttctactgcaacacctccgggctgttcaacgggacctacacccccaacggcaccaagtccaactccagcagcatcatcaccatcccatgcaggatcaagcagatcatcaacatgtggcaggaggtgggccgggccatgtacgccccccccatcgagggcaatatcacctgcaagtccaacatcacggggctgctgctggtgcgggatggggggaccgagcccaacgacaccgagaccttcaggccaggggggggggatatgcggaacaactggcgcagcgagctctacaagtacaaagtggtggagatcaaacccctgggggtggcccccaccacagccaaacgcaggatggtggagcgggagaagcgggcagtgggcattggggccgtgttcttgggcttccttggcgtg

SEQ ID NO: 28 - protein, 504 aa, Artificial sequence
  synthetic polypeptide
MPMGSLQPLATLYLLGMLVASVLAAENLWVTVYYGVPVWKDAETTLFCASDAKAYETEKHNVWATHACVPTDPNPQEIHLENVTEEFNMWKNNMVEQMHTDIISAWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIGDIRQAHCNVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRAKSSVVGSEKSG

SEQ ID NO: 29 - DNA, 1512 bp, Artificial sequence
  synthetic nucleic acid
atgcctatgggcagcctgcagcctctggccacactgtacctgctgggcatgctggtggcctctgtgctggccgccgagaacctgtgggtgacagtgtactacggcgtgcccgtgtggaaggacgccgagacaaccctgttctgcgccagcgacgccaaggcctacgagacagagaagcacaacgtgtgggccacccacgcctgcgtgccaaccgaccctaacccccaggaaatccacctggaaaacgtgaccgaagagttcaacatgtggaagaacaacatggtggaacagatgcacaccgacatcatcagcgcctgggaccagagcctgaagccctgcgtgaagctgacccccctgtgcgtgaccctgcagtgcaccaacgtgaccaacaacatcaccgacgacatgcggggcgagctgaagaactgcagcttcaacatgaccaccgagctgcgggacaagaaacagaaggtgtacagcctgttctaccggctggacgtggtgcagatcaacgagaaccagggcaacagaagcaacaacagcaacaaagagtaccggctgatcaactgcaacaccagcgccatcacccaggcctgccccaaggtgtccttcgagcccatccccatccactactgcgcccctgccggcttcgccatcctgaagtgcaaggacaagaagttcaacggcaccggcccctgccccagcgtgtccacagtgcagtgtacccacggcatcaagcccgtggtgtccacccagctgctgctgaacggcagcctggccgaagaggaagtgatgatcagaagcgagaacatcaccaacaacgccaagaacatcctggtgcagttcaacacccccgtgcagattaactgcacccggcccaacaacaacaccagaaagagcatccggatcggcccaggccaggccttctacgccaccggcgacatcatcggcgacatccggcaggcccactgcaacgtgtccaaggccacctggaacgagacactgggcaaggtggtgaaacagctgcggaagcacttcgggaacaacaccatcatccgcttcgccaacagctctggcggcgacctggaagtgaccacccacagcttcaactgtggcggcgagttcttctactgcaatacctccggcctgttcaacagcacctggatcagcaataccagcgtgcagggcagcaacagcaccggcagcaacgacagcatcaccctgccctgccggatcaagcagatcatcaatatgtggcagcggattggccaggctatgtacgccccacccatccagggcgtgatcagatgcgtgtccaatatcaccggcctgatcctgacccgggacggcggctctaccaacagcaccaccgaaaccttcagacccggcggaggcgacatgagagacaactggcggagcgagctgtacaagtacaaagtggtgaaaatcgagcccctgggcgtggcccccaccagagccaagagcagcgtggtcggaagcgagaagtccggc

SEQ ID NO: 30 - protein, 680 aa, Artificial sequence
  synthetic polypeptide
MRVMGIQRNCQHLFRWGTMILGMIIICSAAENLWVTVYYGVPVWKDAETTLFCASDAKAYETEKHNVWATHACVPTDPNPQEIHLENVTEEFNMWKNNMVEQMHTDIISAWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIGDIRQAHCNVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRAKRRVVGREKRAVGIGAVFLGFLGAAGSTMGAASMTLTVQARNLLSGIVQQQSNLLRAIEAQQHLLKLTVWGIKQLQARVLAVERYLRDQQLLGIWGCSGKLICTTNVPWNSSWSNRNLSEIWDNMTWLQWDKEISNYTQIIYGLLEESQNQQEKNEQDLLALDKWASLWNWFDISNWLWYIK

SEQ ID NO: 31 - DNA, 2040 bp, Artificial sequence
  synthetic nucleic acid
atgagagtgatggggatacagaggaattgtcagcacttattcagatggggaactatgatcttggggatgataataatctgtagtgcagcagaaaacttgtgggtcactgtctactatggggtgcccgtgtggaaggacgccgagacaaccctgttctgcgccagcgacgccaaggcctacgagacagagaagcacaacgtgtgggccacccacgcctgcgtgccaaccgaccctaacccccaggaaatccacctggaaaacgtgaccgaagagttcaacatgtggaagaacaacatggtggaacagatgcacaccgacatcatcagcgcctgggaccagagcctgaagccctgcgtgaagctgacccccctgtgcgtgaccctgcagtgcaccaacgtgaccaacaacatcaccgacgacatgcggggcgagctgaagaactgcagcttcaacatgaccaccgagctgcgggacaagaaacagaaggtgtacagcctgttctaccggctggacgtggtgcagatcaacgagaaccagggcaacagaagcaacaacagcaacaaagagtaccggctgatcaactgcaacaccagcgccatcacccaggcctgccccaaggtgtccttcgagcccatccccatccactactgcgcccctgccggcttcgccatcctgaagtgcaaggacaagaagttcaacggcaccggcccctgccccagcgtgtccacagtgcagtgtacccacggcatcaagcccgtggtgtccacccagctgctgctgaacggcagcctggccgaagaggaagtgatgatcagaagcgagaacatcaccaacaacgccaagaacatcctggtgcagttcaacacccccgtgcagattaactgcacccggcccaacaacaacaccagaaagagcatccggatcggcccaggccaggccttctacgccaccggcgacatcatcggcgacatccggcaggcccactgcaacgtgtccaaggccacctggaacgagacactgggcaaggtggtgaaacagctgcggaagcacttcgggaacaacaccatcatccgcttcgccaacagctctggcggcgacctggaagtgaccacccacagcttcaactgtggcggcgagttcttctactgcaatacctccggcctgttcaacagcacctggatcagcaataccagcgtgcagggcagcaacagcaccggcagcaacgacagcatcaccctgccctgccggatcaagcagatcatcaatatgtggcagcggattggccaggctatgtacgccccacccatccagggcgtgatcagatgcgtgtccaatatcaccggcctgatcctgacccgggacggcggctctaccaacagcaccaccgaaaccttcagacccggcggaggcgacatgagagacaactggcggagcgagctgtacaagtacaaagtggtgaaaatcgagcccctgggcgtggcccccaccagagccaagagaagagtggtggggagagaaaaaagagcagttggaataggagctgtcttccttgggttcttaggagcagcaggaagcactatgggcgcggcgtcaatgacgctgacggtacaggccagaaatttattatctggcatagtgcaacagcaaagcaatttgctgagggctatagaggctcaacaacatctgttgaaactcacggtctggggcattaaacagctccaggcaagggtcctggctgtggaaagatacctaagggatcaacagcttctaggaatttggggctgctctggaaaactcatctgcaccactaatgtgccctggaactctagttggagtaatagaaacctgagtgagatatgggacaacatgacctggctgcaatgggataaagaaattagcaattacacacagataatatatgggctacttgaagaatcgcagaaccagcaggaaaagaatgaacaagacttattggcattggataagtgggcaagtctgtggaattggtttgacatatcaaactggctgtggtatataaaa

SEQ ID NO: 32 - protein, 515 aa, Artificial sequence
  synthetic polypeptide
MRVMGTQKNCQQWWIWGILGFWMLMICNTKDLWVTVYYGVPVWREAKTTLFCASDAKAYETEVHNVWATHACVPTDPNPQEIVLGNVTENFNMWKNDMADQMHEDIISLWDQSLKPCVKLTPLCVTLNCTETNVTGNRTVIGNTNDTNIANATYKYEEMKNCSFNVTTELRNKKHKEYALFYRLDIVPLNENGDNSKYRLINCNTSAITQACPKVSFDPIPIHYCAPAGYAILKCNNKTFNGTGPCYNVSTVQCTHGIKPVVSTQLLLNGSLAEEGMIIRSENLTENTKTIIVHLNESVEINCTRPNNNTRKSVRIGPGQAFYATNDVIGDIRQAHCNISTDRWNKTLQQVMKKLGEHFPNKTIQFKPHAGGDIEITMHSFNCRGEFFYCNTSNLFNSTYHSNNGTYKYNGNSSSPITLQCKIKQIVRMWQGVGQAMYAPPIAGNITCRSNITGILLTRDGGFNTTNNTETFRPGGGDMRDNWRSELYKYKVVEIKPLGIAPTKAKRRVVQREKR

SEQ ID NO: 33 - DNA, 1545 bp, Artificial sequence
  synthetic nucleic acid
atgagagtgatggggacacagaagaattgtcaacaatggtggatatggggcatcttaggcttctggatgctaatgatttgtaatacaaaggacttgtgggtcacagtctattatggggtacctgtgtggagagaagcaaaaactaccctattctgtgcatcagatgctaaagcatatgagacagaagtgcataatgtctgggctacacatgcctgtgtgcccacagaccccaacccacaagaaatagttttgggaaatgtaacagaaaattttaatatgtggaaaaatgacatggcagatcagatgcatgaggatataatcagtttatgggatcaaagcctaaagccatgtgtaaagttgaccccactctgtgtcactttaaactgtacagagacaaatgttacaggtaatagaactgttataggtaatacaaatgataccaatattgcaaatgctacatataagtatgaagaaatgaaaaattgctctttcaatgtaaccacagaactaagaaataagaaacataaggagtatgcactcttttatagacttgacatagtaccacttaatgagaatggtgacaactctaaatatagattgataaattgcaatacctcagccataacacaagcctgtccaaaggtctcttttgacccgattcctatacattactgtgctccagctggttatgcgattctaaagtgtaataataagacattcaatgggacaggaccatgttataatgtcagcacagtacaatgtacacatggaattaagccagtggtatcaactcaactactgttaaatggtagcctagcagaagaagggatgataattagatctgaaaatttgacagaaaataccaaaacaataatagtacatcttaatgaatctgtagagattaattgtacaagacccaacaataatacaagaaaaagtgtaaggataggaccaggacaagccttctatgcaacaaatgatgtaataggagacataagacaagcacattgtaacattagtacagatagatggaacaaaactctacaacaggtaatgaaaaaactaggagagcatttccctaataaaacaatacaatttaaaccacatgcaggaggggatatagaaattacaatgcatagctttaattgtagaggagaatttttctattgcaatacatcaaacctgtttaatagtacataccactctaataatggtacatacaaatataatggtaattcaagctcacccatcacactccaatgcaaaataaaacaaattgtacgcatgtggcaaggggtaggacaagcaatgtatgcccctcccattgcaggaaacataacatgtagatcaaacatcacaggaatactattgacacgcgatggaggatttaacaccacaaacaacacagagacattcagacctggaggaggagatatgagggataactggagaagtgaactatataaatataaagtagtagaaattaagccattgggaatagcacccactaaggcaaaaagaagagtggtgcagagagaaaaaaga

SEQ ID NO: 34 - protein, 687 aa, Artificial sequence
  synthetic polypeptide
MRVMGTQKNCQQWWIWGILGFWMLMICNTKDLWVTVYYGVPVWREAKTTLFCASDAKAYETEVHNVWATHACVPTDPNPQEIVLGNVTENFNMWKNDMADQMHEDIISLWDQSLKPCVKLTPLCVTLNCTETNVTGNRTVIGNTNDTNIANATYKYEEMKNCSFNVTTELRNKKHKEYALFYRLDIVPLNENGDNSKYRLINCNTSAITQACPKVSFDPIPIHYCAPAGYAILKCNNKTFNGTGPCYNVSTVQCTHGIKPVVSTQLLLNGSLAEEGMIIRSENLTENTKTIIVHLNESVEINCTRPNNNTRKSVRIGPGQAFYATNDVIGDIRQAHCNISTDRWNKTLQQVMKKLGEHFPNKTIQFKPHAGGDIEITMHSFNCRGEFFYCNTSNLFNSTYHSNNGTYKYNGNSSSPITLQCKIKQIVRMWQGVGQAMYAPPIAGNITCRSNITGILLTRDGGFNTTNNTETFRPGGGDMRDNWRSELYKYKVVEIKPLGIAPTKAKRRVVQREKRAVGIGAVFLGFLGAAGSTMGAASITLTVQARQLLSGIVQQQSNLLKAIEAQQHMLQLTVWGIKQLQARVLAIERYLKDQQLLGIWGCSGRLICTTAVPWNSSWSNKSEADIWDNMTWMQWDREINNYTEAIFRLLEDSQNQQEKNEKDLLELDKWNSLWNWFNISNWLWYIK

SEQ ID NO: 35 - DNA, 2061 bp, Artificial sequence
  synthetic nucleic acid
atgagagtgatggggacacagaagaattgtcaacaatggtggatatggggcatcttaggcttctggatgctaatgatttgtaatacaaaggacttgtgggtcacagtctattatggggtacctgtgtggagagaagcaaaaactaccctattctgtgcatcagatgctaaagcatatgagacagaagtgcataatgtctgggctacacatgcctgtgtgcccacagaccccaacccacaagaaatagttttgggaaatgtaacagaaaattttaatatgtggaaaaatgacatggcagatcagatgcatgaggatataatcagtttatgggatcaaagcctaaagccatgtgtaaagttgaccccactctgtgtcactttaaactgtacagagacaaatgttacaggtaatagaactgttataggtaatacaaatgataccaatattgcaaatgctacatataagtatgaagaaatgaaaaattgctctttcaatgtaaccacagaactaagaaataagaaacataaggagtatgcactcttttatagacttgacatagtaccacttaatgagaatggtgacaactctaaatatagattgataaattgcaatacctcagccataacacaagcctgtccaaaggtctcttttgacccgattcctatacattactgtgctccagctggttatgcgattctaaagtgtaataataagacattcaatgggacaggaccatgttataatgtcagcacagtacaatgtacacatggaattaagccagtggtatcaactcaactactgttaaatggtagcctagcagaagaagggatgataattagatctgaaaatttgacagaaaataccaaaacaataatagtacatcttaatgaatctgtagagattaattgtacaagacccaacaataatacaagaaaaagtgtaaggataggaccaggacaagccttctatgcaacaaatgatgtaataggagacataagacaagcacattgtaacattagtacagatagatggaacaaaactctacaacaggtaatgaaaaaactaggagagcatttccctaataaaacaatacaatttaaaccacatgcaggaggggatatagaaattacaatgcatagctttaattgtagaggagaatttttctattgcaatacatcaaacctgtttaatagtacataccactctaataatggtacatacaaatataatggtaattcaagctcacccatcacactccaatgcaaaataaaacaaattgtacgcatgtggcaaggggtaggacaagcaatgtatgcccctcccattgcaggaaacataacatgtagatcaaacatcacaggaatactattgacacgcgatggaggatttaacaccacaaacaacacagagacattcagacctggaggaggagatatgagggataactggagaagtgaactatataaatataaagtagtagaaattaagccattgggaatagcacccactaaggcaaaaagaagagtggtgcagagagaaaaaagagcagtgggaataggagctgtgttccttgggttcttgggagcagcaggaagcactatgggcgcagcgtcaataacgctgacggtacaggccagacaactgttgtctggtatagtgcaacagcaaagcaatttgctgaaggctatagaggcgcaacagcatatgttgcaactcacagtctggggcattaagcagctccaggcgagagtcctggctatagaaagatacctaaaggatcaacagctcctagggatttggggctgctctggaagactcatctgcaccactgctgtgccttggaactccagttggagtaataaatctgaagcagatatttgggataacatgacttggatgcagtgggatagagaaattaataattacacagaagcaatattcaggttgcttgaagactcgcaaaaccagcaggaaaagaatgaaaaagatttattagaattggacaagtggaacagtctgtggaattggtttaacatatcaaactggctgtggtatataaaa

SEQ ID NO: 36 - protein, 488 aa, Artificial sequence
  synthetic polypeptide
MRVRGIWKNWPQWLIWSILGFWIGNMEGSWVTVYYGVPVWKEAKTTLFCASDAKAYEKEVHNVWATHACVPTDPNPQEMVLANVTENFNMWKNDMVEQMHEDIISLWDESLKPCVKLTPLCVTLNCTNVKGNESDTSEVMKNCSFKATTELKDKKHKVHALFYKLDVVPLNGNSSSSGEYRLINCNTSAITQACPKVSFDPIPLHYCAPAGFAILKCNNKTFNGTGPCRNVSTVQCTHGIKPVVSTQLLLNGSLAEEEIIIRSENLTNNAKTIIVHLNESVNIVCTRPNNNTRKSIRIGPGQTFYATGDIIGNIRQAHCNINESKWNNTLQKVGEELAKHFPSKTIKFEPSSGGDLEITTHSFNCRGEFFYCNTSDLFNGTYRNGTYNHTGRSSNGTITLQCKIKQIINMWQEVGRAIYAPPIEGEITCNSNITGLLLLRDGGQSNETNDTETFRPGGGDMRDNWRSELYKYKVVEIKPLGVAPTEAK

SEQ ID NO: 37 - DNA, 1464 bp, Artificial sequence
  synthetic nucleic acid
atgagagtgagggggatatggaagaattggccacaatggttgatatggagcatcttaggcttttggataggtaatatggagggctcgtgggtcacagtttactatggagtgcctgtgtggaaagaagcaaaaactactctattctgtgcatcagatgctaaagcatatgagaaagaagtgcataatgtctgggctacacatgcctgtgtgcccacagatcccaacccacaagaaatggttttggcaaatgtaacagaaaattttaacatgtggaaaaatgatatggtagagcagatgcatgaggatataattagtttgtgggatgaaagcctgaagccatgtgtgaagttgaccccactctgtgtcactttaaattgtacaaatgttaaagggaatgagagtgacaccagtgaagtaatgaaaaattgctctttcaaggcaaccacggaactaaaggataaaaaacataaggtgcatgcgcttttttataaacttgatgtagtaccacttaatggaaacagcagcagctctggagagtatagattaataaattgcaatacctcagccataacacaagcctgtccaaaggtctcttttgacccaattcctttacattactgtgcaccagctggttttgcgattctaaagtgtaataataagacattcaatgggacaggaccatgtcgtaatgtcagcacagtacaatgtacacatggaattaagccagtggtatcaactcaactactgttaaatggtagcctagcagaagaagagataataattagatctgaaaatctgacaaacaatgccaaaacaataatagtacacctcaatgaatctgtaaacattgtgtgtacaagacccaataataatacaagaaaaagtataaggataggaccaggacaaacattctatgcaacaggtgacataataggaaacataagacaggcacattgtaacattaatgaaagtaaatggaacaacactttacaaaaggtaggagaagaattagcaaaacacttccctagtaaaacaataaagtttgaaccatcctcaggaggggatctagaaattacaacacatagctttaattgtagaggagagtttttctattgcaatacatcagacctgtttaatggtacatacagaaatggtacatacaatcatacaggaagaagttcaaatggaaccatcaccctccaatgcaaaataaaacaaattataaacatgtggcaggaggtaggaagagcaatatatgcccctcccattgaaggagaaataacatgtaactcaaatatcacaggactactattgctacgtgatggaggtcaatcaaatgaaacaaatgacacagagacattcagacctggaggaggagatatgagggacaattggagaagtgaattatataaatataaagtagtagaaattaaaccattgggagtagcacccactgaggcaaaa

SEQ ID NO: 38 - protein, 669 aa, Artificial sequence
  synthetic polypeptide
MRVRGIWKNWPQWLIWSILGFWIGNMEGSWVTVYYGVPVWKEAKTTLFCASDAKAYEKEVHNVWATHACVPTDPNPQEMVLANVTENFNMWKNDMVEQMHEDIISLWDESLKPCVKLTPLCVTLNCTNVKGNESDTSEVMKNCSFKATTELKDKKHKVHALFYKLDVVPLNGNSSSSGEYRLINCNTSAITQACPKVSFDPIPLHYCAPAGFAILKCNNKTFNGTGPCRNVSTVQCTHGIKPVVSTQLLLNGSLAEEEIIIRSENLTNNAKTIIVHLNESVNIVCTRPNNNTRKSIRIGPGQTFYATGDIIGNIRQAHCNINESKWNNTLQKVGEELAKHFPSKTIKFEPSSGGDLEITTHSFNCRGEFFYCNTSDLFNGTYRNGTYNHTGRSSNGTITLQCKIKQIINMWQEVGRAIYAPPIEGEITCNSNITGLLLLRDGGQSNETNDTETFRPGGGDMRDNWRSELYKYKVVEIKPLGVAPTEAKRRVVEREKRAVGIGAVFLGFLGAAGSTMGAASMTLTVQARQLLSGIVQQQSNLLRAIEAQQHMLQLTVWGIKQLQARVLAIERYLKDQQLLGMWGCSGKLICTTAVPWNSSWSNKSQNEIWGNMTWMQWDREINNYTNTIYRLLEDSQNQQEKNEKDLLALDSWKNLWNWFDISKWLWYIK

SEQ ID NO: 39 - DNA, 2007 bp, Artificial sequence
  synthetic nucleic acid
atgagagtgagggggatatggaagaattggccacaatggttgatatggagcatcttaggcttttggataggtaatatggagggctcgtgggtcacagtttactatggagtgcctgtgtggaaagaagcaaaaactactctattctgtgcatcagatgctaaagcatatgagaaagaagtgcataatgtctgggctacacatgcctgtgtgcccacagatcccaacccacaagaaatggttttggcaaatgtaacagaaaattttaacatgtggaaaaatgatatggtagagcagatgcatgaggatataattagtttgtgggatgaaagcctgaagccatgtgtgaagttgaccccactctgtgtcactttaaattgtacaaatgttaaagggaatgagagtgacaccagtgaagtaatgaaaaattgctctttcaaggcaaccacggaactaaaggataaaaaacataaggtgcatgcgcttttttataaacttgatgtagtaccacttaatggaaacagcagcagctctggagagtatagattaataaattgcaatacctcagccataacacaagcctgtccaaaggtctcttttgacccaattcctttacattactgtgcaccagctggttttgcgattctaaagtgtaataataagacattcaatgggacaggaccatgtcgtaatgtcagcacagtacaatgtacacatggaattaagccagtggtatcaactcaactactgttaaatggtagcctagcagaagaagagataataattagatctgaaaatctgacaaacaatgccaaaacaataatagtacacctcaatgaatctgtaaacattgtgtgtacaagacccaataataatacaagaaaaagtataaggataggaccaggacaaacattctatgcaacaggtgacataataggaaacataagacaggcacattgtaacattaatgaaagtaaatggaacaacactttacaaaaggtaggagaagaattagcaaaacacttccctagtaaaacaataaagtttgaaccatcctcaggaggggatctagaaattacaacacatagctttaattgtagaggagagtttttctattgcaatacatcagacctgtttaatggtacatacagaaatggtacatacaatcatacaggaagaagttcaaatggaaccatcaccctccaatgcaaaataaaacaaattataaacatgtggcaggaggtaggaagagcaatatatgcccctcccattgaaggagaaataacatgtaactcaaatatcacaggactactattgctacgtgatggaggtcaatcaaatgaaacaaatgacacagagacattcagacctggaggaggagatatgagggacaattggagaagtgaattatataaatataaagtagtagaaattaaaccattgggagtagcacccactgaggcaaaaaggagagtggtggagagagaaaaaagagcagtgggaataggagctgtgttccttgggttcttgggagcagccggaagcactatgggcgcagcatcaatgacgctgacggtacaggccaggcaattattgtctggtatagtgcaacagcaaagcaatttgctgagggctatagaggcgcaacagcatatgttgcaactcacggtctggggcattaaacagctccaggcaagagtcctggctatagaaagatacctaaaggatcaacagctcctagggatgtggggctgctctggaaaactcatctgcaccactgctgtgccttggaactccagttggagtaacaaatctcaaaatgaaatttgggggaacatgacctggatgcagtgggacagagaaattaataattacacaaacacaatatataggttacttgaagactcacaaaaccagcaggaaaaaaatgagaaagatttgttagcattggacagttggaaaaatctgtggaattggtttgacatatcaaagtggctgtggtatataaaa

SEQ ID NO: 40 - protein, 492 aa, Artificial sequence
  synthetic polypeptide
MRVRGILRNWPQWWIWSILGFWMLIICRVMGNLWVTVYYGVPVWKEAKATLFCASDARAYEKEVHNVWATHACVPTDPNPQEIYLGNVTENFNMWKNDMVDQMHEDIISLWDQSLKPCVKLTPLCVTLRCTNATINGSLTEEVKNCSFNITTELRDKKQKAYALFYRPDVVPLNKNSPSGNSSEYILINCNTSTITQACPKVSFDPIPIHYCAPAGYAILKCNNKTFNGTGPCNNVSTVQCTHGIKPVVSTQLLLNGSLAEEDIIIKSENLTNNIKTIIVHLNKSVEIVCRRPNNNTRKSIRIGPGQAFYATNDIIGDIRQAHCNINNSTWNRTLEQIKKKLREHFLNRTIEFEPPSGGDLEVTTHSFNCGGEFFYCNTTRLFKWSSNVTNDTITIPCRIKQFINMWQGAGRAMYAPPIEGNITCNSSITGLLLTRDGGKTDRNDTEIFRPGGGNMKDNWRNELYKYKVVEIKPLGVAPTEARRRVVEREKR

SEQ ID NO: 41 - DNA, 1476 bp, Artificial sequence
  synthetic nucleic acid
atgagagtgagggggatactgaggaattggccacaatggtggatatggagcatcttaggcttttggatgctaataatttgtagggtgatggggaacttgtgggtcacagtctattatggggtacctgtgtggaaagaagcaaaagctactctattctgtgcatcagatgctagagcatatgagaaagaagtgcataatgtctgggctacacatgcctgtgtacccacagaccccaacccacaagaaatatacttgggaaatgtaacagaaaattttaacatgtggaaaaatgacatggtggatcagatgcatgaggatataatcagtttatgggatcaaagtctaaagccatgtgtaaagttgaccccactctgtgtcactttaaggtgtacaaatgctactattaatggtagcctgacggaagaagtaaaaaattgctctttcaatataaccacagagctaagagataagaaacagaaagcgtatgcacttttttatagacctgatgtagtaccacttaataagaatagccctagtgggaattctagtgagtatatattaataaattgcaatacctcaaccataacacaagcctgtccaaaggtctcttttgacccaattcctatacattattgtgctccagctggttatgcgattctaaagtgtaataataagacatttaatgggacaggaccatgcaataatgtcagcacagtacaatgtacacatggaattaaaccagtggtatcaactcaactactgttaaatggtagcttagcagaagaagatatcataattaaatctgaaaatctgacaaacaatatcaaaacaataatagtacaccttaataaatctgtagaaattgtgtgtagaagacccaacaataatacaaggaaaagtataaggataggaccaggacaggctttctatgcaacaaatgacataataggagacataagacaagcacattgtaatattaataattctacatggaacagaactttagaacagataaagaaaaaattaagagaacacttccttaatagaacaatagaatttgaaccaccctcagggggggatctagaagttacaacacatagctttaattgtggaggagaatttttctattgcaatacaacacgactgtttaagtggtctagtaatgtcacaaacgacacaatcacaatcccatgcagaataaaacaatttataaacatgtggcaaggggcaggacgagcaatgtatgcccctcccattgaaggaaacataacatgtaactcaagtatcacaggactcctattgacacgtgatggagggaaaacagacaggaatgacacagagatattcagacctggaggaggaaatatgaaggacaattggagaaatgaattatataaatataaagtggtagaaattaagccattgggagtagcacccactgaggcaagaaggagagtggtggagagagaaaaaaga

SEQ ID NO: 42 - protein, 664 aa, Artificial sequence
  synthetic polypeptide
MRVRGILRNWPQWWIWSILGFWMLIICRVMGNLWVTVYYGVPVWKEAKATLFCASDARAYEKEVHNVWATHACVPTDPNPQEIYLGNVTENFNMWKNDMVDQMHEDIISLWDQSLKPCVKLTPLCVTLRCTNATINGSLTEEVKNCSFNITTELRDKKQKAYALFYRPDVVPLNKNSPSGNSSEYILINCNTSTITQACPKVSFDPIPIHYCAPAGYAILKCNNKTFNGTGPCNNVSTVQCTHGIKPVVSTQLLLNGSLAEEDIIIKSENLTNNIKTIIVHLNKSVEIVCRRPNNNTRKSIRIGPGQAFYATNDIIGDIRQAHCNINNSTWNRTLEQIKKKLREHFLNRTIEFEPPSGGDLEVTTHSFNCGGEFFYCNTTRLFKWSSNVTNDTITIPCRIKQFINMWQGAGRAMYAPPIEGNITCNSSITGLLLTRDGGKTDRNDTEIFRPGGGNMKDNWRNELYKYKVVEIKPLGVAPTEARRRVVEREKRAVGIGAVLLGFLGAAGSTMGAASITLTVQARQLLSGIVQQQSNLLRAIEAQQHMLQLTVWGIKQLQTRVLAIERYLKDQQLLGLWGCSGKLICTTNVPWNSSWSNKSQTDIWDNMTWIQWDREISNYSNTIYKLLEGSQNQQEQNEKDLLALDSWNNLWNWFNITNWLWYIK

SEQ ID NO: 43 - DNA, 1992 bp, Artificial sequence
  synthetic nucleic acid
atgagagtgagggggatactgaggaattggccacaatggtggatatggagcatcttaggcttttggatgctaataatttgtagggtgatggggaacttgtgggtcacagtctattatggggtacctgtgtggaaagaagcaaaagctactctattctgtgcatcagatgctagagcatatgagaaagaagtgcataatgtctgggctacacatgcctgtgtacccacagaccccaacccacaagaaatatacttgggaaatgtaacagaaaattttaacatgtggaaaaatgacatggtggatcagatgcatgaggatataatcagtttatgggatcaaagtctaaagccatgtgtaaagttgaccccactctgtgtcactttaaggtgtacaaatgctactattaatggtagcctgacggaagaagtaaaaaattgctctttcaatataaccacagagctaagagataagaaacagaaagcgtatgcacttttttatagacctgatgtagtaccacttaataagaatagccctagtgggaattctagtgagtatatattaataaattgcaatacctcaaccataacacaagcctgtccaaaggtctcttttgacccaattcctatacattattgtgctccagctggttatgcgattctaaagtgtaataataagacatttaatgggacaggaccatgcaataatgtcagcacagtacaatgtacacatggaattaaaccagtggtatcaactcaactactgttaaatggtagcttagcagaagaagatatcataattaaatctgaaaatctgacaaacaatatcaaaacaataatagtacaccttaataaatctgtagaaattgtgtgtagaagacccaacaataatacaaggaaaagtataaggataggaccaggacaggctttctatgcaacaaatgacataataggagacataagacaagcacattgtaatattaataattctacatggaacagaactttagaacagataaagaaaaaattaagagaacacttccttaatagaacaatagaatttgaaccaccctcagggggggatctagaagttacaacacatagctttaattgtggaggagaatttttctattgcaatacaacacgactgtttaagtggtctagtaatgtcacaaacgacacaatcacaatcccatgcagaataaaacaatttataaacatgtggcaaggggcaggacgagcaatgtatgcccctcccattgaaggaaacataacatgtaactcaagtatcacaggactcctattgacacgtgatggagggaaaacagacaggaatgacacagagatattcagacctggaggaggaaatatgaaggacaattggagaaatgaattatataaatataaagtggtagaaattaagccattgggagtagcacccactgaggcaagaaggagagtggtggagagagaaaaaagagcagtgggaataggagctgtactccttgggttcttgggagcagcaggaagcactatgggcgcggcgtcaataacgctgacggtacaggccaggcaactgttgtctggtatagtgcaacagcaaagcaatttgctgagagctatagaggcgcaacagcacatgttgcaactcacggtctggggcattaagcagctccagacaagagtcctggctatagaaaggtacctaaaggatcaacagctcctagggctttggggctgctctggaaaactcatctgcaccactaatgtgccttggaactccagttggagtaataaatctcaaacagatatttgggataacatgacctggatacagtgggatagagaaattagtaattactcaaacacaatatacaagttgcttgaaggctcgcaaaatcagcaggagcaaaatgaaaaagacttattagcattggacagttggaataatctgtggaattggttcaacataacaaattggctgtggtatataaaa

SEQ ID NO: 44 - protein, 25 aa, Artificial sequence
  synthetic polypeptide
MGGAAARLGAVILFVVIVGLHGVRG

SEQ ID NO: 45 - protein, 21 aa, Artificial sequence
  synthetic polypeptide
MAYPAVIVLVCGLFWVPATQG

SEQ ID NO: 46 - protein, 27 aa, Artificial sequence
  synthetic polypeptide
MAPSSPRPALPALLVLLGALFPGPGNA

SEQ ID NO: 47 - protein, 36 aa, Artificial sequence
  synthetic polypeptide
MDAMKRGLCCVLLLCGAVFVSPSQEIHARFRRGARW

SEQ ID NO: 48 - protein, 27 aa, Artificial sequence
  synthetic polypeptide
KYALADASLKMADPNRFRGKDLPVLDQ

SEQ ID NO: 49 - protein, 27 aa, Artificial sequence
  synthetic polypeptide
YVRADPSLSMVNPNRFRGGHLPPLVQQ

SEQ ID NO: 50 - protein, 11 aa, Artificial sequence
  synthetic polypeptide
TDNLWVTVYYG

SEQ ID NO: 51 - protein, 6 aa, Artificial sequence
  synthetic polypeptide
HHHHHH

SEQ ID NO: 52 - protein, 15 aa, Artificial sequence
  synthetic polypeptide
GLNDIFEAQKIEWHE

SEQ ID NO: 53 - protein, 8 aa, Artificial sequence
  synthetic polypeptide
WSHPQFEK

SEQ ID NO: 54 - protein, 16 aa, Artificial sequence
  synthetic polypeptide
HHHHHHSSWSHPQFEK

SEQ ID NO: 55 - protein, 24 aa, Artificial sequence
  synthetic polypeptide
HHHHHHSSWSHPQFEKSSHHHHHH

SEQ ID NO: 56 - protein, 23 aa, Artificial sequence
  synthetic polypeptide
HHHHHHWSHPQFEKHHHHHHQSG

SEQ ID NO: 57 - protein, 8 aa, Artificial sequence
  synthetic polypeptide
KRRVVQRE

SEQ ID NO: 58 - protein, 23 aa, Artificial sequence
  synthetic polypeptide
RRVVEREKRAVGIGAVFLGFLGA

SEQ ID NO: 59 - DNA, 23 bp, Artificial sequence
  synthetic nucleic acid
caggcaagccaaaggcagccttg

SEQ ID NO: 60 - DNA, 23 bp, Artificial sequence
  synthetic nucleic acid
ctcagggactgcaggcctgtctc

SEQ ID NO: 61 - DNA, 20 bp, Artificial sequence
  synthetic nucleic acid
ccctggaacttgcggtggtc

SEQ ID NO: 62 - DNA, 20 bp, Artificial sequence
  synthetic nucleic acid
gggcattccagcccacaaag

SEQ ID NO: 63 - DNA, 20 bp, Artificial sequence
  synthetic nucleic acid
ggcggaacacctcacgggtg

SEQ ID NO: 64 - DNA, 1344 bp, Artificial sequence
  synthetic nucleic acid
atgctgaagaagcagtctgcagggcttgtgctttggggtgctatcctctttgtgggctggaatgccctgctgctcctcttcttctggacacgcccagcccctggcaggcccccctcagatagtgctatcgatgatgaccctgccagcctcacccgtgaggtgttccgcctggctgaggacgctgaggtggagttggagcggcagcgggggctgttgcagcaaatcagggagcatcatgctttgtggagacagaggtggaaagtgcccaccgtggcccctccagcctggccccgtgtgcctgcgaccccctcaccagccgtgatccccatcctggtcattgcctgtgaccgcagcactgtccggcgctgcttggataagttgttgcactatcggccctcagctgagcatttccccatcattgtcagccaggactgcgggcacgaagagacagcacaggtcattgcttcctatggcagtgcagtcacacacatccggcagccagacctgagtaacatcgctgtgcccccagaccaccgcaagttccagggttactacaagatcgccaggcactaccgctgggcactgggccagatcttcaacaagttcaagttcccagcagctgtggtagtggaggacgatctggaggtggcaccagacttctttgagtacttccaggccacctacccactgctgagaacagacccctccctttggtgtgtgtctgcttggaatgacaatggcaaggagcagatggtagactcaagcaaacctgagctgctctatcgaacagacttttttcctggccttggctggctgctgatggctgagctgtggacagagctggagcccaagtggcccaaggccttctgggatgactggatgcgcagacctgagcagcggaaggggcgggcctgtattcgtccagaaatttcaagaacgatgacctttggccgtaagggtgtgagccatgggcagttctttgatcagcatcttaagttcatcaagctgaaccagcagttcgtgtctttcacccagttggatttgtcatacttgcagcgggaggcttatgaccgggatttccttgcccgtgtctatagtgcccccctgctacaggtggagaaagtgaggaccaatgatcagaaggagctgggggaggtgcgggtacagtacactagcagagacagcttcaaggcctttgctaaggccctgggtgtcatggatgacctcaagtctggtgtccccagagctggctaccggggcgttgtcactttccagttcaggggtcgacgtgtccacctggcacccccacaaacctgggaaggctatgatcctagctggaattag

SEQ ID NO: 65 - protein, 447 aa, Artificial sequence
  synthetic polypeptide
MLKKQSAGLVLWGAILFVGWNALLLLFFWTRPAPGRPPSDSAIDDDPASLTREVFRLAEDAEVELERQRGLLQQIREHHALWRQRWKVPTVAPPAWPRVPATPSPAVIPILVIACDRSTVRRCLDKLLHYRPSAEHFPIIVSQDCGHEETAQVIASYGSAVTHIRQPDLSNIAVPPDHRKFQGYYKIARHYRWALGQIFNKFKFPAAVVVEDDLEVAPDFFEYFQATYPLLRTDPSLWCVSAWNDNGKEQMVDSSKPELLYRTDFFPGLGWLLMAELWTELEPKWPKAFWDDWMRRPEQRKGRACIRPEISRTMTFGRKGVSHGQFFDQHLKFIKLNQQFVSFTQLDLSYLQREAYDRDFLARVYSAPLLQVEKVRTNDQKELGEVRVQYTSRDSFKAFAKALGVMDDLKSGVPRAGYRGVVTFQFRGRRVHLAPPQTWEGYDPSWN

SEQ ID NO: 66 - DNA, 1345 bp, Artificial sequence
  synthetic nucleic acid
atgctgaagaagcagtctgcagggcttgtgctttggggtgctatcctcttttgtgggctggaatgccctgctgctcctcttcttctggacacgcccagcccctggcaggcccccctcagatagggctatctctgatgcccctgccagcctcacccgtgaggtgttccgcctggctgaggacgctgaggtggagttggagcggcagcgggggctgttgcagcaaatcagggagcatcatgctttgtggagacagaggtggaaagtgcccaccgtggcccctccagcctggccccgtgtgcctgcgaccccctcaccagccgtgatccccatcctggtcattgcctgtgaccgcagcactgtccggcgctgcttggataagttgttgcactatcggccctcagctgagcatttccccatcattgtcagccgtctctgcgggcacgaggagacagcacaggtcattgcttcctatggcagtgcagtcacacacatccggcagccagacctgagtaacatcgctgtgcccccagaccaccgcaagttccagggttactacaagatcgccaggcactaccgctgggcactgggccagatcttcaacaagttcaagttcccagcagctgtggtagtggaggacgatctggaggtggcaccagacttctttgagtacttccaggccacctacccactgctgagaacagacccctccctttggtgtgtgtctgcttggaatgacaatggcaaggagcagatggtagactcaagcaaacctgagctgctctatcgaacagacttttttcctggccttggctggctgctgatggctgagctgtggacagagctggagcccaagtggcccaaggccttctgggatgactggatgcgcagacctgagcagcggaaggggcgggcctgtattcgtccagaaatttcaagaacgatgacctttggccgtaagggtgtgagccatgggcagttctttgatcagcatcttaagttcatcaagctgaaccagcagttcgtgtctttcacccagttggatttgtcatacttgcagcgggaggcttatgaccgggatttccttgcccgtgtctatagtgcccccctgctacaggtggagaaagtgaggaccaatgatcagaaggagctgggggaggtgcgggtacagtacactagcagagacagcttcaaggcctttgctaaggccctgggtgtcatggatgacctcaagtctggtgtccccagagctggctaccggggcgttgtcactttccagttcaggggtcgacgtgtccacctggcacccccacaaacctgggaaggctatgatcctagctggaattag

SEQ ID NO: 67 - protein, 40 aa, Artificial sequence
  synthetic polypeptide
MLKKQSAGLVLWGAILFCGLECPAAPLLLDTPSPWQAPLR

SEQ ID NO: 68 - DNA, 1640 bp, Artificial sequence
  synthetic nucleic acid
atggggggggctgccgccaggttgggggccgtgattttgtttgtcgtcatagtgggcctccatggggtccgcggcaaatatgccttggcggatgcctctctcaagatggccgaccccaatcgatttcgcggcaaagaccttccggtcctggaccagctgctggaggtaccagtgtggaaggaagccgacacaaccctcttctgcgccagcgatgccaaggcccacgagacggaggtccacaatgtgtgggccacccatgcctgtgtgcccacggaccccaacccccaggagattgacctggagaatgtcacggagaacttcaacatgtggaagaacaacatggtggagcagatgcaggaggacgtcatctccctgtgggaccagagcctgaaaccctgcgtcaaactgacacccccctgtgtgaccctgcactgcacgaacgccaacctgaccaaggccaacctcaccaacgtgaacaatcggaccaacgtgtccaacatcatcgggaacatcacagatgaggtgaggaactgcagcttcaatatgacaaccgagctccgggacaaaaagcagaaggtgcacgcgttgttctacaaactggatatcgtccccatcgaggacaataatgacagctccgagtatcgcctgatcaactgcaacaccagcgtcatcaaacaggcctgccccaaaatttccttcgaccccatccccatccactactgcaccccagctgggtacgcaatcctgaagtgcaatgacaagaacttcaacggcacagggccctgcaagaatgtgagctccgtccagtgcacccacggcatcaagccagtggtctccacccagctcctcctgaatgggagcctggcagaggaagagatcatcatccgctccgagaacctgaccaacaatgccaagaccatcatcgtccacctgaataagtccgtggtcatcaactgcaccagacccagcaacaacacgcggaccagcatcaccatcggcccagggcaggtcttctataggacgggggacatcattggggacatcaggaaggcctactgcaacatcagtgggaccgagtggaacaaagccctgaaacaggtgaccgaaaaactcaaggagcacttcaacaacaagccaatcatcttccagccccccagcgggggggacctggagatcaccatgcaccatttcaactgccggggggaattcttctactgcaacaccacccgcctgttcaacaacacctgcatcgccaacggcaccatcgagggctgcaatggcaacatcaccctcccatgcaaaatcaagcagatcatcaacatgtggcagggggcaggccaggccatgtacgccccccccatctccggcacgatcaactgcgtgtccaacatcacggggatcctgctgacccgggatgggggggctaccaacaatacgaacaatgagaccttcaggccagggggggggaacatcaaagacaactggcgcaatgagctctacaagtacaaagtggtgcagatcgagcccctgggggtggcccccacccgggccaaacgcagggtggtggagcgggagaagcgggcagtgggcattggggccatgttcttgggcttccttggcgcctaacatggggcggccgc

SEQ ID NO: 69 - DNA, 1754 bp, Artificial sequence
  synthetic nucleic acid
atggggggggctgccgccaggttgggggccgtgattttgtttgtcgtcatagtgggcctccatggggtccgcggcaaatatgccttggcggatgcctctctcaagatggccgaccccaatcgatttcgcggcaaagaccttccggtcctggaccagctgctcgaggtacctgtgtggaaagaagcagataccaccctattttgtgcatcagatgccaaagcacatgagacagaagtgcacaatgtctgggccacacatgcctgtgtacccacagaccccaacccacaagaaatagacctggaaaatgtaacagaaaattttaacatgtggaaaaataacatggtagagcagatgcaggaggatgtaatcagtttatgggatcaaagtctaaagccatgtgtaaagttaactcctccctgcgttactttacattgtactaatgctaatttgaccaaagctaatttgaccaatgtcaataacagaaccaatgtctctaacataataggaaatataacagatgaagtaagaaactgttcttttaatatgaccacagaactaagagataagaagcagaaggtccatgcacttttttataagcttgatatagtaccaattgaagataataacgatagtagtgagtataggttaataaattgtaatacttcagtcattaagcaggcttgtccgaagatatcctttgatccaattcctatacattattgtactccagctggttatgcgattttaaagtgtaatgataagaatttcaatgggacagggccatgtaaaaacgtcagctcagtacaatgcacacatggaattaagccagtggtatcaactcaattgctgttaaatggcagtctagcagaagaagagataataatcagatctgaaaatctcacaaacaatgccaaaaccataatagtgcaccttaataaatctgtagtaatcaattgtaccagaccctccaacaatacaagaacaagtataactataggaccaggacaagtattctatagaacaggagacataataggagatataagaaaagcatattgtgagattaatggaacagaatggaataaagctttaaaacaggtaactgaaaagttaaaagagcactttaataataagccaataatctttcaaccaccctcaggaggagatctagaaattacaatgcatcattttaattgtagaggagaatttttctattgcaatacaacacgactgtttaataatacttgcatagcaaatggaaccatagaggggtgtaatggcaatatcacacttccatgcaagataaagcaaattataaacatgtggcagggagcaggacaagcaatgtatgctcctcccatcagtggaacaattaattgtgtatcaaatattacaggaatactattgacaagagatggtggtgctactaataatacgaataacgagaccttcagacctggaggaggaaatataaaggacaattggagaaatgaattatataaatataaagtagtacaaattgaaccactaggagtagcacccaccagggcaaagagaagagtggtggagagagaaaaaagagcagtgggaataggagctatgttccttgggttcttaggagcataaagcttctagagtcgacctgcagaagcttggccgccatggcccaacttgtttattgcagcttataatggttacaaataaagcaatagcatcacaaatttcacaaataaagcatttttttcactgcattc

SEQ ID NO: 70 - protein, 89 aa, Artificial sequence
  synthetic sequence
CVTLHCTNANLTKANLTNVNNRTNVSNIIGNITDEVRNCSFNMTTELRDKKQKVHALFYKLDIVPIEDNNDSSEYRLINCNTSVIKQAC

SEQ ID NO: 71 - protein, 46 aa, Artificial sequence
  synthetic sequence
QINCTRPNNNTRKRIHIGPGRAFYTTKNIKGTIRQAHCNISRAKWN

SEQ ID NO: 72 - protein, 856 aa, Artificial sequence
  synthetic sequence
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL